SEQUENCE LISTING (108 sequences)

SEQ ID NO: 1 - protein, 24 aa, synthetic construct
  SITE 1: note = Acetylated leucine
  SITE 10: note = Nepsilon-Acetyl-lysine, 2-amino-4-acetamido-hexanoic acid
  SITE 12: note = 2-amino-isobutyric acid
  SITE 13: note = 2-amino-isobutyric acid
  SITE 16: note = Lysine 4-[3-[2-[2-(3-aminopropoxy)ethoxy]ethoxy]pro pylamino]-4-oxo-butanoyl - 4-[3-[2-[2-(3-aminopropoxy)etho xy]ethoxy]propylamino]-4-oxo-butanoyl - 4-[3-[2-[2-(3-amin opropoxy)ethoxy]ethoxy]propylamino]-4-oxo-butanoyl ' gamma-Glutamic acid ' gamma-Glutamic acid- gamma-Glutamic acid - palmitoyl
  SITE 21: note = 2-amino-isobutyric acid
  SITE 24: note = Nepsilon-Acetyl-lysine, 2-amino-4-acetamido-hexanoic acid amide
LEGREKVRAKIXXEGKSTWSXRKK

SEQ ID NO: 2 - protein, 24 aa, synthetic construct
  SITE 1: note = Acetylated leucine
  SITE 10: note = Nepsilon-Acetyl-lysine, 2-amino-4-acetamido-hexanoic acid
  SITE 12: note = 2-amino-isobutyric acid
  SITE 13: note = 2-amino-isobutyric acid
  SITE 16: note = Lysine 4-[3-[2-[2-(3-aminopropoxy)ethoxy]ethoxy]pro pylamino]-4-oxo-butanoyl - 4-[3-[2-[2-(3-aminopropoxy)etho xy]ethoxy]propylamino]-4-oxo-butanoyl - 4-[3-[2-[2-(3-amin opropoxy)ethoxy]ethoxy]propylamino]-4-oxo-butanoyl ' gamma-Glutamic acid ' gamma-Glutamic acid- gamma-Glutamic acid - palmitoyl
  SITE 21: note = 2-amino-isobutyric acid
  SITE 24: note = Nepsilon-Acetyl-lysine, 2-amino-4-acetamido-hexanoic acid amide
LEGREKVRAKIXXEGKSTFSXRAK

SEQ ID NO: 3 - protein, 24 aa, synthetic construct
  SITE 1: note = Acetylated leucine
  SITE 10: note = Nepsilon-Acetyl-lysine, 2-amino-4-acetamido-hexanoic acid
  SITE 12: note = 2-amino-isobutyric acid
  SITE 13: note = 2-amino-isobutyric acid
  SITE 16: note = Lysine 4-[3-[2-[2-(3-aminopropoxy)ethoxy]ethoxy]pro pylamino]-4-oxo-butanoyl - 4-[3-[2-[2-(3-aminopropoxy)etho xy]ethoxy]propylamino]-4-oxo-butanoyl - 4-[3-[2-[2-(3-amin opropoxy)ethoxy]ethoxy]propylamino]-4-oxo-butanoyl ' gamma-Glutamic acid ' gamma-Glutamic acid- gamma-Glutamic acid - palmitoyl
  SITE 21: note = Alpha-Methyl-lysine
  SITE 24: note = Nepsilon-Acetyl-lysine, 2-amino-4-acetamido-hexanoic acid amide
LEGREKVRAKIXXEGKSTFSKRAK

SEQ ID NO: 4 - protein, 24 aa, synthetic construct
  SITE 1: note = Acetylated leucine
  SITE 10: note = Nepsilon-Acetyl-lysine, 2-amino-4-acetamido-hexanoic acid
  SITE 12: note = 2-amino-isobutyric acid
  SITE 13: note = 2-amino-isobutyric acid
  SITE 16: note = Lysine 4-[3-[2-[2-(3-aminopropoxy)ethoxy]ethoxy]pro pylamino]-4-oxo-butanoyl - 4-[3-[2-[2-(3-aminopropoxy)etho xy]ethoxy]propylamino]-4-oxo-butanoyl ' gamma-glutamic acid ' gamma-Glutamic acid ' gamma-Glutamic acid ' palmitoyl
  SITE 21: note = 2-amino-isobutyric acid
  SITE 24: note = Nepsilon-Acetyl-lysine, 2-amino-4-acetamido-hexanoic acid amide
LEGREKVRAKIXXEGKSTWSXRKK

SEQ ID NO: 5 - protein, 24 aa, synthetic construct
  SITE 1: note = Acetylated leucine
  SITE 10: note = Nepsilon-Acetyl-lysine, 2-amino-4-acetamido-hexanoic acid
  SITE 12: note = 2-amino-isobutyric acid
  SITE 13: note = 2-amino-isobutyric acid
  SITE 16: note = Lysine 2-[2-(2-aminoethoxy)ethoxy]acetyl - 2-[2-(2-aminoethoxy)ethoxy]acetyl - 2-[2-(2-aminoethoxy)ethoxy]acetyl - 2-[2-(2-aminoethoxy)ethoxy]acetyl ' gamma-Glutamic acid ' gamma-Glutamic acid ' gamma-Glutamic acid - palmitoyl
  SITE 21: note = 2-amino-isobutyric acid
  SITE 24: note = Nepsilon-Acetyl-lysine, 2-amino-4-acetamido-hexanoic acid amide
LEGREKVRAKIXXEGKSTWSXRKK

SEQ ID NO: 6 - protein, 24 aa, synthetic construct
  SITE 1: note = Acetylated leucine
  SITE 10: note = Nepsilon-Acetyl-lysine, 2-amino-4-acetamido-hexanoic acid
  SITE 12: note = 2-amino-isobutyric acid
  SITE 13: note = 2-amino-isobutyric acid
  SITE 16: note = Lysine 4-[3-[2-[2-(3-aminopropoxy)ethoxy]ethoxy]pro pylamino]-4-oxo-butanoyl - 4-[3-[2-[2-(3-aminopropoxy)etho xy]ethoxy]propylamino]-4-oxo-butanoyl ' gamma-Glutamic acid ' gamma-Glutamic acid ' gamma-Glutamic acid - stearoyl
  SITE 21: note = 2-amino-isobutyric acid
  SITE 24: note = Nepsilon-Acetyl-lysine, 2-amino-4-acetamido-hexanoic acid amide
LEGREKVRAKIXXEGKSTWSXRKK

SEQ ID NO: 7 - protein, 24 aa, synthetic construct
  SITE 1: note = Acetylated leucine
  SITE 10: note = Nepsilon-Acetyl-lysine, 2-amino-4-acetamido-hexanoic acid
  SITE 12: note = 2-amino-isobutyric acid
  SITE 13: note = 2-amino-isobutyric acid
  SITE 16: note = Lysine 4-[3-[2-[2-(3-aminopropoxy)ethoxy]ethoxy]pro pylamino]-4-oxo-butanoyl - 4-[3-[2-[2-(3-aminopropoxy)etho xy]ethoxy]propylamino]-4-oxo-butanoyl - 4-[3-[2-[2-(3-amin opropoxy)ethoxy]ethoxy]propylamino]-4-oxo-butanoyl ' gamma-Glutamic acid ' gamma-Glutamic acid ' gamma-Glutamic acid - stearoyl
  SITE 21: note = alpha-methyl lysine
  SITE 24: note = Nepsilon-Acetyl-lysine, 2-amino-4-acetamido-hexanoic acid amide
LEGREKVRAKIXXEGKSTFSKRAK

SEQ ID NO: 8 - protein, 24 aa, synthetic construct
  SITE 1: note = Acetylated leucine
  SITE 10: note = Nepsilon-Acetyl-lysine, 2-amino-4-acetamido-hexanoic acid
  SITE 12: note = 2-amino-isobutyric acid
  SITE 13: note = 2-amino-isobutyric acid
  SITE 16: note = Lysine 2-[2-(2-aminoethoxy)ethoxy]acetyl - 2-[2-(2-aminoethoxy)ethoxy]acetyl - 2-[2-(2-aminoethoxy)ethoxy]acetyl - 2-[2-(2-aminoethoxy)ethoxy]acetyl ' gamma-Glutamic acid ' gamma-Glutamic acid ' gamma-Glutamic acid - palmitoyl)
  SITE 21: note = alpha-methyl lysine
  SITE 24: note = Nepsilon-Acetyl-lysine, 2-amino-4-acetamido-hexanoic acid amide
LEGREKVRAKIXXEGKSTFSKRAK

SEQ ID NO: 9 - protein, 24 aa, synthetic construct
  SITE 1: note = Acetylated leucine
  SITE 10: note = Nepsilon-Acetyl-lysine, 2-amino-4-acetamido-hexanoic acid
  SITE 12: note = 2-amino-isobutyric acid
  SITE 13: note = 2-amino-isobutyric acid
  SITE 16: note = Lysine 4-[3-[2-[2-(3-aminopropoxy)ethoxy]ethoxy]pro pylamino]-4-oxo-butanoyl - 4-[3-[2-[2-(3-aminopropoxy)etho xy]ethoxy]propylamino]-4-oxo-butanoyl ' gamma-Glutamic acid ' gamma-Glutamic acid ' palmitoyl
  SITE 21: note = alpha-methyl lysine
  SITE 24: note = Nepsilon-Acetyl-lysine, 2-amino-4-acetamido-hexanoic acid amide
LEGREKVRAKIXXEGKSTFSKRAK

SEQ ID NO: 10 - protein, 24 aa, synthetic construct
  SITE 1: note = Acetylated leucine
  SITE 10: note = Nepsilon-Acetyl-lysine, 2-amino-4-acetamido-hexanoic acid
  SITE 12: note = 2-amino-isobutyric acid
  SITE 13: note = 2-amino-isobutyric acid
  SITE 16: note = Lysine 4-[3-[2-[2-(3-aminopropoxy)ethoxy]ethoxy]pro pylamino]-4-oxo-butanoyl - 4-[3-[2-[2-(3-aminopropoxy)etho xy]ethoxy]propylamino]-4-oxo-butanoyl ' gamma-Glutamic acid ' gamma-Glutamic acid ' gamma-Glutamic acid ' gamma-Glutamic acid ' palmitoyl
  SITE 21: note = alpha-methyl lysine
  SITE 24: note = Nepsilon-Acetyl-lysine, 2-amino-4-acetamido-hexanoic acid amide
LEGREKVRAKIXXEGKSTFSKRAK

SEQ ID NO: 11 - protein, 24 aa, synthetic construct
  SITE 1: note = Acetylated leucine
  SITE 10: note = Nepsilon-Acetyl-lysine, 2-amino-4-acetamido-hexanoic acid
  SITE 12: note = 2-amino-isobutyric acid
  SITE 13: note = 2-amino-isobutyric acid
  SITE 16: note = Lysine 4-[3-[2-[2-(3-aminopropoxy)ethoxy]ethoxy]pro pylamino]-4-oxo-butanoyl - 4-[3-[2-[2-(3-aminopropoxy)etho xy]ethoxy]propylamino]-4-oxo-butanoyl - 4-[3-[2-[2-(3-amin opropoxy)ethoxy]ethoxy]propylamino]-4-oxo-butanoyl ' gamma-Glutamic acid - gamma-Glutamic acid ' gamma-Glutamic acid ' palmitoyl
  SITE 21: note = alpha-methyl lysine
  SITE 24: note = Nepsilon-Acetyl-lysine, 2-amino-4-acetamido-hexanoic acid amide
LEGREKVRAKIXXEGKSTWSKRAK

SEQ ID NO: 12 - protein, 24 aa, synthetic construct
  SITE 1: note = Acetylated leucine
  SITE 10: note = Citrulline
  SITE 12: note = 2-amino-isobutyric acid
  SITE 13: note = 2-amino-isobutyric acid
  SITE 16: note = Lysine 4-[3-[2-[2-(3-aminopropoxy)ethoxy]ethoxy]pro pylamino]-4-oxo-butanoyl - 4-[3-[2-[2-(3-aminopropoxy)etho xy]ethoxy]propylamino]-4-oxo-butanoyl - 4-[3-[2-[2-(3-amin opropoxy)ethoxy]ethoxy]propylamino]-4-oxo-butanoyl ' gamma-Glutamic acid - gamma-Gllutamic acid ' gamma-Glutamic acid ' palmitoyl
  SITE 21: note = alpha-methyl lysine
  SITE 24: note = Nepsilon-Acetyl-lysine, 2-amino-4-acetamido-hexanoic acid amide
LEGREKVRAXIXXEGKSTFSKRAK

SEQ ID NO: 13 - protein, 24 aa, synthetic construct
  SITE 1: note = Acetylated leucine
  SITE 10: note = Nepsilon-Acetyl-lysine, 2-amino-4-acetamido-hexanoic acid
  SITE 12: note = 2-amino-isobutyric acid
  SITE 13: note = 2-amino-isobutyric acid
  SITE 16: note = Lysine 4-[3-[2-[2-(3-aminopropoxy)ethoxy]ethoxy]pro pylamino]-4-oxo-butanoyl - 4-[3-[2-[2-(3-aminopropoxy)etho xy]ethoxy]propylamino]-4-oxo-butanoyl - 4-[3-[2-[2-(3-amin opropoxy)ethoxy]ethoxy]propylamino]-4-oxo-butanoyl ' gamma-Glutamic acid ' gamma-Glutamic acid ' gamma-Glutamic acid - palmitoyl
  SITE 24: note = Nepsilon-Acetyl-lysine, 2-amino-4-acetamido-hexanoic acid amide
LEGREKVRAKIXXEGKSTFSKRAK

SEQ ID NO: 14 - protein, 24 aa, synthetic construct
  SITE 1: note = Acetylated leucine
  SITE 10: note = Nepsilon-Acetyl-lysine, 2-amino-4-acetamido-hexanoic acid
  SITE 12: note = 2-amino-isobutyric acid
  SITE 13: note = 2-amino-isobutyric acid
  SITE 16: note = Lysine 4-[3-[2-[2-(3-aminopropoxy)ethoxy]ethoxy]pro pylamino]-4-oxo-butanoyl - 4-[3-[2-[2-(3-aminopropoxy)etho xy]ethoxy]propylamino]-4-oxo-butanoyl - 4-[3-[2-[2-(3-amin opropoxy)ethoxy]ethoxy]propylamino]-4-oxo-butanoyl - gamma-Glutamic acid - gamma-Glutamic acid - gamma-Glutamic acid - palmitoyl
  SITE 20: note = D-serine
  SITE 24: note = Nepsilon-Acetyl-lysine, 2-amino-4-acetamido-hexanoic acid amide
LEGREKVRAKIXXEGKSTFSKRAK

SEQ ID NO: 15 - protein, 24 aa, synthetic construct
  SITE 1: note = Acetylated leucine
  SITE 10: note = Nepsilon-Acetyl-lysine, 2-amino-4-acetamido-hexanoic acid
  SITE 12: note = 2-amino-isobutyric acid
  SITE 13: note = 2-amino-isobutyric acid
  SITE 16: note = Lysine 4-[3-[2-[2-(3-aminopropoxy)ethoxy]ethoxy]pro pylamino]-4-oxo-butanoyl - 4-[3-[2-[2-(3-aminopropoxy)etho xy]ethoxy]propylamino]-4-oxo-butanoyl - 4-[3-[2-[2-(3-amin opropoxy)ethoxy]ethoxy]propylamino]-4-oxo-butanoyl - gamma-Glutamic acid - gamma-Glutamic acid - gamma-Glutamic acid - palmitoyl
  SITE 20: note = 2-amino-isobutyric acid
  SITE 24: note = Nepsilon-Acetyl-lysine, 2-amino-4-acetamido-hexanoic acid amide
LEGREKVRAKIXXEGKSTFXKRAK

SEQ ID NO: 16 - protein, 24 aa, synthetic construct
  SITE 1: note = Acetylated leucine
  SITE 10: note = Nepsilon-Acetyl-lysine, 2-amino-4-acetamido-hexanoic acid
  SITE 12: note = 2-amino-isobutyric acid
  SITE 13: note = 2-amino-isobutyric acid
  SITE 16: note = Lysine 4-[3-[2-[2-(3-aminopropoxy)ethoxy]ethoxy]pro pylamino]-4-oxo-butanoyl - 4-[3-[2-[2-(3-aminopropoxy)etho xy]ethoxy]propylamino]-4-oxo-butanoyl - gamma-Glutamic acid - gamma-Glutamic acid - gamma-Glutamic acid - palmitoyl
  SITE 21: note = D-lysine
  SITE 24: note = Nepsilon-Acetyl-lysine, 2-amino-4-acetamido-hexanoic acid amide
LEGREKVRAKIXXEGKSTFSKRAK

SEQ ID NO: 17 - protein, 24 aa, synthetic construct
  SITE 1: note = Acetylated leucine
  SITE 10: note = Nepsilon-Acetyl-lysine, 2-amino-4-acetamido-hexanoic acid
  SITE 12: note = 2-amino-isobutyric acid
  SITE 13: note = 2-amino-isobutyric acid
  SITE 16: note = Lysine 4-[3-[2-[2-(3-aminopropoxy)ethoxy]ethoxy]pro pylamino]-4-oxo-butanoyl - 4-[3-[2-[2-(3-aminopropoxy)etho xy]ethoxy]propylamino]-4-oxo-butanoyl - gamma-Glutamic acid - gamma-Glutamic acid - gamma-Glutamic acid - palmitoyl
  SITE 21: note = D-lysine
  SITE 24: note = Nepsilon-Acetyl-lysine, 2-amino-4-acetamido-hexanoic acid amide
LEGREKVRAKIXXEGKSTWSKRAK

SEQ ID NO: 18 - protein, 24 aa, synthetic construct
  SITE 1: note = Acetylated leucine
  SITE 10: note = Nepsilon-Acetyl-lysine, 2-amino-4-acetamido-hexanoic acid
  SITE 12: note = 2-amino-isobutyric acid
  SITE 13: note = 2-amino-isobutyric acid
  SITE 16: note = Lysine 4-[3-[2-[2-(3-aminopropoxy)ethoxy]ethoxy]pro pylamino]-4-oxo-butanoyl-4-[3-[2-[2-(3-aminopropoxy)ethoxy ]ethoxy]propylamino]-4-oxo-butanoyl-gamma- Glutamic acid-gamma- Glutamic acid-gamma- Glutamic acid-palmitoyl
  SITE 21: note = D-lysine
  SITE 24: note = Nepsilon-Acetyl-lysine, 2-amino-4-acetamido-hexanoic acid amide
LEGREKVRAKIXXEGKSTWSKRKK

SEQ ID NO: 19 - protein, 24 aa, synthetic construct
  SITE 1: note = Acetylated leucine
  SITE 10: note = Nepsilon-Acetyl-lysine, 2-amino-4-acetamido-hexanoic acid
  SITE 12: note = 2-amino-isobutyric acid
  SITE 13: note = 2-amino-isobutyric acid
  SITE 16: note = Lysine 2-[2-(2-aminoethoxy)ethoxy]acetyl-2-[2-(2-am inoethoxy)ethoxy]acetyl-2-[2-(2-aminoethoxy)ethoxy]acetyl- gamma- Glutamic acid-gamma- Glutamic acid-gamma- Glutamic acid-palmitoyl
  SITE 21: note = 2-amino-isobutyric acid
  SITE 24: note = Nepsilon-Acetyl-lysine, 2-amino-4-acetamido-hexanoic acid amide
LEGREKVRAKIXXEGKSTWSXRKK

SEQ ID NO: 20 - protein, 24 aa, synthetic construct
  SITE 1: note = Acetylated leucine
  SITE 10: note = Nepsilon-Acetyl-lysine, 2-amino-4-acetamido-hexanoic acid
  SITE 12: note = 2-amino-isobutyric acid
  SITE 13: note = 2-amino-isobutyric acid
  SITE 16: note = Lysine 4-[3-[2-[2-(3-aminopropoxy)ethoxy]ethoxy]pro pylamino]-4-oxo-butanoyl-4-[3-[2-[2-(3-aminopropoxy)ethoxy ]ethoxy]propylamino]-4-oxo-butanoyl-4-[3-[2-[2-(3-aminopro poxy)ethoxy]ethoxy]propylamino]-4-oxo-butanoyl-gamma- Glutamic acid-gamma- Glutamic acid-gamma- Glutamic acid-stearoyl
  SITE 21: note = 2-amino-isobutyric acid
  SITE 24: note = Nepsilon-Acetyl-lysine, 2-amino-4-acetamido-hexanoic acid amide
LEGREKVRAKIXXEGKSTWSXRKK

SEQ ID NO: 21 - protein, 24 aa, synthetic construct
  SITE 1: note = Acetylated leucine
  SITE 10: note = Nepsilon-Acetyl-lysine, 2-amino-4-acetamido-hexanoic acid
  SITE 12: note = 2-amino-isobutyric acid
  SITE 13: note = 2-amino-isobutyric acid
  SITE 16: note = Lysine 2-[2-(2-aminoethoxy)ethoxy]acetyl-2-[2-(2-am inoethoxy)ethoxy]acetyl-2-[2-(2-aminoethoxy)ethoxy]acetyl- 2-[2-(2-aminoethoxy)ethoxy]acetyl-gamma- Glutamic acid-gamma- Glutamic acid-gamma- Glutamic acid-stearoyl
  SITE 21: note = 2-amino-isobutyric acid
  SITE 24: note = Nepsilon-Acetyl-lysine, 2-amino-4-acetamido-hexanoic acid amide
LEGREKVRAKIXXEGKSTWSXRKK

SEQ ID NO: 22 - protein, 24 aa, synthetic construct
  SITE 1: note = Acetylated leucine
  SITE 10: note = Nepsilon-Acetyl-lysine, 2-amino-4-acetamido-hexanoic acid
  SITE 12: note = 2-amino-isobutyric acid
  SITE 13: note = 2-amino-isobutyric acid
  SITE 16: note = Lysine 2-[2-[3-[2-[2-(3-aminopropoxy)ethoxy]propyla mino]-2-oxo-ethoxy]acetyl - 2-[2-[3-[2-[2-(3-aminopropoxy) ethoxy]propylamino]-2-oxo-ethoxy]acetyl - 2-[2-[3-[2-[2-(3 -aminopropoxy)ethoxy]propylamino]-2-oxo-ethoxy]acetyl - gamma-Glutamic acid - gamma-Glutamic acid - gamma-Glutamic acid - palmitoyl
  SITE 21: note = alpha-methyl lysine
  SITE 24: note = Nepsilon-Acetyl-lysine, 2-amino-4-acetamido-hexanoic acid amide
LEGREKVRAKIXXEGKSTFSKRAK

SEQ ID NO: 23 - protein, 24 aa, synthetic construct
  SITE 1: note = Acetylated leucine
  SITE 10: note = Nepsilon-Acetyl-lysine, 2-amino-4-acetamido-hexanoic acid
  SITE 12: note = 2-amino-isobutyric acid
  SITE 13: note = 2-amino-isobutyric acid
  SITE 16: note = Lysine 4-[3-[2-[2-(3-aminopropoxy)ethoxy]ethoxy]pro pylamino]-4-oxo-butanoyl-4-[3-[2-[2-(3-aminopropoxy)ethoxy ]ethoxy]propylamino]-4-oxo-butanoyl-4-[3-[2-[2-(3-aminopro poxy)ethoxy]ethoxy]propylamino]-4-oxo-butanoyl-gamma- Glutamic acid-gamma- Glutamic acid-gamma- Glutamic acid-palmitoyl
  SITE 21: note = alpha-methyl lysine
  SITE 24: note = Nepsilon-Acetyl-lysine, 2-amino-4-acetamido-hexanoic acid amide
LEGRELVRAKIXXEGKSTFSKRAK

SEQ ID NO: 24 - protein, 24 aa, synthetic construct
  SITE 1: note = Acetylated leucine
  SITE 10: note = Nepsilon-Acetyl-lysine, 2-amino-4-acetamido-hexanoic acid
  SITE 12: note = 2-amino-isobutyric acid
  SITE 13: note = 2-amino-isobutyric acid
  SITE 16: note = Lysine 4-[3-[2-[2-(3-aminopropoxy)ethoxy]ethoxy]pro pylamino]-4-oxo-butanoyl-4-[3-[2-[2-(3-aminopropoxy)ethoxy ]ethoxy]propylamino]-4-oxo-butanoyl-4-[3-[2-[2-(3-aminopro poxy)ethoxy]ethoxy]propylamino]-4-oxo-butanoyl-gamma- Glutamic acid-gamma- Glutamic acid-gamma- Glutamic acid-palmitoyl
  SITE 21: note = alpha-methyl lysine
  SITE 24: note = Nepsilon-Acetyl-lysine, 2-amino-4-acetamido-hexanoic acid amide
LEGREFVRAKIXXEGKSTFSKRAK

SEQ ID NO: 25 - protein, 24 aa, synthetic construct
  SITE 1: note = Acetylated leucine
  SITE 10: note = Nepsilon-Acetyl-lysine, 2-amino-4-acetamido-hexanoic acid
  SITE 12: note = 2-amino-isobutyric acid
  SITE 13: note = 2-amino-isobutyric acid
  SITE 16: note = Lysine 4-[3-[2-[2-(3-aminopropoxy)ethoxy]ethoxy]pro pylamino]-4-oxo-butanoyl-4-[3-[2-[2-(3-aminopropoxy)ethoxy ]ethoxy]propylamino]-4-oxo-butanoyl-4-[3-[2-[2-(3-aminopro poxy)ethoxy]ethoxy]propylamino]-4-oxo-butanoyl-gamma- Glutamic acid-gamma- Glutamic acid-gamma- Glutamic acid-palmitoyl
  SITE 21: note = alpha-methyl lysine
  SITE 24: note = Nepsilon-Acetyl-lysine, 2-amino-4-acetamido-hexanoic acid amide
LEGREQVRAKIXXEGKSTFSKRAK

SEQ ID NO: 26 - protein, 24 aa, synthetic construct
  SITE 1: note = Acetylated leucine
  SITE 10: note = Nepsilon-Acetyl-lysine, 2-amino-4-acetamido-hexanoic acid
  SITE 12: note = 2-amino-isobutyric acid
  SITE 13: note = 2-amino-isobutyric acid
  SITE 16: note = Lysine 2-[2-(2-aminoethoxy)ethoxy]acetyl-2-[2-(2-am inoethoxy)ethoxy]acetyl-2-[2-(2-aminoethoxy)ethoxy]acetyl- 2-[2-(2-aminoethoxy)ethoxy]acetyl-gamma- Glutamic acid-gamma- Glutamic acid-gamma- Glutamic acid-stearoyl
  SITE 21: note = alpha-methyl lysine
  SITE 24: note = Nepsilon-Acetyl-lysine, 2-amino-4-acetamido-hexanoic acid amide
LEGREKVRAKIXXEGKSTFSKRAK

SEQ ID NO: 27 - protein, 24 aa, synthetic construct
  SITE 1: note = Acetylated leucine
  SITE 10: note = Nepsilon-Acetyl-lysine, 2-amino-4-acetamido-hexanoic acid
  SITE 12: note = 2-amino-isobutyric acid
  SITE 13: note = 2-amino-isobutyric acid
  SITE 16: note = Lysine 4-[3-[2-[2-(3-aminopropoxy)ethoxy]ethoxy]pro pylamino]-4-oxo-butanoyl-4-[3-[2-[2-(3-aminopropoxy)ethoxy ]ethoxy]propylamino]-4-oxo-butanoyl-4-[3-[2-[2-(3-aminopro poxy)ethoxy]ethoxy]propylamino]-4-oxo-butanoyl-gamma- Glutamic acid-gamma- Glutamic acid-gamma- Glutamic acid-palmitoyl
  SITE 21: note = alpha-methyl lysine
  SITE 24: note = Nepsilon-Acetyl-lysine, 2-amino-4-acetamido-hexanoic acid amide
LEGRERVRAKIXXEGKSTFSKRAK

SEQ ID NO: 28 - protein, 24 aa, synthetic construct
  SITE 1: note = Acetylated leucine
  SITE 6: note = Homolysine
  SITE 10: note = Nepsilon-Acetyl-lysine, 2-amino-4-acetamido-hexanoic acid
  SITE 12: note = 2-amino-isobutyric acid
  SITE 13: note = 2-amino-isobutyric acid
  SITE 16: note = Lysine 4-[3-[2-[2-(3-aminopropoxy)ethoxy]ethoxy]pro pylamino]-4-oxo-butanoyl-4-[3-[2-[2-(3-aminopropoxy)ethoxy ]ethoxy]propylamino]-4-oxo-butanoyl-4-[3-[2-[2-(3-aminopro poxy)ethoxy]ethoxy]propylamino]-4-oxo-butanoyl-gamma- Glutamic acid-gamma- Glutamic acid-gamma- Glutamic acid-palmitoyl
  SITE 21: note = alpha-methyl lysine
  SITE 24: note = Nepsilon-Acetyl-lysine, 2-amino-4-acetamido-hexanoic acid amide
LEGREXVRAKIXXEGKSTFSKRAK

SEQ ID NO: 29 - protein, 24 aa, synthetic construct
  SITE 1: note = Acetylated leucine
  SITE 9: note = 2-amino-isobutyric acid
  SITE 10: note = Nepsilon-Acetyl-lysine, 2-amino-4-acetamido-hexanoic acid
  SITE 12: note = 2-amino-isobutyric acid
  SITE 13: note = 2-amino-isobutyric acid
  SITE 16: note = Lysine 4-[3-[2-[2-(3-aminopropoxy)ethoxy]ethoxy]pro pylamino]-4-oxo-butanoyl-4-[3-[2-[2-(3-aminopropoxy)ethoxy ]ethoxy]propylamino]-4-oxo-butanoyl-4-[3-[2-[2-(3-aminopro poxy)ethoxy]ethoxy]propylamino]-4-oxo-butanoyl-gamma- Glutamic acid-gamma- Glutamic acid-gamma- Glutamic acid-palmitoyl
  SITE 21: note = alpha-methyl lysine
  SITE 24: note = Nepsilon-Acetyl-lysine, 2-amino-4-acetamido-hexanoic acid amide
LEGREKVRXKIXXEGKSTFSKRAK

SEQ ID NO: 30 - protein, 24 aa, synthetic construct
  SITE 1: note = Acetylated leucine
  SITE 10: note = Nepsilon-Acetyl-lysine, 2-amino-4-acetamido-hexanoic acid
  SITE 12: note = 2-amino-isobutyric acid
  SITE 13: note = 2-amino-isobutyric acid
  SITE 16: note = Lysine 4-[3-[2-[2-(3-aminopropoxy)ethoxy]ethoxy]pro pylamino]-4-oxo-butanoyl-4-[3-[2-[2-(3-aminopropoxy)ethoxy ]ethoxy]propylamino]-4-oxo-butanoyl-4-[3-[2-[2-(3-aminopro poxy)ethoxy]ethoxy]propylamino]-4-oxo-butanoyl-gamma- Glutamic acid-gamma- Glutamic acid-gamma- Glutamic acid-stearoyl
  SITE 21: note = alpha-methyl lysine
  SITE 24: note = Nepsilon-Acetyl-lysine, 2-amino-4-acetamido-hexanoic acid amide
LEGREKVRAKIXXEGKSTWSKRAK

SEQ ID NO: 31 - protein, 24 aa, synthetic construct
  SITE 1: note = Acetylated leucine
  SITE 10: note = Nepsilon-Acetyl-lysine, 2-amino-4-acetamido-hexanoic acid
  SITE 12: note = 2-amino-isobutyric acid
  SITE 13: note = 2-amino-isobutyric acid
  SITE 16: note = Lysine 4-[3-[2-[2-(3-aminopropoxy)ethoxy]ethoxy]pro pylamino]-4-oxo-butanoyl-4-[3-[2-[2-(3-aminopropoxy)ethoxy ]ethoxy]propylamino]-4-oxo-butanoyl-4-[3-[2-[2-(3-aminopro poxy)ethoxy]ethoxy]propylamino]-4-oxo-butanoyl-gamma- Glutamic acid-gamma- Glutamic acid-gamma- Glutamic acid-palmitoyl
  SITE 21: note = 2-amino-isobutyric acid
  SITE 24: note = Nepsilon-Acetyl-lysine, 2-amino-4-acetamido-hexanoic acid amide
LEGRERVRAKIXXEGKSTWSXRRK

SEQ ID NO: 32 - protein, 24 aa, synthetic construct
  SITE 1: note = Acetylated leucine
  SITE 10: note = Nepsilon-Acetyl-lysine, 2-amino-4-acetamido-hexanoic acid
  SITE 12: note = 2-amino-isobutyric acid
  SITE 13: note = 2-amino-isobutyric acid
  SITE 16: note = Lysine 2-[2-[3-[2-[2-(3-aminopropoxy)ethoxy]propyla mino]-2-oxo-ethoxy]acetyl - 2-[2-[3-[2-[2-(3-aminopropoxy) ethoxy]propylamino]-2-oxo-ethoxy]acetyl - 2-[2-[3-[2-[2-(3 -aminopropoxy)ethoxy]propylamino]-2-oxo-ethoxy]acetyl - gamma-Glutamic acid - gamma-Glutamic acid - gamma-Glutamic acid - stearoyl
  SITE 21: note = alpha-methyl lysine
  SITE 24: note = Nepsilon-Acetyl-lysine, 2-amino-4-acetamido-hexanoic acid amide
LEGREKVRAKIXXEGKSTFSKRAK

SEQ ID NO: 33 - protein, 24 aa, synthetic construct
  SITE 1: note = Acetylated leucine
  SITE 10: note = Nepsilon-Acetyl-lysine, 2-amino-4-acetamido-hexanoic acid
  SITE 12: note = 2-amino-isobutyric acid
  SITE 13: note = 2-amino-isobutyric acid
  SITE 16: note = Lysine 4-[3-[2-[2-(3-aminopropoxy)ethoxy]ethoxy]pro pylamino]-4-oxo-butanoyl-4-[3-[2-[2-(3-aminopropoxy)ethoxy ]ethoxy]propylamino]-4-oxo-butanoyl-gamma- Glutamic acid-gamma- Glutamic acid-gamma- Glutamic acid-palmitoyl
  SITE 21: note = alpha-methyl lysine
  SITE 24: note = Nepsilon-Acetyl-lysine, 2-amino-4-acetamido-hexanoic acid amide
LEGREKVRAKIXXEGKSTFSKRAK

SEQ ID NO: 34 - protein, 24 aa, synthetic construct
  SITE 1: note = Acetylated leucine
  SITE 10: note = Nepsilon-Acetyl-lysine, 2-amino-4-acetamido-hexanoic acid
  SITE 12: note = 2-amino-isobutyric acid
  SITE 13: note = 2-amino-isobutyric acid
  SITE 16: note = Lysine 4-[3-[2-[2-(3-aminopropoxy)ethoxy]ethoxy]pro pylamino]-4-oxo-butanoyl-4-[3-[2-[2-(3-aminopropoxy)ethoxy ]ethoxy]propylamino]-4-oxo-butanoyl-gamma- Glutamic acid-gamma- Glutamic acid-gamma- Glutamic acid-stearoyl
  SITE 21: note = alpha-methyl lysine
  SITE 24: note = Nepsilon-Acetyl-lysine, 2-amino-4-acetamido-hexanoic acid amide
LEGREKVRAKIXXEGKSTFSKRAK

SEQ ID NO: 35 - protein, 24 aa, synthetic construct
  SITE 1: note = Acetylated leucine
  SITE 10: note = Nepsilon-Acetyl-lysine, 2-amino-4-acetamido-hexanoic acid
  SITE 12: note = 2-amino-isobutyric acid
  SITE 13: note = 2-amino-isobutyric acid
  SITE 16: note = Lysine 4-[3-[2-[2-(3-aminopropoxy)ethoxy]ethoxy]pro pylamino]-4-oxo-butanoyl-4-[3-[2-[2-(3-aminopropoxy)ethoxy ]ethoxy]propylamino]-4-oxo-butanoyl-4-[3-[2-[2-(3-aminopro poxy)ethoxy]ethoxy]propylamino]-4-oxo-butanoyl-gamma- Glutamic acid-gamma- Glutamic acid-gamma- Glutamic acid-palmitoyl
  SITE 20: note = 2-amino-isobutyric acid
  SITE 24: note = Nepsilon-Acetyl-lysine, 2-amino-4-acetamido-hexanoic acid amide
LEGREKVRAKIXXEGKSTWXKRAK

SEQ ID NO: 36 - protein, 24 aa, synthetic construct
  SITE 1: note = Acetylated leucine
  SITE 10: note = Nepsilon-Acetyl-lysine, 2-amino-4-acetamido-hexanoic acid
  SITE 12: note = 2-amino-isobutyric acid
  SITE 13: note = 2-amino-isobutyric acid
  SITE 16: note = Lysine 4-[3-[2-[2-(3-aminopropoxy)ethoxy]ethoxy]pro pylamino]-4-oxo-butanoyl-4-[3-[2-[2-(3-aminopropoxy)ethoxy ]ethoxy]propylamino]-4-oxo-butanoyl-4-[3-[2-[2-(3-aminopro poxy)ethoxy]ethoxy]propylamino]-4-oxo-butanoyl-gamma- Glutamic acid-gamma- Glutamic acid-gamma- Glutamic acid-palmitoyl
  SITE 21: note = D-lysine
  SITE 24: note = Nepsilon-Acetyl-lysine, 2-amino-4-acetamido-hexanoic acid amide
LEGREKVRAKIXXEGKSTFSKRAK

SEQ ID NO: 37 - protein, 24 aa, synthetic construct
  SITE 1: note = Acetylated leucine
  SITE 10: note = Nepsilon-Acetyl-lysine, 2-amino-4-acetamido-hexanoic acid
  SITE 12: note = 2-amino-isobutyric acid
  SITE 13: note = 2-amino-isobutyric acid
  SITE 16: note = Lysine 4-[3-[2-[2-(3-aminopropoxy)ethoxy]ethoxy]pro pylamino]-4-oxo-butanoyl-4-[3-[2-[2-(3-aminopropoxy)ethoxy ]ethoxy]propylamino]-4-oxo-butanoyl-4-[3-[2-[2-(3-aminopro poxy)ethoxy]ethoxy]propylamino]-4-oxo-butanoyl-gamma- Glutamic acid-gamma- Glutamic acid-gamma- Glutamic acid-palmitoyl
  SITE 21: note = 2-amino-isobutyric acid
  SITE 24: note = Nepsilon-Acetyl-lysine, 2-amino-4-acetamido-hexanoic acid amide
LEGRELVRAKIXXEGKSTWSXRRK

SEQ ID NO: 38 - protein, 24 aa, synthetic construct
  SITE 1: note = Acetylated leucine
  SITE 10: note = Nepsilon-Acetyl-lysine, 2-amino-4-acetamido-hexanoic acid
  SITE 12: note = 2-amino-isobutyric acid
  SITE 13: note = 2-amino-isobutyric acid
  SITE 16: note = Lysine 2-[2-(2-aminoethoxy)ethoxy]acetyl-2-[2-(2-am inoethoxy)ethoxy]acetyl-2-[2-(2-aminoethoxy)ethoxy]acetyl- gamma- Glutamic acid-gamma- Glutamic acid-gamma- Glutamic acid-palmitoyl
  SITE 21: note = alpha-methyl lysine
  SITE 24: note = Nepsilon-Acetyl-lysine, 2-amino-4-acetamido-hexanoic acid amide
LEGREKVRAKIXXEGKSTFSKRAK

SEQ ID NO: 39 - protein, 24 aa, synthetic construct
  SITE 1: note = Acetylated leucine
  SITE 10: note = Nepsilon-Acetyl-lysine, 2-amino-4-acetamido-hexanoic acid
  SITE 12: note = 2-amino-isobutyric acid
  SITE 13: note = 2-amino-isobutyric acid
  SITE 16: note = Lysine 4-[3-[2-[2-(3-aminopropoxy)ethoxy]ethoxy]pro pylamino]-4-oxo-butanoyl-4-[3-[2-[2-(3-aminopropoxy)ethoxy ]ethoxy]propylamino]-4-oxo-butanoyl-4-[3-[2-[2-(3-aminopro poxy)ethoxy]ethoxy]propylamino]-4-oxo-butanoyl-gamma- Glutamic acid-gamma- Glutamic acid-gamma- Glutamic acid-palmitoyl
  SITE 21: note = 2-amino-isobutyric acid
  SITE 24: note = Nepsilon-Acetyl-lysine, 2-amino-4-acetamido-hexanoic acid amide
LEGREFVRAKIXXEGKSTWSXRRK

SEQ ID NO: 40 - protein, 24 aa, synthetic construct
  SITE 1: note = Acetylated leucine
  SITE 10: note = Nepsilon-Acetyl-lysine, 2-amino-4-acetamido-hexanoic acid
  SITE 12: note = 2-amino-isobutyric acid
  SITE 13: note = 2-amino-isobutyric acid
  SITE 16: note = Lysine 4-[3-[2-[2-(3-aminopropoxy)ethoxy]ethoxy]pro pylamino]-4-oxo-butanoyl-4-[3-[2-[2-(3-aminopropoxy)ethoxy ]ethoxy]propylamino]-4-oxo-butanoyl-4-[3-[2-[2-(3-aminopro poxy)ethoxy]ethoxy]propylamino]-4-oxo-butanoyl-gamma- Glutamic acid-gamma- Glutamic acid-gamma- Glutamic acid-palmitoyl
  SITE 21: note = 2-amino-isobutyric acid
  SITE 24: note = Nepsilon-Acetyl-lysine, 2-amino-4-acetamido-hexanoic acid amide
LEGREQVRAKIXXEGKSTWSXRRK

SEQ ID NO: 41 - protein, 24 aa, synthetic construct
  SITE 1: note = Acetylated leucine
  SITE 10: note = Nepsilon-Acetyl-lysine, 2-amino-4-acetamido-hexanoic acid
  SITE 12: note = 2-amino-isobutyric acid
  SITE 13: note = 2-amino-isobutyric acid
  SITE 16: note = Lysine 2-[2-[3-[2-[2-(3-aminopropoxy)ethoxy]propyla mino]-2-oxo-ethoxy]acetyl - 2-[2-[3-[2-[2-(3-aminopropoxy) ethoxy]propylamino]-2-oxo-ethoxy]acetyl - 2-[2-[3-[2-[2-(3 -aminopropoxy)ethoxy]propylamino]-2-oxo-ethoxy]acetyl - gamma-Glutamic acid - gamma-Glutamic acid - gamma-Glutamic acid - palmitoyl
  SITE 21: note = alpha-methyl lysine
  SITE 24: note = Nepsilon-Acetyl-lysine, 2-amino-4-acetamido-hexanoic acid amide
LEGREKVRAKIXXEGKSTWSKRAK

SEQ ID NO: 42 - protein, 24 aa, synthetic construct
  SITE 1: note = Acetylated leucine
  SITE 10: note = Nepsilon-Acetyl-lysine, 2-amino-4-acetamido-hexanoic acid
  SITE 12: note = 2-amino-isobutyric acid
  SITE 13: note = 2-amino-isobutyric acid
  SITE 16: note = Lysine 2-[2-(2-aminoethoxy)ethoxy]acetyl - 2-[2-(2-aminoethoxy)ethoxy]acetyl - 2-[2-(2-aminoethoxy)ethoxy]acetyl - 2-[2-(2-aminoethoxy)ethoxy]acetyl - gamma-Glutamic acid - gamma-Glutamic acid - gamma-Glutamic acid - palmitoyl
  SITE 21: note = alpha-methyl lysine
  SITE 24: note = Nepsilon-Acetyl-lysine, 2-amino-4-acetamido-hexanoic acid amide
LEGREKVRAKIXXEGKSTWSKRAK

SEQ ID NO: 43 - protein, 24 aa, synthetic construct
  SITE 1: note = Acetylated leucine
  SITE 6: note = Homolysine
  SITE 10: note = Nepsilon-Acetyl-lysine, 2-amino-4-acetamido-hexanoic acid
  SITE 12: note = 2-amino-isobutyric acid
  SITE 13: note = 2-amino-isobutyric acid
  SITE 16: note = Lysine 4-[3-[2-[2-(3-aminopropoxy)ethoxy]ethoxy]pro pylamino]-4-oxo-butanoyl-4-[3-[2-[2-(3-aminopropoxy)ethoxy ]ethoxy]propylamino]-4-oxo-butanoyl-4-[3-[2-[2-(3-aminopro poxy)ethoxy]ethoxy]propylamino]-4-oxo-butanoyl-gamma- Glutamic acid-gamma- Glutamic acid-gamma- Glutamic acid-stearoyl
  SITE 21: note = alpha-methyl lysine
  SITE 24: note = Nepsilon-Acetyl-lysine, 2-amino-4-acetamido-hexanoic acid amide
LEGREXVRAKIXXEGKSTWSKRAK

SEQ ID NO: 44 - protein, 24 aa, synthetic construct
  SITE 1: note = Acetylated leucine
  SITE 10: note = Nepsilon-Acetyl-lysine, 2-amino-4-acetamido-hexanoic acid
  SITE 12: note = 2-amino-isobutyric acid
  SITE 13: note = 2-amino-isobutyric acid
  SITE 16: note = Lysine 4-[3-[2-[2-(3-aminopropoxy)ethoxy]ethoxy]pro pylamino]-4-oxo-butanoyl-4-[3-[2-[2-(3-aminopropoxy)ethoxy ]ethoxy]propylamino]-4-oxo-butanoyl-4-[3-[2-[2-(3-aminopro poxy)ethoxy]ethoxy]propylamino]-4-oxo-butanoyl-gamma- Glutamic acid-gamma- Glutamic acid-gamma- Glutamic acid-palmitoyl
  SITE 20: note = D-serine
  SITE 24: note = Nepsilon-Acetyl-lysine, 2-amino-4-acetamido-hexanoic acid amide
LEGREKVRAKIXXEGKSTWSKRAK

SEQ ID NO: 45 - protein, 24 aa, synthetic construct
  SITE 1: note = Acetylated leucine
  SITE 10: note = Nepsilon-Acetyl-lysine, 2-amino-4-acetamido-hexanoic acid
  SITE 12: note = 2-amino-isobutyric acid
  SITE 13: note = 2-amino-isobutyric acid
  SITE 16: note = Lysine 2-[2-(2-aminoethoxy)ethoxy]acetyl-2-[2-(2-am inoethoxy)ethoxy]acetyl-2-[2-(2-aminoethoxy)ethoxy]acetyl- 2-[2-(2-aminoethoxy)ethoxy]acetyl-2-[2-(2-aminoethoxy)etho xy]acetyl-gamma- Glutamic acid-gamma- Glutamic acid-gamma- Glutamic acid-palmitoyl
  SITE 21: note = alpha-methyl lysine
  SITE 24: note = Nepsilon-Acetyl-lysine, 2-amino-4-acetamido-hexanoic acid amide
LEGREKVRAKIXXEGKSTFSKRAK

SEQ ID NO: 46 - protein, 24 aa, synthetic construct
  SITE 1: note = Acetylated leucine
  SITE 10: note = Nepsilon-Acetyl-lysine, 2-amino-4-acetamido-hexanoic acid
  SITE 12: note = 2-amino-isobutyric acid
  SITE 13: note = 2-amino-isobutyric acid
  SITE 16: note = Lysine 2-[2-(2-aminoethoxy)ethoxy]acetyl-2-[2-(2-am inoethoxy)ethoxy]acetyl-2-[2-(2-aminoethoxy)ethoxy]acetyl- 2-[2-(2-aminoethoxy)ethoxy]acetyl-2-[2-(2-aminoethoxy)etho xy]acetyl-gamma- Glutamic acid-gamma- Glutamic acid-gamma- Glutamic acid-gamma- Glutamic acid-palmitoyl
  SITE 21: note = alpha-methyl lysine
  SITE 24: note = Nepsilon-Acetyl-lysine, 2-amino-4-acetamido-hexanoic acid amide
LEGREKVRAKIXXEGKSTFSKRAK

SEQ ID NO: 47 - protein, 24 aa, synthetic construct
  SITE 1: note = Acetylated leucine
  SITE 12: note = 2-amino-isobutyric acid
  SITE 13: note = 2-amino-isobutyric acid
  SITE 16: note = Lysine 4-[3-[2-[2-(3-aminopropoxy)ethoxy]ethoxy]pro pylamino]-4-oxo-butanoyl-4-[3-[2-[2-(3-aminopropoxy)ethoxy ]ethoxy]propylamino]-4-oxo-butanoyl-4-[3-[2-[2-(3-aminopro poxy)ethoxy]ethoxy]propylamino]-4-oxo-butanoyl-gamma- Glutamic acid-gamma- Glutamic acid-gamma- Glutamic acid-palmitoyl
  SITE 21: note = alpha-methyl lysine
  SITE 24: note = Nepsilon-Acetyl-lysine, 2-amino-4-acetamido-hexanoic acid amide
LEGREKVRAKIXXEGKSTFSKRAK

SEQ ID NO: 48 - protein, 24 aa, synthetic construct
  SITE 1: note = Acetylated leucine
  SITE 12: note = 2-amino-isobutyric acid
  SITE 13: note = 2-amino-isobutyric acid
  SITE 16: note = Lysine 4-[3-[2-[2-(3-aminopropoxy)ethoxy]ethoxy]pro pylamino]-4-oxo-butanoyl-4-[3-[2-[2-(3-aminopropoxy)ethoxy ]ethoxy]propylamino]-4-oxo-butanoyl-4-[3-[2-[2-(3-aminopro poxy)ethoxy]ethoxy]propylamino]-4-oxo-butanoyl-gamma- Glutamic acid-gamma- Glutamic acid-gamma- Glutamic acid-palmitoyl
  SITE 21: note = alpha-methyl lysine
  SITE 24: note = Nepsilon-Acetyl-lysine, 2-amino-4-acetamido-hexanoic acid amide
LEGREKVRAQIXXEGKSTFSKRAK

SEQ ID NO: 49 - protein, 24 aa, synthetic construct
  SITE 1: note = Acetylated leucine
  SITE 10: note = Nepsilon-Acetyl-lysine, 2-amino-4-acetamido-hexanoic acid
  SITE 12: note = 2-amino-isobutyric acid
  SITE 13: note = 2-amino-isobutyric acid
  SITE 16: note = Lysine 4-[3-[2-[2-(3-aminopropoxy)ethoxy]ethoxy]pro pylamino]-4-oxo-butanoyl-4-[3-[2-[2-(3-aminopropoxy)ethoxy ]ethoxy]propylamino]-4-oxo-butanoyl-4-[3-[2-[2-(3-aminopro poxy)ethoxy]ethoxy]propylamino]-4-oxo-butanoyl-gamma- Glutamic acid-gamma- Glutamic acid-gamma- Glutamic acid-palmitoyl
  SITE 21: note = alpha-methyl lysine
  SITE 24: note = Lysine amine
LEGREKVRAKIXXEGKSTFSKRAK

SEQ ID NO: 50 - protein, 24 aa, synthetic construct
  SITE 1: note = Acetylated leucine
  SITE 10: note = Nepsilon-Acetyl-lysine, 2-amino-4-acetamido-hexanoic acid
  SITE 12: note = 2-amino-isobutyric acid
  SITE 13: note = 2-amino-isobutyric acid
  SITE 16: note = Lysine 2-[2-[3-[2-[2-(3-aminopropoxy)ethoxy]propyla mino]-2-oxo-ethoxy]acetyl - 2-[2-[3-[2-[2-(3-aminopropoxy) ethoxy]propylamino]-2-oxo-ethoxy]acetyl - 2-[2-[3-[2-[2-(3 -aminopropoxy)ethoxy]propylamino]-2-oxo-ethoxy]acetyl - gamma-Glutamic acid - gamma-Glutamic acid - gamma-Glutamic acid - stearoyl
  SITE 21: note = 2-amino-isobutyric acid
  SITE 24: note = Nepsilon-Acetyl-lysine, 2-amino-4-acetamido-hexanoic acid amide
LEGREKVRAKIXXEGKSTWSXRKK

SEQ ID NO: 51 - protein, 24 aa, synthetic construct
  SITE 1: note = Acetylated leucine
  SITE 10: note = Nepsilon-Acetyl-lysine, 2-amino-4-acetamido-hexanoic acid
  SITE 12: note = 2-amino-isobutyric acid
  SITE 13: note = 2-amino-isobutyric acid
  SITE 16: note = Lysine 4-[3-[2-[2-(3-aminopropoxy)ethoxy]ethoxy]pro pylamino]-4-oxo-butanoyl-4-[3-[2-[2-(3-aminopropoxy)ethoxy ]ethoxy]propylamino]-4-oxo-butanoyl-4-[3-[2-[2-(3-aminopro poxy)ethoxy]ethoxy]propylamino]-4-oxo-butanoyl-gamma- Glutamic acid-gamma- Glutamic acid-gamma- Glutamic acid-gamma- Glutamic acid-stearoyl
  SITE 21: note = 2-amino-isobutyric acid
  SITE 24: note = Nepsilon-Acetyl-lysine, 2-amino-4-acetamido-hexanoic acid amide
LEGREKVRAKIXXEGKSTWSXRKK

SEQ ID NO: 52 - protein, 24 aa, synthetic construct
  SITE 1: note = Acetylated leucine
  SITE 10: note = Nepsilon-Acetyl-lysine, 2-amino-4-acetamido-hexanoic acid
  SITE 12: note = 2-amino-isobutyric acid
  SITE 13: note = 2-amino-isobutyric acid
  SITE 16: note = Lysine 4-[3-[2-[2-(3-aminopropoxy)ethoxy]ethoxy]pro pylamino]-4-oxo-butanoyl-4-[3-[2-[2-(3-aminopropoxy)ethoxy ]ethoxy]propylamino]-4-oxo-butanoyl-4-[3-[2-[2-(3-aminopro poxy)ethoxy]ethoxy]propylamino]-4-oxo-butanoyl-gamma- Glutamic acid-gamma- Glutamic acid-stearoyl
  SITE 21: note = 2-amino-isobutyric acid
  SITE 24: note = Nepsilon-Acetyl-lysine, 2-amino-4-acetamido-hexanoic acid amide
LEGREKVRAKIXXEGKSTWSXRKK

SEQ ID NO: 53 - protein, 24 aa, synthetic construct
  SITE 1: note = Acetylated leucine
  SITE 10: note = Nepsilon-Acetyl-lysine, 2-amino-4-acetamido-hexanoic acid
  SITE 12: note = 2-amino-isobutyric acid
  SITE 13: note = 2-amino-isobutyric acid
  SITE 16: note = Lysine 2-[2-[3-[2-[2-(3-aminopropoxy)ethoxy]propyla mino]-2-oxo-ethoxy]acetyl - 2-[2-[3-[2-[2-(3-aminopropoxy) ethoxy]propylamino]-2-oxo-ethoxy]acetyl - 2-[2-[3-[2-[2-(3 -aminopropoxy)ethoxy]propylamino]-2-oxo-ethoxy]acetyl - gamma-Glutamic acid - gamma-Glutamic acid - gamma-Glutamic acid - gamma-Glutamic acid - stearoyl
  SITE 21: note = 2-amino-isobutyric acid
  SITE 24: note = Nepsilon-Acetyl-lysine, 2-amino-4-acetamido-hexanoic acid amide
LEGREKVRAKIXXEGKSTWSXRKK

SEQ ID NO: 54 - protein, 24 aa, synthetic construct
  SITE 1: note = Acetylated leucine
  SITE 10: note = Nepsilon-Acetyl-lysine, 2-amino-4-acetamido-hexanoic acid
  SITE 12: note = 2-amino-isobutyric acid
  SITE 13: note = 2-amino-isobutyric acid
  SITE 16: note = Lysine 4-[3-[2-[2-(3-aminopropoxy)ethoxy]ethoxy]pro pylamino]-4-oxo-butanoyl - 4-[3-[2-[2-(3-aminopropoxy)etho xy]ethoxy]propylamino]-4-oxo-butanoyl - gamma-Glutamic acid - gamma-Glutamic acid - gamma-Glutamic acid - gamma-Glutamic acid - stearoyl
  SITE 21: note = 2-amino-isobutyric acid
  SITE 24: note = Nepsilon-Acetyl-lysine, 2-amino-4-acetamido-hexanoic acid amide
LEGREKVRAKIXXEGKSTWSXRKK

SEQ ID NO: 55 - protein, 24 aa, synthetic construct
  SITE 1: note = Acetylated leucine
  SITE 10: note = Nepsilon-Acetyl-lysine, 2-amino-4-acetamido-hexanoic acid
  SITE 12: note = 2-amino-isobutyric acid
  SITE 13: note = 2-amino-isobutyric acid
  SITE 16: note = Lysine 4-[3-[2-[2-(3-aminopropoxy)ethoxy]ethoxy]pro pylamino]-4-oxo-butanoyl-4-[3-[2-[2-(3-aminopropoxy)ethoxy ]ethoxy]propylamino]-4-oxo-butanoyl-4-[3-[2-[2-(3-aminopro poxy)ethoxy]ethoxy]propylamino]-4-oxo-butanoyl-gamma- Glutamic acid-gamma- Glutamic acid-gamma- Glutamic acid-palmitoyl
  SITE 21: note = 2-amino-isobutyric acid
  SITE 24: note = Nepsilon-Acetyl-lysine, 2-amino-4-acetamido-hexanoic acid amide
LEGRERVRAKIXXEGKSTWSXRKK

SEQ ID NO: 56 - protein, 24 aa, synthetic construct
  SITE 1: note = Acetylated leucine
  SITE 10: note = Citrulline
  SITE 12: note = 2-amino-isobutyric acid
  SITE 13: note = 2-amino-isobutyric acid
  SITE 16: note = Lysine 4-[3-[2-[2-(3-aminopropoxy)ethoxy]ethoxy]pro pylamino]-4-oxo-butanoyl-4-[3-[2-[2-(3-aminopropoxy)ethoxy ]ethoxy]propylamino]-4-oxo-butanoyl-4-[3-[2-[2-(3-aminopro poxy)ethoxy]ethoxy]propylamino]-4-oxo-butanoyl-gamma- Glutamic acid-gamma- Glutamic acid-gamma- Glutamic acid-stearoyl
  SITE 21: note = alpha-methyl lysine
  SITE 24: note = Nepsilon-Acetyl-lysine, 2-amino-4-acetamido-hexanoic acid amide
LEGREKVRAXIXXEGKSTFSKRAK

SEQ ID NO: 57 - protein, 24 aa, synthetic construct
  SITE 1: note = Acetylated leucine
  SITE 10: note = Nepsilon-Acetyl-lysine, 2-amino-4-acetamido-hexanoic acid
  SITE 12: note = 2-amino-isobutyric acid
  SITE 13: note = 2-amino-isobutyric acid
  SITE 16: note = Lysine 4-[3-[2-[2-(3-aminopropoxy)ethoxy]ethoxy]pro pylamino]-4-oxo-butanoyl-4-[3-[2-[2-(3-aminopropoxy)ethoxy ]ethoxy]propylamino]-4-oxo-butanoyl-4-[3-[2-[2-(3-aminopro poxy)ethoxy]ethoxy]propylamino]-4-oxo-butanoyl-gamma- Glutamic acid-gamma- Glutamic acid-gamma- Glutamic acid-palmitoyl
  SITE 21: note = Homolysine
  SITE 24: note = Nepsilon-Acetyl-lysine, 2-amino-4-acetamido-hexanoic acid amide
LEGREKVRAKIXXEGKSTWSXRAK

SEQ ID NO: 58 - protein, 24 aa, synthetic construct
  SITE 1: note = Acetylated leucine
  SITE 10: note = Nepsilon-Acetyl-lysine, 2-amino-4-acetamido-hexanoic acid
  SITE 12: note = 2-amino-isobutyric acid
  SITE 13: note = 2-amino-isobutyric acid
  SITE 16: note = Lysine 4-[3-[2-[2-(3-aminopropoxy)ethoxy]ethoxy]pro pylamino]-4-oxo-butanoyl - 4-[3-[2-[2-(3-aminopropoxy)etho xy]ethoxy]propylamino]-4-oxo-butanoyl - 4-[3-[2-[2-(3-amin opropoxy)ethoxy]ethoxy]propylamino]-4-oxo-butanoyl - 4-[3-[2-[2-(3-aminopropoxy)ethoxy]ethoxy]propylamino]-4-ox o-butanoyl - gamma-Glutamic acid - gamma-Glutamic acid - gamma-Glutamic acid - stearoyl
  SITE 21: note = 2-amino-isobutyric acid
  SITE 24: note = Nepsilon-Acetyl-lysine, 2-amino-4-acetamido-hexanoic acid amide
LEGREKVRAKIXXEGKSTWSXRKK

SEQ ID NO: 59 - protein, 24 aa, synthetic construct
  SITE 1: note = Acetylated leucine
  SITE 10: note = Nepsilon-Acetyl-lysine, 2-amino-4-acetamido-hexanoic acid
  SITE 12: note = 2-amino-isobutyric acid
  SITE 13: note = 2-amino-isobutyric acid
  SITE 16: note = Lysine 4-[3-[2-[2-(3-aminopropoxy)ethoxy]ethoxy]pro pylamino]-4-oxo-butanoyl - 4-[3-[2-[2-(3-aminopropoxy)etho xy]ethoxy]propylamino]-4-oxo-butanoyl - 4-[3-[2-[2-(3-amin opropoxy)ethoxy]ethoxy]propylamino]-4-oxo-butanoyl - 4-[3-[2-[2-(3-aminopropoxy)ethoxy]ethoxy]propylamino]-4-ox o-butanoyl - gamma-Glutamic acid - gamma-Glutamic acid - gamma-Glutamic acid - stearoyl
  SITE 21: note = 2-amino-isobutyric acid
  SITE 24: note = Nepsilon-Acetyl-lysine, 2-amino-4-acetamido-hexanoic acid amide
LEGREKVRAKIXXEGKSTFSXRKK

SEQ ID NO: 60 - protein, 24 aa, synthetic construct
  SITE 1: note = Acetylated leucine
  SITE 10: note = Nepsilon-Acetyl-lysine, 2-amino-4-acetamido-hexanoic acid
  SITE 12: note = 2-amino-isobutyric acid
  SITE 13: note = 2-amino-isobutyric acid
  SITE 16: note = Lysine 4-[3-[2-[2-(3-aminopropoxy)ethoxy]ethoxy]pro pylamino]-4-oxo-butanoyl-4-[3-[2-[2-(3-aminopropoxy)ethoxy ]ethoxy]propylamino]-4-oxo-butanoyl-gamma- Glutamic acid-gamma- Glutamic acid-gamma- Glutamic acid-stearoyl
  SITE 21: note = 2-amino-isobutyric acid
  SITE 24: note = Nepsilon-Acetyl-lysine, 2-amino-4-acetamido-hexanoic acid amide
LEGREKVRAKIXXEGKSTFSXRKK

SEQ ID NO: 61 - protein, 24 aa, synthetic construct
  SITE 1: note = Acetylated leucine
  SITE 10: note = Nepsilon-Acetyl-lysine, 2-amino-4-acetamido-hexanoic acid
  SITE 12: note = 2-amino-isobutyric acid
  SITE 13: note = 2-amino-isobutyric acid
  SITE 16: note = Lysine 4-[3-[2-[2-(3-aminopropoxy)ethoxy]ethoxy]pro pylamino]-4-oxo-butanoyl-4-[3-[2-[2-(3-aminopropoxy)ethoxy ]ethoxy]propylamino]-4-oxo-butanoyl-4-[3-[2-[2-(3-aminopro poxy)ethoxy]ethoxy]propylamino]-4-oxo-butanoyl-gamma- Glutamic acid-gamma- Glutamic acid-gamma- Glutamic acid-stearoyl
  SITE 21: note = 2-amino-isobutyric acid
  SITE 24: note = Nepsilon-Acetyl-lysine, 2-amino-4-acetamido-hexanoic acid amide
LEGREKVRAKIXXEGKSTFSXRKK

SEQ ID NO: 62 - protein, 24 aa, synthetic construct
  SITE 1: note = Acetylated leucine
  SITE 10: note = Nepsilon-Acetyl-lysine, 2-amino-4-acetamido-hexanoic acid
  SITE 12: note = 2-amino-isobutyric acid
  SITE 13: note = 2-amino-isobutyric acid
  SITE 16: note = Lysine 4-[3-[2-[2-(3-aminopropoxy)ethoxy]ethoxy]pro pylamino]-4-oxo-butanoyl-4-[3-[2-[2-(3-aminopropoxy)ethoxy ]ethoxy]propylamino]-4-oxo-butanoyl-4-[3-[2-[2-(3-aminopro poxy)ethoxy]ethoxy]propylamino]-4-oxo-butanoyl-gamma- Glutamic acid-gamma- Glutamic acid-gamma- Glutamic acid-gamma- Glutamic acid-palmitoyl
  SITE 21: note = 2-amino-isobutyric acid
  SITE 24: note = Nepsilon-Acetyl-lysine, 2-amino-4-acetamido-hexanoic acid amide
LEGREKVRAKIXXEGKSTWSXRKK

SEQ ID NO: 63 - protein, 24 aa, synthetic construct
  SITE 1: note = Acetylated leucine
  SITE 10: note = Nepsilon-Acetyl-lysine, 2-amino-4-acetamido-hexanoic acid
  SITE 12: note = 2-amino-isobutyric acid
  SITE 13: note = 2-amino-isobutyric acid
  SITE 16: note = Lysine 4-[3-[2-[2-(3-aminopropoxy)ethoxy]ethoxy]pro pylamino]-4-oxo-butanoyl-4-[3-[2-[2-(3-aminopropoxy)ethoxy ]ethoxy]propylamino]-4-oxo-butanoyl-4-[3-[2-[2-(3-aminopro poxy)ethoxy]ethoxy]propylamino]-4-oxo-butanoyl-gamma- Glutamic acid-gamma- Glutamic acid-gamma- Glutamic acid-palmitoyl
  SITE 21: note = 2-amino-isobutyric acid
  SITE 24: note = Nepsilon-Acetyl-lysine, 2-amino-4-acetamido-hexanoic acid amide
LEGREKVRAKIXXEGKSQWSXRKK

SEQ ID NO: 64 - protein, 24 aa, synthetic construct
  SITE 1: note = Acetylated leucine
  SITE 10: note = Nepsilon-Acetyl-lysine, 2-amino-4-acetamido-hexanoic acid
  SITE 12: note = 2-amino-isobutyric acid
  SITE 13: note = 2-amino-isobutyric acid
  SITE 16: note = Lysine 4-[3-[2-[2-(3-aminopropoxy)ethoxy]ethoxy]pro pylamino]-4-oxo-butanoyl-4-[3-[2-[2-(3-aminopropoxy)ethoxy ]ethoxy]propylamino]-4-oxo-butanoyl-4-[3-[2-[2-(3-aminopro poxy)ethoxy]ethoxy]propylamino]-4-oxo-butanoyl-gamma- Glutamic acid-gamma- Glutamic acid-gamma- Glutamic acid-palmitoyl
  SITE 21: note = 2-amino-isobutyric acid
  SITE 24: note = Nepsilon-Acetyl-lysine, 2-amino-4-acetamido-hexanoic acid amide
LEGREKVRAKIXXEGKSRWSXRKK

SEQ ID NO: 65 - protein, 24 aa, synthetic construct
  SITE 1: note = Acetylated leucine
  SITE 9: note = Nepsilon-Acetyl-lysine, 2-amino-4-acetamido-hexanoic acid
  SITE 12: note = 2-amino-isobutyric acid
  SITE 13: note = 2-amino-isobutyric acid
  SITE 16: note = Lysine 4-[3-[2-[2-(3-aminopropoxy)ethoxy]ethoxy]pro pylamino]-4-oxo-butanoyl-4-[3-[2-[2-(3-aminopropoxy)ethoxy ]ethoxy]propylamino]-4-oxo-butanoyl-4-[3-[2-[2-(3-aminopro poxy)ethoxy]ethoxy]propylamino]-4-oxo-butanoyl-gamma- Glutamic acid-gamma- Glutamic acid-gamma- Glutamic acid-palmitoyl
  SITE 21: note = 2-amino-isobutyric acid
  SITE 24: note = Nepsilon-Acetyl-lysine, 2-amino-4-acetamido-hexanoic acid amide
LEGREKVRKQIXXEGKSTWSXRKK

SEQ ID NO: 66 - protein, 24 aa, synthetic construct
  SITE 1: note = Acetylated leucine
  SITE 10: note = Nepsilon-Acetyl-lysine, 2-amino-4-acetamido-hexanoic acid
  SITE 12: note = 2-amino-isobutyric acid
  SITE 13: note = 2-amino-isobutyric acid
  SITE 16: note = Lysine 4-[3-[2-[2-(3-aminopropoxy)ethoxy]ethoxy]pro pylamino]-4-oxo-butanoyl-4-[3-[2-[2-(3-aminopropoxy)ethoxy ]ethoxy]propylamino]-4-oxo-butanoyl-4-[3-[2-[2-(3-aminopro poxy)ethoxy]ethoxy]propylamino]-4-oxo-butanoyl-gamma- Glutamic acid-gamma- Glutamic acid-gamma- Glutamic acid-palmitoyl
  SITE 20: note = Nepsilon-Acetyl-lysine, 2-amino-4-acetamido-hexanoic acid
  SITE 21: note = 2-amino-isobutyric acid
  SITE 24: note = Nepsilon-Acetyl-lysine, 2-amino-4-acetamido-hexanoic acid amide
LEGREKVRAKIXXEGKSTWKXRAK

SEQ ID NO: 67 - protein, 24 aa, synthetic construct
  SITE 1: note = Acetylated leucine
  SITE 10: note = Nepsilon-Acetyl-lysine, 2-amino-4-acetamido-hexanoic acid
  SITE 12: note = 2-amino-isobutyric acid
  SITE 13: note = 2-amino-isobutyric acid
  SITE 16: note = Lysine 4-[3-[2-[2-(3-aminopropoxy)ethoxy]ethoxy]pro pylamino]-4-oxo-butanoyl-4-[3-[2-[2-(3-aminopropoxy)ethoxy ]ethoxy]propylamino]-4-oxo-butanoyl-4-[3-[2-[2-(3-aminopro poxy)ethoxy]ethoxy]propylamino]-4-oxo-butanoyl-gamma- Glutamic acid-gamma- Glutamic acid-gamma- Glutamic acid-palmitoyl
  SITE 21: note = 2-amino-isobutyric acid
  SITE 24: note = Nepsilon-Acetyl-lysine, 2-amino-4-acetamido-hexanoic acid amide
LEGRERVRAKIXXEGKSTWSXRKK

SEQ ID NO: 68 - protein, 24 aa, synthetic construct
  SITE 1: note = Acetylated leucine
  SITE 10: note = Nepsilon-Acetyl-lysine, 2-amino-4-acetamido-hexanoic acid
  SITE 12: note = 2-amino-isobutyric acid
  SITE 13: note = 2-amino-isobutyric acid
  SITE 16: note = Lysine 4-[3-[2-[2-(3-aminopropoxy)ethoxy]ethoxy]pro pylamino]-4-oxo-butanoyl-4-[3-[2-[2-(3-aminopropoxy)ethoxy ]ethoxy]propylamino]-4-oxo-butanoyl-4-[3-[2-[2-(3-aminopro poxy)ethoxy]ethoxy]propylamino]-4-oxo-butanoyl-gamma- Glutamic acid-gamma- Glutamic acid-gamma- Glutamic acid-palmitoyl
  SITE 21: note = 2-amino-isobutyric acid
  SITE 23: note = Nepsilon-Acetyl-lysine, 2-amino-4-acetamido-hexanoic acid
  SITE 24: note = Lysine amine
LEGREKVRAKIXXEGKSTWSXRKK

SEQ ID NO: 69 - protein, 24 aa, synthetic construct
  SITE 1: note = Acetylated leucine
  SITE 10: note = Nepsilon-Acetyl-lysine, 2-amino-4-acetamido-hexanoic acid
  SITE 12: note = 2-amino-isobutyric acid
  SITE 13: note = 2-amino-isobutyric acid
  SITE 16: note = Lysine 4-[3-[2-[2-(3-aminopropoxy)ethoxy]ethoxy]pro pylamino]-4-oxo-butanoyl-4-[3-[2-[2-(3-aminopropoxy)ethoxy ]ethoxy]propylamino]-4-oxo-butanoyl-4-[3-[2-[2-(3-aminopro poxy)ethoxy]ethoxy]propylamino]-4-oxo-butanoyl-gamma- Glutamic acid-gamma- Glutamic acid-gamma- Glutamic acid-palmitoyl
  SITE 21: note = 2-amino-isobutyric acid
  SITE 23: note = Nepsilon-Acetyl-lysine, 2-amino-4-acetamido-hexanoic acid
  SITE 24: note = Arginine amine
LEGREKVRAKIXXEGKSTWSXRKR

SEQ ID NO: 70 - protein, 24 aa, synthetic construct
  SITE 1: note = Acetylated leucine
  SITE 10: note = Nepsilon-Acetyl-lysine, 2-amino-4-acetamido-hexanoic acid
  SITE 12: note = 2-amino-isobutyric acid
  SITE 16: note = Lysine 4-[3-[2-[2-(3-aminopropoxy)ethoxy]ethoxy]pro pylamino]-4-oxo-butanoyl-4-[3-[2-[2-(3-aminopropoxy)ethoxy ]ethoxy]propylamino]-4-oxo-butanoyl-4-[3-[2-[2-(3-aminopro poxy)ethoxy]ethoxy]propylamino]-4-oxo-butanoyl-gamma- Glutamic acid-gamma- Glutamic acid-gamma- Glutamic acid-palmitoyl
  SITE 21: note = 2-amino-isobutyric acid
  SITE 24: note = Nepsilon-Acetyl-lysine, 2-amino-4-acetamido-hexanoic acid amide
LEGREKVRAKIXIEGKSTWSXRKK

SEQ ID NO: 71 - protein, 24 aa, synthetic construct
  SITE 1: note = Acetylated leucine
  SITE 10: note = Nepsilon-Acetyl-lysine, 2-amino-4-acetamido-hexanoic acid
  SITE 12: note = 2-amino-isobutyric acid
  SITE 13: note = 2-amino-isobutyric acid
  SITE 16: note = Lysine 4-[3-[2-[2-(3-aminopropoxy)ethoxy]ethoxy]pro pylamino]-4-oxo-butanoyl-4-[3-[2-[2-(3-aminopropoxy)ethoxy ]ethoxy]propylamino]-4-oxo-butanoyl-4-[3-[2-[2-(3-aminopro poxy)ethoxy]ethoxy]propylamino]-4-oxo-butanoyl-gamma- Glutamic acid-gamma- Glutamic acid-gamma- Glutamic acid-palmitoyl
  SITE 21: note = 2-amino-isobutyric acid
  SITE 24: note = Lysine amine
LEGREKVRAKIXXEGKSTWSXRAK

SEQ ID NO: 72 - protein, 24 aa, synthetic construct
  SITE 1: note = Acetylated leucine
  SITE 10: note = Nepsilon-Acetyl-lysine, 2-amino-4-acetamido-hexanoic acid
  SITE 12: note = 2-amino-isobutyric acid
  SITE 13: note = 2-amino-isobutyric acid
  SITE 16: note = Lysine 4-[3-[2-[2-(3-aminopropoxy)ethoxy]ethoxy]pro pylamino]-4-oxo-butanoyl-4-[3-[2-[2-(3-aminopropoxy)ethoxy ]ethoxy]propylamino]-4-oxo-butanoyl-4-[3-[2-[2-(3-aminopro poxy)ethoxy]ethoxy]propylamino]-4-oxo-butanoyl-gamma- Glutamic acid-gamma- Glutamic acid-gamma- Glutamic acid-palmitoyl
  SITE 20: note = 2-amino-isobutyric acid
  SITE 24: note = Nepsilon-Acetyl-lysine, 2-amino-4-acetamido-hexanoic acid amide
LEGREKVRAKIXXEGKSTFXRRAK

SEQ ID NO: 73 - protein, 24 aa, synthetic construct
  SITE 1: note = Acetylated leucine
  SITE 10: note = Nepsilon-Acetyl-lysine, 2-amino-4-acetamido-hexanoic acid
  SITE 12: note = 2-amino-isobutyric acid
  SITE 13: note = 2-amino-isobutyric acid
  SITE 16: note = Lysine 4-[3-[2-[2-(3-aminopropoxy)ethoxy]ethoxy]pro pylamino]-4-oxo-butanoyl-4-[3-[2-[2-(3-aminopropoxy)ethoxy ]ethoxy]propylamino]-4-oxo-butanoyl-4-[3-[2-[2-(3-aminopro poxy)ethoxy]ethoxy]propylamino]-4-oxo-butanoyl-gamma- Glutamic acid-gamma- Glutamic acid-gamma- Glutamic acid-palmitoyl
  SITE 21: note = 2-amino-isobutyric acid
  SITE 24: note = Lysine amine
LEGREKVRAKIXXEGKSKWSXRAK

SEQ ID NO: 74 - protein, 24 aa, synthetic construct
  SITE 1: note = Acetylated leucine
  SITE 10: note = Nepsilon-Acetyl-lysine, 2-amino-4-acetamido-hexanoic acid
  SITE 12: note = 2-amino-isobutyric acid
  SITE 16: note = Lysine 4-[3-[2-[2-(3-aminopropoxy)ethoxy]ethoxy]pro pylamino]-4-oxo-butanoyl-4-[3-[2-[2-(3-aminopropoxy)ethoxy ]ethoxy]propylamino]-4-oxo-butanoyl-4-[3-[2-[2-(3-aminopro poxy)ethoxy]ethoxy]propylamino]-4-oxo-butanoyl-4-[3-[2-[2- (3-aminopropoxy)ethoxy]ethoxy]propylamino]-4-oxo-butanoyl- gamma- Glutamic acid-gamma- Glutamic acid-gamma- Glutamic acid-palmitoyl
  SITE 21: note = 2-amino-isobutyric acid
  SITE 24: note = Nepsilon-Acetyl-lysine, 2-amino-4-acetamido-hexanoic acid amide
LEGREKVRAKIXIEGKSTWSXRKK

SEQ ID NO: 75 - protein, 24 aa, synthetic construct
  SITE 1: note = Acetylated leucine
  SITE 10: note = Nepsilon-Acetyl-lysine, 2-amino-4-acetamido-hexanoic acid
  SITE 12: note = 2-amino-isobutyric acid
  SITE 13: note = 2-amino-isobutyric acid
  SITE 16: note = Lysine 4-[3-[2-[2-(3-aminopropoxy)ethoxy]ethoxy]pro pylamino]-4-oxo-butanoyl-4-[3-[2-[2-(3-aminopropoxy)ethoxy ]ethoxy]propylamino]-4-oxo-butanoyl-4-[3-[2-[2-(3-aminopro poxy)ethoxy]ethoxy]propylamino]-4-oxo-butanoyl-4-[3-[2-[2- (3-aminopropoxy)ethoxy]ethoxy]propylamino]-4-oxo-butanoyl- gamma- Glutamic acid-gamma- Glutamic acid-gamma- Glutamic acid-palmitoyl
  SITE 21: note = 2-amino-isobutyric acid
  SITE 24: note = Nepsilon-Acetyl-lysine, 2-amino-4-acetamido-hexanoic acid amide
LEGRERVRAKIXXEGKSTWSXRRK

SEQ ID NO: 76 - protein, 24 aa, synthetic construct
  SITE 1: note = Acetylated leucine
  SITE 10: note = Nepsilon-Acetyl-lysine, 2-amino-4-acetamido-hexanoic acid
  SITE 12: note = 2-amino-isobutyric acid
  SITE 13: note = 2-amino-isobutyric acid
  SITE 16: note = Lysine 4-[3-[2-[2-(3-aminopropoxy)ethoxy]ethoxy]pro pylamino]-4-oxo-butanoyl-4-[3-[2-[2-(3-aminopropoxy)ethoxy ]ethoxy]propylamino]-4-oxo-butanoyl-4-[3-[2-[2-(3-aminopro poxy)ethoxy]ethoxy]propylamino]-4-oxo-butanoyl-gamma- Glutamic acid-gamma- Glutamic acid-gamma- Glutamic acid-palmitoyl
  SITE 21: note = 2-amino-isobutyric acid
  SITE 24: note = Nepsilon-Acetyl-lysine, 2-amino-4-acetamido-hexanoic acid amide
LEGRERVRAKIXXEGKSTFSXRRK

SEQ ID NO: 77 - protein, 24 aa, synthetic construct
  SITE 1: note = Acetylated leucine
  SITE 10: note = Nepsilon-Acetyl-lysine, 2-amino-4-acetamido-hexanoic acid
  SITE 12: note = 2-amino-isobutyric acid
  SITE 13: note = 2-amino-isobutyric acid
  SITE 16: note = Lysine 4-[3-[2-[2-(3-aminopropoxy)ethoxy]ethoxy]pro pylamino]-4-oxo-butanoyl-4-[3-[2-[2-(3-aminopropoxy)ethoxy ]ethoxy]propylamino]-4-oxo-butanoyl-4-[3-[2-[2-(3-aminopro poxy)ethoxy]ethoxy]propylamino]-4-oxo-butanoyl-4-[3-[2-[2- (3-aminopropoxy)ethoxy]ethoxy]propylamino]-4-oxo-butanoyl- gamma- Glutamic acid-gamma- Glutamic acid-gamma- Glutamic acid-palmitoyl
  SITE 21: note = 2-amino-isobutyric acid
  SITE 24: note = Nepsilon-Acetyl-lysine, 2-amino-4-acetamido-hexanoic acid amide
LEGRERVRAKIXXEGKSTFSXRRK

SEQ ID NO: 78 - protein, 24 aa, synthetic construct
  SITE 1: note = Acetylated leucine
  SITE 10: note = Nepsilon-Acetyl-lysine, 2-amino-4-acetamido-hexanoic acid
  SITE 12: note = 2-amino-isobutyric acid
  SITE 13: note = 2-amino-isobutyric acid
  SITE 16: note = Lysine 4-[3-[2-[2-(3-aminopropoxy)ethoxy]ethoxy]pro pylamino]-4-oxo-butanoyl-4-[3-[2-[2-(3-aminopropoxy)ethoxy ]ethoxy]propylamino]-4-oxo-butanoyl-4-[3-[2-[2-(3-aminopro poxy)ethoxy]ethoxy]propylamino]-4-oxo-butanoyl-4-[3-[2-[2- (3-aminopropoxy)ethoxy]ethoxy]propylamino]-4-oxo-butanoyl- gamma- Glutamic acid-gamma- Glutamic acid-gamma- Glutamic acid-palmitoyl
  SITE 21: note = alpha-methyl lysine
  SITE 24: note = Nepsilon-Acetyl-lysine, 2-amino-4-acetamido-hexanoic acid amide
LEGREKVRAKIXXEGKSTFSKRAK

SEQ ID NO: 79 - protein, 24 aa, synthetic construct
  SITE 1: note = Acetylated leucine
  SITE 10: note = Nepsilon-Acetyl-lysine, 2-amino-4-acetamido-hexanoic acid
  SITE 12: note = 2-amino-isobutyric acid
  SITE 13: note = 2-amino-isobutyric acid
  SITE 16: note = Lysine 4-[3-[2-[2-(3-aminopropoxy)ethoxy]ethoxy]pro pylamino]-4-oxo-butanoyl-4-[3-[2-[2-(3-aminopropoxy)ethoxy ]ethoxy]propylamino]-4-oxo-butanoyl-4-[3-[2-[2-(3-aminopro poxy)ethoxy]ethoxy]propylamino]-4-oxo-butanoyl-gamma- Glutamic acid-gamma- Glutamic acid-gamma- Glutamic acid-palmitoyl
  SITE 21: note = 2-amino-isobutyric acid
  SITE 24: note = Nepsilon-Acetyl-lysine, 2-amino-4-acetamido-hexanoic acid amide
LEGREKVRAKIXXEGKSKFSXRKK

SEQ ID NO: 80 - protein, 24 aa, synthetic construct
  SITE 1: note = Acetylated leucine
  SITE 10: note = Nepsilon-Acetyl-lysine, 2-amino-4-acetamido-hexanoic acid
  SITE 12: note = 2-amino-isobutyric acid
  SITE 13: note = 2-amino-isobutyric acid
  SITE 16: note = Lysine 4-[3-[2-[2-(3-aminopropoxy)ethoxy]ethoxy]pro pylamino]-4-oxo-butanoyl-4-[3-[2-[2-(3-aminopropoxy)ethoxy ]ethoxy]propylamino]-4-oxo-butanoyl-4-[3-[2-[2-(3-aminopro poxy)ethoxy]ethoxy]propylamino]-4-oxo-butanoyl-gamma- Glutamic acid-gamma- Glutamic acid-gamma- Glutamic acid-palmitoyl
  SITE 19: note = 5-Chlorotryptophan
  SITE 21: note = 2-amino-isobutyric acid
  SITE 24: note = Nepsilon-Acetyl-lysine, 2-amino-4-acetamido-hexanoic acid amide
LEGRERVRAKIXXEGKSTWSXRRK

SEQ ID NO: 81 - protein, 24 aa, synthetic construct
  SITE 1: note = Acetylated Nepsilon-Acetyl-lysine, 2-amino-4-acetamido-hexanoic acid
  SITE 10: note = Nepsilon-Acetyl-lysine, 2-amino-4-acetamido-hexanoic acid
  SITE 12: note = 2-amino-isobutyric acid
  SITE 13: note = 2-amino-isobutyric acid
  SITE 16: note = Lysine 4-[3-[2-[2-(3-aminopropoxy)ethoxy]ethoxy]pro pylamino]-4-oxo-butanoyl-4-[3-[2-[2-(3-aminopropoxy)ethoxy ]ethoxy]propylamino]-4-oxo-butanoyl-4-[3-[2-[2-(3-aminopro poxy)ethoxy]ethoxy]propylamino]-4-oxo-butanoyl-gamma- Glutamic acid-gamma- Glutamic acid-gamma- Glutamic acid-palmitoyl
  SITE 21: note = alpha-methyl lysine
  SITE 24: note = Nepsilon-Acetyl-lysine, 2-amino-4-acetamido-hexanoic acid amide
KEGREKVRAKIXXEGKSTFSKRAK

SEQ ID NO: 82 - protein, 24 aa, synthetic construct
  SITE 1: note = Acetylated leucine
  SITE 9: note = 2-amino-isobutyric acid
  SITE 10: note = Nepsilon-Acetyl-lysine, 2-amino-4-acetamido-hexanoic acid
  SITE 12: note = 2-amino-isobutyric acid
  SITE 13: note = 2-amino-isobutyric acid
  SITE 16: note = Lysine 4-[3-[2-[2-(3-aminopropoxy)ethoxy]ethoxy]pro pylamino]-4-oxo-butanoyl-4-[3-[2-[2-(3-aminopropoxy)ethoxy ]ethoxy]propylamino]-4-oxo-butanoyl-4-[3-[2-[2-(3-aminopro poxy)ethoxy]ethoxy]propylamino]-4-oxo-butanoyl-gamma- Glutamic acid-gamma- Glutamic acid-gamma- Glutamic acid-palmitoyl
  SITE 21: note = 2-amino-isobutyric acid
  SITE 24: note = Nepsilon-Acetyl-lysine, 2-amino-4-acetamido-hexanoic acid amide
LEGREKVRXKIXXEGKSTWSXRAK

SEQ ID NO: 83 - protein, 24 aa, synthetic construct
  SITE 1: note = Acetylated leucine
  SITE 10: note = Nepsilon-Acetyl-lysine, 2-amino-4-acetamido-hexanoic acid
  SITE 12: note = 2-amino-isobutyric acid
  SITE 13: note = 2-amino-isobutyric acid
  SITE 16: note = Lysine 4-[3-[2-[2-(3-aminopropoxy)ethoxy]ethoxy]pro pylamino]-4-oxo-butanoyl-4-[3-[2-[2-(3-aminopropoxy)ethoxy ]ethoxy]propylamino]-4-oxo-butanoyl-4-[3-[2-[2-(3-aminopro poxy)ethoxy]ethoxy]propylamino]-4-oxo-butanoyl-gamma- Glutamic acid-gamma- Glutamic acid-gamma- Glutamic acid-palmitoyl
  SITE 21: note = 2-amino-isobutyric acid
  SITE 24: note = Nepsilon-Acetyl-lysine, 2-amino-4-acetamido-hexanoic acid amide
LEGREKVRAKIXXEGKSKWSXRKK

SEQ ID NO: 84 - protein, 24 aa, synthetic construct
  SITE 1: note = Acetylated leucine
  SITE 10: note = Nepsilon-Acetyl-lysine, 2-amino-4-acetamido-hexanoic acid
  SITE 12: note = 2-amino-isobutyric acid
  SITE 16: note = Lysine 4-[3-[2-[2-(3-aminopropoxy)ethoxy]ethoxy]pro pylamino]-4-oxo-butanoyl-4-[3-[2-[2-(3-aminopropoxy)ethoxy ]ethoxy]propylamino]-4-oxo-butanoyl-4-[3-[2-[2-(3-aminopro poxy)ethoxy]ethoxy]propylamino]-4-oxo-butanoyl-gamma- Glutamic acid-gamma- Glutamic acid-gamma- Glutamic acid-palmitoyl
  SITE 21: note = alpha-methyl lysine
  SITE 24: note = Nepsilon-Acetyl-lysine, 2-amino-4-acetamido-hexanoic acid amide
LEGREKVRAKIXIEGKSTFSKRAK

SEQ ID NO: 85 - protein, 24 aa, synthetic construct
  SITE 1: note = Acetylated leucine
  SITE 9: note = 2-amino-isobutyric acid
  SITE 10: note = Nepsilon-Acetyl-lysine, 2-amino-4-acetamido-hexanoic acid
  SITE 12: note = 2-amino-isobutyric acid
  SITE 13: note = 2-amino-isobutyric acid
  SITE 16: note = Lysine 4-[3-[2-[2-(3-aminopropoxy)ethoxy]ethoxy]pro pylamino]-4-oxo-butanoyl-4-[3-[2-[2-(3-aminopropoxy)ethoxy ]ethoxy]propylamino]-4-oxo-butanoyl-4-[3-[2-[2-(3-aminopro poxy)ethoxy]ethoxy]propylamino]-4-oxo-butanoyl-gamma- Glutamic acid-gamma- Glutamic acid-gamma- Glutamic acid-palmitoyl
  SITE 21: note = 2-amino-isobutyric acid
  SITE 24: note = Nepsilon-Acetyl-lysine, 2-amino-4-acetamido-hexanoic acid amide
LEGRERVRXKIXXEGKSTWSXRRK

SEQ ID NO: 86 - protein, 24 aa, synthetic construct
  SITE 1: note = Acetylated 2-amino-isobutyric acid
  SITE 10: note = Nepsilon-Acetyl-lysine, 2-amino-4-acetamido-hexanoic acid
  SITE 12: note = 2-amino-isobutyric acid
  SITE 16: note = Lysine 4-[3-[2-[2-(3-aminopropoxy)ethoxy]ethoxy]pro pylamino]-4-oxo-butanoyl-4-[3-[2-[2-(3-aminopropoxy)ethoxy ]ethoxy]propylamino]-4-oxo-butanoyl-4-[3-[2-[2-(3-aminopro poxy)ethoxy]ethoxy]propylamino]-4-oxo-butanoyl-gamma- Glutamic acid-gamma- Glutamic acid-gamma- Glutamic acid-palmitoyl
  SITE 21: note = alpha-methyl lysine
  SITE 24: note = Nepsilon-Acetyl-lysine, 2-amino-4-acetamido-hexanoic acid amide
XEGREKVRAKIXIEGKSTFSKRAK

SEQ ID NO: 87 - protein, 24 aa, synthetic construct
  SITE 1: note = Acetylated leucine
  SITE 10: note = Nepsilon-Acetyl-lysine, 2-amino-4-acetamido-hexanoic acid
  SITE 12: note = 2-amino-isobutyric acid
  SITE 13: note = 2-amino-isobutyric acid
  SITE 16: note = Lysine 4-[3-[2-[2-(3-aminopropoxy)ethoxy]ethoxy]pro pylamino]-4-oxo-butanoyl-4-[3-[2-[2-(3-aminopropoxy)ethoxy ]ethoxy]propylamino]-4-oxo-butanoyl-4-[3-[2-[2-(3-aminopro poxy)ethoxy]ethoxy]propylamino]-4-oxo-butanoyl-gamma- Glutamic acid-gamma- Glutamic acid-gamma- Glutamic acid-palmitoyl
  SITE 24: note = Nepsilon-Acetyl-lysine, 2-amino-4-acetamido-hexanoic acid amide
LEGREKVRAKIXXEGKSTFSRRAK

SEQ ID NO: 88 - protein, 24 aa, synthetic construct
  SITE 1: note = Acetylated leucine
  SITE 10: note = Nepsilon-Acetyl-lysine, 2-amino-4-acetamido-hexanoic acid
  SITE 12: note = 2-amino-isobutyric acid
  SITE 13: note = 2-amino-isobutyric acid
  SITE 16: note = Lysine 4-[3-[2-[2-(3-aminopropoxy)ethoxy]ethoxy]pro pylamino]-4-oxo-butanoyl-4-[3-[2-[2-(3-aminopropoxy)ethoxy ]ethoxy]propylamino]-4-oxo-butanoyl-4-[3-[2-[2-(3-aminopro poxy)ethoxy]ethoxy]propylamino]-4-oxo-butanoyl-gamma- Glutamic acid-gamma- Glutamic acid-gamma- Glutamic acid-palmitoyl
  SITE 21: note = Homolysine
  SITE 24: note = Nepsilon-Acetyl-lysine, 2-amino-4-acetamido-hexanoic acid amide
LEGREKVRAKIXXEGKSTFSXRAK

SEQ ID NO: 89 - protein, 24 aa, synthetic construct
  SITE 1: note = Acetylated leucine
  SITE 10: note = Nepsilon-Acetyl-lysine, 2-amino-4-acetamido-hexanoic acid
  SITE 12: note = 2-amino-isobutyric acid
  SITE 13: note = 2-amino-isobutyric acid
  SITE 16: note = Lysine 4-[3-[2-[2-(3-aminopropoxy)ethoxy]ethoxy]pro pylamino]-4-oxo-butanoyl-4-[3-[2-[2-(3-aminopropoxy)ethoxy ]ethoxy]propylamino]-4-oxo-butanoyl-4-[3-[2-[2-(3-aminopro poxy)ethoxy]ethoxy]propylamino]-4-oxo-butanoyl-gamma- Glutamic acid-gamma- Glutamic acid-gamma- Glutamic acid-palmitoyl
  SITE 19: note = Alpha-methyl-phenylalanine
  SITE 24: note = Nepsilon-Acetyl-lysine, 2-amino-4-acetamido-hexanoic acid amide
LEGREKVRAKIXXEGKSTFSRRAK

SEQ ID NO: 90 - protein, 24 aa, synthetic construct
  SITE 1: note = Acetylated leucine
  SITE 10: note = Nepsilon-Acetyl-lysine, 2-amino-4-acetamido-hexanoic acid
  SITE 13: note = 2-amino-isobutyric acid
  SITE 16: note = Lysine 4-[3-[2-[2-(3-aminopropoxy)ethoxy]ethoxy]pro pylamino]-4-oxo-butanoyl-4-[3-[2-[2-(3-aminopropoxy)ethoxy ]ethoxy]propylamino]-4-oxo-butanoyl-4-[3-[2-[2-(3-aminopro poxy)ethoxy]ethoxy]propylamino]-4-oxo-butanoyl-gamma- Glutamic acid-gamma- Glutamic acid-gamma- Glutamic acid-palmitoyl
  SITE 21: note = alpha-methyl lysine
  SITE 24: note = Nepsilon-Acetyl-lysine, 2-amino-4-acetamido-hexanoic acid amide
LEGREKVRAKIAXEGKSTFSKRAK

SEQ ID NO: 91 - protein, 24 aa, synthetic construct
  SITE 1: note = Acetylated leucine
  SITE 10: note = Nepsilon-Acetyl-lysine, 2-amino-4-acetamido-hexanoic acid
  SITE 12: note = 2-amino-isobutyric acid
  SITE 13: note = 2-amino-isobutyric acid
  SITE 16: note = Lysine 4-[3-[2-[2-(3-aminopropoxy)ethoxy]ethoxy]pro pylamino]-4-oxo-butanoyl-4-[3-[2-[2-(3-aminopropoxy)ethoxy ]ethoxy]propylamino]-4-oxo-butanoyl-4-[3-[2-[2-(3-aminopro poxy)ethoxy]ethoxy]propylamino]-4-oxo-butanoyl-gamma- Glutamic acid-gamma- Glutamic acid-gamma- Glutamic acid-palmitoyl
  SITE 19: note = 4-fluoro-phenylalanine
  SITE 21: note = 2-amino-isobutyric acid
  SITE 24: note = Nepsilon-Acetyl-lysine, 2-amino-4-acetamido-hexanoic acid amide
LEGRERVRAKIXXEGKSTFSXRRK

SEQ ID NO: 92 - protein, 24 aa, synthetic construct
  SITE 1: note = Acetylated leucine
  SITE 10: note = Nepsilon-Acetyl-lysine, 2-amino-4-acetamido-hexanoic acid
  SITE 12: note = 2-amino-isobutyric acid
  SITE 13: note = 2-amino-isobutyric acid
  SITE 16: note = Lysine 4-[3-[2-[2-(3-aminopropoxy)ethoxy]ethoxy]pro pylamino]-4-oxo-butanoyl-4-[3-[2-[2-(3-aminopropoxy)ethoxy ]ethoxy]propylamino]-4-oxo-butanoyl-4-[3-[2-[2-(3-aminopro poxy)ethoxy]ethoxy]propylamino]-4-oxo-butanoyl-gamma- Glutamic acid-gamma- Glutamic acid-gamma- Glutamic acid-palmitoyl
  SITE 19: note = 5-Fluorotryptophan
  SITE 21: note = 2-amino-isobutyric acid
  SITE 24: note = Nepsilon-Acetyl-lysine, 2-amino-4-acetamido-hexanoic acid amide
LEGRERVRAKIXXEGKSTWSXRRK

SEQ ID NO: 93 - protein, 24 aa, synthetic construct
  SITE 1: note = Acetylated leucine
  SITE 10: note = Nepsilon-Acetyl-lysine, 2-amino-4-acetamido-hexanoic acid
  SITE 12: note = 2-amino-isobutyric acid
  SITE 13: note = 2-amino-isobutyric acid
  SITE 16: note = Lysine 4-[3-[2-[2-(3-aminopropoxy)ethoxy]ethoxy]pro pylamino]-4-oxo-butanoyl-4-[3-[2-[2-(3-aminopropoxy)ethoxy ]ethoxy]propylamino]-4-oxo-butanoyl-4-[3-[2-[2-(3-aminopro poxy)ethoxy]ethoxy]propylamino]-4-oxo-butanoyl-gamma- Glutamic acid-gamma- Glutamic acid-gamma- Glutamic acid-palmitoyl
  SITE 24: note = Nepsilon-Acetyl-lysine, 2-amino-4-acetamido-hexanoic acid amide
LEGREKVRAKIXXEGKSTFTRRAK

SEQ ID NO: 94 - protein, 24 aa, synthetic construct
  SITE 1: note = Acetylated leucine
  SITE 10: note = Nepsilon-Acetyl-lysine, 2-amino-4-acetamido-hexanoic acid
  SITE 12: note = 2-amino-isobutyric acid
  SITE 13: note = 2-amino-isobutyric acid
  SITE 16: note = Lysine 4-[3-[2-[2-(3-aminopropoxy)ethoxy]ethoxy]pro pylamino]-4-oxo-butanoyl-4-[3-[2-[2-(3-aminopropoxy)ethoxy ]ethoxy]propylamino]-4-oxo-butanoyl-4-[3-[2-[2-(3-aminopro poxy)ethoxy]ethoxy]propylamino]-4-oxo-butanoyl-gamma- Glutamic acid-gamma- Glutamic acid-gamma- Glutamic acid-palmitoyl
  SITE 24: note = Nepsilon-Acetyl-lysine, 2-amino-4-acetamido-hexanoic acid amide
LEGREKVRAKIXXEGKSTFVRRAK

SEQ ID NO: 95 - protein, 24 aa, synthetic construct
  SITE 1: note = Acetylated leucine
  SITE 10: note = Nepsilon-Acetyl-lysine, 2-amino-4-acetamido-hexanoic acid
  SITE 12: note = 2-amino-isobutyric acid
  SITE 13: note = 2-amino-isobutyric acid
  SITE 16: note = Lysine 4-[3-[2-[2-(3-aminopropoxy)ethoxy]ethoxy]pro pylamino]-4-oxo-butanoyl-4-[3-[2-[2-(3-aminopropoxy)ethoxy ]ethoxy]propylamino]-4-oxo-butanoyl-4-[3-[2-[2-(3-aminopro poxy)ethoxy]ethoxy]propylamino]-4-oxo-butanoyl-gamma- Glutamic acid-gamma- Glutamic acid-gamma- Glutamic acid-Myristoyl
  SITE 21: note = alpha-methyl lysine
  SITE 24: note = Nepsilon-Acetyl-lysine, 2-amino-4-acetamido-hexanoic acid amide
LEGREKVRAKIXXEGKSTFSKRAK

SEQ ID NO: 96 - protein, 24 aa, synthetic construct
  SITE 1: note = Acetylated leucine
  SITE 10: note = Nepsilon-Acetyl-lysine, 2-amino-4-acetamido-hexanoic acid
  SITE 12: note = 2-amino-isobutyric acid
  SITE 13: note = 2-amino-isobutyric acid
  SITE 16: note = Lysine 4-[3-[2-[2-(3-aminopropoxy)ethoxy]ethoxy]pro pylamino]-4-oxo-butanoyl-4-[3-[2-[2-(3-aminopropoxy)ethoxy ]ethoxy]propylamino]-4-oxo-butanoyl-4-[3-[2-[2-(3-aminopro poxy)ethoxy]ethoxy]propylamino]-4-oxo-butanoyl-gamma- Glutamic acid-gamma- Glutamic acid-gamma- Glutamic acid-Myristoyl
  SITE 21: note = alpha-methyl lysine
  SITE 24: note = Lysine amine
LEGREKVRAKIXXEGKSTFSKRAK

SEQ ID NO: 97 - protein, 24 aa, synthetic construct
  SITE 1: note = Acetylated leucine
  SITE 10: note = Nepsilon-Acetyl-lysine, 2-amino-4-acetamido-hexanoic acid
  SITE 12: note = 2-amino-isobutyric acid
  SITE 13: note = 2-amino-isobutyric acid
  SITE 16: note = Lysine 4-[3-[2-[2-(3-aminopropoxy)ethoxy]ethoxy]pro pylamino]-4-oxo-butanoyl-4-[3-[2-[2-(3-aminopropoxy)ethoxy ]ethoxy]propylamino]-4-oxo-butanoyl-4-[3-[2-[2-(3-aminopro poxy)ethoxy]ethoxy]propylamino]-4-oxo-butanoyl-gamma- Glutamic acid-gamma- Glutamic acid-gamma- Glutamic acid-gamma- Glutamic acid-Myristoyl)
  SITE 21: note = alpha-methyl lysine
  SITE 24: note = Lysine amine
LEGREKVRAKIXXEGKSTFSKRAK

SEQ ID NO: 98-99 - skipped (2 entries)

SEQ ID NO: 100 - protein, 24 aa, synthetic construct
  SITE 1: note = Hydrogen Glutamine
  SITE 24: note = Cysteine Hydroxide
QLYSALANKCCHVGCTKRSLARFC

SEQ ID NO: 101 - protein, 29 aa, synthetic construct
  SITE 1: note = Hydrogen aspartic acid
  SITE 29: note = Serine hydroxide
DSWMEEVIKLCGRELVRAQIAICGMSTWS

SEQ ID NO: 102 - protein, 27 aa, synthetic construct
  SITE 1: note = Hydrogen valine
  SITE 27: note = Leucine amine
VIKLSGRELVRAQIAISGMSTWSKRSL

SEQ ID NO: 103 - protein, 27 aa, synthetic construct
  SITE 1: note = acetylated valine
  SITE 27: note = Leucine amine
VIKLSGRELVRAQIAISGMSTWSKRSL

SEQ ID NO: 104 - protein, 27 aa, synthetic construct
  SITE 1: note = Acetylated valine
  SITE 27: note = Lysine amine
VIKLSGRELVRAQIAISGMSTWSKKKK

SEQ ID NO: 105 - protein, 24 aa, synthetic construct
  VARIANT 1: note = acetylated leucine, 2-amino-isobutyric acid, Nepsilon-acetyl-lysine and alpha-methyl-leucine
  VARIANT 6: note = lysine, arginine, homolysine, homoarginine, ornithine, glutamine, phenylalanine and leucine
  VARIANT 9: note = alanine, 2-amino-isobutyric acid, leucine, Nepsilon-acetyl-lysine and glutamine
  VARIANT 10: note = lysine, Nepsilon-acetyl-lysine, citrulline, glutamine, alanine and 2-amino-isobutyric acid
  VARIANT 12: note = 2-amino-isobutyric acid and alanine
  VARIANT 13: note = 2-amino-isobutyric acid and isoleucine
  SITE 16: note = Lysine modified with [(PEGxx)b(gE)cCd], wherein PEGxx independently represents a polyethylene glycol derivative selected from the group consisting of 2-[2-(2-aminoethoxy)ethoxy]acetyl, 2-[2-[3-[2-[2-(3-aminop ropoxy)ethoxy]propylamino]-2-oxo-ethoxy]acetyl, and 4-[3-[2-[2-(3-aminopropoxy)ethoxy]ethoxy]propylamino]-4-ox o-butanoyl, gE represents gamma-Glutamic acid, and Cd represents a linear saturated C12-C22 acyl group.
  VARIANT 18: note = threonine, lysine, arginine and glutamine
  VARIANT 19: note = tryptophan, phenylalanine, 5-fluoro-tryptophan, 5-chloro-tryptophan, 5-methoxy-tryptophan, tyrosine, 4-fluoro-phenylalanine, 1-naphtylalanine, 2-naphtylalanine, alpha-methyl-tryptophan, alpha-methyl-phenylalanine and 5-hydroxy-tryptophan
  VARIANT 20: note = D-serine, 2-amino-isobutyric acid, threonine, alpha-methyl-serine, Nepsilon-acetyl-lysine and valine;
  VARIANT 21: note = 2-amino-isobutyric acid, alpha-methyl-lysine, D-lysine, lysine, homolysine, ornithine, arginine and alpha-methyl-arginine
  VARIANT 22: note = arginine, Nomega-methyl-arginine, alanine, Nomega,Nomega'-dimethyl-arginine and citrulline
  VARIANT 23: note = lysine, alanine, arginine, Nepsilon-acetyl-lysine and Nepsilon,Nepsilon,Nepsilon-tri-methyl-lysine
  VARIANT 24: note = lysine, Nepsilon-acetyl-lysine, leucine, arginine and alanine amine
XEGREXVRXXIXXEGXSXXXXXXX

SEQ ID NO: 106 - protein, 24 aa, synthetic construct
  SITE 1: note = Acetylated leucine
  VARIANT 6: note = lysine, arginine, homolysine, glutamine, phenylalanine and leucine
  VARIANT 9: note = alanine and 2-amino-isobutyric acid
  VARIANT 10: note = lysine, Nepsilon-lysine, glutamine and citrulline
  SITE 12: note = 2-amino-isobutyric acid
  SITE 13: note = 2-amino-isobutyric acid
  SITE 16: note = Glycine modified with -(TTDS)2-(gE)3-Palm, -(TTDS)3-(gE)3-Palm, -(PEG2DGA)3-(gE)3-Palm, -(PEG2)4-(gE)3-Palm, -(TTDS)2-(gE)2-Palm, -(TTDS)2-(gE)3-Stea, -(TTDS)3-(gE)3-Stea, -(PEG2DGA)3-(gE)3-Stea, -(PEG2DGA)3-(gE)4-Stea, -(PEG2)3-(gE)3-Palm, -(PEG2)4-(gE)3-Stea, -(PEG2)5-(gE)3-Palm, -(PEG2)5-(gE)4-Palm, -(TTDS)3-(gE)4-Stea, -(TTDS)2-(gE)4-Palm, -(TTDS)3-(gE)2-Stea and -(TTDS)2-(gE)4-Stea, wherein gE represents alpha-glutamic acid, Palmepresents Palmitoyl and Stea represents Stearoyl
  VARIANT 19: note = tryptophan and phenylalanine
  VARIANT 20: note = serine, D-serine and 2-amino-isobutyric acid
  VARIANT 21: note = 2-amino-isobutyric acid,alpha-methyl lysine, D-lysine, and lysine
  VARIANT 23: note = lysine, alanine and arginine
  VARIANT 24: note = lysine and Nepsilone-acetyl-lysine; or a salt or solvate thereof amine
LEGREXVRXXIXXEGXSTXXXRXX

SEQ ID NO: 107 - protein, 24 aa, synthetic construct
  VARIANT 1: note = Acetylated leucine, Nepsilon-acetyl-lysine and 2-amino-isobutyric acid
  VARIANT 6: note = lysine, arginine, homolysine, glutamine, phenylalanine and leucine
  VARIANT 9: note = alanine, 2-amino-isobutyric acid and Nepsilone-acetyl-lysine
  VARIANT 10: note = lysine, Nepsilon-acetyl-lysine, gluatmine and citrulline
  VARIANT 12: note = 2-amino-isobutyric acid and alanine
  VARIANT 13: note = 2-amino-isobutyric acid and isoleucine
  SITE 16: note = Lysine modified by -(TTDS)2-(gE)3-Palm, -(TTDS)3-(gE)3-Palm, -(PEG2DGA)3-(gE)3-Palm, -(PEG2)4-(gE)3-Palm, -(TTDS)2-(gE)2-Palm, -(TTDS)2-(gE)3-Stea, -(TTDS)3-(gE)3-Stea, -(PEG2DGA)3-(gE)3-Stea, -(PEG2DGA)3-(gE)4-Stea, -(PEG2)3-(gE)3-Palm, -(PEG2)4-(gE)3-Stea, -(PEG2)5-(gE)3-Palm, -(PEG2)5-(gE)4-Palm, -(TTDS)3-(gE)4-Stea, -(TTDS)2-(gE)4-Palm, -(TTDS)3-(gE)2-Stea, -(TTDS)2-(gE)4-Stea, -(TTDS)4-(gE)3-Stea, -(TTDS)3-(gE)4-Palm, -(TTDS)4-(gE)3-Palm, -(TTDS)3-(gE)3-Myr and -(TTDS)3-(gE)4-Myr wherein gE represents gamma-glutamic acid, Palm represents palmitoyl and Stea represents Stearoyl
  VARIANT 18: note = threonine, glutamine, arginine and lysine
  VARIANT 19: note = tryptophan, phenylalanine, 5-Chlorotryptophan, alpha-methyl-phenylalanine, 4-Fluoro-phenylalanine, and 5-Fluorotryptophan
  VARIANT 20: note = serine, D-serine, 2-amino-isobutyric acid, Nepsilon-acetyl-lysine, threonine and valine
  VARIANT 21: note = 2-amino-isobutyric acid, alpha-methyl lysine. D-lysine, lysine, homolysine and arginine
  VARIANT 23: note = lysine, alanine, arginine and Napepsilon-acetyl-lysine
  VARIANT 24: note = lysine, Nepsilon-acetyl-lysine and arginineor a salt or solvate thereof
XEGREXVRXXIXXEGXSXXXXRXX

SEQ ID NO: 108 - protein, 12 aa, synthetic construct
  SITE 12: note = Glycine amine
GGGCYFQNCPKG

(19) United States
(12) Patent Application Publication
(10) Pub. No.: US 2024/0400638 A1
(43) Pub. Date: Dec. 5, 2024

(54) COMBINATION OF RELAXIN AND VASOPRESSIN ANALOGUES FOR TREATMENT OF RENAL DISORDERS OR CONDITIONS

(71) Applicants: Guido MAGNI, Basel (CH); River 2 Renal Corp., New York, NY (US)

(72) Inventor: Guido Magni, Basel (CH)

(21) Appl. No.: 18/685,694

(22) PCT Filed: Aug. 22, 2022
(86) PCT No.: PCT/US2022/041095

§ 371 Date: Feb. 22, 2024

Related U.S. Application Data
(60) Provisional application No. 63/332,994, filed on Apr. 20, 2022.
(60) Provisional application No. 63/236,090, filed on Aug. 23, 2021.

(51) Int. Cl. C07K 14/64; A61K 38/00; A61P 1/16; A61P 13/12; C07K 7/06

(52) U.S. Cl. CPC C07K 14/64; A61P 1/16; A61P 13/12; C07K 7/06; A61K 38/00

(57) ABSTRACT

The present disclosure relates to a combination therapy comprising co-administration of a relaxin analogue for example, peptide analogues of the B-chain of human relaxin-2 able to activate the RXFP1 receptor and (b) an analogue of vasopressin (also termed arginne vasopressin (AVP), antidiuretic hormone (ADH), and agripressin) able to activate the V1 receptor, for example terlipressin, to an individual in need thereof in the treatment of a renal disorder, such as renal dysfunction in cirrhosis, hepatorenal syndrome type 1 (HRS-AKI) and type 2 (HRS-NAKI), chronic kidney disease and acute kidney injury, and to preserve renal function in peri-operative liver transplantation. This disclosure also relates to compositions comprising relaxin analogues and/or vasopressin analogues for co-administration to an individual in need thereof; preparation of such compositions, and use of such compositions for co-administration to an individual in need thereof, and commercial packages thereto.

### CROSS-REFERENCE TO RELATED APPLICATIONS

This application claims the benefit of and priority to U.S. Provisional Patent Application No. 63/236,090, filed Aug. 23, 2021, and U.S. Provisional Patent Application No. 63/332,994, filed Apr. 20, 2022, the entire disclosure of each of which is hereby incorporated by reference in its entirety for all purposes.

### FIELD OF THE INVENTION

The present disclosure relates to a combination therapy comprising co-administration of (a) a relaxin analogue able to activate the RXFP1 receptor, for example peptide analogues of the B-chain of human relaxin-2, and (b) an analogue of vasopressin (also termed arginine vasopressin (AVP), antidiuretic hormone (ADH), and agripressin) able to activate the V1 receptor, for example terlipressin, administered to an individual in need thereof in the treatment of a renal disorder, such as renal dysfunction in cirrhosis, hepatorenal syndrome type 1 (HRS-AKI) and type 2 (HRS-NAKI), chronic kidney disease and acute kidney injury, and to preserve renal function in peri-operative liver transplantation. This disclosure also relates to compositions comprising relaxin analogues and/or vasopressin analogues for co-administration to an individual in need thereof, preparation of such compositions, and use of such compositions for co-administration to an individual in need thereof, and commercial packages thereto.

### BACKGROUND OF THE INVENTION

Terlipressin is a synthetic vasopressin that is approved in many countries outside of the United States to treat the life-threatening complications of cirrhosis, including hepatorenal syndrome (HRS) and esophageal bleeding (EVB). Its use is limited to the hospital setting due to its short half-life (Nilsson, et al., (1990) Drugs Explt Clin. Res., XVI (6):307-314), and it is typically administered as an intravenous bolus usually every 4 to 6 hours. However, although terlipressin may have clinical utility, a recent study found that at 3 months, mortality in patients receiving terlipressin and standard of care (e.g., albumin) was 51%, as compared to 45% for patients receiving standard of care alone (Wong, F. et al., Terlipressin plus Albumin for the Treatment of Type 1 Hepatorenal Sydrome, NEJM (2021), 384: 818-828. Additionally, terlipressin can cause side effects in up to 40% of patients. Severe side effects—including respiratory failure, myocardial infarction, angina, cardiac arrhythmia, severe hypertension and intestinal ischemia/infarction/bleeding—have been reported and can require discontinuation of treatment in up to 10% of the patients (Angeli, (2011) Ascites, Hyponatremia and Hepatorenal Syndrome: Progress in Treatment, 28:189-197). Indeed, due to its rapid vasoconstrictor properties, IV bolus dosed terlipressin is to be used with caution or might not be recommended in patients with severe asthma, severe hypertension, advanced atherosclerosis, cardiac dysrhythmias, and coronary insufficiency.

Therefore, there is a need for improved therapies for treating hepatorenal syndrome (HRS) and related conditions, e.g., to improve their efficacy and/or safety profile.

### SUMMARY OF THE INVENTION

In some embodiments, provided herein is a method of preventing or treating renal failure in an individual in need thereof, comprising co-administering an effective amount of a relaxin analogue and a vasopressin analogue to the individual. In some embodiments, the renal failure is selected from the group consisting of: renal dysfunction induced by liver cirrhosis, renal dysfunction induced by liver transplantation, chronic kidney disease, and acute kidney injury.

Also provided herein, according to some embodiments, is a method of preventing or treating hepatorenal syndrome in an individual in need thereof, comprising co-administering an effective amount of a relaxin analogue and a vasopressin analogue to the individual. In some embodiments, the hepatorenal syndrome is HRS-AKI (hepatorenal syndrome type 1).

In some embodiments, the relaxin analogue is an RXFP1 agonist. In some embodiments, the relaxin analogue is a long-acting peptidyl RXFP1 agonist. In some embodiments, the vasopressin analogue is a V1a receptor agonist. In some embodiments, the vasopressin analogue is terlipressin or a pharmaceutically acceptable salt thereof.

In some embodiments, the terlipressin is administered intravenously at a dosage of 0.5 to 2 mg per administration. In some embodiments, the terlipressin is administered intravenously at a dosage of 0.5 to 2 mg every 4 to 6 hours. In some embodiments, the terlipressin is administered via an intravenous infusion. In some embodiments, the terlipressin is administered at a rate of 0.5 to 2 mg per 4 to 6 hours. In some embodiments, the terlipressin is administered at a rate of 0.5 to 2 mg per 8 to 36 hours. In some embodiments, the terlipressin is administered at a rate of 0.5 to 2 mg per 10 to 30 hours. In some embodiments, the terlipressin is administered at a rate of 0.5 to 2 mg per 15 to 28 hours. In some embodiments, the terlipressin is administered at a rate of 0.5 to 2 mg per 20 to 25 hours. In some embodiments, the terlipressin is administered at a rate of 0.5 to 2 mg per 24 hours.

In some embodiments, the relaxin analogue is administered at a dose of from about 0.01 mg/kg to about 0.5 mg/kg. In some embodiments, the relaxin analogue is administered, parenterally, intravenously, subcutaneously, rectally, transdermally, or by inhalation. In some embodiments, the relaxin analogue has an EC50 for activation RXFP1 in the in vitro OVCAR5 cAMP assay of less than 15 nM, less than 1 nM, less than 0.5 nM, or less than 0.1 nM.

In some embodiments, the method of preventing or treating further comprises administration of midodrine or octreotide to the individual.

In some embodiments, the relaxin analogue and the vasopressin analogue are administered simultaneously. In some embodiments, the relaxin analogue and the vasopressin analogue are administered in a single composition. In some embodiments, the relaxin analogue and the vasopressin analogue are administered in a separate composition. In some embodiments, the vasopressin analogue and the relaxin analogue are administered sequentially.

In some embodiments, the combination therapy has a synergistic therapeutic effect. In some embodiments, the administration of the relaxin analogue mitigates adverse effects associated with vasopressin analogue treatment in the individual. In some embodiments, the administration of the vasopressin analogue reduces risk of hypotension in said individual associated with treatment with the relaxin analogue. In some embodiments, the administration of the vasopressin analogue or relaxin analogue increases renal pressure in said individual.

Also provided herein, according to some embodiments, is a method of treating renal failure in an individual in need thereof, comprising: administering a relaxin analogue to the individual, wherein the individual previously has been administered a vasopressin analogue.

Also provided herein, according to some embodiments, is a method of treating hepatorenal syndrome in an individual with liver cirrhosis comprising a) administering a relaxin analogue to the individual, wherein the individual previously has been administered a vasopressin analogue.

In some embodiments, the administration of the relaxin analogue mitigates adverse effects associated with vasopressin analogue treatment in the individual.

Also provided herein, according to some embodiments, is a method of treating renal failure in an individual in need thereof, comprising: administering a vasopressin analogue to the individual, wherein the individual previously has been administered a relaxin analogue.

Also provided herein, according to some embodiments, is a method of treating hepatorenal syndrome in an individual with liver cirrhosis comprising a) administering a vasopressin analogue to the individual, wherein the individual previously has been administered an effective amount of a relaxin analogue.

In some embodiments, the administration of the vasopressin analogue reduces risk of hypotension in said individual associated with treatment with the relaxin analogue. In some embodiments, the administration of the vasopressin analogue or relaxin analogue increases renal pressure in said individual.

In some embodiments, the hepatorenal syndrome is HRS-AKI (hepatorenal syndrome type 1).

In some embodiments, also provided herein is a pharmaceutical composition comprising separately or together a relaxin analogue, a vasopressin analogue, and one or more pharmaceutically acceptable excipients.

In some embodiments, also provided herein is a kit comprising a relaxin analogue in a pharmaceutically acceptable composition and a vasopressin analogue in a pharmaceutically acceptable composition.

In some embodiments, the relaxin analogue is an RXFP1 agonist. In some embodiments, the relaxin analogue is a long-acting peptidyl RXFP1 agonist. In some embodiments, the vasopressin analogue is a V1a receptor agonist. In some embodiments, the vasopressin analogue is terlipressin or a pharmaceutically acceptable salt thereof.

In some embodiments, the relaxin analogue is a modified relaxin B chain peptide comprising formula (I) (SEQ ID NO: 105):

Nₜₑᵣ-Ac-X₁₀-E-G-R-E-X₁₅-V-R-X₁₈-X₁₉-I-X₂₁-X₂₂-E-G-
X₂₅-S-X₂₇-X₂₈-X₂₉-X₃₀-X₃₁-X₃₂-X₃₃-NH₂-Cₜₑᵣ,

wherein:
Nₜₑᵣ represents the N-terminal end of the peptide;
Cₜₑᵣ represents the C-terminal end of the peptide;
Ac represents acetyl group;
X₁₀ represents an amino acid selected from the group consisting of leucine, 2-amino-isobutyric acid, Nε-acetyl-lysine and α-methyl-leucine;
E represents glutamic acid;
G represents glycine;
R represents arginine;
X₁₅ represents an amino acid selected form the group consisting of lysine, arginine, homolysine, homoarginine, ornithine, glutamine, phenylalanine and leucine;
V represents valine;
X₁₈ represents an amino acid selected from the group consisting of alanine, 2-amino-isobutyric acid, leucine, Nε-acetyl-lysine and glutamine;
X₁₉ represents an amino acid selected from the group consisting of lysine, Nε-acetyl-lysine, citrulline, glutamine, alanine and 2-amino-isobutyric acid;
I represents isoleucine;
X₂₁ represents an amino acid selected from the group consisting of 2-amino-isobutyric acid and alanine;
X₂₂ represents an amino acid selected from the group consisting of 2-amino-isobutyric acid and isoleucine;
X₂₅ represents the following structure:

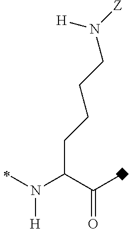

in which:
* represents a covalent bond with the glycine preceding X₂₅ in formula (I);
♦ represents a covalent bond with the serine following X₂₅ in formula (I); and
Z represents a group of formula (II):

-[(PEGₓₓ)_{b}(gE)_{c}C_{d}],
b and c independently represent 1, 2, 3, 4 or 5;
PEGₓₓ independently represents a polyethylene glycol derivative selected from the group consisting of PEG₂, PEG₂DGA, and TTDS;
gE represents γ-glutamic acid; and
C_{d} represents a linear saturated C₁₂-C₂₂ acyl group;
S represents serine;
X₂₇ represents an amino acid selected from the group consisting of threonine, lysine, arginine and glutamine;
X₂₈ represents an amino acid selected from the group consisting of tryptophan, phenylalanine, 5-fluoro-tryptophan, 5-chloro-tryptophan, 5-methoxy-tryptophan, tyrosine, 4-fluoro-phenylalanine, 1-naphtylalanine, 2-naphtylalanine, α-methyl-tryptophan, α-methyl-phenylalanine and 5-hydroxy-tryptophan;
X₂₉ represents an amino acid selected from the group consisting of serine, D-serine, 2-amino-isobutyric acid, threonine, α-methyl-serine, Nε-acetyl-lysine and valine;
X₃₀ represents an amino acid selected from the group consisting of 2-amino-isobutyric acid, α-methyl-lysine, D-lysine, lysine, homolysine, ornithine, arginine and α-methyl-arginine;
X₃₁ represents an amino acid selected from the group consisting of arginine, Nω-methyl-arginine, alanine, Nω,Nω′-dimethyl-arginine and citrulline;
X₃₂ represents an amino acid selected from the group consisting of lysine, alanine, arginine, Nε-acetyl-lysine and Nε,Nε,Nε-tri-methyl-lysine; and
X₃₃ represents an amino acid selected from lysine, Nε-acetyl-lysine, leucine, arginine and alanine;
or a salt or solvate thereof.

In some embodiments, b represents 2, 3, 4 or 5 and c represents 2, 3 or 4; or a salt or solvate thereof.

In some embodiments, C_{d} represents a linear saturated C₁₂-C₂₂ acyl group, for example a linear saturated acyl group selected from the group consisting of C₁₂ (Lau), C₁₄ (Myr), C₁₅ (Penta), C₁₆ (Palm), C₁₇ (Hepta), C₁₈ (Stea), C₂₀ (Eico) and C₂₂ (Doco) acyl group.

In one embodiment C_{d} represents a linear saturated acyl group selected from the group consisting of C₁₂ (Lau), C₁₄ (Myr), C₁₅ (Penta), C₁₆ (Palm), C₁₇ (Hepta) or C₁₈ (Stea) acyl group, for example a linear saturated C₁₄, C₁₆ or C₁₈ acyl group, or for example a linear C₁₆ or C₁₈ acyl group; or a salt or solvate thereof.

In some embodiments, C_{d} represents a linear saturated acyl group selected from the group consisting of: C₁₂ (Lau), C₁₄ (Myr), C₁₅ (Penta), C₁₆ (Palm), C₁₇ (Hepta) and Cis (Stea) acyl group; or a salt or solvate thereof;

In some embodiments, C_{d} represents a linear C₁₆ or C₁₈ acyl group; or a salt or solvate thereof.

In various embodiments, the relaxin analogue comprises formula (Ib):

Nₜₑᵣ-Ac-X₁₀-E-G-R-E-X₁₅-V-R-X₁₈-X₁₉-I-X₂₁-X-₂₂-E-G-X₂₅-
S-X₂₇-X₂₈-X₂₉-X₃₀-R-X₃₂-X₃₃-NH₂-Cₜₑᵣ,

wherein:
Nₜₑᵣ represents the N-terminal end of the peptide;
Cₜₑᵣ represents the C-terminal end of the peptide;
Ac represents acetyl group;
X₁₀ represents an amino acid selected form the group consisting of leucine, Nε-acetyl-lysine and 2-amino-isobutyric acid;
E represents glutamic acid;
G represents glycine;
R represents arginine;
X₁₅ represents an amino acid selected form the group consisting of lysine, arginine, homolysine, glutamine, phenylalanine and leucine;
V represents valine;
X₁₈ represents an amino acid selected from the group consisting of alanine, 2-amino-isobutyric acid and Nε-acetyl-lysine;
X₁₉ represents an amino acid selected from the group consisting of lysine, Nε-acetyl-lysine, glutamine and citrulline; I represents isoleucine;
X₂₁ represents an amino acid selected from the group consisting of 2-amino-isobutyric acid and alanine;
X₂₂ represents an amino acid selected from the group consisting of 2-amino-isobutyric acid and isoleucine;
X₂₅ represents the following structure:

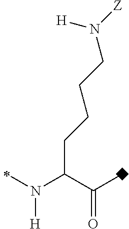

in which:
   * represents a covalent bond with the glycine preceding X₂₅ in formula (Ia);
   ♦ represents a covalent bond with the serine following X₂₅ in formula (Ia); and Z is selected from the group consisting of a -(TTDS)₂-(gE)₃-Palm, -(TTDS)₃-(gE)₃-Palm, -(PEG₂DGA)₃-(gE)₃-Palm, -(PEG₂)₄-(gE)₃-Palm, -(TTDS)₂-(gE)₂-Palm, -(TTDS)₂-(gE)₃-Stea, -(TTDS)₃-(gE)₃-Stea, -(PEG₂DGA)₃-(gE)₃-Stea, -(PEG₂DGA)₃-(gE)₄-Stea, -(PEG₂)₃-(gE)₃-Palm, -(PEG₂)₄-(gE)₃-Stea, -(PEG₂)₅-(gE)₃-Palm, -(PEG₂)s-(gE)₄-Palm, -(TTDS)₃-(gE)₄-Stea, -(TTDS)₂-(gE)₄-Palm, -(TTDS)₃-(gE)₂-Stea, -(TTDS)₂-(gE)₄-Stea, -(TTDS)₄-(gE)₃-Stea, -(TTDS)₃-(gE)₄-Palm, -(TTDS)₄-(gE)₃-Palm, -(TTDS)₃-(gE)₃-Myr and -(TTDS)₃-(gE)₄-Myr, wherein gE represents γ-glutamic acid, Palm represents Palmitoyl, and Stea represents Stearoyl;
S represents serine;
X₂₇ represents an amino acid selected from the group consisting of threonine, glutamine, arginine and lysine;
X₂₈ represents an amino acid selected from the group consisting of tryptophan, phenylalanine, 5-Chlorotryptophan, α-Methyl-phenylalanine, 4-Fluoro-phenylalanine and 5-Fluorotryptophan;
X₂₉ represents an amino acid selected from the group consisting of serine, D-serine, 2-amino-isobutyric acid, Nε-acetyl-lysine, threonine and valine;
X₃₀ represents an amino acid selected from the group consisting of 2-amino-isobutyric acid, α-methyl-lysine, D-lysine, lysine, homolysine and arginine;
X₃₂ represents an amino acid selected from the group consisting of lysine, alanine, arginine and Nε-acetyl-lysine; and
X₃₃ represents an amino acid selected from lysine, Nε-acetyl-lysine and arginine; or a salt or solvate thereof.

In some embodiments, the relaxin analogue comprises an amino acid sequence selected from the group consisting of: SEQ ID NO: 1 to 97.

In some embodiments, Z is selected from the group consisting of: -(TTDS)₂-(gE)₃-Palm, -(TTDS)₃-(gE)₃-Palm, -(PEG₂DGA)₃-(gE)₃-Palm, -(PEG₂)₄-(gE)₃-Palm, -(TTDS)₂-(gE)₂-Palm, -(TTDS)₂-(gE)₃-Stea, -(TTDS)₃-(gE)₃-Stea, -(PEG₂DGA)₃-(gE)₃-Stea, -(PEG₂)₃-(gE)₃-Palm, -(PEG₂)₄-(gE)₃-Stea, -(PEG₂)₅-(gE)₃-Palm, -(TTDS)₃-(gE)₄-Stea, -(TTDS)₂-(gE)₄-Palm, -(TTDS)₂-(gE)₄-Stea, -(TTDS)₄-(gE)₃-Stea, -(TTDS)₃-(gE)₄-Palm, -(TTDS)₄-(gE)₃-Palm, -(TTDS)₃-(gE)₃-Myr and -(TTDS)₃-(gE)₄-Myr.

In some embodiments, the relaxin analogue comprises an amino acid sequence selected from the group consisting of SEQ ID NO: 3, 6, 7, 9-12, 20-22, 26, 28, 30-34, 45, 47-49, 51, 54-62, 64, 67-69, 71-86, 91, 93 and 96.

In some embodiments, the relaxin analogue comprises an amino acid sequence selected from the group consisting of SEQ ID NO: 3, 6, 7, 20, 26, 30-34, 45, 48, 49, 51, 54-61, 67, 71, 73, 75-79, 81, 83-92 and 97.

In some embodiments, the relaxin analogue comprises an amino acid sequence selected from the group consisting of SEQ ID NO: 3, SEQ ID NO: 6, SEQ ID NO: 7, AND SEQ ID NO: 20.

In some embodiments, the relaxin analogue comprises the amino acid sequence of SEQ ID NO: 3.

Additionally disclosed herein is a method of preventing or treating renal failure in an individual in need thereof, comprising co-administering a dose of a relaxin analogue from about 1.0 mg to about 10.0 mg and an effective amount of a vasopressin analogue to the individual. In various embodiments, the renal failure is selected from the group consisting of: renal dysfunction induced by liver cirrhosis, renal dysfunction induced by liver transplantation, chronic kidney disease, and acute kidney injury.

Additionally disclosed herein is a method of preventing or treating hepatorenal syndrome in an individual in need thereof, comprising co-administering a dose of a relaxin analogue from about 1.0 mg to about 10.0 mg and an effective amount of a vasopressin analogue to the individual. In various embodiments, the hepatorenal syndrome is HRS-AKI (hepatorenal syndrome type 1).

In various embodiments, the relaxin analogue is an RXFP1 agonist. In various embodiments, the relaxin analogue is a long-acting peptidyl RXFP1 agonist.

In various embodiments, from about 1.0 mg to about 3.0 mg of the relaxin analogue is administered to the individual. In various embodiments, about 1.0 mg of the relaxin analogue is administered to the individual. In various embodiments, about 2.0 mg of the relaxin analogue is administered to the individual. In various embodiments, from about 3.0 mg to about 5.0 mg of the relaxin analogue is administered to the individual. In various embodiments, about 4.0 mg of the relaxin analogue is administered to the individual. In various embodiments, from about 5.0 mg to about 10.0 mg of the relaxin analogue is administered to the individual. In various embodiments, about 5.0 mg of the relaxin analogue is administered to the individual. In various embodiments, about 10.0 mg of the relaxin analogue is administered to the individual. In various embodiments, the relaxin analogue is administered intravenously. In various embodiments, the relaxin analogue is administered intravenously over from about 1 hour to about 10 hours. In various embodiments, the relaxin analogue is administered intravenously over from about 2 hours to about 8 hours. In various embodiments, the relaxin analogue is administered intravenously over from about 3 hours to about 6 hours. In various embodiments, the relaxin analogue is administered intravenously over about 4 hours.

In various embodiments, methods disclosed herein further comprise administering an additional dose of the relaxin analogue to the individual. In various embodiments, the additional dose of the relaxin analogue is administered between 5 hours and 18 hours after administration of the about 1.0 mg to about 5.0 mg of the relaxin analogue. In various embodiments, the additional dose of the relaxin analogue is administered between 8 hours and 15 hours after administration of the dose from about 1.0 mg to about 10.0 mg of the relaxin analogue. In various embodiments, the additional dose of the relaxin analogue is administered between 10 hours and 13 hours after administration of the dose from about 1.0 mg to about 10.0 mg of the relaxin analogue. In various embodiments, the additional dose of the relaxin analogue is administered about 12 hours after administration of the dose from about 1.0 mg to about 10.0 mg of the relaxin analogue. In various embodiments, the additional dose of the relaxin analogue is administered subcutaneously to the individual. In various embodiments, the additional dose of the relaxin analogue comprises from about 1 mg to about 50 mg of the relaxin analogue. In various embodiments, the additional dose of the relaxin analogue comprises from about 5 mg to about 15 mg of the relaxin analogue. In various embodiments, the additional dose of the relaxin analogue comprises from about 8 mg to about 12 mg of the relaxin analogue. In various embodiments, the additional dose of the relaxin analogue comprises about 10 mg of the relaxin analogue.

In various embodiments, methods disclosed herein further comprise administering a yet additional dose of the relaxin analogue to the individual. In various embodiments, the yet additional dose of the relaxin analogue is administered between 18 hours and 30 hours after administration of the about 1.0 mg to about 5.0 mg of the relaxin analogue. In various embodiments, the yet additional dose of the relaxin analogue is administered between 20 hours and 26 hours after administration of the dose from about 1.0 mg to about 10.0 mg of the relaxin analogue. In various embodiments, the yet additional dose of the relaxin analogue is administered about 24 hours after administration of the dose from about 1.0 mg to about 10.0 mg of the relaxin analogue. In various embodiments, the yet additional dose of the relaxin analogue is administered subcutaneously to the individual. In various embodiments, the yet additional dose of the relaxin analogue comprises from about 1 mg to about 50 mg of the relaxin analogue. In various embodiments, the yet additional dose of the relaxin analogue comprises from about 2 mg to about 15 mg of the relaxin analogue. In various embodiments, the yet additional dose of the relaxin analogue comprises from about 3 mg to about 8 mg of the relaxin analogue. In various embodiments, the yet additional dose of the relaxin analogue comprises about 5 mg of the relaxin analogue. In various embodiments, the yet additional dose of the relaxin analogue comprises from about 2 mg to about 5 mg of the relaxin analogue. In various embodiments, the yet additional dose of the relaxin analogue comprises about 2.5 mg of the relaxin analogue. In various embodiments, the yet additional dose of the relaxin analogue comprises from about 6 mg to about 15 mg of the relaxin analogue. In various embodiments, the yet additional dose of the relaxin analogue comprises from about 8 mg to about 12 mg of the relaxin analogue. In various embodiments, the yet additional dose of the relaxin analogue comprises about 10 mg of the relaxin analogue.

In various embodiments, methods disclosed herein further comprise administering daily the yet additional dose of the relaxin analogue. In various embodiments, the yet additional dose of the relaxin analogue is administered daily from about 10 days to about 20 days. In various embodiments, the yet additional dose of the relaxin analogue is administered daily from about 12 days to about 16 days. In various embodiments, the yet additional dose of the relaxin analogue is administered daily for about 14 days or more. In various embodiments, the yet additional dose of the relaxin analogue is administered daily from about 3 days to about 15 days. In various embodiments, the yet additional dose of the relaxin analogue is administered daily from about 4 days to about 13 days. In various embodiments, the yet additional dose of the relaxin analogue is administered daily from about 5 days to about 11 days. In various embodiments, the yet additional dose of the relaxin analogue is administered daily from about 6 days to about 9 days.

In various embodiments, the relaxin analogue has an EC₅₀ for activation RXFP1 in the in vitro OVCAR5 cAMP assay of less than 15 nM, less than 1 nM, less than 0.5 nM, or less than 0.1 nM. In various embodiments, the vasopressin analogue is a V1a receptor agonist. In various embodiments, the vasopressin analogue is terlipressin or a pharmaceutically acceptable salt thereof. In various embodiments, the terlipressin is administered intravenously at a dosage from about 0.5 to about 10 mg. In various embodiments, the terlipressin is administered intravenously at a dosage from about 0.5 to about 2 mg every 4 to 6 hours. In various embodiments, the terlipressin is administered intravenously at a dosage of about 1 mg every 6 hours. In various embodiments, the terlipressin is administered intravenously at a dosage from about 2 to about 6 mg every 4 to 6 hours. In various embodiments, the terlipressin is administered intravenously at a dosage of about 4 mg every 6 hours. In various embodiments, the terlipressin is administered intravenously at a dosage from about 6 to about 10 mg every 4 to 6 hours. In various embodiments, the terlipressin is administered intravenously at a dosage of about 6 mg every 6 hours. In various embodiments, the terlipressin is administered intravenously at a dosage of about 8 mg every 6 hours. In various embodiments, terlipressin is administered intravenously via a bolus injection. In various embodiments, terlipressin is administered intravenously via a bolus injection over from about 1 minute to about 5 minutes. In various embodiments, terlipressin is administered intravenously via a bolus injection over from about 2 minutes to about 3 minutes. In various embodiments, terlipressin is administered intravenously via a bolus injection over about 2 minutes. In various embodiments, the terlipressin is administered intravenously at a dosage from about 1 mg to about 10 mg. In various embodiments, the terlipressin is administered intravenously at a dosage from about 1.5 mg to about 4 mg. In various embodiments, the terlipressin is administered intravenously at a dosage of about 2 mg. In various embodiments, the terlipressin is administered intravenously at a dosage from about 5 mg to about 8 mg. In various embodiments, the terlipressin is administered intravenously at a dosage of about 6 mg. In various embodiments, the terlipressin is administered intravenously over from about 10 hours to about 30 hours. In various embodiments, the terlipressin is administered intravenously over from about 20 hours to about 25 hours. In various embodiments, the terlipressin is administered intravenously over about 24 hours.

In various embodiments, methods disclosed herein further comprise administering midodrine or octreotide to the individual. In various embodiments, methods disclosed herein further comprise administering albumin to the individual. In various embodiments, the dose of the relaxin analogue and the vasopressin analogue are administered simultaneously. In various embodiments, the dose of the relaxin analogue and the vasopressin analogue are administered in a single composition. In various embodiments, the dose of the relaxin analogue and the vasopressin analogue are administered in separate compositions. In various embodiments, the dose of the vasopressin analogue and the relaxin analogue are administered sequentially. In various embodiments, the combination therapy has a synergistic therapeutic effect.

In various embodiments, the combination therapy achieves an improved response rate incidence, wherein responders are defined according to at least International Club of Acites (ICA) criteria. In various embodiments, responders comprise full or partial responders, as defined according to ICA criteria, and are alive without renal replacement therapy (RRT) for at least 30 days after start of treatment. In various embodiments, responders comprise full or partial responders, as defined according to ICA criteria, and are alive without renal replacement therapy (RRT) for at least 10 days after start of treatment. In various embodiments, full responders are defined as two serum creatinine levels returning to a value within 0.3 mg/dL (26.5 micromolar/L) of a baseline serum creatinine value at least 2 hours apart. In various embodiments, partial responders are defined as a regression of at least 1 acute kidney injury (AKI) stage with a reduction of serum creatinine greater than or equal to 0.3 mg/dL above a baseline serum creatinine value. In various embodiments, the combination therapy achieves an improved response rate incidence, wherein response rate incidence is measured according to a return of serum creatinine to a value within 0.3 mg/dL (26.5 micro molar/L) of a baseline value. In various embodiments, the combination therapy achieves an improved response rate incidence, wherein response rate incidence is measured according to a regression of acute kidney injury (AKI) stage with a reduction of serum creatinine greater than or equal to 0.3 mg/dL above a baseline value. In various embodiments, the combination therapy achieves an improved response rate incidence, wherein response rate incidence is measured according to two consecutive serum creatinine values at least 2 hours apart being below 1.5 mg/dL. In various embodiments, the administration of the relaxin analogue mitigates adverse effects associated with vasopressin analogue treatment in the individual. In various embodiments, the administration of the vasopressin analogue reduces risk of hypotension in the individual associated with treatment with the relaxin analogue. In various embodiments, the administration of the vasopressin analogue increases renal pressure in the individual.

Additionally disclosed herein is a method of preventing or treating renal failure in an individual in need thereof, comprising: A) intravenously administering to the individual about a 4.0 mg dose of a relaxin analogue; B) on a same day that step (A) is performed, subcutaneously administering to the individual about a 5.0 mg dose of the relaxin analogue; and C) on a subsequent day different from the day that steps (A) and (B) are performed, subcutaneously administering, to the individual, about a 10 mg dose of the relaxin analogue.

Additionally disclosed herein is a method of preventing or treating hepatorenal syndrome in an individual in need thereof, comprising: A) intravenously administering to the individual about a 4.0 mg dose of a relaxin analogue; B) on a same day that step (A) is performed, subcutaneously administering to the individual about a 5.0 mg dose of the relaxin analogue; and C) on a subsequent day different from the day that steps (A) and (B) are performed, subcutaneously administering, to the individual, about a 10 mg dose of the relaxin analogue. In various embodiments, methods disclosed herein further comprise repeating step (c) on a daily basis for up to 13 days. In various embodiments, methods disclosed herein further comprise: on the same day that step (A) and step (b) are performed, intravenously administering a 1 mg bolus of terlipressin every 6 hours. In various embodiments, methods disclosed herein further comprise: repeating intravenously administering a 1 mg bolus of terlipressin every 6 hours for up to 14 days.

### BRIEF DESCRIPTION OF THE DRAWINGS

FIGS. 1 and 2 are the two parts of a schema representing the general method used for synthesizing relaxin peptide analogues.

FIGS. 3A and 3B show the change from baseline effective renal plasma flow following administration of Relaxin Agonist.

FIG. 4 depicts an overall study design of a Phase II Trial Assessing Safety, Tolerability, Efficacy, and Pharmacokinetics of Relaxin Agonist in Combination with Terlipressin.

FIG. 5 depicts a design of the open label safety run-in part of the overall study design shown in FIG. 4.

FIG. 6 depicts a design of the single-blind placebo-controlled randomized treatment part of the overall study design shown in FIG. 4.

FIG. 7 depicts a design of the open-label terlipressin non-responder part of the overall study design shown in FIG. 4.

### DEFINITIONS

As used herein, the term “X_{y}” in relaxin peptide analogue formulas is used with y having different values represents an amino acid as defined in the definition of said formulae. y indicates the position of said amino acid in the native B-chain of relaxin-2. For example, X₁₀ represents the amino acid in position 10 of the amino acid sequence of the native B-chain of relaxin-2.

As used herein, the term “pharmaceutically acceptable carrier” is intended for a fluid, especially a liquid comprising a pharmaceutical compound or combination of pharmaceutical compounds of the invention, such that the pharmaceutical composition is physiologically tolerable, i.e., can be administered to the individual body without toxicity or undue discomfort.

As used herein, the term a “relaxin analogue” refers to a peptide or other compound that is a functional variant of relaxin, e.g., that is able to activate the RXFP1 receptor, and/or a structural analogue of native relaxin-2, e.g., a modified relaxin B chain peptide. In addition to modified relaxin peptides, relaxin analogues can include small molecules that are RXFP1 receptor agonists or display RXFP1 agonistic properties. In some embodiments, relaxin analogues can also include other modalities such as relaxin coupled to Fc fragments, and RXFP1 agonists such as nanobodies, or monoclonal antibodies.

As used herein, the term a “vasopressin analogue” refers to prodrugs of vasopressin, and peptides structurally similar to vasopressin and/or functional variants of vasopressin, e.g., terlipressin (triglycyl lysine vasopressin), argipressin, desmopressin, felypressin, lypressin, or ornipressin. Vasopressin analogues can activate vasopressin receptors Via, V2, and/or V3 (also called V1b). Small molecule vasopressin analogues are also known to those of ordinary skill in the art and are included within the term ‘vasopressin analogue,’ as used herein.

The term “agonist” in the present context refers to a peptide or small molecule as defined herein, capable of binding to and activating a receptor. Full agonists bind to and activate a receptor with the maximum response that an agonist can elicit at the receptor. Partial agonists also bind and activate a given receptor but have partial efficacy at the receptor relative to a full agonist, even at maximal receptor occupancy. A selective agonist is selective for a specific type or subtype of receptor.

A “functional variant” of a peptide is a peptide capable of performing essentially the same functions as the peptide it is a functional variant of. In particular, a functional variant can bind the same molecules, preferably with a similar affinity, as the peptide it is a functional variant of.

As used herein, the term “native” as used in the present text in connection with relaxin refers to naturally occurring or wild type molecules.

As used herein, the term “Preventing” is intended to mean reducing the risk of manifestation of the phenomenon under consideration. This reduction may be total or partial, i.e., results in a degree of risk that is lower than that pre-existing the use according to the invention.

As used herein, the term “treating” is intended to mean reducing or even eliminating the undesirable condition or disease under consideration. A “treatment effect” or “therapeutic effect” is manifested if there is a change in the condition being treated, as measured by the criteria constituting the definition of the terms “treating” and “treatment.” There is a “change” in the condition being treated if there is at least 5% improvement, preferably 10% improvement, more preferably at least 25%, even more preferably at least 50%, such as at least 75%, and most preferably at least 100% improvement. The change can be based on improvements in the severity of the treated condition in an individual, or on a difference in the frequency of improved conditions in populations of individuals with and without treatment with the bioactive agent or bioactive agents. A treatment according to the invention can be prophylactic, ameliorating and/or curative.

A “bioactive agent” (i.e., a biologically active substance/agent) is any agent, drug, compound, composition of matter or mixture which provides some pharmacologic, often beneficial, effect that can be demonstrated in vivo or in vitro. It refers to the peptide sequences defined herewith, compounds or compositions comprising these and nucleic acid constructs encoding said peptides. As used herein, this term further includes any physiologically or pharmacologically active substance that produces a localized or systemic effect in an individual. A ‘bioactive agent’ as used herein denotes collectively a peptide, a nucleic acid construct encoding said peptide, and a composition comprising a peptide.

“Pharmacologically effective amount”, “pharmaceutically effective amount”, “physiologically effective amount”, or “effective amount” of a “bioactive agent” is the amount of a bioactive agent or combination of bioactive agents present in one or more pharmaceutical compositions as described herein that is needed to provide a desired level of active agent or agents in the bloodstream or at the site of action in an individual (e.g. the hepatic system, the renal system, the circulatory system, the lungs, the gastrointestinal system, the colorectal system, etc.) to be treated to give an anticipated physiological response when such composition is administered.

“Co-administering” or “co-administration” as used herein refers to the administration of two or more bioactive agents. The two or more components can be administered separately, sequentially, or simultaneously.

The term “individual” refers to vertebrates, particular members of the mammalian species, preferably primates including humans. As used herein, ‘subject’ and ‘individual’ may be used interchangeably. Treatment of animals, such as mice, rats, dogs, cats, cows, horses, sheep and pigs, is, however, also within the scope of the present invention.

As used herein, an “individual in need thereof” refers to an individual who may benefit from treatment. In one embodiment, said individual in need thereof is a diseased individual, wherein said disease may be a renal disorder.

Reference to “about” a value or parameter herein includes (and describes) embodiments that are directed to that value or parameter per se. For example, description referring to “about X” includes description of “X.”

As used herein, the singular form of the articles “a,” “an,” and “the” includes plural references unless indicated otherwise.

It is understood that aspects and embodiments of the invention described herein include “comprising,” “consisting,” and/or “consisting essentially of” aspects and embodiments.

### DETAILED DESCRIPTION

### Vasopressin Analogue and Relaxin Analogue Combination Therapy

Disclosed herein, according to some embodiments, is a method of preventing or treating a renal disorder, e.g., hepatorenal syndrome (HRS), in an individual in need thereof, comprising co-administering to the individual a relaxin peptide analogue and a vasopressin analogue as bioactive agents.

A vasopressin analogue, such as terlipressin, functions as a vasoconstrictor. Terlipressin causes vasoconstriction in the splanchnic circulation, and also in the systemic circulation. As discussed in the background, terlipressin has a poor safety profile. For example, as described in Snowdon, Victoria K et al. “Serelaxin as a potential treatment for renal dysfunction in cirrhosis: Preclinical evaluation and results of a randomized phase 2 trial.” PLoS medicine vol. 14, 2 e1002248. 28 Feb. 2017, “splanchnic vasoconstrictors such as terlipressin . . . further compromise hepatic perfusion in cirrhosis and may impair organ function.” Terlipressin has limited selectivity for splanchnic vasoconstriction over systemic vasoconstriction including hepatic and renal vasoconstriction, which would further exacerbate HRS. In general, use of splanchnic vasoconstrictors such as terlipressin can result in various adverse effects that could compromise kidney function and patient safety, and is therefore not optimal for treatment of renal dysfunction in cirrhosis.

A relaxin analogue, such as a long-lasting peptidyl RXFP1 agonist disclosed herein, functions as a vasodilator with a degree of functional selectivity for renal vasodilation.

The combination therapy of a relaxin analogue and vasopressin analogue herein relies on additive and synergistic interaction between a vasoconstrictor (e.g., a vasopressin analogue, such as terlipressin) and a vasodilator (e.g., a relaxin analogue) acting at different regional vascular beds (primarily splanchnic for terlipressin and primarily renal for a relaxin analogue) to improve safety and effectiveness of treatment of renal conditions associated with renal vasoconstriction in an individual as described herein.

Disclosed herein, according to some embodiments, is a pharmaceutical composition comprising, separately or together, a relaxin peptide analogue and a vasopressin analogue, for use in a method of treating a renal disorder, for example HRS, such as hepatorenal syndrome type 1 (HRS-AKI) and type 2 (HRS-NAKI), or preserving renal function in peri-operative liver transplantation.

In some embodiments, also disclosed herein is a pharmaceutical composition comprising, separately or together, a peptide analogue of the B-chain of relaxin-2 able to activate the RXFP1 receptor and a vasopressin analogue able to activate the vasopressin V1 (V1a) receptor, for use in a method of treating a renal disorder, such as HRS, e.g., hepatorenal syndrome type 1 (HRS-AKI) and type 2 (HRS-NAKI), or preserving renal function in peri-operative liver transplantation.

Disclosed herein, according to some embodiments, is a pharmaceutical composition comprising, separately or together, a relaxin peptide analogue and terlipressin, for use in a method of treating a renal disorder, including HRS, such as hepatorenal syndrome type 1(HRS-AKI) and type 2 (HRS-NAKI), or preserving renal function in peri-operative liver transplantation.

In some embodiments, also disclosed herein is a pharmaceutical composition comprising, separately or together, a peptide analogue of the B-chain of relaxin-2 able to activate the RXFP1 receptor and terlipressin, for use in a method of treating a renal disorder, including HRS, such as hepatorenal syndrome type 1 (HRS-AKI) and type 2 (HRS-NAKI), or preserving renal function in peri-operative liver transplantation.

In some embodiments, also disclosed herein is a method of treating a renal disorder, including hepatorenal syndrome type 1 (HRS-AKI) and type 2 (HRS-NAKI), comprising administering a therapeutically effective amount of a composition comprising, separately or together, a relaxin peptide analogue, such as peptide analogue of the B-chain of relaxin-2 able to activate the RXFP1 receptor, and a vasopressin analogue, such as terlipressin able to activate the V1 (V1a) receptor, to an individual in need thereof.

In one embodiment, the renal disorder is renal dysfunction in cirrhosis, including HRS, such as hepatorenal syndrome type 1 (HRS-AKI) and type 2 (HRS-NAKI), and/or peri-operative liver transplantation, chronic kidney disease and acute kidney injury.

Vasopressin Analogues, such as Terlipressin

As described herein, vasopressin analogues, such as terlipressin, are used in combination with relaxin analogues for treatment of disorders associated with renal dysfunction. In some embodiments, a vasopressin analogue is terlipressin, argipressin, desmopressin, felypressin, lypressin, or ornipressin.

In some embodiments, the vasopressin analogue is terlipressin, or a pharmaceutically acceptable salt of terlipressin. Terlipressin (also known as triglycyl lysine vasopressin) is a synthetic analogue of the human neuropeptide hormone vasopressin. Terlipressin is a prohormone of lysine-vasopressin (triglycyl lysine vasopressin TGLVP), for example as described in Rittig et al., Movement Disorders, 1991, Vol. 6(1), p 21-28. Following administration and absorption to the circulation, the glycyl residues are cleaved from the prohormone by endothelial peptidases, allowing release of lysine-vasopressin. Thus, terlipressin itself has weak intrinsic vasopressive activity but is transformed to the fully active lysine vasopressin (LVP) by endothelial endopeptidases. Terlipressin is also known by its tradenames Teripress and Glypressin. Terlipressin has a molecular weight of 1227.37 g/mol and is represented by the formula:

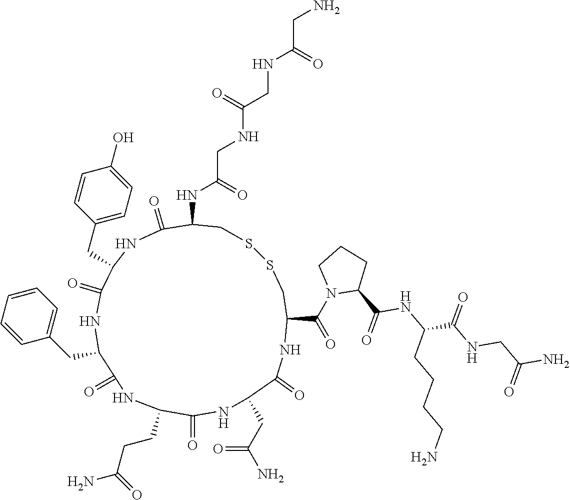 [SMILES: NCCCC[C@H](NC(=O)[C@@H]1CCCN1C(=O)[C@@H]1CSSC[C@H](NC(=O)CNC(=O)CNC(=O)CN)C(=O)N[C@@H](Cc2ccc(O)cc2)C(=O)N[C@@H](Cc2ccccc2)C(=O)N[C@@H](CCC(N)=O)C(=O)N[C@@H](CC(N)=O)C(=O)N1)C(=O)NCC(N)=O]

Terlipressin administered intravenously has been used, for example as a vasoactive drug in the management of hypotension (low blood pressure) and for example for treatment of bleeding esophageal varices, septic shock, HRS and ascites.

Outside the United States, individuals with cirrhosis exhibiting type 1 hepatorenal syndrome (HRS-1) have been treated with terlipressin administered by continuous IV infusion. Dosage ranged from 2.0-12.0 mg per 24 hours (Angeli, et al., (2009) Journal of Hepatology, 50:S73: 2.0-12.0 mg/24h; Gerbes, (2009) Gastroenterology, 137:1 179-1 189: starting dose 3.0 mg/day; Robertson, et al., (2014) Hepatology, 60(6):21 25-21 26: 3.0 mg/day; Ding, et al., (2013), Gastroenterology and Hepatology, 28:1242-1246: 4.0 mg/day; Cavallin, et al., (2015), Hepatology, 62(2):567-574: 3-12 mg/day).

Currently terlipressin is available in two forms: lyophilized powder for reconstitution or as a liquid (0.2 milligrams/mL) in vials. The lyophilized version is typically supplied in vials containing 1 mg terlipressin powder for reconstitution using the provided ampoule of 5 mL of saline solution (e.g., Glypressin, Ferring Pharmaceuticals is supplied as one vial containing 1 mg terlipressin acetate for reconstitution in 5 mL solution) to deliver 0.17 mg/mL terlipressin (0.2 mg/mL terlipressin acetate) solution for injection. Administering this product requires two or three prior steps: reconstituting the powder with diluent, withdrawing the solution, and possible further dilution, then injection by slow bolus dose directly into the patient or into the patient's intravenous line or IV bag. Glypressin requires refrigerated storage at a temperature of 2° C. to 8° C.

The liquid, terlipressin acetate 0.2 milligrams/mL solution for injection (Terlipressin acetate, Ever Pharma) is also not stable at room temperature (RT) and requires refrigerated storage at a temperature of 2° C. to 8° C. It is supplied in vials containing 5 mL or 10 mL of solution. This solution is withdrawn into a syringe for administration via bolus injection. Current formulations use acetic acid to adjust the pH of the terlipressin acetate solution.

One aspect of the disclosure provides an aqueous composition comprising terlipressin acetate or a pharmaceutically acceptable salt thereof, wherein the composition comprises from about 0.2 to about 10.0 mg/ml terlipressin acetate or a pharmaceutically acceptable salt thereof, wherein the pH of the composition is from about 3.4 to about 5.0.

As used herein, the term “aqueous” refers to a solution in which the solvent is water. The solvent can be sterile water suitable for injection. In one embodiment, the solvent can be bacteriostatic water. In other embodiments, the solvent can be mixtures of water with other pharmaceutically acceptable solvents or pharmaceutically acceptable alcohols or other bacteriostatic agents (e.g., benzyl alcohol).

The concentration of terlipressin in the liquid (e.g., aqueous composition) can be, for example, 0.2 mg/ml, 0.5 mg/ml, 1.0 mg/ml, 2.0 mg/ml, 5.0 mg/ml, or 10.0 mg/ml. In some embodiments, the composition comprises from about 0.2 mg/ml to about 10.0 mg/ml, about 0.5 mg/ml to about 10.0 mg/ml, about 1.0 mg/ml to about 9.0 mg/ml, about 1.5 mg/ml to about 8.5 mg/ml, from about 2.0 mg/ml to about 8.0 mg/ml, about 2.5 mg/ml to about 7.5 mg/ml, about 3.0 mg/ml to about 7.0 mg/ml, about 3.5 mg/ml to about 6.5 mg/ml, about 0.5 mg/ml to about 2.0 mg/ml, or about 0.5 mg/ml to about 1.0 mg/ml, terlipressin acetate or a pharmaceutically acceptable salt thereof.

In some embodiments, the pH of the composition can be from about 3.4 to about 5.0, from about 3.5 to about 5.0, from about 3.6 to about 5.0, from about 3.7 from about 5.0, from about 3.8 to about 5.0, from about 3.9 to about 5.0, from about 4.0 to about 5.0, from about 4.1 to about 5.0, from about 4.2 to about 5.0, from about 4.3 to about 5.0, from about 4.4 to about 5.0, from about 4.5 to about 5.0, from about 4.6 to about 5.0, from about 4.7 to about 5.0, from about 4.8 to about 5.0, or from about 4.9 to about 5.0. In other embodiments, the pH of the composition can be about 3.4, 3.5, 3.6, 3.7, 3.8, 3.9, 4.0, 4.1, 4.2, 4.3, 4.4, 4.5, 4.6, 4.7, 4.8, 4.9, 5.0, or 5.1.

Further information on terlipressin formulations and administration can be found, e.g., in PCT Publication No. WO2020/237170 “Formulations of Terlipressin” incorporated by reference in its entirety.

The composition can be for any route of drug administration, e.g., buccal, nasal, transdermal (e.g., patch technology), parenteral, intravenous, intramuscular or subcutaneous injection, intracisternal, intraperitoneal. In some embodiments, the composition is for intravenous administration, e.g., by continuous infusion or by a bolus IV dose.

In some embodiments, the methods described herein provide about 1 to about 1000 μg/mL terlipressin concentration in a subject. In some embodiments, the methods described herein provide about 10 to about 600 μg/mL terlipressin concentration in a subject. In some embodiments, the methods described herein provide about 20 to about 200 μg/mL terlipressin concentration in a subject. In some embodiments, the methods described herein provide about 60 μg/mL terlipressin concentration in a subject. In some embodiments, the methods described herein provide about 1 to about 200 μg/mL (lysine-)vasopressin concentration in a subject; where (lysine-)vasopressin is the active metabolite of terlipressin.

In some embodiments, the methods described herein provide about 2 to about 100 μg/mL vasopressin analogue concentration in a subject, e.g., in the plasma of the subject. In some embodiments, the methods described herein provide about 3 to about 30 μg/mL vasopressin analogue concentration in a subject, e.g., in the plasma of the subject. In some embodiments, the methods described herein provide about 10 μg/mL vasopressin analogue concentration in a subject, e.g., in the plasma of the subject.

In some embodiments, the method comprises administration up to 2 times per day. In some embodiments, the method comprises administration up to 3 or 4 times per day. The dosage regimen utilizing the compounds is selected in accordance with a variety of factors including age, weight, sex and medical condition of the patient; the severity of the condition to be treated; the route of administration; the renal and hepatic function of the patient; and the particular compound or salt thereof employed. An ordinarily skilled physician can readily determine and prescribe the effective amount of the drug required to prevent, counter or arrest the progress of the condition.

Another aspect of this invention is a method for treating a subject suffering from ascites (e.g., caused by liver cirrhosis), the method comprising administration to the subject of a therapeutically effective amount of a vasopressin analogue, such as terlipressin and a relaxin analogue. Optionally such treatment is given in combination with another drug used for treatment of ascites. An aspect of the invention is a method of treating a subject suffering from ascites, the method comprising administration to the subject of a therapeutically effective amount of a vasopressin analogue, such as terlipressin, and a relaxin analogue.

### Relaxin Analogues

Relaxin analogues or pharmaceutically acceptable salts thereof are useful in combination with vasopressin analogues, such as terlipressin, in the present invention. In some embodiments, the relaxin analogue is a long-acting, relaxin peptide analogue that functions as an RXFP1 receptor agonist. The disclosure further relates to compositions including the same and their use in treating renal conditions, diseases or disorders in combination with vasopressin analogues.

When applicable, amino acids in the relaxin peptide analogues disclosed herein can each independently be L-amino acids or D-amino acids. In a particular embodiment, amino acids are L-amino acids. In the present text, if no information is indicated regarding the L- or D-form of a given amino acid, then this amino acid is an L-amino acid. Nₜₑᵣ and Cₜₑᵣ are conventional labels used to indicate, respectively, the N-terminal end of the peptide and the C-terminal end of the relaxin peptide analogues.

### Peptide Structural Analogues

In some embodiments, a relaxin peptide analogue is a modified relaxin-2 B chain peptide.

In some embodiments, the modified relaxin B chain peptide comprises formula (I) (SEQ ID NO 105):

Nₜₑᵣ-Ac-X₁₀-E-G-R-E-X₁₅-V-R-X₁₈-X₁₉-I-X₂₁-X₂₂-E-G-X₂₅-
S-X₂₇-X₂₈-X₂₉-X₃₀-X₃₁-X₃₂-X₃₃-NH₂-Cₜₑᵣ,

wherein:
Nₜₑᵣ represents the N-terminal end of the peptide;
Cₜₑᵣ represents the C-terminal end of the peptide;
Ac represents acetyl group;
X₁₀ represents an amino acid selected from the group consisting of leucine, 2-amino-isobutyric acid, Nε-acetyl-lysine and α-methyl-leucine;
E represents glutamic acid;
G represents glycine;
R represents arginine;
X₁₅ represents an amino acid selected form the group consisting of lysine, arginine, homolysine, homoarginine, ornithine, glutamine, phenylalanine and leucine;
V represents valine;
X₁₈ represents an amino acid selected from the group consisting of alanine, 2-amino-isobutyric acid, leucine, Nε-acetyl-lysine and glutamine;
X₁₉ represents an amino acid selected from the group consisting of lysine, Nε-acetyl-lysine, citrulline, glutamine, alanine and 2-amino-isobutyric acid;
I represents isoleucine;
X₂₁ represents an amino acid selected from the group consisting of 2-amino-isobutyric acid and alanine;
X₂₂ represents an amino acid selected from the group consisting of 2-amino-isobutyric acid and isoleucine;
X₂₅ represents the following structure:

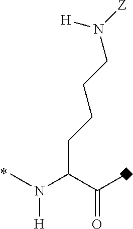

in which:
* represents a covalent bond with the glycine preceding X₂₅ in formula (I);
♦ represents a covalent bond with the serine following X₂₅ in formula (I); and
Z represents a group of formula (II):

-[(PEGₓₓ)_{b}(gE)_{c}C_{d}],
b and c independently represent 1, 2, 3, 4 or 5;
PEGₓₓ independently represents a polyethylene glycol derivative selected from the group consisting of PEG₂, PEG₂DGA, and TTDS;
gE represents γ-glutamic acid; and
C_{d} represents a linear saturated C12-C22 acyl group;
S represents serine;
X₂₇ represents an amino acid selected from the group consisting of threonine, lysine, arginine and glutamine;
X₂₈ represents an amino acid selected from the group consisting of tryptophan, phenylalanine, 5-fluoro-tryptophan, 5-chloro-tryptophan, 5-methoxy-tryptophan, tyrosine, 4-fluoro-phenylalanine, 1-naphtylalanine, 2-naphtylalanine, α-methyl-tryptophan, a-methyl-phenylalanine and 5-hydroxy-tryptophan;
X₂₉ represents an amino acid selected from the group consisting of serine, D-serine,
2-amino-isobutyric acid, threonine, α-methyl-serine, Nε-acetyl-lysine and valine;
X₃₀ represents an amino acid selected from the group consisting of 2-amino-isobutyric acid, α-methyl-lysine, D-lysine, lysine, homolysine, ornithine, arginine and α-methyl-arginine;
X₃₁ represents an amino acid selected from the group consisting of arginine, Nω-methyl-arginine, alanine, Nω,Nω′-dimethyl-arginine and citrulline;
X₃₂ represents an amino acid selected from the group consisting of lysine, alanine, arginine, Nε-acetyl-lysine and Nε,Nε,Nε-tri-methyl-lysine; and
X₃₃ represents an amino acid selected from lysine, Nε-acetyl-lysine, leucine, arginine and alanine;
or a salt or solvate thereof.

The relaxin peptide analogues disclosed herein also includes salts of the peptides of the formulae (I) or (Ia) defined herein, in one embodiment pharmaceutically acceptable salts, for example salts as acid adduct with inorganic acids such as hydrochloric acid, sulphuric acid, nitric acid, hydrobromic acid, phosphoric acid, perchloric acid, thiocyanic acid and boric acid; or with organic acid such as formic acid, acetic acid, trifluoroacetic acid, propionic acid, glycolic acid, citric acid, tartaric acid, succinic acid, gluconic acid, lactic acid, malonic acid, fumaric acid, anthranilic acid, benzoic acid, cinnamic acid, benzenesulfonic acid, p-toluenesulfonic acid, naphthalenesulfonic acid and sulfanilic acid; and salts with metals such as alkali metal, e.g. sodium, potassium, lithium, zinc, and aluminium.

In one embodiment the salts of the peptides are pharmaceutically acceptable salts, for example acid adducts with hydrochloric acid, sulphuric acid, phosphoric acid, formic acid, acetic acid, propionic acid, glycolic acid, citric acid, tartaric acid, succinic acid, gluconic acid, lactic acid, malonic acid, fumaric acid, anthranilic acid, benzoic acid, cinnamic acid, benzenesulfonic acid, p-toluenesulfonic acid, naphthalenesulfonic acid and sulfanilic acid; and salts with metals such as alkali metal, e.g. sodium, potassium, lithium and zinc.

The relaxin peptide analogues disclosed herein also includes solvates, and in one embodiment pharmaceutically acceptable solvates, of the peptides of the above formulae (I) or (Ia).

Solvates mean complexes of the compounds of the relaxin peptide analogues or salts thereof with solvent molecules, e.g., organic solvent molecules and/or water.

In keeping with standard polypeptide nomenclature (J. Biol. Chem., 243:3552-59 (1969)) abbreviations for α-amino acid residues are as follows:

| One letter code | Three letter code | Amino acid |
|---|---|---|
| A | Ala | Alanine |
| C | Cys | Cysteine |
| D | Asp | Aspartic acid or aspartate |
| E | Glu | Glutamic acid or glutamate |
| F | Phe | Phenylalanine |
| G | Gly | Glycine |
| H | His | Histidine |
| I | Ile | Isoleucine |
| K | Lys | Lysine |
| L | Leu | Leucine |
| M | Met | Methionine |
| N | Asn | Asparagine |
| P | Pro | Proline |
| Q | Gln | Glutamine |
| R | Arg | Arginine |
| S | Ser | Serine |
| T | Thr | Threonine |
| V | Val | Valine |
| W | Trp | Tryptophan |
| Y | Tyr | Tyrosine |

For non natural or modified amino acids the following abbreviations are used:

| | |
|---|---|
| Aib | 2-amino-isobutyric acid |
| Cit | Citruline |
| Dser | D-serino |
| Dlys | D-lysine |
| Har | Homoarginine |
| Hly | Homolysine |
| Hph | Homophenylalanine |
| gE | γ-Glutamic acid (glutamic acid connected by its γ- |
| | carboxylic acid function) |
| Nle | Norleucine, 2-amino hexanoic acid |
| K(Ac) | Nε-Acetyl-lysine, 2-amino-4-acetamido-hexanoic acid |
| Mar | α-Methyl-arginine |
| Mel | α-Methyl-leucine |
| Mly | α-Methyl-lysine |
| Mph | α-Methyl-phenylalanine |
| Mse | α-Methyl-serine |
| Mtr | α-Methyl-tryptophane |
| 1-Nal | 1-Naphtylalanine |
| 2-Nal | 2-Naphtylalanine |
| Orn | Ornithine |
| Pfp | 4-Fluoro-phenylalanine |
| Rds | Nω,Nω′-dimethyl-arginine (symmetrical) |
| Rme | Nω-methyl-arginine |
| Tml | Nε,Nε,Nε-trimethyl-lysine |
| Trp(5-Cl) or | 5-Chlorotryptophan |
| Trp(5Cl) | |
| Trp(5-F) or | 5-Fluorotryptophan |
| Trp(5F) | |
| Trp(5-OMe) or | 5-Methoxytryptophan |
| Trp(5OMe) | |
| Wox | 5-Hydroxy-tryptophane |

In all the formulae of relaxin peptide analogues, where the amino acid sequence is represented by using the above-mentioned abbreviations and X_{y} representations such as X₁₈ for example, the left and right orientation is in the conventional direction of amino-terminus to carboxy-terminus.

Accordingly, for example, with X₂₈ representing an amino acid selected from the group of tryptophan, 5-fluoro-tryptophan, 5-chloro-tryptophan, 5-methoxy-tryptophan, tyrosine, phenylalanine, 4-fluoro-phenylalanine, 1-naphtylalanine, 2-naphtylalanine, a-methyl-tryptophan, α-methyl-phenylalanine and 5-hydroxy-tryptophan, the N-terminus or amine group of said amino acid is linked to the amino acid represented by X₂₇ and the C-terminus or carboxyl group of said amino acid is linked to the amino acid represented by X₂₉.

On its N-terminal (Nₜₑᵣ) extremity, a relaxin peptide analogue is substituted with an acetyl group (Ac): CH₃C(O)—.

On its C-terminal (Cₜₑᵣ) extremity, a relaxin peptide analogue is substituted with an —NH₂ group.

On its position X₂₅, a relaxin peptide analogue comprises the following structure:

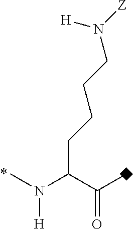

This structure corresponds to a lysine amino acid wherein:
the alpha (α) nitrogen atom (*) is bound to the previous part of the peptide, i.e., with the group in Nₜₑᵣ of X₂₅ on the basis of the Nₜₑᵣ-Cₜₑᵣ orientation represented in the relaxin peptide analogue formulae, through a covalent bond, for example through a peptide bond, i.e., with the glycine in position 24 (X₂₄);
the carbon atom of the carboxyl group (♦) is bound to the part of the peptide following position X₂₅, i.e., with the group in Cₜₑᵣ of X₂₅ on the basis of the Nₜₑᵣ-Cₜₑᵣ orientation represented in the relaxin peptide analogue formulae, through a covalent bond, for example through a peptide bond, i.e., with the serine in position 26 (X₂₆);
and the nitrogen atom of its lateral chain is bound to a Z group.

This Z group is defined as being of formula (II):

-[(PEGₓₓ)_{b}(gE)_{c}C_{d}],

In formula (II), the — represents a covalent bond with the nitrogen atom of the lateral chain of the lysine structure in X₂₅

b and c independently represent 1, 2, 3, 4 or 5, in particular 2, 3, 4 or 5.

In a particular embodiment, b represents 2, 3, 4 or 5.

In a particular embodiment, c represents 2, 3 or 4.

In a preferred embodiment, b represents 2, 3, 4 or 5 and c independently represents 2, 3 or 4.

PEGₓₓ in the relaxin peptide analogue formulae independently represents a polyethylene glycol derivative selected from the group consisting of PEG₂, PEG₂DGA and TTDS

Said groups are defined as follows:

| Abbreviation | Structure | IUPAC name |
|---|---|---|
| PEG₂ | 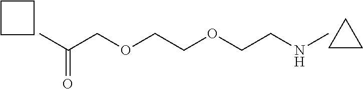 | 2-[2-(2- aminoethoxy) ethoxy] acetyl |
| PEG₂DGA | 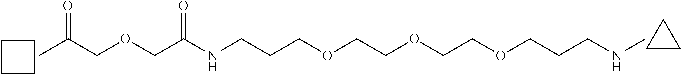 | 2-[2-[3-[2-[2-(3- aminopropoxy) ethoxy] ethoxy] propylamino]-2-oxo- |
| | | ethoxy]acetyl |
| TTDS | 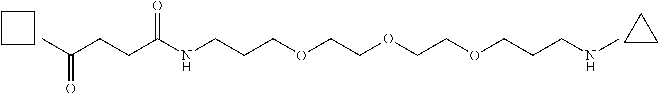 | 4-[3-[2-[2-(3- aminopropoxy) ethoxy] ethoxy] propylamino]- 4-oxo-butanoyl |

in which, in (PEGₓₓ)_{b} of formula (II), represents a covalent bond with the (gE), group and represents a covalent bond linking the group of formula (II) with the nitrogen atom of lateral chain of the lysine structure in X₂₅.

In a particular embodiment, (PEGₓₓ)_{b} represents a polyethylene glycol derivative selected from the group consisting of (TTDS)₂, (TTDS)₃, (PEG₂DGA)₃, (PEG₂)₃, (PEG₂)₄ and (PEG₂)₅.

As indicated previously, gE, which can also be represented as γE, gGlu or γGlu, represents a γ-glutamic acid. This amino acid has the following structure:

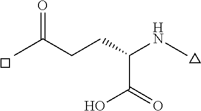

in which, when represented in formula (II) as (gE)_{c}, Δ represents a covalent bond with C_{d} and □ represents a covalent bond with the (PEGₓₓ)_{b} group.

C_{d} represents a linear saturated C12-C₂₂ acyl group, for example a linear saturated acyl group selected from the group consisting of C₁₂ (Lau), C₁₄ (Myr), C₁₅ (Penta), C₁₆ (Palm), C₁₇ (Hepta), C₁₈ (Stea), C₂₀ (Eico) and C₂₂ (Doco) acyl group. In one embodiment Ca represents a linear saturated acyl group selected from the group consisting of C₁₂ (Lau), C₁₄ (Myr), C₁₅ (Penta), C₁₆ (Palm), C₁₇ (Hepta) or C₁₈ (Stea) acyl group, for example a linear saturated C₁₄, C₁₆ or C₁₈ acyl group, or for example a linear C₁₆ or C₁₈ acyl group.

In a particular embodiment, C_{d} represents a linear saturated C₁₂ acyl group. A linear saturated C₁₂ acyl group is a lauroyl group (also represented as “Lau” in the present text).

In a particular embodiment, C_{d} represents a linear saturated C₁₄ acyl group. A linear saturated C₁₄ acyl group is a Myristoyl group (also represented as “Myr” in the present text).

In a particular embodiment, C_{d} represents a linear saturated C₁₅ acyl group. A linear saturated Cis acyl group is a pentadecanoyl group (also represented as “Penta” in the present text).

In a particular embodiment, C_{d} represents a linear saturated C₁₆ acyl group. A linear saturated C₁₆ acyl group is a palmitoyl group (also represented as “Palm” in the present text).

In a particular embodiment, C_{d} represents a linear saturated C₁₇ acyl group. A linear saturated C₁₇ acyl group is a heptadecanoyl group (also represented as “Hepta” in the present text).

In another particular embodiment, C_{d} represents a linear saturated C₁₈ acyl group. A linear saturated C₁₈ acyl group is a stearoyl group (also represented as “Stea” in the present text).

In another particular embodiment, C_{d} represents a linear saturated C₂₀ acyl group. A linear saturated C₂₀ acyl group is a Eicosanoyl group (also represented as “Eico” in the present text).

In another particular embodiment, C_{d} represents a linear saturated C₂₂ acyl group. A linear saturated C₂₂ acyl group is a Docosanoyl group (also represented as “Doco” in the present text).

In a particular embodiment, Z is selected from the group consisting of -(TTDS)₂-(T glutamic acid)₃-Palmitoyl (-(TTDS)₂-(gE)₃-Palm), -(TTDS)₃-(γ glutamic acid)₃-Palmitoyl (-(TTDS)₃-(gE)₃-Palm), -(PEG₂DGA)₃-(γ glutamic acid)₃-Palmitoyl (-(PEG₂DGA)₃-(gE)₃-Palm), -(PEG₂)₄-(γ glutamic acid)₃-Palmitoyl (-(PEG₂)₄-(gE)₃-Palm), -(TTDS)₂-(γ glutamic acid)₂-Palmitoyl (-(TTDS)₂-(gE)₂-Palm), -(TTDS)₂-(γ glutamic acid)₃-Stearoyl (-(TTDS)₂-(gE)₃-Stea), -(TTDS)₃-(γ glutamic acid)₃-Stearoyl (-(TTDS)₃-(gE)₃-Stea), -(PEG₂DGA)₃-(T glutamic acid)₃-Stearoyl (-(PEG₂DGA)₃-(gE)₃-Stea), -(PEG₂DGA)₃-(γ glutamic acid)₄-Stearoyl (-(PEG₂DGA)₃-(gE)₄-Stea), -(PEG₂)₃-(γ glutamic acid)₃-Palmitoyl (-(PEG₂)₃-(gE)₃-Palm), -(PEG₂)₄-(γ glutamic acid)₃-Stearoyl (-(PEG₂)₄-(gE)₃-Stea), -(PEG₂)₅-(γ glutamic acid)₃-Palmitoyl (-(PEG₂)₅-(gE)₃-Palm), -(PEG₂)₅-(γ glutamic acid)₄-Palmitoyl (-(PEG₂)₅-(gE)₄-Palm), -(TTDS)₃-(γ glutamic acid)₄-Stearoyl (-(TTDS)₃-(gE)₄-Stea), -(TTDS)₂-(y glutamic acid)₄-Palmitoyl (-(TTDS)₂-(gE)₄-Palm), -(TTDS)₃-(γ glutamic acid)₂-Stearoyl (-(TTDS)₃-(gE)₂-Stea) and -(TTDS)₂-(γ glutamic acid)₄-Stearoyl (-(TTDS)₂-(gE)₄-Stea), in another embodiment Z is selected from the group consisting of -(TTDS)₂-(gE)₃-Palm, -(TTDS)₃-(gE)₃-Palm, -(PEG₂DGA)₃-(gE)₃-Palm, -(PEG₂)₄-(gE)₃-Palm, -(TTDS)₂-(gE)₂-Palm, -(TTDS)₂-(gE)₃-Stea, -(TTDS)₃-(gE)₃-Stea, -(PEG₂)₃-(gE)₃-Palm, -(PEG₂)₄-(gE)₃-Stea and -(TTDS)₂-(gE)₄-Palm, in a special embodiment Z is selected from the group consisting of -(TTDS)₃-(gE)₃-Palm, -(TTDS)₂-(gE)₃-Stea and -(TTDS)₃-(gE)₃-Stea, for example -(TTDS)₃-(gE)₃-Palm and -(TTDS)₃-(gE)₃-Stea.

In all these Z groups, the first—symbol represents the covalent bond between the Z group and nitrogen atom of the lateral chain of the lysine X₂₅ structure.

Accordingly, when the Z group is for example represented as being -(TTDS)₂-(γ glutamic acid)₃-Palmitoyl (also represented as -(TTDS)₂-(gE)₃-Palm), the first TTDS group is bound to the nitrogen atom of the lateral chain of the X₂₅ structure. A covalent bond also binds the second TTDS group while another covalent bond binds this second TTDS group to the first γ-glutamic acid (gE). This first γ-glutamic acid (gE) is itself bonded through a covalent bond to the second γ-glutamic acid (gE), this second γ-glutamic acid (gE) is bonded through another covalent bond to the third γ-glutamic acid (gE) and this third γ-glutamic acid (gE) group is further linked by a covalent bond to a palmitoyle (Palm) group.

Furthermore, it is to be understood from the present application that a Z group represented for example as -(TTDS)₂-(γ glutamic acid)₃-Palmitoyl (also represented as -(TTDS)₂-(gE)₃-Palm) could also have been represented -TTDS-TTDS-gE-gE-gE-Palm.

The same applies mutatis mutandis for the other represented Z groups according to the invention.

According to a particular embodiment, a relaxin peptide analogue is of formula (Ia) (SEQ ID NO 106):

Nₜₑᵣ-Ac-L-E-G-R-E-X₁₅-V-R-X₁₈-X₁₉-I-Aib-Aib-E-G-X₂₅-
S-T-X₂₈-X₂₉-X₃₀-R-X₃₂-X₃₃-NH₂-Cₜₑᵣ

wherein:
Nₜₑᵣ represents the N-terminal end of the peptide;
Cₜₑᵣ represents the C-terminal end of the peptide;
Ac represents acetyl group;
L represents leucine;
E represents glutamic acid;
G represents glycine;
R represents arginine;
X₁₅ represents an amino acid selected form the group consisting of lysine, arginine, homolysine, glutamine, phenylalanine and leucine;
V represents valine;
X₁₈ represents an amino acid selected from the group consisting of alanine and 2-amino-isobutyric acid;
X₁₉ represents an amino acid selected from the group consisting of lysine, Nε-acetyl-lysine, glutamine and citrulline;
I represents isoleucine; Aib represents 2-amino-isobutyric acid;
X₂₅ represents the following structure:

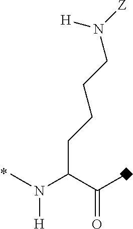

in which:
   * represents a covalent bond with the glycine preceding X₂₅ in formula (Ia);
   ♦ represents a covalent bond with the serine following X₂₅ in formula (Ia); and Z is selected from the group consisting of a -(TTDS)₂-(gE)₃-Palm, -(TTDS)₃-(gE)₃-Palm, -(PEG₂DGA)₃-(gE)₃-Palm, -(PEG₂)₄-(gE)₃-Palm, -(TTDS)₂-(gE)₂-Palm, -(TTDS)₂-(gE)₃-Stea, -(TTDS)₃-(gE)₃-Stea, -(PEG₂DGA)₃-(gE)₃-Stea, -(PEG₂DGA)₃-(gE)₄-Stea, -(PEG₂)₃-(gE)₃-Palm, -(PEG₂)₄-(gE)₃-Stea, -(PEG₂)₅-(gE)₃-Palm, -(PEG₂)₅-(gE)₄-Palm, -(TTDS)₃-(gE)₄-Stea, -(TTDS)₂-(gE)₄-Palm, -(TTDS)₃-(gE)₂-Stea and -(TTDS)₂-(gE)₄-Stea, wherein gE represents γ-glutamic acid
   Palm represents Palmitoyl and
   Stea represents Stearoyl;
S represents serine;
T represents threonine;
X₂₈ represents an amino acid selected from the group consisting of tryptophan and phenylalanine;
X₂₉ represents an amino acid selected from the group consisting of serine, D-serine and 2-amino-isobutyric acid;
X₃₀ represents an amino acid selected from the group consisting of 2-amino-isobutyric acid, α-methyl-lysine, D-lysine and lysine;
X₃₂ represents an amino acid selected from the group consisting of lysine, alanine and arginine; and
X₃₃ represents an amino acid selected from lysine and Nε-acetyl-lysine; or a salt or solvate thereof.

According to a particular embodiment, a relaxin peptide analogue has an amino acid sequence selected from the group consisting of the amino acid sequences of reference SEQ ID NO: 1-97. In particular embodiments, a relaxin peptide analogue has an amino acid sequence selected from the group consisting of the amino acid sequences of reference SEQ ID NO: 1-32, 34-37, 39, 42, 44, 45, 47-49, 51 and 54-97.

In a particular embodiment for a peptide having the following formula (Ia) as described above:
X₁₉ represents an amino acid selected from the group consisting of Nε-acetyl-lysine and citrulline;
X₂₅ represents the following structure:

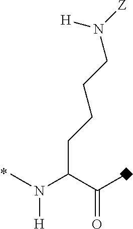

in which:
   * represents a covalent bond with the glycine preceding X₂₅ in formula (Ia);
   ♦ represents a covalent bond with the serine following X₂₅ in formula (Ia); and Z is selected from the group consisting of a -(TTDS)₂-(gE)₃-Palm, -(TTDS)₃-(gE)₃-Palm, -(PEG₂DGA)₃-(gE)₃-Palm, -(PEG₂)₄-(gE)₃-Palm, -(TTDS)₂-(gE)₂-Palm, -(TTDS)₂-(gE)₃-Stea, -(TTDS)₃-(gE)₃-Stea, -(PEG₂)₃-(gE)₃-Palm, -(PEG₂)₄-(gE)₃-Stea and -(TTDS)₂-(gE)₄-Palm, wherein gE represents γ-glutamic acid Palm represents Palmitoyl and Stea represents Stearoyl; X₃₂ represents an amino acid selected from the group consisting of lysine and alanine; and X₃₃ represents Nε-acetyl-lysine or a salt or solvate thereof.

According to another embodiment, a relaxin peptide analogue has an amino acid sequence selected from the group consisting of the amino acid sequences of reference SEQ ID NO: 1-30.

In a particular embodiment, a relaxin peptide analogue of formula (Ia) is such that:
Nₜₑᵣ represents the N-terminal end of the peptide;
Cₜₑᵣ represents the C-terminal end of the peptide;
Ac represents acetyl group;
L represents leucine;
E represents glutamic acid;
G represents glycine;
R represents arginine;
X₁₅ represents an amino acid selected form the group consisting of lysine and homolysine;
V represents valine;
X₁₈ represents alanine;
X₁₉ represents an amino acid selected from the group consisting of Nε-acetyl-lysine and citrulline;
I represents isoleucine;
Aib represents 2-amino-isobutyric acid;
X₂₅ represents the following structure:

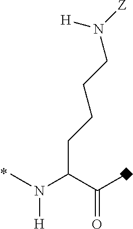

in which:
   * represents a covalent bond with the glycine preceding X₂₅ in formula (Ia);
   ♦ represents a covalent bond with the serine following X₂₅ in formula (Ia); and Z is selected from the group consisting of -(TTDS)₂-(gE)₂-Palm, -(TTDS)₂-(gE)₃-Palm, -(TTDS)₂-(gE)₄-Palm, -(TTDS)₃-(gE)₃-Palm, -(PEG₂DGA)₃-(gE)₃-Palm, -(TTDS)₂-(gE)₃-Stea, -(TTDS)₃-(gE)₃-Stea and -(PEG₂)₄-(gE)₃-Stea, wherein gE represents γ-glutamic acid Palm represents Palmitoyl and Stea represents Stearoyl;
S represents serine;
T represents threonine;
X₂₈ represents an amino acid selected from the group consisting of tryptophan and phenylalanine;
X₂₉ represents serine;
X₃₀ represents an amino acid selected from the group consisting of 2-amino-isobutyric acid and α-methyl-lysine;
X₃₂ represents an amino acid selected from the group consisting of lysine, alanine and arginine; and
X₃₃ represents an amino acid selected from lysine and Nε-acetyl-lysine;
or a salt or solvate thereof.

In a particular embodiment, a relaxin peptide analogue of formula (Ia) is such that:
Nₜₑᵣ represents the N-terminal end of the peptide;
Cₜₑᵣ represents the C-terminal end of the peptide;
Ac represents acetyl group;
L represents leucine;
E represents glutamic acid;
G represents glycine;
R represents arginine;
X₁₅ represents lysine;
V represents valine;
X₁₈ represents alanine;
X₁₉ represents Nε-acetyl-lysine;
I represents isoleucine;
Aib represents 2-amino-isobutyric acid;
X₂₅ represents the following structure:

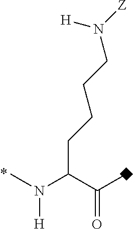

In which:
   * represents a covalent bond with the glycine preceding X₂₅ in formula (Ia);
   ♦ represents a covalent bond with the serine following X₂₅ in formula (Ia); and Z is selected from the group consisting of -(TTDS)₃-(gE)₃-Palm, -(TTDS)₂-(gE)₃-Stea, -(TTDS)₃-(gE)₃-Stea and -(PEG₂)₄-(gE)₃-Stea, wherein gE represents γ-glutamic acid Palm represents Palmitoyl and Stea represents Stearoyl;
S represents serine;
T represents threonine;
X₂₈ represents an amino acid selected from the group consisting of tryptophan and phenylalanine;
X₂₉ represents serine;
X₃₀ represents an amino acid selected from the group consisting of 2-amino-isobutyric acid and α-methyl-lysine;
X₃₂ represents an amino acid selected from the group consisting of lysine, alanine and arginine; and
X₃₃ represents an amino acid selected from lysine and Nε-acetyl-lysine;
or a salt or solvate thereof.

According to a particular embodiment, a relaxin peptide analogue is of formula (Ib):

Nₜₑᵣ-Ac-X₁₀-E-G-R-E-X₁₅-V-R-X₁₈-X₁₉-I-X₂₁-X-₂₂-E-G-X₂₅-
S-X₂₇-X₂₈-X₂₉-X₃₀-R-X₃₂-X₃₃-NH₂-Cₜₑᵣ

herein:
Nₜₑᵣ represents the N-terminal end of the peptide;
Cₜₑᵣ represents the C-terminal end of the peptide;
Ac represents acetyl group;
X₁₀ represents an amino acid selected form the group consisting of leucine, Nε-acetyl-lysine and 2-amino-isobutyric acid;
E represents glutamic acid;
G represents glycine;
R represents arginine;
X₁₅ represents an amino acid selected form the group consisting of lysine, arginine, homolysine, glutamine, phenylalanine and leucine;
V represents valine;
X₁₈ represents an amino acid selected from the group consisting of alanine, 2-amino-isobutyric acid and N&-acetyl-lysine;
X₁₉ represents an amino acid selected from the group consisting of lysine, Nε-acetyl-lysine, glutamine and citrulline; I represents isoleucine;
X₂₁ represents an amino acid selected from the group consisting of 2-amino-isobutyric acid and alanine;
X₂₂ represents an amino acid selected from the group consisting of 2-amino-isobutyric acid and isoleucine;
X₂₅ represents the following structure:

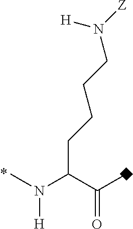

in which:
   * represents a covalent bond with the glycine preceding X₂₅ in formula (Ia);
   ♦ represents a covalent bond with the serine following X₂₅ in formula (Ia); and Z is selected from the group consisting of a -(TTDS)₂-(gE)₃-Palm, -(TTDS)₃-(gE)₃-Palm, -(PEG₂DGA)₃-(gE)₃-Palm, -(PEG₂)₄-(gE)₃-Palm, -(TTDS)₂-(gE)₂-Palm, -(TTDS)₂-(gE)₃-Stea, -(TTDS)₃-(gE)₃-Stea, -(PEG₂DGA)₃-(gE)₃-Stea, -(PEG₂DGA)₃-(gE)₄-Stea, -(PEG₂)₃-(gE)₃-Palm, -(PEG₂)₄-(gE)₃-Stea, -(PEG₂)₅-(gE)₃-Palm, -(PEG₂)₅-(gE)₄-Palm, -(TTDS)₃-(gE)₄-Stea, -(TTDS)₂-(gE)₄-Palm, -(TTDS)₃-(gE)₂-Stea, -(TTDS)₂-(gE)₄-Stea, -(TTDS)₄-(gE)₃-Stea, -(TTDS)₃-(gE)₄-Palm, -(TTDS)₄-(gE)₃-Palm, -(TTDS)₃-(gE)₃-Myr and -(TTDS)₃-(gE)₄-Myr wherein gE represents γ-glutamic acid Palm represents Palmitoyl and Stea represents Stearoyl;
S represents serine;
X₂₇ represents an amino acid selected from the group consisting of threonine, glutamine, arginine and lysine;
X₂₈ represents an amino acid selected from the group consisting of tryptophan, phenylalanine, 5-Chlorotryptophan, α-Methyl-phenylalanine, 4-Fluoro-phenylalanine and 5-Fluorotryptophan;
X₂₉ represents an amino acid selected from the group consisting of serine, D-serine,
2-amino-isobutyric acid, Nε-acetyl-lysine, threonine and valine;
X₃₀ represents an amino acid selected from the group consisting of 2-amino-isobutyric acid, α-methyl-lysine, D-lysine, lysine, homolysine and arginine;
X₃₂ represents an amino acid selected from the group consisting of lysine, alanine, arginine and Nε-acetyl-lysine; and
X₃₃ represents an amino acid selected from lysine, Nε-acetyl-lysine and arginine; or a salt or solvate thereof.

In some embodiments, Z can be selected from the group consisting of a -(TTDS)₂-(gE)₃-Palm, -(TTDS)₃-(gE)₃-Palm, -(PEG₂DGA)₃-(gE)₃-Palm, -(PEG₂)₄-(gE)₃-Palm, -(TTDS)₂-(gE)₂-Palm, -(TTDS)₂-(gE)₃-Stea, -(TTDS)₃-(gE)₃-Stea, -(PEG₂DGA)₃-(gE)₃-Stea, -(PEG₂)₃-(gE)₃-Palm, -(PEG₂)₄-(gE)₃-Stea, -(PEG₂)₅-(gE)₃-Palm, -(TTDS)₃-(gE)₄-Stea, -(TTDS)₂-(gE)₄-Palm, -(TTDS)₂-(gE)₄-Stea, -(TTDS)₄-(gE)₃-Stea, -(TTDS)₃-(gE)₄-Palm, -(TTDS)₄-(gE)₃-Palm, -(TTDS)₃-(gE)₃-Myr and -(TTDS)₃-(gE)₄-Myr.

In a particular embodiment, a relaxin peptide analogue has an amino acid sequence selected from the group consisting of SEQ ID NO: 3, 6, 7, 9, 10, 11, 12, 13, 20, 21, 22, 26, 28 and 30.

In a particular embodiment, a relaxin peptide analogue an amino acid sequence selected from the group consisting of SEQ ID NO: 3, 6, 7, 9-12, 20-22, 26, 28, 30-34, 45, 47-49, 51, 54-62, 64, 67-69, 71-93, 96 and 97. As indicated here-above and illustrated in the enclosed examples, these peptides have an EC₅₀ lower or equal to 1 nM.

In particular, a relaxin peptide analogue has an amino acid sequence selected from the group consisting of SEQ ID NO: 3, 6, 7, 13, 20, 26 and 30.

In particular, a relaxin peptide analogue has an amino acid sequence selected from the group consisting of SEQ ID NO: 3, 6, 7, 20, 26, 30-34, 45, 48, 49, 51, 54-61, 67, 71, 73, 75-79, 81, 83-92 and 97. As illustrated in the enclosed examples, the peptides according to this embodiment all have an EC₅₀≤0.5 nM in the in vitro OVCAR5 cell line cyclic-adenosine monophosphate (cAMP) assay, where OVCAR5 cells express endogenous human RXFP1 (see Example 3).

In addition, compared to RXFP1 agonist peptides of the prior art, in some embodiments, relaxin peptide analogues display improved solubility at pH 4.5 or pH 7.5, improved rat and human plasma or blood stability and in-vivo pharmacokinetic half-lives.

Such properties allow relaxin peptide analogue formulations in broad concentration ranges for use as medicament that will retain in-vivo efficacy for longer period of time (i.e., long-acting), permitting once a day administration by the intravenous or subcutaneous route.

In a particular embodiment, a relaxin peptide analogue has an amino acid sequence selected from the group consisting of the amino acid sequences of reference SEQ ID NO: 3, SEQ ID NO: 6, SEQ ID NO: 7 and SEQ ID NO: 20, and particularly has the amino acid sequence of reference SEQ ID NO: 3.

In some embodiments, further description and related embodiments of the relaxin analogues described herein can be used in combination with vasopressin analogues, as described herein. Such disclosure of relaxin analogues can be found in PCT Publication No.

WO 2019/149782, “Modified Lipidated Relaxin B Chain Peptides and Their Therapeutic Use,” published Aug. 8, 2019 as a publication of International Application No. PCT/EP2019/052298; PCT Publication No. WO 2019/149780, “Modified Relaxin B Chain Peptides and Their Therapeutic Use,” published Aug. 8, 2019 as a publication of International Application No. PCT/EP2019/052296; and PCT Publication No. WO 2019/149781, “Modified Lipidated Relaxin B Chain Peptides and Their Therapeutic Use,” published Aug. 8, 2019 as a publication of International Application No. PCT/EP2019/052297, incorporated by reference in its entirety. In some embodiments, other relaxin peptide analogues known in the art can be used in combination with a vasopressin analogue, such as terlipressin, for treating an individual in need thereof as described herein. For example, RXFP1 agonist relaxin peptide analogues are disclosed in PCT Publication WO 2015/157829, “Modified relaxin B chain peptides,” published Oct. 22, 2015 as a publication of International Application No. PCT/AU2015/050184, incorporated by reference herein in its entirety.

A relaxin peptide analogue may be produced by any technique known per se in the art, such as, without limitation, any chemical, biological, genetic or enzymatic technique, either alone or in combination. Knowing the amino acid sequence of the desired sequence, one skilled in the art can readily produce said polypeptides, by standard techniques for production of polypeptides, even if non-natural amino acids are used or according to the methods described herein.

For instance, the relaxin peptide analogues can be synthesized using well-known solid phase method, for example using a commercially available peptide synthesis apparatus (such as that made by Applied Biosystems, Foster City, Calif., Gyros Protein technologies, Tucson, Ariz. or CEM corporation, Matthews, N.C.) and following the manufacturer's instructions.

Examples of appropriate methods are illustrated in the enclosed examples.

Relaxin Analogues—Small Molecule RXFP1 agonists

In some embodiments, the relaxin analogue is a small molecule with orthosteric or allosteric agonist activity at RXFP1. Such small molecule agonists are known to those of ordinary skill in the art, such as ML290 and ML290 analogues (see, e.g., McBride, A., Hoy, A. M., Bamford, M. J. et al. In search of a small molecule agonist of the relaxin receptor RXFP1 for the treatment of liver fibrosis. Sci Rep 7, 10806 (2017)), and Kocan, M., Sarwar, M., Ang, S. Y. et al. ML290 is a biased allosteric agonist at the relaxin receptor RXFP1. Sci Rep 7, 2968 (2017)).

Dosing Regimen of Relaxin Analogues and/or Vasopressin Analogues

Disclosed herein are methods for preventing or treating renal failure in an individual in need thereof, comprising co-administering a dose of a relaxin analogue from about 1.0 mg to about 5.0 mg and an effective amount of a vasopressin analogue to the individual. In various embodiments, the renal failure is selected from the group consisting of: renal dysfunction induced by liver cirrhosis, renal dysfunction induced by liver transplantation, chronic kidney disease, and acute kidney injury. Additionally disclosed herein are methods for preventing or treating hepatorenal syndrome in an individual in need thereof, comprising co-administering a dose of a relaxin analogue from about 1.0 mg to about 5.0 mg and an effective amount of a vasopressin analogue to the individual. In various embodiments, the hepatorenal syndrome is HRS-AKI (hepatorenal syndrome type 1). In particular embodiments, the relaxin analogue is an RXFP1 agonist, such as a long-acting peptidyl RXFP1 agonist.

In various embodiments, one or more doses of the relaxin analogue are administered to an individual. In various embodiments, two or more doses, three or more doses, four or more doses, five or more doses, six or more doses, seven or more doses, eight or more doses, nine or more doses, ten or more doses, eleven or more doses, twelve or more doses, thirteen or more doses, fourteen or more doses, fifteen or more doses, sixteen or more doses, seventeen or more doses, eighteen or more doses, nineteen or more doses, or twenty or more doses of the relaxin analogue are administered to an individual. In various embodiments, a single dose of the relaxin analogue is administered to an individual. In various embodiments, two or more doses of the relaxin analogue are administered on the same day. In various embodiments, two doses of the relaxin analogue are administered to the individual on the same day, followed by administration of one or more doses of the relaxin analogue on subsequent days.

The subsequent description refers to a dose of the relaxin analogue. In embodiments, where multiple doses of the relaxin analogue are administered to the individual, the subsequent description is similarly applicable to each of the individual doses of the relaxin analogue administered to the individual.

In particular embodiments, the relaxin analogue is administered to an individual at a fixed dose. In various embodiments, the relaxin analogue is administered to an individual at a dose from about 0.1 mg to about 100 mg. In various embodiments, the relaxin analogue is administered to an individual at a dose from about 0.5 mg to about 50 mg. In various embodiments, the relaxin analogue is administered to an individual at a dose from about 1.0 mg to about 50 mg. In various embodiments, the relaxin analogue is administered to an individual at a dose from about 1 mg to about 25 mg, from about 2 mg to about 20 mg, from about 3 mg to about 19 mg, from about 4 mg to about 18 mg, from about 5 mg to about 15 mg, from about 6 mg to about 14 mg, from about 7 mg to about 13 mg, from about 8 mg to about 12 mg, or from about 9 mg to about 11 mg.

In various embodiments, the relaxin analogue is administered to an individual at a dose from about 1 mg to about 10 mg, from about 1 mg to about 9 mg, from about 1 mg to about 8 mg, from about 1 mg to about 7 mg, from about 1 mg to about 6 mg, from about 1 mg to about 5 mg, from about 1 mg to about 4 mg, from about 1 mg to about 6 mg, or from about 1 mg to about 2 mg. In various embodiments, the relaxin analogue is administered to an individual at a dose from about 1 mg to about 10 mg, from about 2 mg to about 8 mg, from about 3 mg to about 6 mg, from about 3 mg to about 5 mg, or from about 4 mg to about 5 mg. In various embodiments, the relaxin analogue is administered to an individual at a dose from about 1 mg to about 9 mg, from about 2 mg to about 6 mg, from about 3 mg to about 5 mg, or from about 3.5 mg to about 4.5 mg.

In various embodiments, the relaxin analogue is administered to an individual at a dose from about 10 mg to about 20 mg, from about 10.5 mg to about 18 mg, from about 11 mg to about 16 mg, from about 11.5 mg to about 14 mg, or from about 12 mg to about 13 mg. In various embodiments, the relaxin analogue is administered to an individual at a dose from about 20 mg to about 30 mg, from about 21 mg to about 29 mg, from about 22 mg to about 28 mg, from about 23 mg to about 26 mg, or from about 24 mg to about 25 mg. In various embodiments, the relaxin analogue is administered to an individual at a dose from about 40 mg to about 50 mg, from about 42 mg to about 49.5 mg, from about 44 mg to about 49 mg, from about 46 mg to about 48.5 mg, or from about 47 mg to about 48 mg.

In particular embodiments, the relaxin analogue is administered to an individual at a dose of about 2.0 mg. In particular embodiments, the relaxin analogue is administered to an individual at a dose of about 4.0 mg. In particular embodiments, the relaxin analogue is administered to an individual at a dose of about 5.0 mg. In particular embodiments, the relaxin analogue is administered to an individual at a dose of about 10.0 mg. In particular embodiments, the relaxin analogue is administered to an individual at a dose of about 12.0 mg. In particular embodiments, the relaxin analogue is administered to an individual at a dose of about 15.0 mg. In particular embodiments, the relaxin analogue is administered to an individual at a dose of about 24.0 mg. In particular embodiments, the relaxin analogue is administered to an individual at a dose of about 48.0 mg.

In various embodiments, the relaxin analogue is administered subcutaneously. In various embodiments, the relaxin analogue is administered intravenously. In various embodiments, the relaxin analogue is administered intravenously as a bolus injection. In various embodiments, the relaxin analogue is administered intravenously over from about 10 minutes to about 24 hours, from about 30 minutes to about 16 hours, from about 45 minutes to about 12 hours, from about 1 hour to about 10 hours, from about 1.5 hours to about 9 hours, from about 2 hours to about 8 hours, from about 2.5 hours to about 7 hours, from about 3 hours to about 6 hours, from about 3.5 hours to about 5 hours, or from about 4 hours to about 4.5 hours. In particular embodiments, the relaxin analogue is administered intravenously over about 4 hours.

In various embodiments, at least two doses of the relaxin analogue are administered to the individual. In various embodiments, the second dose (e.g., an additional dose) of the relaxin analogue is administered to the individual between from about 1 hour to about 24 hours after administration of the first dose of the relaxin analogue. In various embodiments, the second dose of the relaxin analogue is administered to the individual between from about 2 hours to about 22 hours, from about 3 hours to about 20 hours, from about 4 hours to about 19 hours, from about 5 hours to about 18 hours, from about 6 hours to about 17 hours, from about 7 hours to about 16 hours, from about 8 hours to about 15 hours, from about 9 hours to about 14 hours, from about 10 hours to about 13 hours, or from about 11 hours to about 12.5 hours after administration of the first dose of the relaxin analogue.

In various embodiments, timing of the second dose of the relaxin analogue is measured in relation to the completion of the administration of the first dose of the relaxin analogue. For example, assuming the first dose of the relaxin analogue is a 4 hour continuous intravenous administration, the timing of the second dose of the relaxin analogue is measured in relation to the completion of the four hour continuous intravenous administration. In various embodiments, timing of the second dose of the relaxin analogue is measured in relation to the initiation of the administration of the first dose of the relaxin analogue. For example, assuming the first dose of the relaxin analogue is a 4 hour continuous intravenous administration, the timing of the second dose of the relaxin analogue is measured in relation to the initiation of the four hour continuous intravenous administration.

In various embodiments, the first dose and second dose of the relaxin analogue are administered to the individual through different administration routes. For example, the first dose of the relaxin analogue may be administered intravenously to the individual and the second dose of the relaxin analogue may be administered subcutaneously to the individual.

As another example, the first dose of the relaxin analogue may be administered subcutaneously to the individual and the second dose of the relaxin analogue may be administered intravenously to the individual. In various embodiments, the first dose and the second dose of the relaxin analogue are administered to the individual through the same administration route. For example, both the first dose and the second dose of the relaxin analogue are administered to the individual intravenously. As another example, both the first and the second dose of the relaxin analogue are administered to the individual subcutaneously.

In various embodiments, the second dose of the relaxin analogue is higher than the first dose of the relaxin analogue. In various embodiments, the second dose of the relaxin analogue is at least 100% higher than the first dose of the relaxin analogue. For example, the first dose may be about 2.0 mg and the second dose may be about 5.0 mg. As another example, the first dose may be about 4.0 mg and the second dose may be about 10.0 mg. In various embodiments, the second dose of the relaxin analogue is less than 30% higher than the first dose of the relaxin analogue. In one embodiment, the second dose of the relaxin analogue may be 25% higher than the first dose of the relaxin analogue. For example, the first dose may be about 4.0 mg and the second dose may be about 5.0 mg.

In various embodiments, at least a third dose (e.g., a yet additional dose) of the relaxin analogue is administered to the individual. In various embodiments, the third dose of the relaxin analogue is administered to the individual In various embodiments, the third dose of the relaxin analogue is administered to the individual between from about 12 hours to about 48 hours after administration of the first dose of the relaxin analogue. In various embodiments, the third dose of the relaxin analogue is administered to the individual between from about 13 hours to about 45 hours, from about 14 hours to about 42 hours, from about 15 hours to about 39 hours, from about 16 hours to about 36 hours, from about 17 hours to about 33 hours, from about 18 hours to about 30 hours, from about 19 hours to about 28 hours, from about 20 hours to about 26 hours, from about 22 hours to about 25 hours, or from about 23 hours to about 24.5 hours after administration of the first dose of the relaxin analogue.

In various embodiments, timing of the third dose of the relaxin analogue is measured in relation to the completion of the administration of the first dose of the relaxin analogue. For example, assuming the first dose of the relaxin analogue is a 4 hour continuous intravenous administration, the timing of the third dose of the relaxin analogue is measured in relation to the completion of the four hour continuous intravenous administration. In various embodiments, timing of the third dose of the relaxin analogue is measured in relation to the initiation of the administration of the first dose of the relaxin analogue. For example, assuming the first dose of the relaxin analogue is a 4 hour continuous intravenous administration, the timing of the third dose of the relaxin analogue is measured in relation to the initiation of the four hour continuous intravenous administration.

In various embodiments, the first dose and the third dose of the relaxin analogue are administered to the individual through different administration routes. For example, the first dose of the relaxin analogue may be administered intravenously to the individual and the third dose of the relaxin analogue may be administered subcutaneously to the individual. As another example, the first dose of the relaxin analogue may be administered subcutaneously to the individual and the third dose of the relaxin analogue may be administered intravenously to the individual. In various embodiments, the first dose and the third dose of the relaxin analogue are administered to the individual through the same administration route. For example, both the first dose and the third dose of the relaxin analogue are administered to the individual intravenously. As another example, both the first and the third dose of the relaxin analogue are administered to the individual subcutaneously.

In various embodiments, the third dose of the relaxin analogue is higher than the first dose of the relaxin analogue. In various embodiments, the third dose of the relaxin analogue is at least 100% higher than the first dose of the relaxin analogue. For example, the first dose may be about 2.0 mg and the third dose may be about 5.0 mg. As another example, the first dose may be about 4.0 mg and the third dose may be about 10.0 mg. In various embodiments, the third dose of the relaxin analogue is less than 30% higher than the first dose of the relaxin analogue. In one embodiment, the third dose of the relaxin analogue may be 25% higher than the first dose of the relaxin analogue. For example, the first dose may be about 4.0 mg and the third dose may be about 5.0 mg.

In various embodiments, a dose beyond the third dose is administered to the individual. In various embodiments, the dose beyond the third dose replicates the quantity and administration route of the third dose. For example, assuming the third dose includes a 10 mg subcutaneous administration of the relaxin analogue, the dose beyond the third dose also includes a 10 mg subcutaneous administration of the relaxin analogue.

In various embodiments, the dose beyond the third dose is administered daily. In various embodiments, the dose beyond the third dose is administered daily from about 5 days to about 25 days. In various embodiments, the dose beyond the third dose is administered daily from about 6 days to about 24 days, from about 7 days to about 23 days, from about 8 days to about 22 days, from about 9 days to about 21 days, from about 10 days to about 20 days, from about 11 days to about 18 days, from about 12 days to about 16 days, or from about 13 days to about 15 days. In particular embodiments, the dose beyond the third dose is administered daily for 13 days. In particular embodiments, the dose beyond the third dose is administered daily for 14 days. In particular embodiments, the dose beyond the third dose is administered daily for 15 days.

In various embodiments, the dose beyond the third dose is administered daily. In various embodiments, the dose beyond the third dose is administered daily from about 3 days to about 15 days. In various embodiments, the dose beyond the third dose is administered daily from about 4 days to about 13 days, from about 5 days to about 11 days, or from about 6 days to about 9 days. In particular embodiments, the dose beyond the third dose is administered daily for 6 days. In particular embodiments, the dose beyond the third dose is administered daily for 7 days. In particular embodiments, the dose beyond the third dose is administered daily for 8 days. In particular embodiments, the dose beyond the third dose is administered daily for 9 days.

In various embodiments, methods for preventing or treating renal failure in an individual in need thereof and/or methods for preventing or treating hepatorenal syndrome in an individual comprise: about 4 hour intravenous administration of a first dose of 2.0 mg of a relaxin analogue, a subcutaneous administration of 5.0 mg of the relaxin analogue at 12 hours after initiation of the first dose, a subcutaneous administration of 5.0 mg of the relaxin analogue at 24 hours after initiation of the first dose, and thereafter daily subcuitaneous administration of 5.0 mg of the relaxin analogue for up to 12 to 15 days.

In various embodiments, methods for preventing or treating renal failure in an individual in need thereof and/or methods for preventing or treating hepatorenal syndrome in an individual comprise: about 4 hour intravenous administration of a first dose of 4.0 mg of a relaxin analogue, a subcutaneous administration of 10.0 mg of the relaxin analogue at 12 hours after initiation of the first dose, a subcutaneous administration of 10.0 mg of the relaxin analogue at 24 hours after initiation of the first dose, and thereafter daily subcuitaneous administration of 5.0 mg of the relaxin analogue for up to 12 to 15 days.

In various embodiments, methods for preventing or treating renal failure in an individual in need thereof and/or methods for preventing or treating hepatorenal syndrome in an individual comprise: about 4 hour intravenous administration of a first dose of 4.0 mg of a relaxin analogue, a subcutaneous administration of 5.0 mg of the relaxin analogue at 12 hours after initiation of the first dose, a subcutaneous administration of 10.0 mg of the relaxin analogue at 24 hours after initiation of the first dose, and thereafter daily subcuitaneous administration of 5.0 mg of the relaxin analogue for up to 12 to 15 days.

In various embodiments, methods for preventing or treating renal failure in an individual in need thereof and/or methods for preventing or treating hepatorenal syndrome in an individual comprise co-administering an effective amount of a relaxin analogue and a vasopressin analogue to the individual. In particular embodiments, the vasopressin analogue is terlipressin or a pharmaceutically acceptable salt thereof.

In various embodiments, the terlipressin is administered intravenously at a dosage from about 0.5 to about 10 mg. In various embodiments, the terlipressin is administered intravenously at a dosage from about 0.6 mg to about 5 mg, from about 0.7 mg to about 3 mg, from about 0.8 mg to about 2 mg, or from about 0.9 mg to about 1.5 mg. In particular embodiments, the terlipressin is administered intravenously at a dosage of about 1 mg. In various embodiments, the terlipressin is administered intravenously at a dosage from about 0.8 mg to about 6 mg, from about 1 mg to about 5 mg, from about 1.2 mg to about 4 mg, from about 1.4 mg to about 3 mg, from about 1.6 mg to about 2.5 mg, from about 1.8 mg to about 2.2 mg, or from about 1.9 mg to about 2.1 mg. In particular embodiments, the terlipressin is administered intravenously at a dosage of about 2 mg. In various embodiments, the terlipressin is administered intravenously at a dosage from about 1 mg to about 8 mg, from about 2 mg to about 6 mg, from about 3 mg to about 5 mg, from about 3.2 mg to about 4.8 mg, from about 3.4 mg to about 4.6 mg, from about 3.6 mg to about 4.4 mg, or from about 3.8 mg to about 4.2 mg. In particular embodiments, the terlipressin is administered intravenously at a dosage of about 4 mg. In various embodiments, the terlipressin is administered intravenously at a dosage from about 2 mg to about 9 mg, from about 4 mg to about 8 mg, from about 5 mg to about 7 mg, from about 5.2 mg to about 6.8 mg, from about 5.4 mg to about 6.6 mg, from about 5.6 mg to about 6.4 mg, or from about 5.8 mg to about 6.2 mg. In particular embodiments, the terlipressin is administered intravenously at a dosage of about 6 mg.

In various embodiments, the terlipressin is administered intravenously every 2 to 10 hours. In various embodiments, the terlipressin is administered intravenously every 3 to 8 hours, or every 4 to 6 hours. In various embodiments, the terlipressin is administered intravenously every 6 hours. In various embodiments, terlipressin is administered intravenously via a bolus infusion. In various embodiments, terlipressin is administered intravenously via a bolus injection over from about 30 seconds to about 10 minutes, from about 1 minute to about 5 minutes, or from about 2 minutes to about 3 minutes. In various embodiments, terlipressin is administered intravenously via a bolus injection over about 2 minutes.

In various embodiments, from about 0.5 mg to about 2 mg terlipressin is administered intravenously every 4 to 6 hours. In particular embodiments, about 1.0 mg terlipressin is administered intravenously every 6 hours. In various embodiments, from about 2 mg to about 6 mg terlipressin is administered intravenously every 4 to 6 hours. In particular embodiments, about 4.0 mg terlipressin is administered intravenously every 6 hours. In various embodiments, from about 6 mg to about 10 mg of terlipressin is administered intravenously every 4 to 6 hours. In particular embodiments, about 6.0 mg terlipressin is administered intravenously every 6 hours. In various embodiments, about 8.0 mg terlipressin is administered every 8 hours.

In various embodiments, the terlipressin is administered intravenously over from about 8 hours to about 36 hours. In various embodiments, the terlipressin is administered intravenously over from about 10 hours to about 30 hours, from about 15 hours to about 28 hours, or from about 20 hours to about 25. In particular embodiments, the terilipressin is administered intravenously over about 24 hours.

In various embodiments, the terlipressin is administered intravenously at a dosage from about 1 mg to about 10 mg. In various embodiments, the terlipressin is administered intravenously at a dosage from about 1.5 mg to about 4.0 mg. In various embodiments, the terlipressin is administered intravenously at a dosage of about 2.0 mg. In various embodiments, the terlipressin is administered intravenously at a dosage from about 5.0 mg to about 8.0 mg. In various embodiments, the terlipressin is administered intravenously at a dosage of about 6.0 mg.

In particular embodiments, methods for preventing or treating renal failure in an individual in need thereof and/or methods for preventing or treating hepatorenal syndrome in an individual comprises: one of (1A), (1), or (1C) in combination with (2), where:
(1A) 1.0 mg bolus intravenous infusion of terlipressin every 6 hours,
(1B) 2.0 mg intravenous infusion of terlipressin over 24 hours,
(1C) 6.0 mg intravenous infusion of terlipressin over 24 hours, and
(2) about 4 hour intravenous administration of a first dose of 2.0 mg of a relaxin analogue, a subcutaneous administration of 5.0 mg of the relaxin analogue at 12 hours after initiation of the first dose, a subcutaneous administration of 5.0 mg of the relaxin analogue at 24 hours after initiation of the first dose, and thereafter daily subcuitaneous administration of 5.0 mg of the relaxin analogue for up to 12 to 15 days.

In particular embodiments, methods for preventing or treating renal failure in an individual in need thereof and/or methods for preventing or treating hepatorenal syndrome in an individual comprises: one of (1A), (1), or (1C) in combination with (2), where:
(1A) 1.0 mg bolus intravenous infusion of terlipressin every 6 hours,
(1B) 2.0 mg intravenous infusion of terlipressin over 24 hours,
(1C) 6.0 mg intravenous infusion of terlipressin over 24 hours, and
(2) about 4 hour intravenous administration of a first dose of 4.0 mg of a relaxin analogue, a subcutaneous administration of 10.0 mg of the relaxin analogue at 12 hours after initiation of the first dose, a subcutaneous administration of 10.0 mg of the relaxin analogue at 24 hours after initiation of the first dose, and thereafter daily subcuitaneous administration of 5.0 mg of the relaxin analogue for up to 12 to 15 days.

In particular embodiments, methods for preventing or treating renal failure in an individual in need thereof and/or methods for preventing or treating hepatorenal syndrome in an individual comprises: one of (1A), (1), or (1C) in combination with (2), where:
(1A) 1.0 mg bolus intravenous infusion of terlipressin every 6 hours,
(1B) 2.0 mg intravenous infusion of terlipressin over 24 hours,
(1C) 6.0 mg intravenous infusion of terlipressin over 24 hours, and
(2) about 4 hour intravenous administration of a first dose of 4.0 mg of a relaxin analogue, a subcutaneous administration of 5.0 mg of the relaxin analogue at 12 hours after initiation of the first dose, a subcutaneous administration of 10.0 mg of the relaxin analogue at 24 hours after initiation of the first dose, and thereafter daily subcuitaneous administration of 5.0 mg of the relaxin analogue for up to 12 to 15 days.

### Compositions and Medicaments

The present application also relates to a medicament or a pharmaceutical composition comprising separately or in combination, vasopressin analogues and relaxin analogues as described above, or pharmaceutically acceptable salts or solvates thereof, and at least one pharmaceutically acceptable carrier.

In some embodiments, the relaxin analogue and/or vasopressin analogue is present in a medicament or pharmaceutical composition of the invention as active principle.

A composition or a medicament of the invention is in a form suitable for administration to an individual in need thereof.

A composition or a medicament of the invention can be administered, for example, parenterally, intravenously, subcutaneously, rectally, transdermally, topically or by inhalation. In particular, a composition according to the invention is administered by the intravenous or subcutaneous route.

According to a particular embodiment, the pharmaceutically acceptable carrier of a composition of the invention is suitably selected from the group consisting of an injectable carrier liquid such as sterile water for injection; and an aqueous solution such as saline.

A composition or a medicament of the invention can comprise a content of peptides of the invention comprised between 0.01 mg/mL and 30 mg/mL, in particular between 0.3 mg/mL and 3 mg/mL.

A medicament or a pharmaceutical composition of the invention can comprise at least one peptide of the invention as sole active principle or can also comprise at least one other active principle, as long as said other active principle does not prevent the biological activity of the peptide according to the invention.

A pharmaceutical composition or a medicament according to the invention can further comprise at least one antioxidant, dispersant, emulsifier, antifoam, flavouring, preservative, solubilizer and/or colour, as long as this/these additional substances do not prevent the biological properties of the peptides according to the invention.

Sterile compositions of the invention for parenteral administration may in particular embodiments be aqueous or non-aqueous solutions, suspensions or emulsions. Solvents or vehicles that can be used include water, propylene glycol, a polyethylene glycol, plant oils, for example olive oil, injectable organic esters, for example ethyl oleate, or other suitable organic solvents. These compositions may also comprise adjuvants, for example wetting agents, tonicity agents, emulsifiers, dispersants and stabilisers. The sterilisation may be performed in several ways, for example by aseptic filtration, by incorporating sterilising agents into the composition, by irradiation or by heating. They may also be prepared in the form of sterile solid compositions that may be dissolved at the time of use in sterile water or any other injectable sterile medium.

The compositions for topical administration may be, for example, nasal drops or aerosols.

For subcutaneous, intramuscular or intravenous administration, the peptides of the invention used are converted, if desired with the substances customary for this purpose, such as solubilizers, emulsifiers or other excipients, into a solution, suspension or emulsion.

Examples of suitable solvents are: water, physiological saline or alcohols, e.g. ethanol, propanol, glycerol, as well as sugar solutions such as glucose or mannitol solutions, or else a mixture of the various solvents mentioned.

In a particular embodiment, a composition of the invention, a medicament of the invention, or a peptide of the invention, or one of its pharmaceutically acceptable salt or solvate thereof, is administered to an individual by the parenteral route, and is in particular transdermaly, intravenously, subcutaneously or intramuscularly, in particular intravenously or subcutaneously administered.

Methods for preparing parenterally administrable compositions are apparent to those skilled in the art, and are for example described in more detail in Remington's Pharmaceutical Science, 15^{th} ed., Mack Publishing Company, Easton, Pa.

The administration of a composition of the invention or of a peptide of the invention to an individual can be a systemic administration or an administration localized to a tissue, organ and/or site of the individual organism.

### Use of the Relaxin Analogue and Vasopressin Analogue Peptides and Compositions

The present invention relates to a combination therapy for treatment of an individual in need thereof of a vasopressin analogue and a relaxin analogue, and associated compositions comprising these bioactive agents, as well as their pharmaceutically acceptable salts or solvates thereof.

Moreover, the invention also relates to one or more pharmaceutical compositions according to the invention for use as a combination therapy including a vasopressin analogue and relaxin analogue bioactive agents.

Furthermore, the present invention relates to peptides of the invention, pharmaceutically acceptable salts or solvates thereof, or a pharmaceutical composition of the invention for its use in combination therapy for the treatment and/or prevention of various diseases or conditions implicating the RXFP1 receptor and/or the V1a vasopressin receptor, more particularly in the treatment and/or prevention of diseases or conditions associated with renal failure, including renal dysfunction induced by liver cirrhosis, renal dysfunction induced by liver transplantation, chronic kidney disease, and acute kidney injury. Such combination therapy in particular can be used for the treatment and/or prevention of HRS, in particular HRS-AKI (hepatorenal syndrome type 1) or HRS-NAKI (hepatorenal syndrome type 2).

In one aspect, a combination therapy of a relaxin analogue and vasopressin analogue peptide, pharmaceutically acceptable salt or solvate thereof, or pharmaceutical composition or compositions including one or more of these bioactive agents, is administered once a day, in particular by the intravenous or subcutaneous route.

The dosage of the relaxin analogue and vasopressin analogue peptide, or of its pharmaceutically acceptable salt or solvate thereof, to be administered, and the frequency of administration, depend on the desired effect, the potency and duration of action of the compounds used; additionally, also on the nature and severity of the disease or condition to be treated and on the sex, age, weight and individual responsiveness of the individual to be treated. In general, the physician determines the appropriate dosage as a function of the age and weight and all the other factors specific to the individual to be treated.

Also provided herein is a method for preventing and/or treating a disease or condition associated with renal failure, including renal dysfunction induced by liver cirrhosis, renal dysfunction induced by liver transplantation, chronic kidney disease, and acute kidney injury comprising co-administering to an individual in need of said prevention and/or treatment a combination of relaxin analogue and vasopressin analogue peptides of the invention, pharmaceutically acceptable salts or solvates thereof or a pharmaceutical composition or compositions of the invention comprising vasopressin analogue and/or relaxin analogue bioactive agents according to the invention or a therapeutically effective amount of the combination of vasopressin analogue and relaxin analogue peptides, pharmaceutically acceptable salts or solvates thereof, or a pharmaceutical composition or compositions of the invention comprising vasopressin analogue and/or relaxin analogue bioactive agents according to the invention.

It is further described the use of a combination of the relaxin analogue and vasopressin analogue bioactive agents described herein, pharmaceutically acceptable salts or solvates thereof or pharmaceutical compositions of the invention comprising separately or in combination a relaxin analogue and a vasopressin analogue peptide according to the invention or a therapeutically effective amount of the combination thereof, pharmaceutically acceptable salts or solvates thereof or pharmaceutical compositions of the invention comprising separately or in combination a relaxin analogue and a vasopressin analogue peptide according to the invention for the manufacture of a medicament for the prevention and/or treatment in an individual of a disease or condition associated with renal failure, including renal dysfunction induced by liver cirrhosis, renal dysfunction induced by liver transplantation, chronic kidney disease, and acute kidney injury.

It is further described the use of a combination of the relaxin analogue and vasopressin analogue bioactive agents described herein, pharmaceutically acceptable salts or solvates thereof or pharmaceutical compositions of the invention comprising separately or in combination a relaxin analogue and a vasopressin analogue peptide according to the invention or a therapeutically effective amount of the combination thereof, pharmaceutically acceptable salts or solvates thereof or pharmaceutical compositions of the invention comprising separately or in combination a relaxin analogue and a vasopressin analogue peptide according to the invention for the manufacture of a medicament for the prevention and/or treatment in an individual of HRS, such as HRS-AKI (hepatorenal syndrome type 1) and HRS-NAKI (hepatorenal syndrome type 2).

### EQUIVALENTS AND SCOPE

Those skilled in the art will recognize, or be able to ascertain using no more than routine experimentation, many equivalents to the specific embodiments in accordance with the invention described herein. The scope of the present invention is not intended to be limited to the above Description, but rather is as set forth in the appended claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process.

The invention includes embodiments in which more than one, or all of the group members are present in, employed in, or otherwise relevant to a given product or process.

It is also noted that the term “comprising” is intended to be open and permits but does not require the inclusion of additional elements or steps. When the term “comprising” is used herein, the term “consisting of” is thus also encompassed and disclosed.

Where ranges are given, endpoints are included. Furthermore, it is to be understood that, unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise.

All cited sources, for example, references, publications, databases, database entries, and art cited herein, are incorporated into this application by reference, even if not expressly stated in the citation. In case of conflicting statements of a cited source and the instant application, the statement in the instant application shall control.

Section and table headings are not intended to be limiting.

### EXAMPLES

Below are examples of specific embodiments for carrying out the present invention. The examples are offered for illustrative purposes only and are not intended to limit the scope of the present invention in any way. Efforts have been made to ensure accuracy with respect to numbers used (e.g., amounts, temperatures, etc.), but some experimental error and deviation should, of course, be allowed for.

The practice of the present invention will employ, unless otherwise indicated, conventional methods of protein chemistry, biochemistry, recombinant DNA techniques and pharmacology, within the skill of the art. Such techniques are explained fully in the literature. See, e.g., T. E. Creighton, Proteins: Structures and Molecular Properties (W.H. Freeman and Company, 1993); A. L. Lehninger, Biochemistry (Worth Publishers, Inc., current addition); Sambrook, et al., Molecular Cloning: A Laboratory Manual (2nd Edition, 1989); Methods In Enzymology (S. Colowick and N. Kaplan eds., Academic Press, Inc.); Remington's Pharmaceutical Sciences, 18th Edition (Easton, Pennsylvania: Mack Publishing Company, 1990); Carey and Sundberg Advanced Organic Chemistry 3rd Ed. (Plenum Press) Vols A and B(1992).

### Example 1: Synthesis of the Relaxin Peptide Analogues

### Material Used

Various rink amide resins were used for the synthesis of C-terminal amides: 4-(2′, 4′-Dimethoxyphenyl-Fmoc-aminomethyl)phenoxy resin, sold by Chem-Impex; or 4-(2′, 4′-Dimethoxyphenyl-Fmoc-aminomethyl)phenoxy acetamido methyl resin, sold by Millipore Merck;

They were loaded in the range of 0.2 to 0.4 mmol/g.

Fmoc (fluorenylmethyloxycarbonyl) protected natural amino acids were purchased from different sources, i.e., Protein Technologies Inc., Merck Biosciences, Novabiochem, Iris Biotech, Bachem, Chem-Impex International or MATRIX Innovation.

The following standard amino acids were used throughout the syntheses: Fmoc-L-Ala-OH, Fmoc-L-Arg(Pbf)-OH, Fmoc-L-Gln(Trt)-OH, Fmoc-L-Glu(OtBu)-OH, Fmoc-L-Glu-OtBu, Fmoc-Gly-OH, Fmoc-L-Ile-OH, Fmoc-L-Leu-OH, Fmoc-L-Lys(Boc)-OH, Fmoc-L-Met-OH, Fmoc-L-Phe-OH, Fmoc-L-Ser(tBu)-OH, Fmoc-L-Thr(tBu)-OH, Fmoc-L-Trp(Boc)-OH, Fmoc-L-Tyr(tBu)-OH and Fmoc-L-Val-OH.

In addition, the following special amino acids were purchased from the same suppliers as above: Fmoc-L-hArg(Pbf)-OH, Fmoc-L-Cit-OH, Fmoc-L-Arg(Me,Pbf)-OH, Fmoc-L-hLys(Boc)-OH, Fmoc-L-Orn(Boc)-OH, Fmoc-L-Lys(Dde)-OH, Fmoc-L-Lys(ivDde)-OH, Fmoc-L-Lys(Mtt)-OH, Fmoc-L-Lys(aloc)-OH, Fmoc-L-Lys(Ac)-OH, Fmoc-Aib-OH, Fmoc-L-α-Me-Ser(tBu)-OH, Fmoc-L-α Me-Lys(Boc)-OH, Fmoc-L-α-Me-Arg(Pbf)-OH, Fmoc-L-α-Me-Leu-OH, Fmoc-L-Nle-OH and Fmoc-L-1-Nal-OH, Fmoc-L-2-Nal-OH Fmoc-5-Wox-OH, Fmoc-Pfp-OH, Fmoc-L-α-Me-Trp-OH, Fmoc-L-α-Me-Phe-OH.

| | |
|---|---|
| Ac | Acetyl |
| Aloc | Allyloxycarbonyl |
| Boc | Tert-Butyloxycarbonyl |
| tBu | Tert-Butyl |
| cAMP | Cyclic adenosine monophosphate |
| Dde | 1-(4,4-Dimethyl-2,6-dioxocyclohex-1-ylidene)ethyl |
| ivDde | 1-(4,4-dimethyl-2,6-dioxocyclohex-1-ylidene)-3-methylbutyl |
| DCM | Dichloromethane |
| DIC | N,N′-Diisopropylcarbodiimide |
| DIEA, | N,N-Diisopropyl ethylamine |
| DIPEA | |
| DMF | N,N-Dimethylformamide |
| DODT | 2,2′-(Ethylenedioxy)diethanethiol |
| EDT | 1,2-ethanedithiol |
| Fmoc | Fluorenylmethyloxycarbonyl |
| TFA | Trifluoroacetic acid |
| TIS, | Tri-isopropyl Silane |
| TIPS | |
| HATU | (2-(7-Aza-1H-benzotriazol-1-y))-N,N,N′,N′- |
| | tetramethylaminium hexafluorophosphate) |
| HCTU | O-(1H-6-Chlorobenzotriazole-1-yl)-1,1,3,3-tetrameth- |
| | yluronium hexafluorophosphate |
| HPLC | High Performance Liquid Chromatography |
| HTRF | HTRF Homogenous Time Resolved Fluorescence |
| LC/MS | Liquid Chromatography/Mass Spectrometry |
| Laur | Lauroyl |
| Mmt | Monomethoxy-trityl |
| Mtl | 4-Methyl-trityl |
| Myr | Myristoyl |
| NMP | 1-methylpyrrolidin-2-one |
| Oxyma | Ethyl 2-cyano-2-(hydroximino)acetate (oxyma pureTM) |
| Pbf | 2,2,4,6,7-pentamethyldihydrobenzofuran-5-sulfonyl |
| RP-HPLC | Reversed-phase high performance liquid chromatography |
| RT | Retention Time |
| TFA | Trifluoroacetic acid |
| Trt | Trityl, Triphenylmethyl |
| UV | Ultraviolet |
| UPLC | Ultra high Performance Chromatography |

For synthesizing the C-terminally lipidated relaxin peptide analogues, orthogonally protected lysine was used as indicated here-after.

### 1.A. General Method Used for Synthesizing Selected Relaxin Peptide Analogues

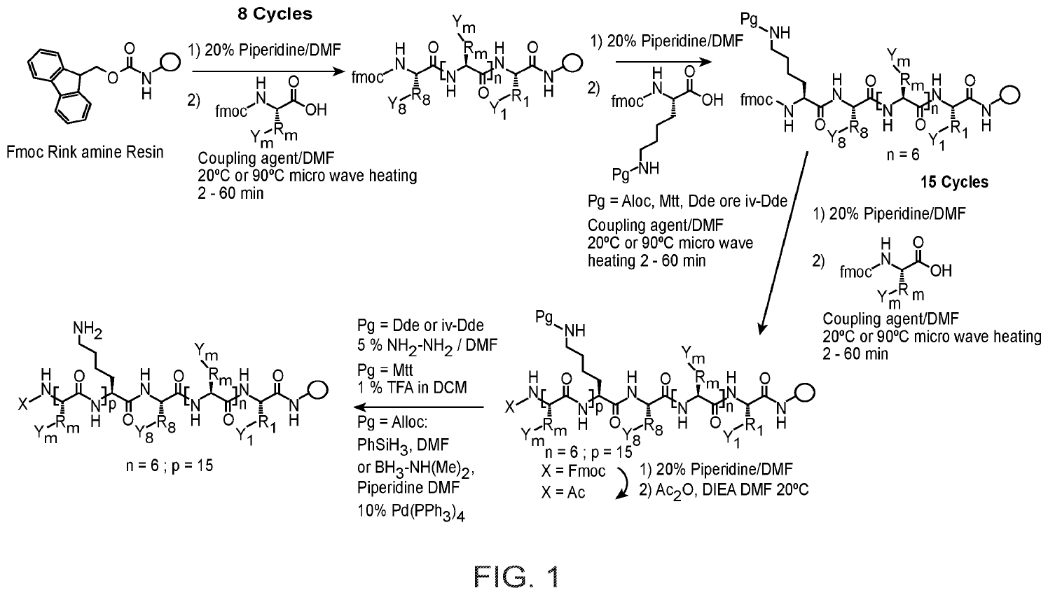

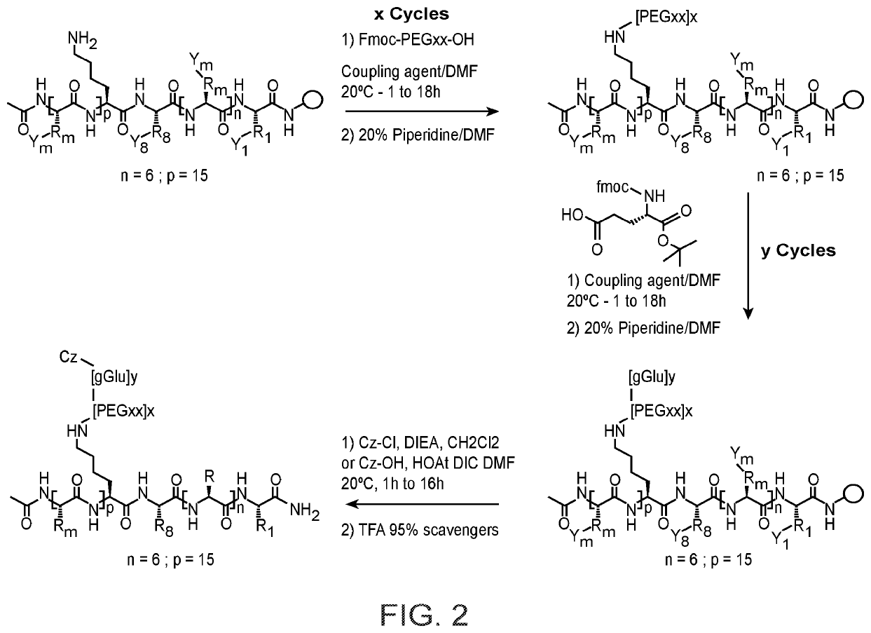

Relaxin peptide analogue of sequence SEQ TD NO: 1-97 have been synthesized on the basis of the method represented in FIGS. 1 and 2.

0.2 mmol of Rink amide AM resin were placed in a CEM Liberty blue microwave peptide synthesizer to assemble the full peptide sequence. (FIG. 1).

The entire synthesis was run in DMF as solvent. The peptide was synthesized using the standard heating protocol for 0.1 to 0.2 mmol scale:

Standard heating protocol: irradiation at 170 watts, 75° C., 15 sec. then irradiation at 30 watts, 90° C., 120 sec.

Deprotection was performed with 20% v/v piperidine in DMF, followed by 3 DMF washing steps.

Heating protocol for deprotection: irradiation at 170 watts, 75° C., 15 sec. then irradiation at 30 watts, 90° C., 50 sec.

Amino acid couplings were performed using 5 eq. of Fmoc-AA as 0.2 M solutions in DMF using 5 eq. N,N′-Diisopropylcarbodiimide (DIC) 0.5M and 5 eq. Oxyma (ethyl 2-cyano-2-(hydroximino)acetate (Oxyma Pure™)) 1M as coupling reagents.

For better final yield, each amino-acid required double couplings at 90° C. for 120 seconds. For 2-aminoisobutyric acid at positions X₂₁ and X₂₂ and serine at position X₂₉ of the formula (I), a triple coupling at 90° C. for 2 minutes was used.

In the case of expensive amino acid derivatives, e.g. Fmoc-α-Methyl Lysine(Boc)-OH, it may be advantageous to perform a manual coupling using 3 eq. of amino acid and 3 eq. of coupling agent such as HATU or HCTU at room temperature for 1 to 18 h or using DIC and Oxyma Pure™ with microwave heating for 120 sec.

At position X₂₅, a derivative of Fmoc-Lysine-OH bearing on the side chain nitrogen an orthogonal protecting group was used. Selective deprotection allowed modification of this amino acid side chain.

At the end of the synthesis, Fmoc deprotection was performed manually with 20% v/v piperidine in DMF two times 30 minutes at room temperature.

Acetylation was performed at N-terminus by treatment with 5-10 eq. of acetic anhydride and 5-10 eq. DIEA in DMF for 15 min. Then the resin containing the fully protected peptide was washed with DCM/DMF/DCM, 3 times each and dried under vacuum.

Then Ne-protecting group of lysine at position X₂₅ (Dde, ivDde, Aloc, or Mtt) was removed using diluted hydrazine in DMF (Dde, ivDde), phenylsilane or dimethyl amino borane in the presence of palladium(0) catalyst in DCM (aloc) or 1% TFA in DCM (Mtt).

Then the Z groups was attached to the side chain nitrogen of the X₂₅ lysine on solid support according to the following steps:
When the lysine in position X₂₅ was protected on its side chain by Dde or iv-Dde, resin-peptide was transferred into a 50 ml polypropylene syringe. 80 ml of a solution of hydrazine 5% in DMF was percolated through the resin followed by DMF wash (3 times). The reaction was monitored by Kaiser Test.

When the lysine in position X₂₅ was protected on its side chain by the allyl-oxy-carbonyl group (aloc), resin-peptide was transferred into a 50 ml polypropylene syringe and swelled in dichloromethane and treated with 20 eq. phenylsilane (PhSiH3) (or borane dimethylamine complex ((CH₃)₂NH·BH₃) and piperidine) and 10% (mol/mol) tetrakis-(triphenylphosphine)palladium (Pd(PPh₃)₄) for 2 h under argon. This treatment was repeated until no starting aloc protected peptide could be detected by UPLC/MS analysis after cleavage of an aliquot part of the resin.

When the reaction was complete, the resin was washed with dichloromethane, 1% DIEA in DMF, 5% diethyl-dithio-carbamate in DMF, DMF, 10% DIEA in DMF, DMF and dichloromethane (3 times each).

When the lysine in position X₂₅ was protected on its side chain by the methyl-trityl group (Mtt), resin-peptide was transferred into a 50 ml polypropylene syringe and swelled in dichloromethane and treated with 10 mL DCM/TIS/TFA (88/5/2) mixture. After 1 h shaking, the solvent was drained, resin washed with DCM, shaken with 10 ml DCM/DIEA (90/10) mixture and washed several times with DCM.

The PEGₓₓ group(s) of the Z group was introduced, if applicable for a given relaxin peptide analogue, by single acylation with 3 eq. of Fmoc-PEGₓₓ-OH for 18 h with 3 eq. of DIC and 3 eq of HOAt monitoring the reactions by ninhydrin (Kaiser) Test. Then the resin was treated with 20% v/v piperidine in DMF to remove Fmoc protecting group (2×30 min) and washed 3 times with DMF. The Fmoc-PEGₓₓ-OH coupling, Fmoc removal and washing steps were repeated x times (x=0-5).

Then Fmoc-Glu-OtBu ((4S)-5-tert-butoxy-4-(9H-fluoren-9-yl methoxy carbonyl amino)-5-oxo-pentanoic acid), if gE present in the Z group of the relaxin peptide analogue, was introduced by performing single coupling with 3 eq. of amino acid for 18 h with 3 eq. of DIC and 3 eq. of HOAt monitoring the reactions by Kaiser Test.

Then resin was treated with 20% v/v piperidine in DMF as to remove Fmoc protecting group (2×30 min) and washed 3 times with DMF. The Fmoc removal and Fmoc-Glu-OtBu coupling steps were repeated y times (y=1-5).

Then, the side chain was lipidated using 3 eq. of Ck lauric acid (C₁₂), myristic acid (C₁₄), pentadecanoic acid (C₁₅), palmitic acid (C₁₆), heptadecanoic acid (C₁₇), stearic acid (Cis), eicosanoic acid (C₂₀) or docosanoic acid (C₂₂) using 3 eq. DIC and 3 eq. HOAt in NMP, using the corresponding acyl chlorides and DIEA as a base in dichloromethane or using the corresponding N-succinimidyl esters and DIEA as base in DMF.

The reaction was monitored by Kaiser Test and left overnight.

Then the resin containing the fully protected peptide was washed with DCM/DMF/DCM 3 times each and dried under vacuum.

### 1.B. Peptide-Resin Cleavage

Upon completion of solid phase synthesis, the peptide was cleaved from the solid support by treatment with cleavage reagent B: TFA/phenol/H₂O/TIPS (87.5%/5%/5%/2.5%/25 ml) for 3 h. (TIPS stands for tri-isopropylsilane)

In certain instances, addition of a dithiol such as 1,2-ethane dithiol or DODT (2,2′-(ethylenedioxy)diethanethiol) may be advantageous (e.g. cleavage reagent K). The TFA solution containing the peptide was filtered and concentrated under reduced pressure at T<30° C.

The desired product was precipitated with ice-cold MTBE (methyl tert-butyl ether) or diethyl ether and centrifuged at 3000 rpm for 30 min. The centrifuged pellet was then washed with ice-cold diethyl ether and centrifuged. This process was repeated three times.

In some instances, it may be necessary to process the crude peptide to remove undesired by-products such as TFA esters, CO₂ adduct on indole nitrogen of tryptophan (carbamic acid) and 2-t-butyl-sulfanylethyl adduct on methionine residues.

To remove CO₂, adduct on indole nitrogen of tryptophan, the crude peptide was taken up in water containing 10-20% CH₃CN, 5 mg/ml and lyophilized.

To remove 2-t-butyl-sulfanylethyl adduct on Met, the crude peptide was dissolved (2 mg/ml) in a solution of H₂O/CH₃CN (50:50 v/v) containing 0.1% formic acid.

The mixture was gently shaken at 37° C. overnight.

To remove TFA esters, the crude peptide was dissolved (2 mg/ml) in a solution of H₂O/CH₃CN (50:50 v/v) containing 0.1% formic acid. The mixture was gently shaken at 37° C. for 1-4 h.

In both cases, the crude peptide solution thus obtained was partially concentrated under reduced pressure and at T<30° C. and lyophilized.

### 1.C. Purification Step

Following any of the above method indicated for synthesizing a relaxin peptide analogue, said peptide was purified before being used.

80 mg of peptide were dissolved in 1.5 mL DMSO and purified by Reverse Phase High Pressure Liquid Chromatography (RP-HPLC). The RP-HPLC was described here-under.

A GX271 Liquid Handler, 333/334 pumps, and UV/VIS 151 Gilson system was used.

Two different systems were used in order to purify said peptides:

### System A

Column Waters Delta-Pack C4 15 m 300.ANG. 250×20 mm;
Solution A: 0.1% trifluoroacetic acid (TFA) in H₂O;
Solution B: 0.1% TFA in Acetonitrile;
Gradient: 15% B for 5 min; 15% B to 50% B in 20 min;
Flow rate: 80 ml/min.

### System B

Column Waters CSH C18 5 m 250×50 mm, or Waters Sunfire C18 10 μM 250×50 mm;
Solution A=0.1% TFA in water;
Solution B=0.1% TFA in acetonitrile;
Gradient: from 1% B to 18% B in 5 min; from 18% B to 28% B in 10 min; 28% B for 15 min; from 28% B to 48% B in 10 min;
then column wash from 48% B to 90% B in 10 min;
Flow rate: 150 ml/min.

The peptide of interest was eluted in the 35-40 min time window.

The gradient was slightly adjusted according to the polarity of each peptide as characterized by its retention time on an analytical UHPLC System.

The fractions containing the pure peptide were then partially concentrated under reduced pressure at T<35° C. and lyophilized until constant weight.

For certain uses (eg in-vivo testing), it was advantageous to exchange the TFA salt to the acetate salt. Three corresponding methods were used and are described in the following part 1.D.

### 1.D. Acetate Exchange

(i) Acetate Exchange with TOYOPEARL® DEAE 650 C (Tosoh Corporation)

The ion exchange was performed using a TOYOPEARL® DEAE 650 C grade resin (a weak anion exchange resin).

120 ml of resin was washed sequentially with 15 volumes of NaOH 1M, 5 volumes of H₂O, 5 volumes of acetic acid 1.6 M, 5 volumes of acetic acid 0.16 M and finally 5 volumes of H₂O.

41.8 mg of peptide was then dissolved in 4 ml of distilled water, downloaded to the resin and gently mixed for 2 h.

Finally, the peptide was collected by elution and washed with water and lyophilized.

Peptide Recovery: 35 mg (as acetate salt by ¹⁹F NMR (400 MHz) ns 1028).

(ii) Acetate Exchange with Sepharose HiTrap Q HP Column (Strong Anionic Exchange Column)

In this second method, the ion exchange was performed using a HiTrap Q HP.

The column (5 ml bed volume) was connected to a peristaltic pump set at 48 (4.5 ml/min) and before loading the peptide it was washed with 50 ml (10 column volumes) of H₂O, 100 ml (20 column volumes) of a 1 M solution of sodium acetate, 150 ml (30 column volumes) of H₂O and with 50 ml (10 column volumes) of 0.16 M solution of acetic acid.

The pure peptide was dissolved in a 0.16 M acetic acid solution at 2 mg/ml, slowly loaded on the column and eluted at 4.5 ml/min.

The collected solution was freeze-dried.

The effectiveness of the ion exchange was attested by ¹⁹F NMR (400 MHz) ns 1028.

(iii) Acetate Exchange with BIO RAD AG1X4® Anion Exchange Resin

In a 125 ml reactor equipped with sintered glass at the bottom was charged with 6.2 g BIO RAD AG1X4® anion exchange resin, 100-200 dry mesh size (OH— form).

The resin was shaken with 3×100 ml 1.6 M aqueous acetic acid (10% v/v) and with 3×50 ml 0.16 M aqueous acetic acid (1% v/v) on a stirring plate for 20 min each.

The purified peptide as TFA salt (100 mg) was dissolved in 50 ml distilled water and poured on the exchange resin and shaken on a stirring plate for 120 min.

The aqueous solution was drained in a 100 ml round bottom flask and the resin was washed with 2×15 ml 0.16 M aqueous acetic acid (1% v/v).

The combined solutions containing the peptide as acetate salt were lyophilized until constant weight. Yield=80 mg of peptide as acetate salt.

The effectiveness of the ion exchange was attested by ¹⁹F NMR (400 MHz, ns 1028) and/or ionic chromatography.

### Example 2: Illustrative Synthesis of Relaxin Peptide Analogues

### 2.A Loading of Fmoc-Lys(Ac)-OH on Rink Amide Resin

In a 100 ml reactor equipped with a sintered glass at the bottom, 6 g of Novabiochem or ChemImpex Rink amide AM resin (Low Loading 0.47 mmol/g) was swelled in 40 ml of DMF. The solvent was drained and 30 ml of 20% piperidine in DMF solution were added. After 15 min shaking, the solvent was drained. This was repeated twice to ensure complete Fmoc protecting group removal. The resin was washed with 5×30 ml DMF.

In a separate flask a solution containing Fmoc-Lys(Ac)-OH (3.5 g, 8 mmol, 3 eq.) HOBT·H₂O (1.3 g 8.5 mmol) in 30 ml DMF was prepared. Diisopropylcarbodiimide (DIC) (1 g, 8.5 mmol) was added to this solution and after 5 min the resulting mixture was added to the resin. The suspension was shaken on a stirring plate for 4 h or until completion of the reaction as judged by Kaiser Test (Ninhidrin test) on an aliquot part of the resin.

The solvent was then drained, and the resin washed 3 times with 30 ml DMF. Fmoc-Lys(Ac)-NH₂ loaded resin was used immediately for subsequent steps or stored wet at 4° C. until needed.

### 2.B. Synthesis of Peptide Having the SEQ ID NO: 3

The following synthesis was performed using 5 times an amount of resin obtained at step 2.A. corresponding to 0.2 mmol of Fmoc-Lys(Ac)-NH₂ each. The syntheses were performed separately on each individual batches using a CEM Liberty Blue microwave peptide synthesizer to assemble the second and third residue of the peptide sequence (starting from the C-terminus).

Peptide synthesis was performed by using DIC 0.5M/Oxyma 1M in DMF.

All amino acids were introduced with double couplings using standard heating protocol.

The resin was removed from the synthesizer and Fmoc-α-methyl-lysine(Boc)-OH (3 eq.) was coupled manually using 3 eq. Oxyma™ and 3 eq. DIC with microwave heating (75° C. 15 sec. and 90° C. 110 sec). The completion of the reaction was controlled by Kaiser test. If positive, DIC 3 eq. was added followed by microwave heating as above.

When coupling of Fmoc-α-methyl-lysine(Boc)-OH was complete the rest of the peptide sequence was assembled using a CEM Liberty Blue microwave peptide synthesizer.

All amino acids were introduced with double couplings at 90° C. as above, with the exception of amino-isobutyric acid at position 21 and serine at position 29 for which a triple coupling at 90° for 2 minutes was performed. Fmoc-Lys(Dde)-OH was used at position 25.

At the end of the 5 syntheses, the 5 batches of resin were combined and transferred into a 50 mL polypropylene syringe and the peptide was acetylated at N-terminus with acetic anhydride (944 μL, 10 mmol) in DMF (30 mL) for 20 minutes, repeating the cycle twice.

Then, Dde protecting group on Lysine 25 side chain was removed by percolating 50 mL of a solution of hydrazine 5% w/v in DMF, followed by DMF washes (5×20 ml). The reaction was monitored by Kaiser Test and cleavage of an aliquot part of resin and UPLC/MS analysis.

Three TTDS spacer units were introduced by single coupling by performing three times the following procedure: To the resin a solution of Fmoc-TTDS-OH (1.62 g, 3 mmol) in 30 mL of DMF were added followed by HOAt (5 ml of a 0.6 ml solution in DMF, 3 mmol) and DIC (1 ml, 6 mmol). The syringe was agitated on an orbital table for 18 h. The reaction was monitored by Kaiser Test. The resin was washed with DMF (2×30 mL). Then to the resin, 30 mL of 20% v/v of piperidine in DMF was added. The syringe was agitated on an orbital table for 20 min. This deprotection procedure was repeated a second time and the resin was washed with DMF (2×30 mL) and dichloromethane (3×30 mL).

The three γ-glutamic acids spacers were introduced by performing a double coupling of each Fmoc-Glu-OtBu. Thus, the following procedure was applied three times:

To the resin a solution (4S)-5-tert-butoxy-4-(9H-fluoren-9-yl methoxy carbonylamino)-5-oxo-pentanoic acid (Fmoc-Glu-OtBu) (1.275 g, 3 mmol) in of 30 mL of DMF were added followed by HOAt (5 ml of a 0.6 ml solution in DMF 3 mmol) and DIC (1 ml, 6 mmol). The syringe was agitated on an orbital table for 4 h. The resin was washed with DMF (2×20 mL), and the coupling was repeated a second time. The reaction was monitored by Kaiser Test. The resin was washed with DMF (2×30 mL). Then to the resin, 30 mL of 20% v/v of piperidine in DMF was added. The syringe was agitated on an orbital table for 20 min. This deprotection procedure was repeated a second time and the resin was washed with DMF (3×30 mL) and dichloromethane (3×30 mL).

Finally, the peptide was acylated with palmitic acid (768 mg, 3 mmol), HOAt (5 ml of a 0.6 M solution in DMF, 3 mmol) and DIC (1 ml, 6 mmol) activation in DMF (30 mL) for 2.5 h. The resin was washed with DMF (2×30 mL) and dichloromethane (3×30 mL) and dried under vacuum.

The cleavage of the peptide from the resin was performed using a solution phenol (6.25 g), water (6.25 mL) and TIPS (3 mL) in TFA (QSP 125 mL) for 2.5 hours at room temperature. The resin was filtered off, and washed with 2×30 mL TFA. The combined filtrates were transferred to a 250 mL round bottom flask and partially concentrated under vacuum at T<30° C. and the peptide was precipitated by the addition of 100 mL ice-cold MTBE and centrifuged at 3600 rpm for 30 minutes.

The centrifuged pellet was then washed with ice-cold diethyl ether and centrifuged. This process was repeated three times. 4.3 g of crude peptide were obtained. The crude peptide was dissolved (10 mg/mL) in a solution of H₂O/CH₃CN (50:50 v/v) containing 0.1% formic acid and the mixture was gently shaken at 37° for 1 h, partially concentrated and lyophilized.

Purification was performed using purification system A in 10 injection of 350 mg each and the fractions containing pure desired peptide were lyophilized. The peptide as trifluoroacetate salt was obtained as a white solid.
m=428 mg (17.5%)
UPLC/MS:
RT: 4.98 min. (Analytical condition A), purity 99% (UV)
Observed mass m/z (ion type): 1454.5 (M+3H); 1091.1 (M+4H); 873.1 (M+5H).

### 2.C. Synthesis of Peptide Having the SEQ ID NO: 7

2 batches of resin obtained in 2.A. corresponding to 0.2 mmol of Fmoc-Lys(Ac)-NH2 each (0.4 mmol in total) were processed in parallel following the same procedure as for example 2.B. After coupling the third (4S)-5-tert-butoxy-4-(9H-fluoren-9-yl methoxy carbonylamino)-5-oxo-pentanoic acid (Fmoc-Glu-OtBu) the combined resin batches weighing 2.6 g were transferred to a 50 mL polypropylene syringe.

Then to the resin, 30 mL of 20% v/v of piperidine in DMF was added. The syringe was agitated on an orbital table for 20 min. This deprotection procedure was repeated a second time and the resin was washed with DMF (3×30 mL) and dichloromethane (3×30 mL).

The peptide was acylated by adding a solution of stearoyl chloride (362 mg, 1.2 mmol) and DIPEA (0.255 ml, 1.5 mmol) in 20 ml of DCM for 2.5 h. The resin was washed with DMF (2×30 mL) and dichloromethane (3×30 mL) and dried under vacuum.

The cleavage of the peptide from the resin was performed using a solution phenol (1.5 g), water (1.5 mL) and TIPS (0.6 mL) in TFA (QSP 30 mL) for 2.5 hours at room temperature. The resin was filtered off, and washed with 2×10 mL TFA. The combined filtrates were transferred to a 250 mL round bottom flask and partially concentrated under vacuum at T<30° C., the peptide was precipitated by the addition of 100 mL ice-cold MTBE and centrifuged at 3600 rpm for 30 minutes.

The centrifuged pellet was then washed with ice-cold diethyl ether and centrifuged. This process was repeated three times. 720 mg of crude peptide were obtained. Purification was performed using purification system B in 3 injection of 240 mg each. The fractions containing pure desired peptide were lyophilized. The peptide as trifluoroacetate salt was obtained as a white solid.
m=72 mg (3.6%)
UPLC/MS:
RT: 5.86 min. (Analytical condition A), purity 98% (UV)
Observed mass m/z (ion type): 1463.9 (M+3H); 1098.2 (M+4H); 878.7
(M+5H).

### 2.D. Synthesis of Peptide Having the SEQ ID NO: 6

A batch of resin obtained in 2.A. corresponding to 0.1 mmol of Fmoc-Lys(Ac)-NH2 was placed in the reactor of a CEM Liberty Blue microwave peptide synthesizer. Peptide synthesis was performed by using DIC 0.5M/Oxyma 1M in DMF.

All amino acids were introduced with double couplings at 90° C. as above, with the exception of amino-isobutyric acid at position 21 and serine at position 29 for which a triple coupling at 90° for 2 minutes was performed. Fmoc-Lys(ivDde)-OH was used at position 25.

At the end of peptide assembly, the resin was transferred to a 20 ml polypropylene syringe and peptide was acetylated at N-terminus with of acetic anhydride (95 μL, 1 mmol) and DIPEA (174 μL, 1 mmol) in DMF (10 mL) for 20 minutes, repeating the cycle twice.

Then, ivDde protecting group on lysine 25 side chain was removed by stirring with 10 mL of a solution of hydrazine 5% w/v in DMF for 20 min as many times as necessary until no starting material could be detected after cleavage of an aliquot part of resin and UPLC/MS analysis. When deprotection was judged complete, resin was washed with DMF (5×10 ml).

Two TTDS spacer units were introduced by single coupling by performing twice the following procedure: To the resin a solution of Fmoc-TTDS-OH (163 mg, 0.3 mmol) HOAt (42 mg, 0.3 mmol) and DIC (77 μL, 0.5 mmol) 7 mL of DMF was added. The syringe was agitated on an orbital table for 18 h. The reaction was monitored by Kaiser Test. When needed, a double coupling was performed. The resin was washed with DMF (2×10 mL). Then to the resin, 10 mL of 20% v/v of piperidine in DMF was added. The syringe was agitated on an orbital table for 20 min. This deprotection procedure was repeated a second time and the resin was washed with DMF (2×10 mL) and dichloromethane (3×10 mL).

The three γ-glutamic acids spacers were introduced by performing a double coupling of each Fmoc-Glu-OtBu. Thus, the following procedure was applied three times:

To the resin a solution (4S)-5-tert-butoxy-4-(9H-fluoren-9-yl methoxy carbonylamino)-5-oxo-pentanoic acid (Fmoc-Glu-OtBu) (127 mg, 0.3 mmol), HOAt (42 mg, 0.3 mmol) and DIC (77 μL, 0.5 mmol) in 7 mL of DMF. The syringe was agitated on an orbital table for 18 h. The reaction was monitored by Kaiser Test. The resin was washed with DMF (2×10 mL). Then to the resin, 10 mL of 20% v/v of piperidine in DMF was added. The syringe was agitated on an orbital table for 20 min. This deprotection procedure was repeated a second time and the resin was washed with DMF (3×10 mL) and dichloromethane (3×30 mL).

Finally, the peptide was acylated with Stearoyl chloride (62 mg, 0.2 mmol) and DIPEA (54 μL, 0.3 mmol) in 5 ml DCM for 2.5 h. The resin was washed with DMF (2×30 mL) and dichloromethane (3×30 mL) and dried under vacuum.

The cleavage of the peptide from the resin was performed using a solution phenol (0.5 g), water (0.5 mL) and TIPS (0.2 mL) in TFA (QSP 10 mL) for 2.5 hours at room temperature. The resin was filtered off and washed with 2×4 mL TFA. The combined filtrates were transferred to a 100 mL round bottom flask and partially concentrated under vacuum at T<30° C. and the peptide was precipitated by the addition of 50 mL ice-cold MTBE and centrifuged at 3600 rpm for 30 minutes.

The centrifuged pellet was then washed with ice-cold diethyl ether and centrifuged. This process was repeated three times. 240 g of crude peptide were obtained. Purification was performed using purification system B and fractions containing pure desired peptide were lyophilized. The peptide as trifluoroacetate salt was obtained as a white solid. m=38 mg (8%) UPLC/MS: RT: 5.40 min. (Analytical condition A), purity 97% (UV) Observed mass m/z (ion type): 1376.1 (M+3H); 1032.0 (M+4H); 826.0 (M+5H).

### 2.E. Synthesis of Peptide Having the SEQ ID NO: 20

This compound was obtained following the same procedure as the one used in example 2.D except that three TTDS were introduced on lysine 25 side chain.

Thus from 250 mg of resin obtained in 2.A. corresponding to 0.1 mmol of Fmoc-Lys(Ac)-NH2 a white solid was obtained. m=20 mg (4.5%) UPLC/MS: RT: 5.44 min. (Analytical condition A), purity 99% (UV) Observed mass m/z (ion type): 2215.0 (M+2H); 1476.8 (M+3H); 1107.9 (M+4H); 886.7 (M+5H).

### 2.F. Synthesis of Peptide Having the SEQ ID NO: 22

This compound was obtained following the same procedure as the one used in example 2.B. except that Fmoc-Lys(Aloc)-OH was used in position 25 (X₂₅) instead of Fmoc-Lys(Dde)-OH.

Thus 250 mg of resin obtained in 2.A. corresponding to 0.1 mmol of Fmoc-Lys(Ac)-NH2 was processed as in 2.B. until the N-Terminal acetylation step.

The Aloc group on Lys25 side chain was removed by adding to the resin, under argon atmosphere, a solution of 1 ml (8.33 mmol) of phenyl silane in 2 ml of degazed DCM and a solution of 10 mg (25.96 moles) of tetrakis-(triphenylphosphine) palladium in 4 ml DCM. The resin was shaken on an orbital table for 60 min and the reaction media was replaced with fresh reagents twice and shaken 60 min each time.

When the reaction was complete, the resin was washed with dichloromethane, 1% DIEA in DMF, 5% diethyl-dithio-carbamate in DMF, DMF, 10% DIEA in DMF, DMF and dichloromethane (3 times each).

Three PEG₂DGA spacer units were introduced by single coupling by performing three times the following procedure: To the resin a solution of Fmoc-PEG₂DGA-OH (170 mg, 0.3 mmol) in 8 mL of DCM were added followed by HOAt (0.5 ml of a 0.6 ml solution in DMF, 0.3 mmol) and DIC (100 μl, 0.642 mmol). The syringe was agitated on an orbital table for 18 h. The reaction was monitored by Kaiser Test. The resin was washed with DMF (2×10 mL). Then to the resin, 10 mL of 20% v/v of piperidine in DMF was added. The syringe was agitated on an orbital table for 20 min. This deprotection procedure was repeated a second time and the resin was washed with DMF (2×30 mL) and dichloromethane (3×30 mL).

The three γ-glutamic acids spacers were introduced by performing a double coupling of each Fmoc-Glu-OtBu. Thus, the following procedure was applied three times:

To the resin a solution (4S)-5-tert-butoxy-4-(9H-fluoren-9-yl methoxy carbonylamino)-5-oxo-pentanoic acid (Fmoc-Glu-OtBu) (0.13 g, 0.3 mmol) in of 8 mL of DMF were added followed by HOAt (0.5 ml of a 0.6 ml solution in DMF 0.3 mmol) and DIC (0.1 ml, 0.6 mmol). The syringe was agitated on an orbital table for 4 h. The resin was washed with DMF (2×20 mL) and the coupling was repeated a second time. The reaction was monitored by Kaiser Test. The resin was washed with DMF (2×10 mL). Then to the resin, 10 mL of 20% v/v of piperidine in DMF was added. The syringe was agitated on an orbital table for 20 min. This deprotection procedure was repeated a second time and the resin was washed with DMF (3×30 mL) and dichloromethane (3×30 mL).

Finally, the peptide was acylated with palmitic acid (80 mg, 0.3 mmol), HOAt (0.5 ml of a 0.6 M solution in DMF, 3 mmol) and DIC (0.1 ml, 6 mmol) activation in DMF (10 mL) for 2.5 h. The resin was washed with DMF (2×30 mL) and dichloromethane (3×30 mL) and dried under vacuum.

The cleavage of the peptide from the resin was performed using a solution phenol (0.5 g), water (0.5 mL) and TIPS (0.25 mL) in TFA (QSP 10 mL) for 2.5 hours at room temperature. The resin was filtered off, and washed with 2×5 mL TFA. The combined filtrates were transferred to a 250 mL round bottom flask and partially concentrated under vacuum at T<30° C. and the peptide was precipitated by the addition of 100 mL ice-cold MTBE and centrifuged at 3600 rpm for 30 minutes.

The centrifuged pellet was then washed with ice-cold diethyl ether and centrifuged. This process was repeated three times. 220 mg of crude peptide were obtained.

Purification was performed using purification system B. Fractions containing pure desired peptide were lyophilized. The peptide as trifluoroacetate salt was obtained as a white solid.
m=37 mg (8%)
UPLC/MS:
RT: 4.99 min. (Analytical condition A), purity 99% (UV)
Observed mass m/z (ion type): 2205.2 (M+2H); 1470.5 (M+3H); 1103.1 (M+4H); 882.7 (M+5H).

### 2.G. Results

The results obtained with SEQ ID NO: 1-32, 34-37, 39, 44, 45, 47-49, 51 and 54-97 were indicated in the following Table 1.

TABLE 1

| SEQ | UPLC Ret. | | Observed mass | | | | Mono |
|---|---|---|---|---|---|---|---|
| ID | Time Min. | | Ion type | | | | isotopic |
| NO | (Conditions) | | M + 5H | M + 4H | M + 3H | M + 2H | mass |
| 1 | 3.81 | (C) | 881.3 | 1101.3 | 1467.7 | | 4397.54 |
| 2 | 3.85 | (C) | 861.9 | 1077.2 | 1436.3 | | 4301.47 |
| 3 | 4.98 | (A) | 873.1 | 1091.1 | 1454.5 | 2181.5 | 4358.53 |
| 4 | 5.02 | (A) | 820.5 | 1023.3 | 1366.7 | 2049.4 | 4095.36 |
| 5 | 4.82 | (A) | 815.7 | 1019.3 | 1358.8 | 2037.3 | 4071.28 |
| 6 | 5.40 | (A) | 826.0 | 1032.0 | 1376.1 | 2063.8 | 4123.39 |
| 7 | 5.86 | (A) | 878.7 | 1098.2 | 1463.9 | | 4386.56 |
| 8 | 4.83 | (A) | 807.8 | 1009.5 | 1345.7 | 2018.3 | 4032.27 |
| 9 | 4.97 | (A) | 786.9 | 983.3 | 1310.7 | 1965.7 | 3927.30 |
| 10 | 4.90 | (A) | 838.5 | 1047.8 | 1396.8 | 2094.8 | 4185.39 |
| 11 | 5.03 | (A) | 880.9 | 1100.9 | 1467.5 | 2200.9 | 4397.54 |
| 12 | 4.98 | (A) | 870.5 | 1087.9 | 1450.2 | 2174.9 | 4345.5 |
| 13 | 4.99 | (A) | 870.3 | 1087.6 | 1449.8 | 2174.7 | 4344.5 |
| 14 | 4.96 | (A) | 870.3 | 1087.6 | 1449.8 | | 4344.5 |
| 15 | 5.00 | (A) | 869.9 | 1087.1 | 1449.2 | 2173.3 | 4342.5 |
| 16 | 4.92 | (A) | 809.9 | 1012.1 | 1349.1 | 2023.2 | 4042.33 |
| 17 | 5.00 | (A) | 817.6 | 1021.8 | 1362.1 | 2042.6 | 4081.34 |
| 18 | 4.73 | (A) | 829.3 | 1036.1 | 1381.2 | | 4138.40 |
| 19 | 4.88 | (A) | 786.6 | 983.0 | 1310.0 | 1965.3 | 3926.21 |
| 20 | 5.44 | (A) | 886.7 | 1107.9 | 1476.8 | 2215.0 | 4425.57 |
| 21 | 5.27 | (A) | 821.4 | 1026.3 | 1369.1 | 2051.6 | 4999.31 |
| 1 | 3.81 | (C) | 881.3 | 1101.3 | 1467.7 | | 4397.54 |
| 22 | 4.99 | (A) | 882.7 | 1103.1 | 1470.5 | 2205.2 | 4406.5 |
| 23 | 5.24 | (A) | 870.1 | 1087.4 | 1449.5 | 2173.8 | 4343.52 |
| 24 | 5.25 | (A) | 876.9 | 1095.1 | 1460.8 | 2190.8 | 4377.50 |
| 25 | 5.17 | (A) | 873.1 | 10914 | 1454.5 | 21813 | 4358.49 |
| 26 | 5.24 | (A) | 813.4 | 1016.5 | 1355.0 | 2032.1 | 4060.3 |
| 27 | 4.97 | (A) | 878.7 | 1098.1 | 1463.8 | 2194.9 | 4386.54 |
| 28 | 4.97 | (A) | 875.9 | 1094.6 | 1459.1 | 2188.2 | 4372.55 |
| 29 | 5.00 | (A) | 875.9 | 1094.6 | 1459.2 | 2188.3 | 4372.54 |
| 30 | 5.44 | (A) | 886.5 | 1107.9 | 1476.9 | | 4425.57 |
| 31 | 5.07 | (A) | 886.5 | 1107.9 | 1476.8 | | 4425.5 |
| 32 | 5.42 | (A) | 888.3 | 1110.3 | 1479.8 | 2219.5 | 4434.5 |
| 34 | 5.45 | (A) | 818.2 | 1022.5 | 1363.4 | | 4084.4 |
| 35 | 5.03 | (A) | 877.8 | 1096.8 | 1462.4 | 21928 | 4381.5 |
| 36 | 4.96 | (A) | 870.1 | 1087.6 | 1450.1 | 21743 | 43445 |
| 37 | 5.1 | (A) | 883.3 | 1103.6 | 1471.5 | 2207.4 | 4410.5 |
| 39 | 5.20 | (A) | 890.1 | 112.8 | 1483.1 | 2224.6 | 4444.52 |
| 44 | 4.80 | (A) | 877.8 | 1097.1 | 1462.8 | | 4383.5 |
| 45 | 4.81 | (A) | 836.6 | 1045.7 | 1393.7 | 2090.6 | 4177.3 |
| 47 | 4.85 | (A) | 864.6 | 1080.3 | 1440.4 | 2160.5 | 4316.5 |
| 48 | 9.23 | (D) | 864.6 | 1080.6 | 1440.8 | 2160.2 | 4316.5 |
| 49 | 9.00 | (D) | 864.6 | 1080.6 | 1440.4 | 2160.3 | 4316.5 |
| 51 | 5.38 | (A) | 912.3 | 1140.1 | 1519.8 | 2279.2 | 4554.6 |
| 54 | 5.41 | (A) | 851.8 | 1064.5 | 1419.1 | 2128.2 | 4252.4 |
| 55 | 5.03 | (A) | 892.1 | 1114.8 | 1486.1 | | 4453.6 |
| 56 | 5.42 | (A) | 876.1 | 1094.8 | 1439.3 | | 4373.5 |
| 57 | 4.90 | (A) | 880.9 | 1100.9 | 1467.1 | 2201.2 | 4316.5 |
| 58 | 5.48 | (A) | 946.8 | 1183.3 | 1577.5 | | 4727.7 |
| 59 | 5.44 | (A) | 972.7 | 1215.6 | 1620.3 | | 4856.8 |
| 60 | 5.34 | (A) | 818.2 | 1022.9 | 1363.0 | 2044.0 | 4084.4 |
| 61 | 3.41 | (A) | 878.4 | 1098.1 | 1463.7 | 2195.1 | 4386.6 |
| 62 | 5.34 | (A) | 906.6 | 1133.1 | 1510.5 | | 4526.6 |
| 63 | 4.98 | (A) | 886.3 | 1107.6 | 1476.5 | | 4424.6 |
| 64 | 4.8 | (A) | 891.9 | 1114.6 | 1485.9 | | 4452.6 |
| 65 | 4.97 | (A) | 892.2 | 1115.1 | 1486.4 | 2228.7 | 4434.6 |
| 66 | 4.97 | (A) | 897.3 | 1121.7 | 1495.2 | | 4480.6 |
| 67 | 4.98 | (A) | 886.3 | 1107.8 | 1476.8 | | 4425.5 |
| 68 | 5.05 | (A) | 880.9 | 1100.9 | 1467.5 | | 4397.5 |
| 1 | 3.81 | (C) | 881.3 | 1101.3 | 1467.7 | | 4397.54 |
| 69 | 5.06 | (A) | 886.5 | 1107.8 | 1476.8 | | 4425.5 |
| 70 | 9.29 | (D) | 886.5 | 1107.8 | 1476.8 | | 4425.6 |
| 71 | 4.84 | (A) | 860.9 | 1076.3 | 1434.4 | | 4298.5 |
| 72 | 7.86 | (D) | 875.4 | 1094.1 | 1458.5 | 2187.4 | 4370.5 |
| 73 | 4.87 | (A) | 866.5 | 1082.8 | 1443.4 | | 4325.5 |
| 74 | 5.12 | (A) | 946.9 | 1183.4 | 1577.5 | | 4727.8 |
| 75 | 5.28 | (A) | 952.4 | 1190.6 | 1590.9 | | 4755.7 |
| 76 | 5.28 | (A) | 884.2 | 1105.1 | 1473.1 | | 4414.5 |
| 77 | 5.20 | (A) | 944.5 | 1180.7 | 1573.8 | | 4716.7 |
| 78 | 4.09 | (A) | 933.5 | 1166.7 | 1554.9 | | 4660.7 |
| 79 | 4.66 | (A) | 878.5 | 1098.1 | 1463.5 | | 4385.6 |
| 80 | 4.90 | (A) | 898.8 | 1123.5 | 1497.7 | | 4487.5 |
| 81 | 4.84 | (A) | 884.5 | 1105.3 | 1473.4 | 2210.1 | 4415.6 |
| 82 | 4.80 | (A) | 883.7 | 1104.4 | 1422.1 | | 4411.6 |
| 83 | 4.71 | (A) | 886.5 | 1107.6 | 1476.7 | 2214.4 | 4424.6 |
| 84 | 5.12 | (A) | 878.7 | 1097.9 | 1463.8 | 2195.1 | 4386.6 |
| 85 | 4.98 | (A) | 894.7 | 1118.6 | 1490.8 | | 4467.6 |
| 86 | 5.14 | (A) | 873.1 | 1091.1 | 1454.4 | 2181.6 | 4358.5 |
| 87 | 4.81 | (A) | 875.7 | 1094.6 | 1459.1 | 2187.7 | 4372.5 |
| 88 | 4.82 | (A) | 873.1 | 1090.9 | 1454.2 | 2181.3 | 4358.5 |
| 89 | 4.28 | (A) | 732.4 | 878.7 | 1098.2 | 1436.9 | 4386.5 |
| 90 | 4.82 | (A) | 870.1 | 1087.3 | 1449.5 | 2174.2 | 4344.5 |
| 91 | 4.89 | (A) | 887.7 | 1109.6 | 1479.2 | 2218.9 | 4432.5 |
| 92 | 4.20 | (A) | 895.7 | 1119.4 | 1492.2 | | 4471.5 |
| 93 | 4.83 | (A) | 878.5 | 1097.7 | 1463.9 | 2194.9 | 4386.5 |
| 94 | 4.84 | (A) | 878.2 | 1097.5 | 1463.3 | 2194.5 | 4384.6 |
| 95 | 4.40 | (A) | 867.8 | 1084.2 | 1445.3 | 2167.9 | 4330.5 |
| 96 | 4.24 | (A) | 859.0 | 1073.5 | 1431.3 | 2146.5 | 4288.5 |
| 97 | 3.77 | (A) | 884.0 | 1105.9 | 1474.2 | | 4417.5 |

### Condition a was the Following:

Column: Acquity Peptide CSH, C18, 130.ANG., 2.1×100 mm, 1.7 um;
Column Temperature: 50° C., Flow=0.6 ml/min;
Solvent A: 0.100 TFA in H₂O;
Solvent B: 0.100 TFA in CH₃CN;
Gradient: from 0 to 1 min B=2%, from 1 to 7 min B=2% to 70%, from 7 to 8 min B=70% to 100%;
UV detector: wavelength: 220 nm; MS acquisition: ESI+200 to 3000 uma.

### Condition B was the Following:

Column: Acquity BEH C18, 130.ANG., 2.1×50 mm, 1.7 m
Column temperature 60° C.;
Flow 0.6 ml/mn Solvent A: 0.05% TFA in H2O Solvent B: 0.05% TFA in CH₃CN
Gradient: from 0 to 1 min B=2%, from 1 to 16 min B=2% to 100%, from 16 to 17 min B=100%
UV detector: wavelength: 220 nm; MS acquisition: ESI+200 to 3000 uma

### Condition C was the Following:

Column: ACQUITY BEH C4, 130.ANG., 2.1×50 mm, 1.7 μm;
Column temperature 45° C.;
Flow rate: 0.4 ml/min;
Solvent A: 0.1% TFA in H₂O; Solvent B: 0.05% TFA in CH₃CN
Gradient: from 0 to 1 min B=30%, from 1 to 5 min B=30% to 50%, from 5 to 6 min B=80%
UV detector: wavelength: 220 nm; MS acquisition: ESI+200 to 3000 uma

### Condition D was the Following:

Column: Acquity Peptide CSH, C18, 130.ANG., 2.1×150 mm, 1.7 um;
Column Temperature: 60° C.;
Flow=0.4 ml/min;
Solvent A: 0.1% TFA in H₂O; Solvent B: 0.1% TFA in CH₃CN;
Gradient: from 0 to 1 min B=2%, from 1 to 14 min B=2% to 70%, from 14 to 16 min B=70% to 100%;
UV detector: wavelength: 220 nm; MS acquisition: ESI+200 to 3000 uma.

The following relaxin peptide analogue can be prepared respectively as described above: SEQ ID NO 33, 38, 40, 41, 43, 46, 50, 52 and 53.

### Example 3: In Vitro Analysis of Relaxin Peptide Analogues on RXFP1 Receptors (OVCAR5 cAMP Assay)

### A. Method

OVCAR5 cells expressing endogenous human RXFP1 were used to test RXFP1 agonist properties of relaxin peptide analogues, and in particular of the peptides of sequence SEQ ID NO: 1-97.

Since RXFP1 is a Gs coupled GPCR, increases in cAMP was used as readout of RXFP1 activation.

Isobutyl methyl xanthine (IBMX) was used to inhibit phosphodiesterase activity facilitating cAMP measurements. HTRF (Homogenous Time Resolved Fluorescence) technology was used to detect cAMP due to its high sensitivity.

In summary, OVCAR5 were grown in regular medium (RPMI) containing 10% fetal calf serum (FCS) and 1% antibiotics (penicillin/streptomycin).

Before the experiments, cells were detached with accutase and incubated for 40 minutes at 37° C. with 1 mM (3-isobutyl-1-methylxanthine) IBMX.

Cells were then distributed in 384 black well plates containing increasing concentrations of the different peptides in a fix volume of medium (without FCS).

After an incubation of 30 min at 37° C. in a humid incubator in 5% CO₂, the reaction was stopped by adding a fixed volume of a solution containing a lysis buffer and cAMP-D2 (cAMP labeled with the dye d2) and the anti-cAMP antibody linked to Europium and used for cAMP detection.

Readout of the experiment were performed on a fluorimeter allowing HTRF measurement. Activation curves were generated by plotting the intracellular value of cAMP versus log 10 of the compound concentration.

The 50% activation concentration (EC₅₀) was calculated by nonlinear regression using the sigmoidal dose-response (variable slope) equation with Prism 5 software.

Emax % was determined as the maximal intracellular value of cAMP for test compound (upper limit of cAMP vs concentration curve) divided by the maximal intracellular value of cAMP for human relaxin-2 (H2-R1x) determined in the same test occasion multiplied by 100.

$$E max \% = 100 \times [ {cAMP}_{test cpd} ] / [ {cAMP}_{H 2 - R 1 x} ]$$

### B. Results

The results obtained with relaxin peptide analogues are represented in the following Table 2.

TABLE 2

OVCAR 5 cells (cAMP)

| SEQ ID NO | EC50 (nM) | EMax |
|---|---|---|
| 1 | 1.50 | 99% |
| 2 | 2.40 | 99% |
| 3 | 0.34 | 97% |
| 4 | 1.50 | 89% |
| 5 | 9.10 | 85% |
| 6 | 0.27 | 95% |
| 7 | 0.07 | 95% |
| 8 | 1.86 | 92% |
| 9 | 0.70 | 93% |
| 10 | 0.79 | 95% |
| 11 | 0.83 | 93% |
| 12 | 0.93 | 90% |
| 13 | 2.12 | 91% |
| 14 | 3.20 | 90% |
| 15 | 2.10 | 85% |
| 16 | 6.80 | 88% |
| 17 | 4.60 | 91% |
| 18 | 1.54 | 92% |
| 19 | 11.5 | 94% |
| 20 | 0.15 | 96% |
| 21 | 0.65 | 93% |
| 22 | 1.00 | 92% |
| 23 | 11.1 | 92% |
| 24 | 36.4 | 100% |
| 25 | 28.7 | 100% |
| 26 | 0.43 | 96% |
| 27 | 1.80 | 98% |
| 28 | 0.62 | 88% |
| 29 | 1.60 | 88% |
| 30 | 0.21 | 89% |
| 31 | 0.19 | 94% |
| 32 | 0.10 | 97% |
| 34 | 0.14 | 90% |
| 35 | 2.33 | 91% |
| 36 | 3.60 | 93% |
| 37 | 5.60 | 98% |
| 39 | 8.70 | 93% |
| 42 | 1.50 | 84% |
| 44 | 2.2 | 97% |
| 45 | 0.45 | 96% |
| 47 | 0.64 | 95% |
| 48 | 0.45 | 96% |
| 49 | 0.19 | 97% |
| 51 | 0.10 | 98% |
| 54 | 0.16 | 96% |
| 55 | 0.18 | 93% |
| 56 | 0.15 | 91% |
| 57 | 0.44 | 98% |
| 58 | 0.08 | 98% |
| 59 | 0.09 | 99% |
| 60 | 0.15 | 95% |
| 61 | 0.10 | 97% |
| 62 | 0.60 | 98% |
| 63 | 1.45 | 97% |
| 64 | 0.52 | 95% |
| 65 | 1.19 | 95% |
| 66 | 6.31 | 93% |
| 67 | 0.48 | 95% |
| 68 | 0.88 | 98% |
| 69 | 0.78 | 97% |
| 70 | 1.34 | 94% |
| 71 | 0.36 | 95% |
| 72 | 0.56 | 91% |
| 73 | 0.20 | 95% |
| 74 | 0.54 | 94% |
| 75 | 0.19 | 97% |
| 76 | 0.18 | 96% |
| 77 | 0.11 | 98% |
| 78 | 0.18 | 99% |
| 79 | 0.45 | 95% |
| 80 | 0.63 | 93% |
| 81 | 0.16 | 98% |
| 82 | 0.54 | 95% |
| 83 | 0.25 | 94% |
| 84 | 0.10 | 95% |
| 85 | 0.30 | 92% |
| 86 | 0.30 | 95% |
| 87 | 0.10 | 96% |
| 88 | 0.17 | 96% |
| 89 | 0.10 | 97% |
| 90 | 0.14 | 97% |
| 91 | 0.09 | 97% |
| 92 | 0.10 | 99% |
| 93 | 0.77 | 86% |
| 94 | 1.74 | 88% |
| 95 | 1.80 | 91% |
| 96 | 0.88 | 92% |
| 97 | 0.29 | 98% |

Relaxin peptide analogues described herein are potent agonists of human RXFP1, with consistently high Eₘₐₓ values in the in vitro OVCAR5 cAMP assay, comparable to native human relaxin-2.

The same experiment has been performed with peptides from the prior art (Bathgate et al. WO2015/157829, incorporated by reference in its entirety) known under the names B7-33 C11.23S, AcB7-33 C11.23S and KKKK(AcB7-29 C11.23S).

The following EC₅₀ results in the OVCAR5 cAMP assay have been obtained for these three peptides.

TABLE 3

cAMP assay in OVCAR 5 cells

| Name | EC50 (nM) | Emax |
|---|---|---|
| B7-33 C11.238 | 1641 | 90% |
| AcB7-33 C11.238 | 929 | 70% |
| KKKK(AcB7-29 C11.238) | 206 | 84% |

As demonstrated above, the relaxin peptide analogues in Table 2 possess very interesting RXFP1 agonist properties and are very effective in activating RXFP1. They are moreover all significantly and unexpectedly superior to the peptides from the prior art.

### Example 4: Phase 1, Randomized, Double-Blind, Placebo-Controlled, Single and Multiple Ascending Dose Ranging Study in Healthy Volunteers to Assess Safety, Tolerability, and Evaluate the Pharmacokinetics and Pharmacodynamics of Relaxin Agonist

Described in this Example is a Phase 1, randomized, double-blind, pacebo controlled trial for assessing safety, tolerability, PK, and PD of a Relaxin Agonist (i.e., Relaxin peptide analogue of SEQ ID NO: 3).

### Study Design and Safety Results of Study

The SAD part A was a SAD study with a total of 40 subjects (n=8 per cohort; 6 to Relaxin Agonist and 2 to placebo) to be enrolled sequentially in up to 5 cohorts (Cohorts A1 to A5). Four cohorts received a dose via SC injection only and 1 cohort (Cohort A2, 4.0 mg) was dosed via both SC and IV injection, with at least 2-week washout period between SC and IV injections. The 4.0 mg dosing cohort (Cohort A2) received the IV dose after the 12.0 mg SC cohort (Cohort A3) was completed. Subjects in each cohort received 1 of 5 dose levels (1.0 mg, 4.0 mg, 12.0 mg, 24.0 mg, and 48.0 mg) or matching placebo as follows:
Cohort A1: a single SC dose of 1.0 mg Relaxin Agonist (n=6) or matching placebo (n=2)
Cohort A2: Period 1 a single SC dose of 4.0 mg Relaxin Agonist (n=6) or matching placebo (n=2)
Cohort A2: Period 2 a single 15-min IV dose of 4.0 mg Relaxin Agonist (n=6) or matching placebo (n=2)
Cohort A3: a single SC dose of 12.0 mg Relaxin Agonist (n=6) or matching placebo (n=2)
Cohort A4: a single SC dose of 24.0 mg Relaxin Agonist (n=6) or matching placebo (n=2)
Cohort A5: a single SC dose of 48.0 mg Relaxin Agonist (n=6) or matching placebo (n=2)

In this first-in-human study, the subjects in all SAD cohorts were dosed according to a sentinel dosing design to ensure optimal safety. This meant that initially 2 subjects were dosed: 1 subject with Relaxin Agonist and 1 subject with placebo. If at the lowest dose level (Cohort A1) the safety and tolerability results of the first 48 h following dosing for the initial subjects was acceptable to the Investigator, the other 6 subjects (5 active and 1 placebo) were to be dosed. In Cohorts A2 to A5, the safety and tolerability results of the first 24 h following dose administration were evaluated in the first 2 subjects before the remainder of the subjects in that cohort are dosed.

In the MAD part B of the study, a total of 24 subjects (n=8 per cohort; 6 to Relaxin Agonist and 2 to placebo) were enrolled sequentially (Cohorts B1 to B3) with subjects within a cohort being enrolled in a parallel fashion. Cohorts B1 to B3 receive one of 3 SC doses (5.0 mg, 15.0 mg, 30.0 mg once daily [QD]) as follows:
Cohort B1: 5.0 mg SC Relaxin Agonist (n=6) or matching placebo (n=2) QD for 14 days
Cohort B2: 15.0 mg SC Relaxin Agonist (n=6) or matching placebo (n=2) QD for 14 days
Cohort B3: 30.0 mg SC Relaxin Agonist (n=6) or matching placebo (n=2) QD for 14 days

In summary, single subcutaneous (sc) doses of 1.0 mg, 4.0 mg, 12.0 mg, 24 mg, and 48 mg, and a single intravenous (iv) dose of 4.0 mg were administered in part A. All doses were well tolerated and safe. All reported adverse events were mild (grade 1) and most resolved spontaneously. The most frequently reported adverse events included injection site erythema, itching at the injection site, and burning sensation at injection site, all reported as related to study drug.

During part B, single and multiple once daily subcutaneous doses of 5.0 mg, 15.0 mg, and 30 mg were administered for 14 days. All doses were well tolerated and safe. All reported adverse events were mild (grade 1) and most resolved spontaneously. The most frequently reported adverse events included injection site erythema, itching at the injection site, and burning sensation at injection site, all reported as related to study drug and all resolved rapidly (most on the same day).

### Clinical Pharmacokinetics of Study

Preliminary PK results after single ascending doses of Relaxin Agonist administered subcutaneously (sc) are summarized in Table 4. Exposures increased dose-proportionally up to a dose of 12 mg Relaxin Agonist. After 24 mg and 48 mg sc doses, higher than dose-proportional exposures were observed (approximately 15% higher than expected).

TABLE 4
Key PK parameters after sc SAD of Relaxin Agonist in Study

| | Group | | | | | | | | | |
|---|---|---|---|---|---|---|---|---|---|---|
| | A1 | | A2 | | A3 | | A4 | | A5 | |
| | Dose | | | | | | | | | |
| | 1.0 mg sc | | 4.0 mg sc | | 12.0 mg sc | | 24.0 mg sc | | 48.0 mg sc | |
| | Cmax | AUCinf | Cmax | AUCinf | Cmax | AUCinf | Cmax | AUCinf | Cmax | AUCinf |
| | ng/mL | h · ng/mL | ng/mL | h · ng/mL | ng/mL | h · ng/mL | ng/mL | h · ng/mL | ng/mL | h · ng/mL |
| N | 6 | 6 | 6 | 6 | 6 | 6 | 6 | 6 | 6 | 6 |
| Mean | 82.87 | 3216.79 | 355.67 | 14951.72 | 1000.00 | 37106.80 | 2364.00 | 78610.32 | 4561.67 | 178322.87 |
| CV % | 35.48 | 19.83 | 24.88 | 22.06 | 25.32 | 32.75 | 15.23 | 18.81 | 17.59 | 27.99 |

Preliminary PK results after a single intravenous (iv) dose of 4 mg Relaxin Agonist are summarized in Table 5. Observed Cmax and AUCinf after iv administration were approximately 3.5 and 1.3-fold higher as compared to sc administration. Therefore, after sc administration, the absolute bioavailability (F) is approximately 0.76.

TABLE 5
Key PK parameters after single iv dose of Relaxin Agonist

Group
A2
Dose
4.0 mg iv

| | Cmax | AUCinf |
|---|---|---|
| | ng/mL | h · ng/mlL |
| N | 6 | 6 |
| Mean | 1245.0 | 19739.48 |
| CV % | 13.69 | 19.34 |

Preliminary PK results after single and multiple ascending doses of Relaxin Agonist administered subcutaneously (sc) are summarized in Table 6. For all dose regimens (5 mg, 15 mg, and 30 mg), an accumulation of approximately 2.6-fold was observed on Day 14. Exposures increased dose-proportionally up to dose regimen of 30 mg Relaxin Agonist. Terminal half life was observed around 23-24 hours.

TABLE 6
Key PK parameters after sc MAD doses of Relaxin Agonist in Study

| | | Group | | | | | |
|---|---|---|---|---|---|---|---|
| | | B1 | | B2 | | B3 | |
| | | Dose | | | | | |
| | | 5 mg sc | | 15 mg sc | | 30 mg sc | |
| | | Cmax | AUC0-24 | Cmax | AUC0-24 | Cmax | AUC0-24 |
| | | ng/mL | h · ng/mL | ng/mL | h · ng/mL | ng/mL | h · ng/mL |
| Day 1 | N | 6 | 6 | 6 | 6 | 6 | 6 |
| | Mean | 384.3 | 7788.9 | 1631.7 | 29622.7 | 2011.7 | 38410.8 |
| | CV % | 15.2 | 14.0 | 22.8 | 19.8 | 22.9 | 14.9 |
| Day 14 | N | 6 | 6 | 6 | 6 | 6 | 6 |
| | Mean | 1018.7 | 21579.2 | 3631.7 | 67244.2 | 6011.7 | 117650.0 |
| | CV % | 13.3 | 14.3 | 19.0 | 21.7 | 21.6 | 19.3 |

TABLE 7
Key dose linearity parameters after multiple ascending doses of Relaxin Agonist

| | | Group | | | | | | | | |
|---|---|---|---|---|---|---|---|---|---|---|
| | | B1 | | | B2 | | | B3 | | |
| | | Dose | | | | | | | | |
| | | 5 mg sc | | | 15 mg sc | | | 30 mg sc | | |
| | | Cmax | AUCinf | T½ | Cmax | AUCinf | T½ | Cmax | AUCinf | T½ |
| | | ng/mL | h · ng/mL | h | ng/mL | h · ng/mL | h | ng/mL | h · ng/mL | h |
| Day 14 | N | 6 | 6 | 6 | 6 | 6 | 6 | 6 | 6 | 6 |
| | Mean | 1018.7 | 50345.3 | 23.4 | 3631.7 | 142883.3 | 22.9 | 6011.7 | 278278.6 | 24.3 |
| | CV % | 13.3 | 22.9 | 11.7 | 19.0 | 26.7 | 11.0 | 21.6 | 28.9 | 22.9 |

### Renal Plasma Flow (PAH)

Renal plasma flow (RPF) measurements were approximated based on Para-aminohippurate (PAH) clearance. PAH measurements were performed in MAD (Part B) study only. Baseline measurements were taken on Day 1, and Day 13 measurements were taken at −2.5, −1, and −0.5 h predose and at 4, 5.5, and 6 h postdose, which was the predicted Tmax.

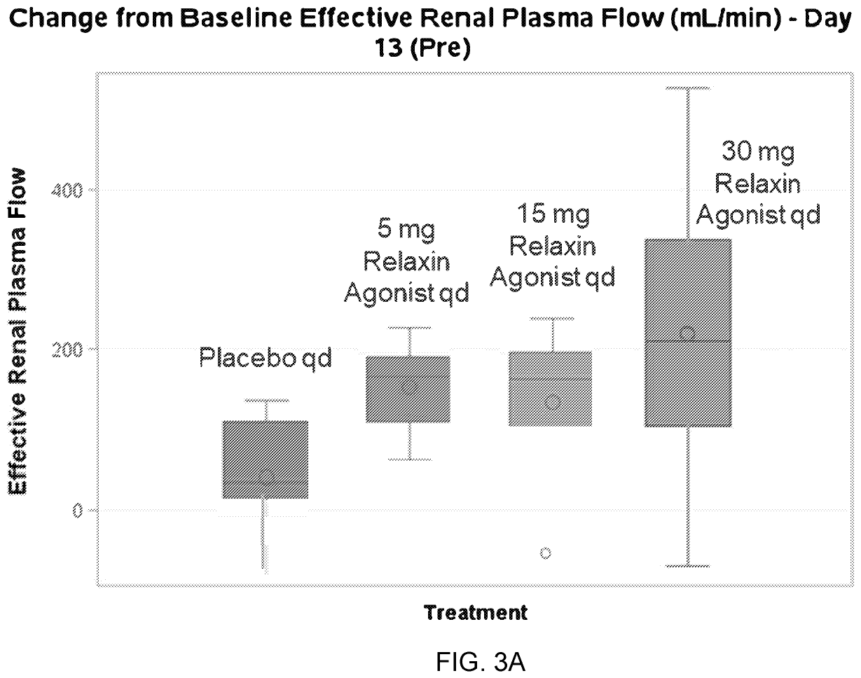

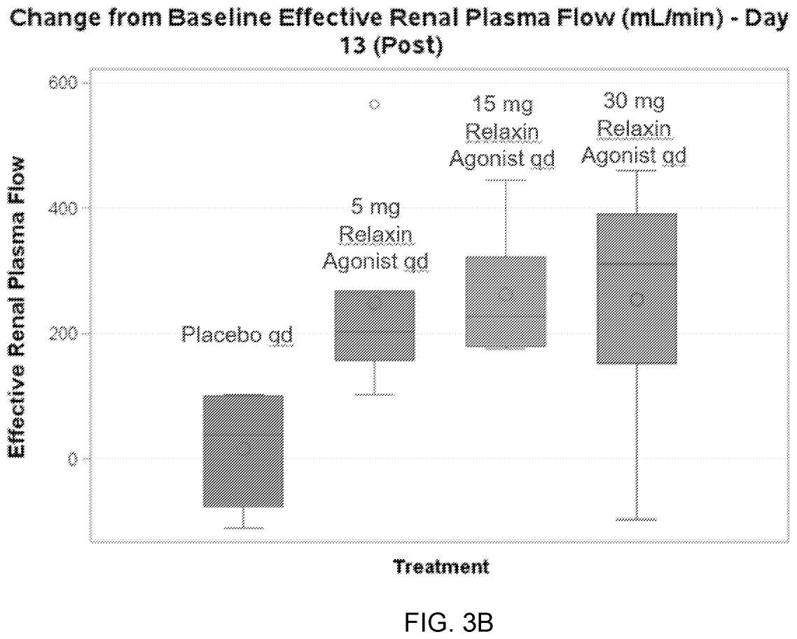

Reference is made to FIGS. 3A and 3B, which show the change from baseline effective renal plasma flow following administration of Relaxin Agonist. After multiple dosing with Relaxin Agonist or placebo, RPF assessed using the PAH biomarker tended to be increased in the active dose groups compared to placebo, with a more pronounced effect on Day 13 postdose compared to Day 13 predose for all treatment groups.

Change from baseline values for PAH clearance after multiple dosing with 5 mg QD to 30 mg QD varied between 118 mL/min and 187 mL/min at predose Day 13 and between 212 mL/min and 223 mL/min at postdose Day 13. There was no clear trend in change from baseline of PAH clearance related to dose. The change from baseline of PAH clearance in the placebo group was lower compared to the active groups with a change from baseline PAH clearance of 40 mL/min at predose Day 13 and 13 mL/min at postdose Day 13. Consistently with PAH clearance, effective RPF (eRPF) was higher in the active dose groups compared to the placebo group, without a clear relationship with dose.

### Study Design and Safety Results of Study

This study is an open label, phase 1, multi-centre, single dose study in severe renal impaired subjects and in matched subjects with normal renal function. In this preliminary evaluation, five subjects with severe renal impairment and two subjects with normal renal function were included.

Two adverse events (palpitation and headache) in one subject were reported. One subject reported palpitation, grade 1 (mild), possibly related to study drug. ECGs were performed with no clinically significant abnormalities. No action was taken with study drug, and the event resolved spontaneously. The same subject also reported headache, grade 1, not related, no action taken with study drug. This event also resolved spontaneously. In summary, single sc doses of 4.0 mg Relaxin Agonist were administered to 5 subjects with severe renal impairment and 2 subjects with normal renal function. All doses were well tolerated and safe. Two AEs were reported in subject, both grade 1 (mild) and resolving spontaneously.

### Clinical Pharmacokinetics of Study

This study is a Phase 1, multi-center, single dose study in severe renal impaired subjects and in matched subjects with normal renal function. Five subjects with severe renal impairment and two subjects with normal renal function were included. PK sampling was performed predose and at 1, 2, 3, 4, 6, 8, 10, 12, 24, 48, 72, 96, and 120 h postdose.

Mean Cmax in the severe renal impaired group is 391.8 ng/mL. Mean Cmax in the normal group is 302.0 and in the SAD study after 4 mg it was 355.7. For mean AUCinf, there is a similar increase in the severe renal impaired group as compared to the normal group and compared to the SAD 4 mg group. For mean Cmax there is a 10-30% increase. For mean AUCinf there is a 14-31% increase.

TABLE 8
Pharmacokinetics of Study

| | Normal renal function group | | Severe renal impairment group | |
|---|---|---|---|---|
| | Cmax | AUCINF_pred | Cmax | AUCINF_pred |
| N | 2 | 2 | 5 | 5 |
| Mean | 302 | 12920.598 | 391.8 | 16979.141 |
| CV % | 46.8 | 32.9 | 58 | 42.1 |

### Example 5: Phase II Trial Assessing Safety, Tolerability, Efficacy, and Pharmacokinetics of Relaxin Agonist in Combination with Terlipressin

Described in this example is a randomized, single blind controlled, two groups, multicenter trial preceeded by a safety run-in, in patients with hepatorenal syndrome (HRS) -Acute Kidney Injury (AKI). The purpose of this randomized single blind, active-controlled study is to evaluate the safety and efficacy of a Relaxin Agonist (i.e., Relaxin peptide analogue of SEQ ID NO: 3) combined with terlipressin as compared to terlipressin alone in the treatment of patients with HRS-AKI. Efficacy is assessed through the primary endpoint of established hepatorenal syndrome (HRS) reversal. Other important efficacy parameters are assessed. Safety data are collected and incorporated into the overall safety assessment of the Relaxin Agonist.

For this study, responders are defined according to the International Club of Ascites (ICA) criteria. Full response is defined as two serum creatinine levels returning to a value within 0.3 mg/dL (26.5 micromolar/L) of a baseline serum creatinine value at least 2 hours apart. Partial response is defined as a regression of at least 1 acute kidney injury (AKI) stage with a reduction of serum creatinine greater than or equal to 0.3 mg/dL above a baseline serum creatinine value. Established HRS reversal (clinical responders) are defined as patients with a Full or Partial response based on serum creatinine levels and AKI stage and are alive without renal replacement therapy (RRT) for at least 30 days after original treatment start.

The primary objectives of the study include:
To evaluate the tolerability and safety of Relaxin Agonist in combination with terlipressin versus terlipressin alone.
To evaluate the efficacy of Relaxin Agonist in combination with terlipressin versus terlipressin alone based on the number of responding patients (responder rate for Established HRS reversal, considering Full and Partial responses as separate outcome measures and combining them in a single group.)
To evaluate the efficacy of the addition of Relaxin Agonist to terlipressin in terlipressin-non-responders.

The secondary objectives of the study include:
To evaluate the efficacy of Relaxin Agonist in combination with terlipressin versus terlipressin alone on mortality at days 30, 60, and 90.
To evaluate the efficacy of Relaxin Agonist in combination with terlipressin versus terlipressin alone on liver transplant rates at days 30, 60, and 90.
To evaluate the efficacy of Relaxin Agonist in combination with terlipressin versus terlipressin alone on the incidence of RRT at days 30, 60, and 90.
To evaluate the efficacy of Relaxin Agonist in combination with terlipressin versus terlipressin alone on HRS response based on SCr/AKI stage (total responses=Full Response+Partial Response, Full Response, or Partial Response).
To evaluate the efficacy of Relaxin Agonist in combination with terlipressin versus terlipressin alone on the durability of Established HRS reversal (number of patients responding and without RRT by days 45, 60, and 90.
To evaluate the efficacy of Relaxin Agonist in combination with terlipressin versus terlipressin alone on HRS recurrence.
To evaluate the efficacy of Relaxin Agonist in combination with terlipressin versus terlipressin alone on worsening of acute or chronic liver failure (ACLF) to stage 3, or reduction in ACLF stage.
To evaluate the efficacy of Relaxin Agonist in combination with terlipressin versus terlipressin alone on change in Model for End-Stage Liver Disease (MELD) score at days 30, 60, and 90.
To evaluate the efficacy of Relaxin Agonist in combination with terlipressin versus terlipressin alone on the number of patients with Full and Partial HRS response based on SCr/AKI stage, and where the patient is alive without RRT 10 days after achievement of HRS response (instead of 30 days from the start of therapy).

The exploratory objectives of the study include:
To evaluate change from baseline in serum and urine biomarkers (Cystatin C, Endothelin-1, von Willebrand Factor (vWF), Neutrophil Gelatinase Associated Lipocalin (NGAL), and Kidney Injury Molecule-1 (KIM 1))
To evaluate the population pharmacokinetics (PK) of Relaxin Agonist in combination with terlipressin.

### Study Design

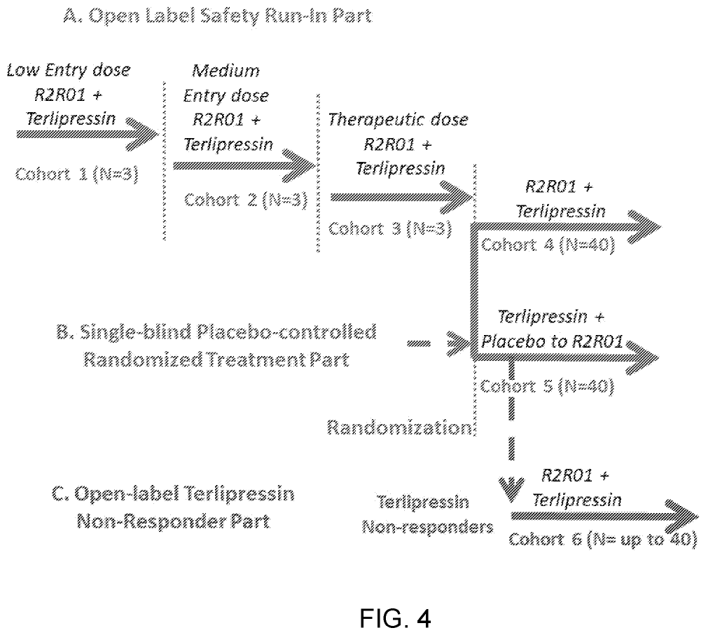

This is a randomized, single blind controlled, two groups, multicenter trial preceeded by a safety run-in, in patients with hepatorenal syndrome—Acute Kidney Injury (AKI). Reference is now made to FIG. 4, which depicts an example study design of a Phase II Trial Assessing Safety, Tolerability, Efficacy, and Pharmacokinetics of Relaxin Agonist in Combination with Terlipressin.

As shown in FIG. 4, the study consists of:
A. an Open-Label Safety Run-In Part with 3 Cohorts of patients, followed by
B. a Single-Blind Placebo-Controlled Randomized Part with two Cohorts of patients treated in parallel, and
C. an Open-Label Terlipressin Non-Responder Cohort.

All patients in all Cohorts are treated with terlipressin, administered as a 1 mg bolus IV infusion every 6 hours (h), to be increased if clinically appropriate up to 2.0 mg infusion over 6h. Terlipressin dosing should continue up to 24 h after achievement of an HRS response (either Partial or Full response) based on Serum Creatinine (SCr)/AKI stage or up to day 14.

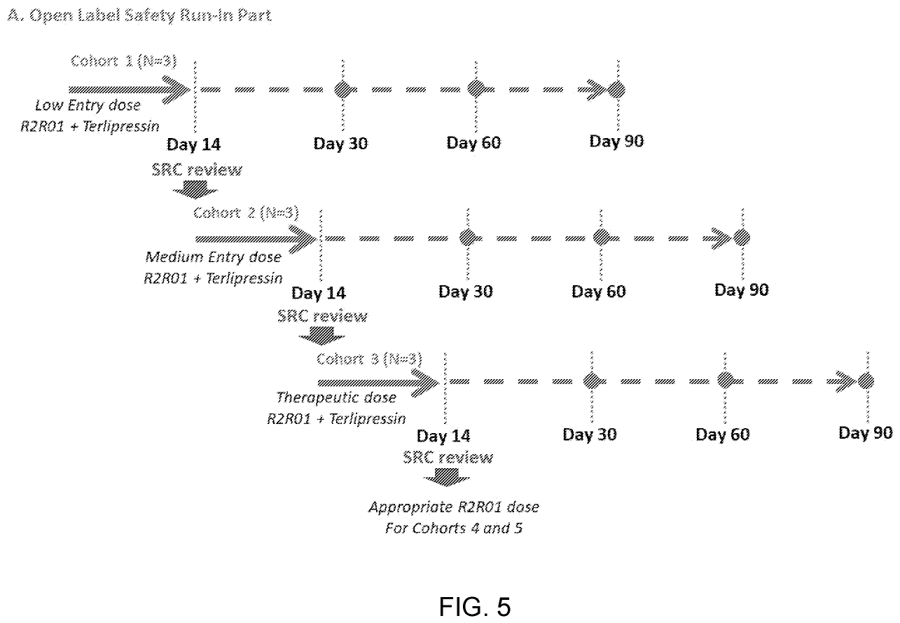

Reference is now made to FIG. 5, which depicts a design of the open label safety run-in part of the overall study design shown in FIG. 4. Here, three initial cohorts of 3 patients (labeled in FIG. 4 as Cohorts 1, 2, and 3) each are treated open label with the combination of terlipressin and Relaxin Agonist to ascertain its safety.

Cohort 1 (N=3) receives terlipressin and 1.0 mg Relaxin Agonist iv over 4 hrs, followed by 2.5 mg Relaxin Agonist sc at 12 hours after initiation of the iv, and 2.5 mg Relaxin Agonist sc at 24 hours, and thereafter once a day up to 24 h after achievement of an HRS response (either Partial or Full) based on SCr/AKI stage or up to Day 14. Cohort 1 finishes dosing before starting Cohort 2.

Cohort 2 (N=3) receives terlipressin and 2.0 mg Relaxin Agonist IV over 4 h, followed by 5.0 mg Relaxin Agonist SC at 12 h after initiation of the IV, and 5.0 mg Relaxin Agonist SC at 24 h, and thereafter the same dose once a day up to 24 h after achievement of an HRS response (either Partial or Full) based on SCr/AKI stage or up to Day 14. Cohort 2 finishes dosing before starting Cohort 3.

Cohort 3 (N=3) receives terlipressin and 4.0 mg Relaxin Agonist IV over 4 h, followed by 5.0 mg Relaxin Agonist SC at 12 h after initiation of the IV, and 10.0 mg Relaxin Agonist SC at 24 h, and thereafter the same dose (10.0 mg) once a day up to 24 h after achievement of an HRS response (either Partial or Full) based on SCr/AKI stage or up to Day 14; i.e., the same dose and schedule as to be used for Cohort 4 in the randomized part of the trial.

Cohort 3 finishes dosing before Part B, the Single-Blind Placebo-Controlled Randomized Part starts. Based on safety and tolerability of the various Relaxin Agonist dose schedules in Cohorts 1, 2, and 3, the SRC determines the appropriate Relaxin Agonist dose schedule to be taken forward for Cohorts 4 and 5. This Relaxin Agonist schedule is one of the three treatment schedules from Cohorts 1, 2, or 3.

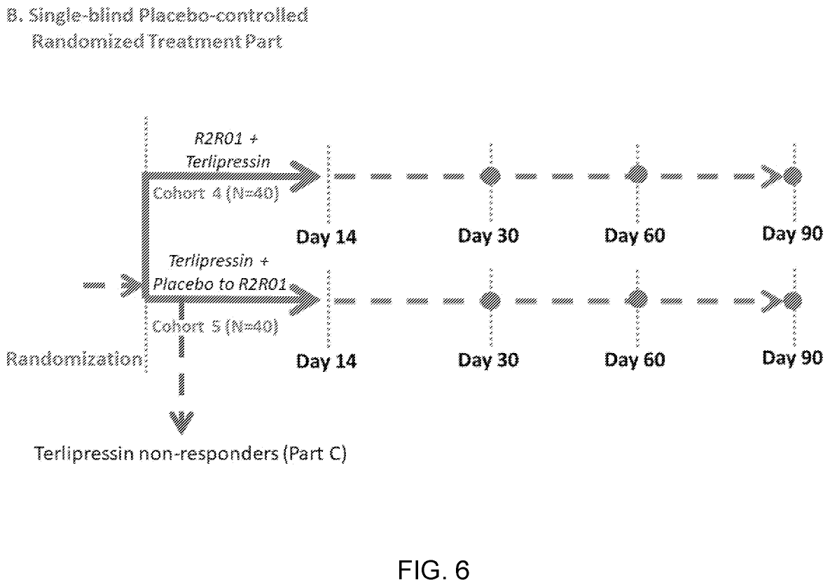

Reference is now made to FIG. 6, which depicts a design of the single-blind placebo-controlled randomized treatment part of the overall study design shown in FIG. 4. After conclusion of the Open-Label Safety Run-In Part (shown in FIG. 5), and after the appropriate Relaxin Agonist dose schedule is determined, approximately 80 patients are randomized 1:1 to Relaxin Agonist plus terlipressin (Cohort 4) or terlipressin with placebo (Cohort 5).

At randomization, patients are stratified by the presence of systemic inflammatory response syndrome (SIRS), since patients with SIRS have shown a better response to terlipressin than patients without SIRS.

Provided that the highest dose schedule of Relaxin Agonist (as investigated in Cohort 3) is safe and well tolerated, Relaxin Agonist is administered to patients in Cohort 4 on the first day as an infusion of 4.0 mg over 4 h, followed by 5.0 mg SC at 12 h after initiation of the infusion, followed by 10.0 mg SC at 24 h and thereafter once a day up to 24 h after achievement of an HRS response (either Partial or Full) based on SCr/AKI stage or up to Day 14.

Patients in Cohort 4 may be treated with a lower dose schedule. In Cohort 5, patients receive IV terlipressin and IV/SC placebo, with the same schedule as in Cohort 4.

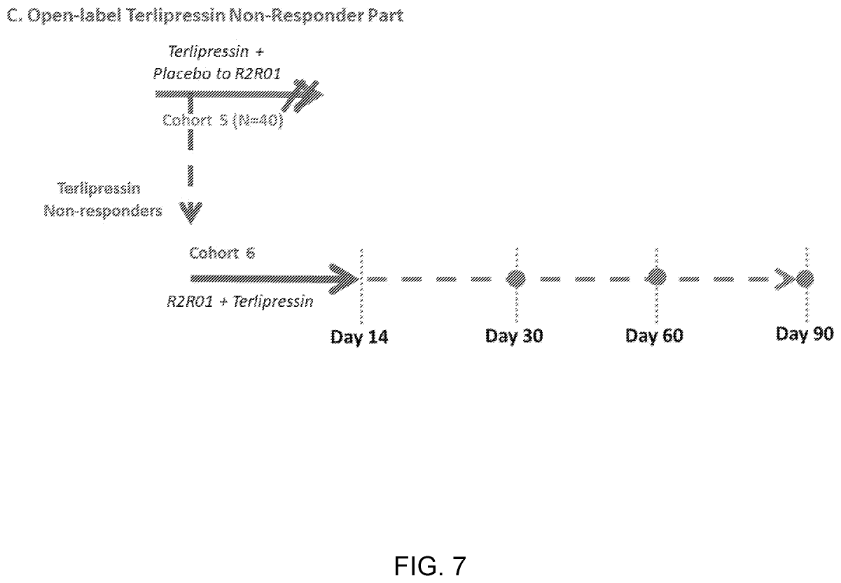

Reference is now made to FIG. 7, which depicts a design of the open-label terlipressin non-responder part of the overall study design shown in FIG. 4. Patients that do not respond to terlipressin in Cohort 5 are discontinued. After discontinuation, patients are allowed to enter Cohort 6 (Terlipressin Non-Responder Part) to receive Relaxin Agonist with the same dosing and schedule Cohort 4. No patient from any Cohort other than Cohort 5 is allowed in Cohort 6. Terlipressin non-responders are defined as follows: if on day 4, the serum creatinine has improved by less than 10% or is at the same level or higher than baseline the patient are considered a non-responder and drop out of Cohort 5. These patients are eligible for entry in Cohort 6. All patients receive albumin standard of care.

### Inclusion Criteria

Patients are evaluated according to the following inclusion criteria for eligibility in taking part in the study:
1. AKI stage 2 or 3 (see Table 11; AKI defined by any of the followings: 1) increase in SCr (SCr)≥0.3 mg/dl (or ≥26.5 micromolar/L) within 48 h, or 2) increase ≥50% in baseline SCr, which is known or presumed to have occurred within the prior seven days.
2. QLY SCr≥to 1.5 mg/dl.
3. No sustained improvement in renal function (less than 20% decrease in SCr and SCr =>1.5 mg/dL) after 48 h of diuretic withdrawal and the beginning of plasma volume expansion with albumin.
4. Female patients as well as female partners of male patients must be willing to avoid pregnancy for the duration of the study (>90 days).

### Exclusion Criteria

Patients who meet any of the following exclusion criteria are not be eligible to take part in the study:
1. Significant co-morbidities that in the opinion of the Investigator would preclude study participation.
2. QLY SCr level >5 mg/dL.
3. AKI stage 1
4. ACLF stage 3.
5. Model for End-Stage Liver Disease (MELD) score >35.
6. At least one event of large volume paracentesis (LVP)>4 Liters in the last 4 days before randomization.
7. Current or recent (within 4 weeks) treatment with nephrotoxic drugs (e.g., aminoglycosides, amphotericin, cyclosporine, NSAIDS (e.g., ibuprofen, naproxen, celecoxib), significant exposure to radiographic contrast agents (large doses or multiple injections of iodinated contrast media).
8. Shock (hypovolemic-, cardiogenic-, or vasodilatory/distributive shock) with mean arterial blood pressure (MAP)≤70 mmHg or systolic blood pressure ≤90 mmHg along with hypoperfusion.
9. Sepsis or uncontrolled bacterial infection (e.g., persisting bacteremia, persisting ascitic fluid leucocytosis, fever, increasing leucocytosis with vasomotor instability) as measured with the quick sepsis-related organ dysfunction assessment (qSOFA) score.
10. Fewer than two days of anti-infective therapy for documented or suspected infection.
11. Superimposed acute liver injury induced by drugs, herbal preparation or dietary supplements, with the exception of alcoholic hepatitis.
12. Estimated life expectancy less than 5 days.
13. Proteinuria >500 mg/day.
14. Tubular epithelial casts, heme granular casts.
15. Haematuria or microhaematuria (more than 50 red blood cells per high power field).
16. Abnormal renal ultra-sonography unless there is a known chronic structural disease (e.g., diabetic or hypertensive nephropathy).
17. Current or recent (within 4 weeks) renal replacement therapy (RRT).
18. Severe cardiovascular and pulmonary diseases including, but not limited to, unstable angina, pulmonary edema, congestive heart failure requiring increasing doses of drug therapy, persisting symptomatic peripheral vascular disease, or any other cardiovascular disease judged by the Investigator to be severe.
19. Transjugular intra-hepatic systemic shunt (TIPS) unless it is known to be non-functioning or occluded.
20. Ongoing use of vasopressors including midodrine, unless used for only 48 h before screening; in this case a wash-out period of 8 h before randomization is used.
21. Known allergy or hypersensitivity to terlipressin or other component of the study treatment.
22. Subject is not suitable to participate in the study for any reason (including, but not limited to co-morbidities, history of non-compliance with study visits, procedures, or drug administration) in the opinion of the Investigator.
23. Females of childbearing potential (those who are not surgically sterilized or post-menopausal for at least 1 year) are excluded from participation in the study unless they agree to use adequate contraception as described in Section 11.3.
24. Males who have no sterilization history and whose female partners have child-bearing potential must agree to use highly effective method of contraception during the period from the time of signing the informed consent form (ICF) through 90 days after the last dose of study drug. A male patient must agree to immediately inform the Investigator if his partner becomes pregnant during the study.

### Study Treatment

The treatment details of the various cohorts of the study design are shown below in Table 9.

TABLE 9
Cohorts and treatment details

| | | | |
|---|---|---|---|
| | Cohort | Terlipressin | |
| Cohort N | Description | Dose* | Relaxin Agonist Dose ** |

A. Open-Label Safety Run-In Part

| | | | |
|---|---|---|---|
| Cohort 1 | Relaxin Agonist | 1.0 mg IV | 1.0 mg IV × 1, 2.5 mg @ 12 h and |
| (N = 3) | Low dose | every 6 hours | 24 h, then 2.5 mg SC daily |
| | combination | | |
| Cohort 2 | Relaxin Agonist | 1.0 mg IV | 2.0 mg IV × 1, 5.0 mg @ 12 h and |
| (N = 3) | Medium dose | every 6 hours | 24 h, then 5.0 mg SC daily |
| | combination | | |
| Cohort 3 | Relaxin Agonist | 1.0 mg IV | 4.0 mg IV × 1, 5.0 mg @ 12 h & 10 |
| (N = 3) | Therapeutic dose | every 6 hours | mg @ 24 h, then 10.0 mg SC daily |
| | combination | | |

B. Single-Blind Placebo-Controlled Randomized Part

| | | | |
|---|---|---|---|
| Cohort 4 | Relaxin Agonist | 1.0 mg IV | Therapeutic dose determined from |
| (N = 40) | Therapeutic dose | every 6 hours | cohorts 1, 2, and 3 (e.g., 4.0 mg |
| | combination | | IV × 1, 5.0 mg @ 12 h & 10 |
| | | | mg @ 24 h, then 10.0 mg SC daily) |
| Cohort 5 | Terlipressin | 1.0 mg IV | Placebo to Relaxin Agonist |
| (N = 40) | monotherapy | every 6 hours | |

C. Open-Label Terlipressin Non-Responder Part

| | | | |
|---|---|---|---|
| Cohort 6 | Terlipressin | 1.0 mg IV | Therapeutic dose determined from |
| Up to | monotherapy non- | every 6 hours | cohorts 1, 2, and 3 (e.g., 4.0 mg |
| N = 40 | responders | | IV × 1, 5.0 mg @ 12 h & 10 |
| | receive Relaxin | | mg @ 24 h, then 10.0 mg SC daily) |
| | Agonist | | |
| | Therapeutic Dose | | |
| | combination | | |

*If SCr has decreased by less than 25% of QLY SCr at day 3 (after 2 days of terlipressin treatment), and if terlipressin shows acceptable safety, terlipressin can be increased up to 8.0 mg per day; i.e., 2.0 mg every six hours.
** Relaxin Agonist initial dose is IV over 4 hours, followed by SC administration for remaining doses.

Regarding terlipressin dose administration across all Cohorts, all patients in all Cohorts (1-6) are treated with terlipressin, administered as a 1.0 mg bolus IV infusion (1 mg over 2 minutes) every 6 h. Terlipressin dosing should continue up to 24 h after achievement of an HRS response (either Partial or Full) based on SCr/AKI stage or up to day 14.

Regarding terlipressin dose modification across all Cohorts, if SCr has decreased by less than 25% of QLY SCr (i.e., less than 25% of the SCr value when the patient was randomized) at day 3 (after 2 days of terlipressin treatment), and if terlipressin shows acceptable safety, terlipressin can be increased to 8.0 mg per day; i.e., 2.0 mg every six hours. The dose should not be increased in patients with coronary artery disease or if circulatory overload, pulmonary edema or bronchospasm is present. If dosing is interrupted because of an adverse event (AE), terlipressin can be re-started in the Investigator's judgment, at an equal or lower dose as per protocol. If the AE consists of cardiac ischemia or mesenteric ischemia, dosing should NOT be restarted.

Regarding terlipressin non-responder definition (only applicable for Cohort 5), if on day 4, SCr has decreased by less than 10% of QLY SCr or is at the same level or higher than QLY SCr, the patient is considered a non-responder and drops out of Cohort 5. These patients are eligible for entry into Cohort 6.

Regarding combination treatment of terlipressin with Relaxin Agonist, for those Cohorts where terlipressin is administered combined with Relaxin Agonist (Cohorts 1, 2, 3, 4, and 6), the first Relaxin Agonist administration commences immediately following the first terlipressin administration. Relaxin Agonist should be dosed up to 24 h after achievement of an HRS response (either Partial or Full) based on SCr/AKI stage or up to Day 14.

### Pre-Treatment Period (Cohorts 1, 2, 3, 4, and 5)

The pre-treatment period occurs prior to administration of study drug and includes performing baseline assessments and collection of prior medication information. The qualifying SCr value (SCr value at least 48 h after both diuretic withdrawal and the beginning of albumin fluid challenge) is considered the QLY SCr value and is drawn no more than 8 h prior to start of study drug. The QLY SCr value should be >1.5 mg/dL. No subjects should be randomized unless their QLY SCr has been obtained within 8 h prior to randomization and start of study drug. If there is a delay in subject randomization, then the QLY SCr value is redrawn so that the value is collected within 8 h prior to randomization and start of study drug to verify that the subject still meets the inclusion criterion for QLY SCr. Other baseline assessments are performed no more than 24 h prior to start of study drug.

### Active Study Period (All Cohorts)

The active study period extends from the initiation of study treatment through Day 14 or discharge from the hospital for any reason, whichever occurs first. Study drug is administered as described above in the Study Design.

Terlipressin dosing should continue up to 24 h after achievement of an HRS response based on SCr/AKI stage, or up to Day 14. Following the first SCr level returning to a value within 0.3 mg/dl (26.5 micromol/L) of the BL SCr value, or a regression of AKI stage with a reduction of SCr >0.3 mg/dl above the BL SCr value, the second SCr value is obtained a minimum of 2 h after the first SCr value. Efforts are made to collect these SCr values. All information regarding RRT, TIPS, liver transplant, or open-label vasopressor use are collected.

The follow-up period begins after the end of the study treatment and concludes 90 days following the start of treatment. All subjects return for follow-up on Day 30 (±2), and contacted by telephone for follow-up on Days 60 (±7), and 90 (±7) to assess survival, RRT, TIPS, and liver transplant status. Study days are counted from first day of study drug administration (or from randomization for those subjects who do not receive study drug). In addition, during the Day 30 follow-up, a physical examination is performed, and updated data on medical history, vital signs, concomitant medications, and SAE assessments are collected.

Efficacy assessments are evaluated as followed: serum creatinine are collected at baseline, once daily during treatment; and then once daily (regardless of treatment status) until Day 14 or hospital discharge, whichever occurs first. If SCr assessments are performed more than once daily as part of the subject's medical care, all values obtained each day are recorded on the eCRF. SCr values obtained after RRT, TIPS, liver transplant, or open-label vasopressor use are excluded from the efficacy evaluation.

The primary efficacy variables include:
Safety and tolerability are assessed by occurrence of AEs, changes in physical examinations, vital signs, ECGs and clinical laboratory parameters.
The incidence of Responders (Established HRS reversal defined as patients with a Full or Partial HRS response (based on SCr/AKI stage) AND are alive without Renal Replacement Therapy (RRT) for at least 30 days after the first dose of study medication), evaluated separately as two different outcome groups and combined.
Patients who are undergoing liver transplant during the first 30 days after treatment start are evaluated for their SCr and AKI stage before the transplant and considered responders if meeting the criteria for SCr/AKI stage for HRS response (Full or Partial) before the liver transplant and are alive and without RRT at day 30 after treatment start.
In case of recurrence and retreatment during the first 30 days, the second treatment period is evaluated for response.

The secondary efficacy variables include:
Number of patients who died (mortality rate) at day 30, 60, and 90.
Number of patients with renal and/or liver transplant rate at day 30, 60, and 90.
Number of patients with RRT at day 30, 60, and 90.
Number of patients with durable HRS reversal (number of patients responding and without RRT by day 45, 60, and 90).
Number of patients with HRS recurrence.
Number of patients with worsening of ACLF 1 or 2 to stage 3.
Mean change from baseline in MELD score at day 30, 60, and 90
The number of responding patients with an Established HRS reversal where the clinical definition is patient alive without RRT 10 days after the achievement of HRS response (i.e., as per the CONFIRM study).

The exploratory evaluation criteria include:
Mean change from baseline in serum and urine biomarkers (including Cystatin C, Endothelin-1, vWF, NGAL, KIM 1
Population pharmacokinetic analysis is performed to derive primary PK parameters (e.g. absorption rate (ka), apparent Clearance (Cl/F) and apparent Volume of distribution (V/F)) and secondary PK parameters (maximum concentration (Cₘₐₓ), time of Cₘₐₓ (tₘₐₓ), minimum plasma concentrations (Cₘᵢₙ), area under the concentration time curve over the dosing interval (AUC₀₋₂₄ₕ), elimination half-life (t_{1/2}), and accumulation Ratio (Racc).

### Safety Assessments

Physical Examination: A physical examination including assessment of the following is performed by the investigator or his/her designee: Head, ears, nose and throat, Neck/thyroid, Extremities and the Cardiovascular, Integumentary, Lymphatic, Nervous, Musculoskeletal, Respiratory systems is evaluated at specific timepoints. Any clinically significant change in the physical examination findings during the study is considered an AE and recorded on the eCRF.

Vital Signs: The assessment of vital signs (heart rate, blood pressure, respiratory rate, body temperature, height and weight are recorded.

Safety/12-lead ECGs: 12-lead ECGs are performed in triplicate, one minute apart, at specific timepoints. The ECG results are recorded as normal or abnormal on the eCRF. All abnormal results are be evaluated as either clinically significant or not clinically significant by the investigator. Any new, abnormal, clinically significant ECG result is recorded as an AE. A copy of each ECG tracing remains with the source documents.

Clinical safety laboratory assessments: With the exception of urine pregnancy tests, serum creatinine, CBC, INR and electrolytes, all protocol specified laboratory tests are performed at a central study laboratory. Detailed instructions for the collection, handling and reporting of clinical laboratory samples are provided to sites in a laboratory manual provided by the central laboratory prior to site initiation.

If any abnormal, clinically significant laboratory results occur, the abnormal tests may be repeated, if appropriate, to ensure the validity of the abnormal result. If the abnormal, clinically significant abnormal results are valid, the laboratory test(s) are to be repeated every two weeks until the results are within normal limits or are no longer considered clinically significant by the investigator. Any new, abnormal, clinically significant laboratory result is recorded as AE.

Laboratoratory assessment include: Haemoglobin, haematocrit, RBC, MCV, MCH, MCHC, reticulocytes, haptoglobin, WBC with differential, platelet count and platelet aggregation test, BUN, serum creatinine, Cystatin C, AST, ALT, alkaline phosphatase, LDH, total bilirubin, indirect and direct bilirubin, sodium, potassium, chloride, calcium, phosphate, glucose, total protein, albumin, total cholesterol, LDL, HDL, triglycerides, C-reactive protein, uric acid, cortisol ACTH, lactate, ammonia, Urinalysis: Specific gravity (females only), protein, blood, ketones, glucose, assessment of multi-organ dysfunction (e.g., according to CLIF-SOFA score), Encephalopathy Score, Systemic Inflammatory Response Syndrome (SIRS) assessment, Quick sepsis-related organ dysfunction assessment (qSOFA) (calculated using three criteria, assigning one point each to: low systolic blood pressure (≤100 mmHg), high respiratory rate (≥22 breaths per min), and altered mental state (Glasgow coma scale <15), and Model for End-stage Liver Disease Score (based on SCr, bilirubin and INR values).

Sequence Listing

SEQ ID NO: 1
Ac-L-E-G-R-E-K-V-R-A-K(Ac)-I-Aib-Aib-E-G-K(TTDS-TTDS-TTDS-gE-gE-gE-Palm)-S-
-T-W-S-Aib-R-K-K(Ac)-NH₂
SEQ ID NO: 2
Ac-L-E-G-R-E-K-V-R-A-K(Ac)-I-Aib-Aib-E-G-K(TTDS-TTDS-TTDS-gE-gE-gE-Palm)-S-
-T-F-S-Aib-R-A-K(Ac)-NH₂
SEQ ID NO: 3
Ac-L-E-G-R-E-K-V-R-A-K(Ac)-I-Aib-Aib-E-G-K(TTDS-TTDS-TTDS-gE-gE-gE-Palm)-S-
-T-F-S-Mly-R-A-K(Ac)-NH₂
SEQ ID NO: 4
Ac-L-E-G-R-E-K-V-R-A-K(Ac)-I-Aib-Aib-E-G-K(TTDS-TTDS-gE-gE-gE-Palm)-S-T-W--
S-Aib-R-K-K(Ac)-NH₂
SEQ ID NO: 5
Ac-L-E-G-R-E-K-V-R-A-K(Ac)-I-Aib-Aib-E-G-K(PEG₂-PEG₂-PEG₂-P-EG₂-gE-gE-gE-
Palm)-S-T-W-S-Aib-R-K-K(Ac)-NH₂
SEQ ID NO: 6
Ac-L-E-G-R-E-K-V-R-A-K(Ac)-I-Aib-Aib-E-G-K(TTDS-TTDS-gE-gE-gE-Stea)-S-T-W--
S-Aib-R-K-K(Ac)-NH₂
SEQ ID NO: 7
Ac-L-E-G-R-E-K-V-R-A-K(Ac)-I-Aib-Aib-E-G-K(TTDS-TTDS-TTDS-gE-gE-gE-Stea)-S-
-T-F-S-Mly-R-A-K(Ac)-NH₂
SEQ ID NO: 8
Ac-L-E-G-R-E-K-V-R-A-K(Ac)-I-Aib-Aib-E-G-K(PEG₂-PEG₂-PEG₂-P-EG₂-gE-gE-gE-
Palm)-S-T-F-S-Mly-R-A-K(Ac)-NH₂
SEQ ID NO: 9
Ac-L-E-G-R-E-K-V-R-A-K(Ac)-I-Aib-Aib-E-G-K(TTDS-TTDS-gE-gE-Palm)-S-T-F-S-M-
ly-R-A-K(Ac)-NH₂
SEQ ID NO: 10
Ac-L-E-G-R-E-K-V-R-A-K(Ac)-I-Aib-Aib-E-G-K(TTDS-TTDS-gE-gE-gE-gE-Palm)-S-T--
F-S-Mly-R-A-K(Ac)-NH₂
SEQ ID NO: 11
Ac-L-E-G-R-E-K-V-R-A-K(Ac)-I-Aib-Aib-E-G-K(TTDS-TTDS-TTDS-gE-gE-gE-Palm)-S-
-T-W-S-Mly-R-A-K(Ac)-NH₂
SEQ ID NO: 12
Ac-L-E-G-R-E-K-V-R-A-Cit-I-Aib-Aib-E-G-K(TTDS-TTDS-TTDS-gE-gE-gE-Palm)-S-T--
F-S-Mly-R-A-K(Ac)-NH₂
SEQ ID NO: 13
Ac-L-E-G-R-E-K-V-R-A-K(Ac)-I-Aib-Aib-E-G-K(TTDS-TTDS-TTDS-gE-gE-gE-Palm)-S-
-T-F-S-K-R-A-K(Ac)-NH₂
SEQ ID NO: 14
Ac-L-E-G-R-E-K-V-R-A-K(Ac)-I-Aib-Aib-E-G-K(TTDS-TTDS-TTDS-gE-gE-gE-Palm)-S-
-T-F-Dser-K-R-A-K(Ac)-NH₂
SEQ ID NO: 15
Ac-L-E-G-R-E-K-V-R-A-K(Ac)-I-Aib-Aib-E-G-K(TTDS-TTDS-TTDS-gE-gE-gE-Palm)-S-
-T-F-Aib-K-R-A-K(Ac)-NH₂
SEQ ID NO: 16
Ac-L-E-G-R-E-K-V-R-A-K(Ac)-I-Aib-Aib-E-G-K(TTDS-TTDS-gE-gE-gE-Palm)-S-T-F--
S-Dlys-R-A-K(Ac)-NH₂
SEQ ID NO: 17
Ac-L-E-G-R-E-K-V-R-A-K(Ac)-I-Aib-Aib-E-G-K(TTDS-TTDS-gE-gE-gE-Palm)-S-T-W--
S-Dlys-R-A-K(Ac)-NH₂
SEQ ID NO: 18
Ac-L-E-G-R-E-K-V-R-A-K(Ac)-I-Aib-Aib-E-G-K(TTDS-TTDS-gE-gE-gE-Palm)-S-T-W--
S-Dlys-R-K-K(Ac)-NH₂
SEQ ID NO: 19
Ac-L-E-G-R-E-K-V-R-A-K(Ac)-I-Aib-Aib-E-G-K(PEG₂-PEG₂-PEG₂-g-E-gE-gE-Palm)-S-
T-W-S-Aib-R-K-K(Ac)-NH₂
SEQ ID NO: 20
Ac-L-E-G-R-E-K-V-R-A-K(Ac)-I-Aib-Aib-E-G-K(TTDS-TTDS-TTDS-gE-gE-gE-Stea)-S-
-T-W-S-Aib-R-K-K(Ac)-NH₂
SEQ ID NO: 21
Ac-L-E-G-R-E-K-V-R-A-K(Ac)-I-Aib-Aib-E-G-K(PEG₂-PEG₂-PEG₂-P-EG₂-gE-gE-gE-
Stea)-S-T-W-S-Aib-R-K-K(Ac)-NH₂
SEQ ID NO: 22
Ac-L-E-G-R-E-K-V-R-A-K(Ac)-I-Aib-Aib-E-G-K(PEG₂DGA-PEG₂DGA-PEG.s-
ub.2DGA-gE-gE-gE-Palm)-S-T-F-S-Mly-R-A-K(Ac)-NH₂
SEQ ID NO: 23
Ac-L-E-G-R-E-L-V-R-A-K(Ac)-I-Aib-Aib-E-G-K(TTDS-TTDS-TTDS-gE-gE-gE-Palm)-S-
-T-F-S-Mly-R-A-K(Ac)-NH₂
SEQ ID NO: 24
Ac-L-E-G-R-E-F-V-R-A-K(Ac)-I-Aib-Aib-E-G-K(TTDS-TTDS-TTDS-gE-gE-gE-Palm)-S-
-T-F-S-Mly-R-A-K(Ac)-NH₂
SEQ ID NO: 25
Ac-L-E-G-R-E-Q-V-R-A-K(Ac)-I-Aib-Aib-E-G-K(TTDS-TTDS-TTDS-gE-gE-gE-Palm)-S-
-T-F-S-Mly-R-A-K(Ac)-NH₂
SEQ ID NO: 26
Ac-L-E-G-R-E-K-V-R-A-K(Ac)-I-Aib-Aib-E-G-K(PEG₂-PEG₂-PEG₂-PEG₂-gE-gE-gE-St-
ea)-S-T-F-S-Mly-R-A-K(Ac)-NH₂
SEQ ID NO: 27
Ac-L-E-G-R-E-R-V-R-A-K(Ac)-I-Aib-Aib-E-G-K(TTDS-TTDS-TTDS-gE-gE-gE-Palm)-S-
-T-F-S-Mly-R-A-K(Ac)-NH₂
SEQ ID NO: 28
Ac-L-E-G-R-E-Hly-V-R-A-K(Ac)-I-Aib-Aib-E-G-K(TTDS-TTDS-TTDS-gE-gE-gE-Palm)-
-S-T-F-S-Mly-R-A-K(Ac)-NH₂
SEQ ID NO: 29
Ac-L-E-G-R-E-K-V-R-Aib-K(Ac)-I-Aib-Aib-E-G-K(TTDS-TTDS-TTDS-gE-gE-gE-Palm)-
-S-T-F-S-Mly-R-A-K(Ac)-NH₂
SEQ ID NO: 30
Ac-L-E-G-R-E-K-V-R-A-K(Ac)-I-Aib-Aib-E-G-K(TTDS-TTDS-TTDS-gE-gE-gE-Stea)-S-
-T-W-S-Mly-R-A-K(Ac)-NH₂
SEQ ID NO: 31
Ac-L-E-G-R-E-R-V-R-A-K(Ac)-I-Aib-Aib-E-G-K(TTDS-TTDS-TTDS-gE-gE-gE-Palm)-S-
-T-W-S-Aib-R-R-K(Ac)-NH₂
SEQ ID NO: 32
Ac-L-E-G-R-E-K-V-R-A-K(Ac)-I-Aib-Aib-E-G-K(PEG₂DGA-PEG₂DGA-PEG.S-
ub.2DGA-gE-gE-gE-Stea)-S-T-F-S-Mly-R-A-K(Ac)-NH₂
SEQ ID NO: 33
Ac-L-E-G-R-E-K-V-R-A-K(Ac)-I-Aib-Aib-E-G-K(TTDS-TTDS-gE-gE-gE-Palm)-S-T-F--
S-Mly-R-A-K(Ac)-NH₂
SEQ ID NO: 34
Ac-L-E-G-R-E-K-V-R-A-K(Ac)-I-Aib-Aib-E-G-K(TTDS-TTDS-gE-gE-gE-Stea)-S-T-F--S-
Mly-R-A-K(Ac)-NH₂
SEQ ID NO: 35
Ac-L-E-G-R-E-K-V-R-A-K(Ac)-I-Aib-Aib-E-G-K(TTDS-TTDS-TTDS-gE-gE-gE-Palm)-S-
-T-W-Aib-K-R-A-K(Ac)-NH₂
SEQ ID NO: 36
Ac-L-E-G-R-E-K-V-R-A-K(Ac)-I-Aib-Aib-E-G-K(TTDS-TTDS-TTDS-gE-gE-gE-Palm)-S-
-T-F-S-Dlys-R-A-K(Ac)-NH₂
SEQ ID NO: 37
Ac-L-E-G-R-E-L-V-R-A-K(Ac)-I-Aib-Aib-E-G-K(TTDS-TTDS-TTDS-gE-gE-gE-Palm)-S-
-T-W-S-Aib-R-R-K(Ac)-NH₂
SEQ ID NO: 38
Ac-L-E-G-R-E-K-V-R-A-K(Ac)-I-Aib-Aib-E-G-K(PEG₂-PEG₂-PEG₂-g-E-gE-gE-Palm)-S-
T-F-S-Mly-R-A-K(Ac)-NH₂
SEQ ID NO: 39
Ac-L-E-G-R-E-F-V-R-A-K(Ac)-I-Aib-Aib-E-G-K(TTDS-TTDS-TTDS-gE-gE-gE-Palm)-S-
-T-W-S-Aib-R-R-K(Ac)-NH₂
SEQ ID NO: 40
Ac-L-E-G-R-E-Q-V-R-A-K(Ac)-I-Aib-Aib-E-G-K(TTDS-TTDS-TTDS-gE-gE-gE-Palm)-S-
-T-W-S-Aib-R-R-K(Ac)-NH₂
SEQ ID NO: 41
Ac-L-E-G-R-E-K-V-R-A-K(Ac)-I-Aib-Aib-E-G-K(PEG₂DGA-PEG₂DGA-PEG.s-
ub.2DGA-gE-gE-gE-Palm)-S-T-W-S-Mly-R-A-K(Ac)-NH₂
SEQ ID NO: 42
Ac-L-E-G-R-E-K-V-R-A-K(Ac)-I-Aib-Aib-E-G-K(PEG₂-PEG₂-PEG₂-P-EG₂-gE-gE-gE-
Palm)-S-T-W-S-Mly-R-A-K(Ac)-NH₂
SEQ ID NO: 43
Ac-L-E-G-R-E-Hly-V-R-A-K(Ac)-I-Aib-Aib-E-G-K(TTDS-TTDS-TTDS-gE-gE-gE-Stea)--
S-T-W-S-Mly-R-A-K(Ac)-NH₂
SEQ ID NO: 44
Ac-L-E-G-R-E-K-V-R-A-K(Ac)-I-Aib-Aib-E-G-K(TTDS-TTDS-TTDS-gE-gE-gE-Palm)-S-
-T-W-Dser-K-R-A-K(Ac)-NH₂
SEQ ID NO: 45
Ac-L-E-G-R-E-K-V-R-A-K(Ac)-I-Aib-Aib-E-G-K(PEG₂-PEG₂-PEG₂-P-EG₂-PEG₂-gE-gE-
gE-Palm)-S-T-F-S-Mly-R-A-K(Ac)-NH₂
SEQ ID NO: 46
Ac-L-E-G-R-E-K-V-R-A-K(Ac)-I-Aib-Aib-E-G-K(PEG₂-PEG₂-PEG₂-P-EG₂-PEG₂-gE-gE-
gE-gE-Palm)-S-T-F-S-Mly-R-A-K(Ac)-NH₂
SEQ ID NO: 47
Ac-L-E-G-R-E-K-V-R-A-K-I-Aib-Aib-E-G-K(TTDS-TTDS-TTDS-gE-gE-gE-Palm)-S-T-F-
-S-Mly-R-A-K(Ac)-NH₂
SEQ ID NO: 48
Ac-L-E-G-R-E-K-V-R-A-Q-I-Aib-Aib-E-G-K(TTDS-TTDS-TTDS-gE-gE-gE-Palm)-S-T-F-
-S-Mly-R-A-K(Ac)-NH₂
SEQ ID NO: 49
Ac-L-E-G-R-E-K-V-R-A-K(Ac)-I-Aib-Aib-E-G-K(TTDS-TTDS-TTDS-gE-gE-gE-Palm)-S-
-T-F-S-Mly-R-A-K-NH₂
SEQ ID NO: 50
Ac-L-E-G-R-E-K-V-R-A-K(Ac)-I-Aib-Aib-E-G-K(PEG₂DGA-PEG₂DGA-PEG.S-
ub.2DGA-gE-gE-gE-Stea)-S-T-W-S-Aib-R-K-K(Ac)-NH₂
SEQ ID NO: 51
Ac-L-E-G-R-E-K-V-R-A-K(Ac)-I-Aib-Aib-E-G-K(TTDS-TTDS-TTDS-gE-gE-gE-gE-Stea-
)-S-T-W-S-Aib-R-K-K(Ac)-NH₂
SEQ ID NO: 52
Ac-L-E-G-R-E-K-V-R-A-K(Ac)-I-Aib-Aib-E-G-K(TTDS-TTDS-TTDS-gE-gE-Stea)-S-T--
W-S-Aib-R-K-K(Ac)-NH₂
SEQ ID NO: 53
Ac-L-E-G-R-E-K-V-R-A-K(Ac)-I-Aib-Aib-E-G-K(PEG₂DGA-PEG₂DGA-PEG.s-
ub.2DGA-gE-gE-gE-gE-Stea)-S-T-W-S-Aib-R-K-K(Ac)-NH₂
SEQ ID NO: 54
Ac-L-E-G-R-E-K-V-R-A-K(Ac)-I-Aib-Aib-E-G-K(TTDS-TTDS-gE-gE-gE-gE-Stea)-S-T-
W-S-Aib-R-K-K(Ac)-NH₂
SEQ-ID-NO: 55
Ac-L-E-G-R-E-R-V-R-A-K(Ac)-I-Aib-Aib-E-
G-K(TTDS-TTDS-TTDS-gE-gE-gE-Palm)-S--T-W-S-Aib-R-R-K(Ac)-NH₂
SEQ-ID-NO: 55
Ac-L-E-G-R-E-R-V-R-A-K(Ac)-I-Aib-Aib-E-G-K(TTDS-TTDS-TTDS-gE-gE-gE-Palm)-S-
T-W-S-Aib-R-K-K(Ac)-NH₂
SEQ ID NO: 56
Ac-L-E-G-R-E-K-V-R-A-Cit-I-Aib-Aib-E-G-K(TTDS-TTDS-TTDS-gE-gE-gE-Stea)-S-T--
F-S-Mly-R-A-K(Ac)-NH₂
SEQ ID NO: 57
Ac-L-E-G-R-E-K-V-R-A-K(Ac)-I-Aib-Aib-E-G-K(TTDS-TTDS-TTDS-gE-gE-gE-Palm)-S-
-T-W-S-Hly-R-A-K(Ac)-NH₂
SEQ ID NO: 58
Ac-L-E-G-R-E-K-V-R-A-K(Ac)-I-Aib-Aib-E-G-K(TTDS-TTDS-TTDS-TTDS-gE-gE-gE-
St-ea)-S-T-W-S-Aib-R-K-K(Ac)-NH₂
SEQ ID NO: 59
Ac-L-E-G-R-E-K-V-R-A-K(Ac)-I-Aib-Aib-E-G-K(TTDS-TTDS-TTDS-TTDS-gE-gE-gE-
St-ea)-S-T-F-S-Aib-R-K-K(Ac)-NH₂
SEQ ID NO: 60
Ac-L-E-G-R-E-K-V-R-A-K(Ac)-I-Aib-Aib-E-G-K(TTDS-TTDS-gE-gE-gE-Stea)-S-T-F--S-
Aib-R-K-K(Ac)-NH₂
SEQ ID NO: 61
Ac-L-E-G-R-E-K-V-R-A-K(Ac)-I-Aib-Aib-E-G-K(TTDS-TTDS-TTDS-gE-gE-gE-Stea)-S-
-T-F-S-Aib-R-K-K(Ac)-NH₂
SEQ ID NO: 62
Ac-L-E-G-R-E-K-V-R-A-K(Ac)-I-Aib-Aib-E-G-K(TTDS-TTDS-TTDS-gE-gE-gE-gE-Palm-
)-S-T-W-S-Aib-R-K-K(Ac)-NH₂
SEQ ID NO: 63
Ac-L-E-G-R-E-K-V-R-A-K(Ac)-I-Aib-Aib-E-G-K(TTDS-TTDS-TTDS-gE-gE-gE-Palm)-S-
-Q-W-S-Aib-R-K-K(Ac)-NH₂
SEQ ID NO: 64
Ac-L-E-G-R-E-K-V-R-A-K(Ac)-I-Aib-Aib-E-G-K(TTDS-TTDS-TTDS-gE-gE-gE-Palm)-S-
-R-W-S-Aib-R-K-K(Ac)-NH₂
SEQ ID NO: 65
Ac-L-E-G-R-E-K-V-R-K(Ac)-Q-I-Aib-Aib-E-G-K(TTDS-TTDS-TTDS-gE-gE-gE-Palm)-S-
-T-W-S-Aib-R-K-K(Ac)-NH₂
SEQ ID NO: 66
Ac-L-E-G-R-E-K-V-R-A-K(Ac)-I-Aib-Aib-E-G-K(TTDS-TTDS-TTDS-gE-gE-gE-Palm)-S-
-T-W-K(Ac)-Aib-R-A-K(Ac)-NH₂
SEQ ID NO: 67
Ac-L-E-G-R-E-R-V-R-A-K(Ac)-I-Aib-Aib-E-G-K(TTDS-TTDS-TTDS-gE-gE-gE-Palm)-S-
-T-W-S-Aib-R-K-K(Ac)-NH₂
SEQ ID NO: 68
Ac-L-E-G-R-E-K-V-R-A-K(Ac)-I-Aib-Aib-E-G-K(TTDS-TTDS-TTDS-gE-gE-gE-Palm)-S-
-T-W-S-Aib-R-K(Ac)-K-NH₂
SEQ ID NO: 69
Ac-L-E-G-R-E-K-V-R-A-K(Ac)-I-Aib-Aib-E-G-K(TTDS-TTDS-TTDS-gE-gE-gE-Palm)-S-
-T-W-S-Aib-R-K(Ac)-R-NH₂
SEQ ID NO: 70
Ac-L-E-G-R-E-K-V-R-A-K(Ac)-I-Aib-I-E-G-K(TTDS-TTDS-TTDS-gE-gE-gE-Palm)-S-T-
W-S-Aib-R-K-K(Ac)-NH₂
SEQ ID NO: 71
Ac-L-E-G-R-E-K-V-R-A-K(Ac)-I-Aib-Aib-E-G-K(TTDS-TTDS-TTDS-gE-gE-gE-Palm)-S-
-T-W-S-Aib-R-A-K-NH₂
SEQ ID NO: 72
Ac-L-E-G-R-E-K-V-R-A-K(Ac)-I-Aib-Aib-E-G-K(TTDS-TTDS-TTDS-gE-gE-gE-Palm)-S-
-T-F-Aib-R-R-A-K(Ac)-NH₂
SEQ ID NO: 73
Ac-L-E-G-R-E-K-V-R-A-K(Ac)-I-Aib-Aib-E-G-K(TTDS-TTDS-TTDS-gE-gE-gE-Palm)-S-
-K-W-S-Aib-R-A-K-NH₂
SEQ ID NO: 74
Ac-L-E-G-R-E-K-V-R-A-K(Ac)-I-Aib-I-E-G-K(TTDS-TTDS-TTDS-TTDS-gE-gE-gE-
Palm)-S-T-W-S-Aib-R-K-K(Ac)-NH₂
SEQ ID NO: 75
Ac-L-E-G-R-E-R-V-R-A-K(Ac)-I-Aib-Aib-E-G-K(TTDS-TTDS-TTDS-TTDS-gE-gE-gE-
Palm)-S-T-W-S-Aib-R-R-K(Ac)-NH₂
SEQ ID NO: 76
Ac-L-E-G-R-E-R-V-R-A-K(Ac)-I-Aib-Aib-E-G-K(TTDS-TTDS-TTDS-gE-gE-gE-Palm)-S-
-T-F-S-Aib-R-R-K(Ac)-NH₂
SEQ ID NO: 77
Ac-L-E-G-R-E-R-V-R-A-K(Ac)-I-Aib-Aib-E-G-K(TTDS-TTDS-TTDS-TTDS-gE-gE-gE-
Palm)-S-T-F-S-Aib-R-R-K(Ac)-NH₂
SEQ ID NO: 78
Ac-L-E-G-R-E-K-V-R-A-K(Ac)-I-Aib-Aib-E-G-K(TTDS-TTDS-TTDS-TTDS-gE-gE-gE-
Palm)-S-T-F-S-Mly-R-A-K(Ac)-NH₂
SEQ ID NO: 79
Ac-L-E-G-R-E-K-V-R-A-K(Ac)-I-Aib-Aib-E-G-K(TTDS-TTDS-TTDS-gE-gE-gE-Palm)-S-
-K-F-S-Aib-R-K-K(Ac)-NH₂
SEQ ID NO: 80
Ac-L-E-G-R-E-R-V-R-A-K(Ac)-I-Aib-Aib-E-G-K(TTDS-TTDS-TTDS-gE-gE-gE-Palm)-S-
-T-Trp(5-C1)-S-Aib-R-R-K(Ac)-NH₂
SEQ ID NO: 81
Ac-K(Ac)-E-G-R-E-K-V-R-A-K(Ac)-I-Aib-Aib-E-G-K(TTDS-TTDS-TTDS-gE-gE-gE-Pal-
m)-S-T-F-S-Mly-R-A-K(Ac)-NH₂
SEQ ID NO: 82
Ac-L-E-G-R-E-K-V-R-Aib-K(Ac)-I-Aib-Aib-E-G-K(TTDS-TTDS-TTDS-gE-gE-gE-Palm)-
-S-T-W-S-Aib-R-A-K(Ac)-NH₂
SEQ ID NO: 83
Ac-L-E-G-R-E-K-V-R-A-K(Ac)-I-Aib-Aib-E-G-K(TTDS-TTDS-TTDS-gE-gE-gE-Palm)-S-
-K-W-S-Aib-R-K-K(Ac)-NH₂
SEQ ID NO: 84
Ac-L-E-G-R-E-K-V-R-A-K(Ac)-I-Aib-I-E-G-K(TTDS-TTDS-TTDS-gE-gE-gE-Palm)-S-T-
-F-S-Mly-R-A-K(Ac)-NH₂
SEQ ID NO: 85
Ac-L-E-G-R-E-R-V-R-Aib-K(Ac) I Aib Aib E G K(TTDS-TTDS-TTDS-gE-gE-gE-Palm)S
TW S Aib R R K(Ac)-NH,
SEQ ID NO: 86
Ac-Aib-E-G-R-E-K-V-R-A-K(Ac)-I-Aib-I-E-G-K(TTDS-TTDS-TTDS-gE-gE-gE-Palm)-S-
-T-F-S-Mly-R-A-K(Ac)-NH₂
SEQ ID NO: 87
Ac-L-E-G-R-E-K-V-R-A-K(Ac)-I-Aib-Aib-E-G-K(TTDS-TTDS-TTDS-gE-gE-gE-Palm)-S-
-T-F-S-R-R-A-K(Ac)-NH₂
SEQ ID NO: 88
Ac-L-E-G-R-E-K-V-R-A-K(Ac)-I-Aib-Aib-E-G-K(TTDS-TTDS-TTDS-gE-gE-gE-Palm)-S-
-T-F-S-Hly-R-A-K(Ac)-NH₂
SEQ ID NO: 89
Ac-L-E-G-R-E-K-V-R-A-K(Ac)-I-Aib-Aib-E-G-K(TTDS-TTDS-TTDS-gE-gE-gE-Palm)-S-
-T-Mph-S-R-R-A-K(Ac)-NH₂
SEQ ID NO: 90
Ac-L-E-G-R-E-K-V-R-A-K(Ac)-I-A-Aib-E-G-K(TTDS-TTDS-TTDS-gE-gE-gE-Palm)-S-T-
-F-S-Mly-R-A-K(Ac)-NH₂
SEQ ID NO: 91
Ac-L-E-G-R-E-R-V-R-A-K(Ac)-I-Aib-Aib-E-G-K(TTDS-TTDS-TTDS-gE-gE-gE-Palm)-S-
-T-Pfp-S-Aib-R-R-K(Ac)-NH₂
SEQ ID NO: 92
Ac-L-E-G-R-E-R-V-R-A-K(Ac)-I-Aib-Aib-E-G-K(TTDS-TTDS-TTDS-gE-gE-gE-Palm)-S-
-T-Trp(5-F)-S-Aib-R-R-K(Ac)-NH₂
SEQ ID NO: 93
Ac-L-E-G-R-E-K-V-R-A-K(Ac)-I-Aib-Aib-E-G-K(TTDS-TTDS-TTDS-gE-gE-gE-Palm)-S-
-T-F-T-R-R-A-K(Ac)-NH₂
SEQ ID NO: 94
Ac-L-E-G-R-E-K-V-R-A-K(Ac)-I-Aib-Aib-E-G-K(TTDS-TTDS-TTDS-gE-gE-gE-Palm)-S-
-T-F-V-R-R-A-K(Ac)-NH₂
SEQ ID NO: 95
Ac-L-E-G-R-E-K-V-R-A-K(Ac)-I-Aib-Aib-E-G-K(TTDS-TTDS-TTDS-gE-gE-gE-Myr)-S--
T-F-S-Mly-R-A-K(Ac)-NH₂
SEQ ID NO: 96
Ac-L-E-G-R-E-K-V-R-A-K(Ac)-I-Aib-Aib-E-G-K(TTDS-TTDS-TTDS-gE-gE-gE-Myr)-S--
T-F-S-Mly-R-A-K-NH₂
SEQ ID NO: 97
Ac-L-E-G-R-E-K-V-R-A-K(Ac)-I-Aib-Aib-E-G-K(TTDS-TTDS-TTDS-gE-gE-gE-gE-Myr)-
-S-T-F-S-Mly-R-A-K-NH₂
SEQ ID NO: 100 = H2-relaxin chain A
H-Gln-Leu-Tyr-Ser-Ala-Leu-Ala-Asn-Lys-Cys-Cys-His-Val-Gly-Cys-Thr-Lys-Arg--Ser-
Leu-Ala-Arg-Phe-Cys-OH
SEQ ID NO: 101 = H2-relaxin chain B
H-Asp-Ser-Trp-Met-Glu-Glu-Val-Ile-Lys-Leu-Cys-Gly-Arg-Glu-Leu-Val-Arg-Ala--Gln-
Ile-Ala-Ile-Cys-Gly-Met-Ser-Thr-Trp-Ser-OH
SEQ ID NO: 102 = SEQ ID B7-33 C11.235*
H-Val-Ile-Lys-Leu-Ser-Gly-Arg-Glu-Leu-Val-Arg-Ala-Gln-Ile-Ala-Ile-Ser-Gly--Met-Ser-
Thr-Trp-Ser-Lys-Arg-Ser-Leu-NH₂
SEQ ID NO: 103 = SEQ ID AcB7-33 C11.235*
Ac-Val-Ile-Lys-Leu-Ser-Gly-Arg-Glu-Leu-Val-Arg-Ala-Gln-Ile-Ala-Ile-Ser-Gly--Met-Ser-
Thr-Trp-Ser-Lys-Arg-Ser-Leu-NH₂
SEQ ID NO: 104 = SEQ ID KKKK(AcB7-29 C11.235)*
Ac-Val-Ile-Lys-Leu-Ser-Gly-Arg-Glu-Leu-Val-Arg-Ala-Gln-Ile-Ala-Ile-Ser-Gly--Met-Ser-
Thr-Trp-Ser-Lys-Lys-Lys-Lys-NH₂

### OTHER EMBODIMENTS

It is to be understood that the words which have been used are words of description rather than limitation, and that changes may be made within the purview of the appended claims without departing from the true scope and spirit of the invention in its broader aspects.

While the present invention has been described at some length and with some particularity with respect to the several described embodiments, it is not intended that it should be limited to any such particulars or embodiments or any particular embodiment, but it is to be construed with references to the appended claims so as to provide the broadest possible interpretation of such claims in view of the prior art and, therefore, to effectively encompass the intended scope of the invention.

All publications, patent applications, patents, and other references mentioned herein are incorporated by reference in their entirety. In case of conflict, the present specification, including definitions, controls. In addition, section headings, the materials, methods, and examples are illustrative only and not intended to be limiting.

1. A method of preventing or treating renal failure in an individual in need thereof, comprising co-administering an effective amount of a relaxin analogue and a vasopressin analogue to the individual.

2. The method of claim 1, wherein the renal failure is selected from the group consisting of: renal dysfunction induced by liver cirrhosis, renal dysfunction induced by liver transplantation, chronic kidney disease, and acute kidney injury.

3. The method of claim 1, wherein the relaxin analogue is an RXFP1 agonist.

4. The method of claim 3, wherein the relaxin analogue is a long-acting peptidyl RXFP1 agonist.

5. The method of claim 1, wherein the vasopressin analogue is a V1a receptor agonist.

6. The method of claim 1, wherein the vasopressin analogue is terlipressin or a pharmaceutically acceptable salt thereof.

7. A method of preventing or treating hepatorenal syndrome in an individual in need thereof, comprising co-administering an effective amount of a relaxin analogue and a vasopressin analogue to the individual.

8. The method of claim 7, wherein the hepatorenal syndrome is HRS-AKI (hepatorenal syndrome type 1).

9. The method of claim 7, wherein the relaxin analogue is a RXFP1 agonist.

10. The method of claim 9, wherein the relaxin analogue is a long acting peptidyl RXFP1 agonist.

11. The method of claim 7, wherein the vasopressin analogue is a V1a receptor agonist.

12. The method of claim 7, wherein the vasopressin analogue is terlipressin or a pharmaceutically acceptable salt thereof.

13. The method of claim 6 or claim 12, wherein said terlipressin is administered intravenously at a dosage of 0.5 to 2 mg per administration.

14. The method of claim 6 or claim 12, wherein said terlipressin is administered intravenously at a dosage of 0.5 to 2 mg every 4 to 6 hours.

15. The method of claim 6 or claim 12, wherein said terlipressin is administered via an intravenous infusion.

16. The method of claim 15, wherein said terlipressin is administered at a rate of 0.5 to 2 mg per 4 to 6 hours.

17. The method of claim 7, further comprising administration of midodrine or octreotide to the individual.

18. The method of any one of claims 1-14, wherein the relaxin analogue has an EC₅₀ for activation RXFP1 in the in vitro OVCAR5 cAMP assay of less than 15 nM, less than 1 nM, less than 0.5 nM, or less than 0.1 nM.

19. The method of any one of the preceding claims, wherein the relaxin analogue is administered at a dose of from about 0.01 mg/kg to about 0.5 mg/kg.

20. The method of any one of claims 1-19, wherein the relaxin analogue and the vasopressin analogue are administered simultaneously.

21. The method of any one of claims 1-19, wherein the relaxin analogue and the vasopressin analogue are administered in a single composition.

22. The method of any one of claims 1-19, wherein the relaxin analogue and the vasopressin analogue are administered in a separate composition.

23. The method of any one of claims 1-19, wherein the vasopressin analogue and relaxin analogue are administered sequentially.

24. The method of any one of the preceding claims, wherein the combination therapy has a synergistic therapeutic effect.

25. The method of any one of the preceding claims, wherein the administration of the relaxin analogue mitigates adverse effects associated with vasopressin analogue treatment in the individual.

26. The method of any one of the preceding claims, wherein the administration of the vasopressin analogue reduces risk of hypotension in said individual associated with treatment with the relaxin analogue.

27. The method of any one of the preceding claims, wherein the administration of the vasopressin analogue or relaxin analogue increases renal pressure in said individual.

28. The method of any one of the preceding claims, wherein the relaxin analogue is administered, parenterally, intravenously, subcutaneously, rectally, transdermally, or by inhalation.

29. A method of treating renal failure in an individual in need thereof, comprising: administering a relaxin analogue to the individual, wherein the individual previously has been administered a vasopressin analogue.

30. A method of treating hepatorenal syndrome in an individual with liver cirrhosis comprising a) administering a relaxin analogue to the individual, wherein the individual previously has been administered a vasopressin analogue.

31. The method of claim 29 or claim 30, wherein the administration of the relaxin analogue mitigates adverse effects associated with vasopressin analogue treatment in the individual.

32. A method of treating renal failure in an individual in need thereof, comprising: administering a vasopressin analogue to the individual, wherein the individual previously has been administered a relaxin analogue.

33. A method of treating hepatorenal syndrome in an individual with liver cirrhosis comprising a) administering a vasopressin analogue to the individual, wherein the individual previously has been administered an effective amount of a relaxin analogue.

34. The method of claim 32 or claim 33, wherein the administration of the vasopressin analogue reduces risk of hypotension in said individual associated with treatment with the relaxin analogue.

35. The method of any one of claims 32-34, wherein the administration of the vasopressin analogue or relaxin analogue increases renal pressure in said individual.

36. The method of any one of claims 7-35, wherein said hepatorenal syndrome is HRS-AKI (hepatorenal syndrome type 1).

37. The method of any one of claims 1-36, wherein the relaxin analogue is a modified relaxin B chain peptide comprising formula (I) (SEQ ID NO: 105):
Nₜₑᵣ-Ac-X₁₀-E-G-R-E-X₁₅-V-R-X₁₈-X₁₉-I-X₂₁-X₂₂-E-G-X₂₅-
S-X₂₇-X₂₈-X₂₉-X₃₀-X₃₁-X₃₂-X₃₃-NH₂-Cₜₑᵣ,
wherein:
Nₜₑᵣ represents the N-terminal end of the peptide;
Cₜₑᵣ represents the C-terminal end of the peptide;
Ac represents acetyl group;
X₁₀ represents an amino acid selected from the group consisting of leucine, 2-amino-isobutyric acid, Nε-acetyl-lysine and α-methyl-leucine;
E represents glutamic acid;
G represents glycine;
R represents arginine;
X₁₅ represents an amino acid selected form the group consisting of lysine, arginine, homolysine, homoarginine, ornithine, glutamine, phenylalanine and leucine;
V represents valine;
X₁₈ represents an amino acid selected from the group consisting of alanine, 2-amino-isobutyric acid, leucine, Nε-acetyl-lysine and glutamine;
X₁₉ represents an amino acid selected from the group consisting of lysine, Nε-acetyl-lysine, citrulline, glutamine, alanine and 2-amino-isobutyric acid;
I represents isoleucine;
X₂₁ represents an amino acid selected from the group consisting of 2-amino-isobutyric acid and alanine;
X₂₂ represents an amino acid selected from the group consisting of 2-amino-isobutyric acid and isoleucine;
X₂₅ represents the following structure: 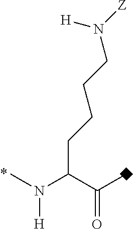
in which:
* represents a covalent bond with the glycine preceding X₂₅ in formula (I);
♦ represents a covalent bond with the serine following X₂₅ in formula (I); and
Z represents a group of formula (II):
-[(PEGₓₓ)_{b}(gE)_{c}C_{d}],
b and c independently represent 1, 2, 3, 4 or 5;
PEGₓₓ independently represents a polyethylene glycol derivative selected from the group consisting of PEG₂, PEG₂DGA, and TTDS;
gE represents γ-glutamic acid; and
C_{d} represents a linear saturated C12-C22 acyl group;
S represents serine;
X₂₇ represents an amino acid selected from the group consisting of threonine, lysine, arginine and glutamine;
X₂₈ represents an amino acid selected from the group consisting of tryptophan, phenylalanine, 5-fluoro-tryptophan, 5-chloro-tryptophan, 5-methoxy-tryptophan, tyrosine, 4-fluoro-phenylalanine, 1-naphtylalanine, 2-naphtylalanine, α-methyl-tryptophan, α-methyl-phenylalanine and 5-hydroxy-tryptophan;
X₂₉ represents an amino acid selected from the group consisting of serine, D-serine,
2-amino-isobutyric acid, threonine, α-methyl-serine, Nε-acetyl-lysine and valine;
X₃₀ represents an amino acid selected from the group consisting of 2-amino-isobutyric acid, α-methyl-lysine, D-lysine, lysine, homolysine, ornithine, arginine and α-methyl-arginine;
X₃₁ represents an amino acid selected from the group consisting of arginine, Nω-methyl-arginine, alanine, Nω,Nω′-dimethyl-arginine and citrulline;
X₃₂ represents an amino acid selected from the group consisting of lysine, alanine, arginine, Nε-acetyl-lysine and Nε,Nε,Nε-tri-methyl-lysine; and
X₃₃ represents an amino acid selected from lysine, Nε-acetyl-lysine, leucine, arginine and alanine;
or a salt or solvate thereof.

38. The method of claim 37, wherein b represents 2, 3, 4 or 5 and c represents 2, 3 or 4; or a salt or solvate thereof.

39. The method of claim 37 or 38, wherein C_{d} represents a linear saturated C12-C22 acyl group, for example a linear saturated acyl group selected from the group consisting of C₁₂ (Lau), C₁₄ (Myr), C₁₅ (Penta), C₁₆ (Palm), C₁₇ (Hepta), C₁₈ (Stea), C₂₀ (Eico) and C₂₂ (Doco) acyl group. In one embodiment C_{d} represents a linear saturated acyl group selected from the group consisting of C₁₂ (Lau), C₁₄ (Myr), C₁₅ (Penta), C₁₆ (Palm), C₁₇ (Hepta) or C₁₈ (Stea) acyl group, for example a linear saturated C₁₄, C₁₆ or C₁₈ acyl group, or for example a linear C₁₆ or C₁₈ acyl group; or a salt or solvate thereof.

40. The method of claim 39, wherein C_{d} represents a linear saturated acyl group selected from the group consisting of: C₁₂ (Lau), C₁₄ (Myr), C₁₅ (Penta), C₁₆ (Palm), C₁₇ (Hepta) and C₁₈ (Stea) acyl group;
or a salt or solvate thereof.

41. The method of claim 40, wherein C_{d} represents a linear C₁₆ or C₁₈ acyl group;
or a salt or solvate thereof.

42. The method of any one of claims 37-41, wherein the relaxin analogue comprises formula (Ib):
Nₜₑᵣ-Ac-X₁₀-E-G-R-E-X₁₅-V-R-X₁₈-X₁₉-I-X₂₁-X-₂₂-E-G-
X₂₅-S-X₂₇-X₂₈-X₂₉-X₃₀-R-X₃₂-X₃₃-NH₂-Cₜₑᵣ,
wherein:
Nₜₑᵣ represents the N-terminal end of the peptide;
Cₜₑᵣ represents the C-terminal end of the peptide;
Ac represents acetyl group;
X₁₀ represents an amino acid selected form the group consisting of leucine, Nε-acetyl-lysine and 2-amino-isobutyric acid;
E represents glutamic acid;
G represents glycine;
R represents arginine;
X₁₅ represents an amino acid selected form the group consisting of lysine, arginine, homolysine, glutamine, phenylalanine and leucine;
V represents valine;
X₁₈ represents an amino acid selected from the group consisting of alanine, 2-amino-isobutyric acid and Nε-acetyl-lysine;
X₁₉ represents an amino acid selected from the group consisting of lysine, Nε-acetyl-lysine, glutamine and citrulline; I represents isoleucine;
X₂₁ represents an amino acid selected from the group consisting of 2-amino-isobutyric acid and alanine;
X₂₂ represents an amino acid selected from the group consisting of 2-amino-isobutyric acid and isoleucine;
X₂₅ represents the following structure: 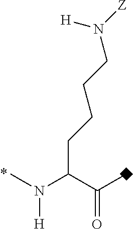
in which:
* represents a covalent bond with the glycine preceding X₂₅ in formula (Ia);
♦ represents a covalent bond with the serine following X₂₅ in formula (Ia); and
Z is selected from the group consisting of: -(TTDS)₂-(gE)₃-Palm, -(TTDS)₃-(gE)₃-Palm, -(PEG₂DGA)₃-(gE)₃-Palm, -(PEG₂)₄-(gE)₃-Palm, (TTDS)₂-(gE)₂-Palm, -(TTDS)₂-(gE)₃-Stea, -(TTDS)₃-(gE)₃-Stea, -(PEG₂DGA)₃-(gE)₃-Stea, -(PEG₂DGA)₃-(gE)₄-Stea, -(PEG₂)₃-(gE)₃-Palm, -(PEG₂)₄-(gE)₃-Stea, -(PEG₂)₅-(gE)₃-Palm, -(PEG₂)₅-(gE)₄-Palm, -(TTDS)₃-(gE)₄-Stea, -(TTDS)₂-(gE)₄-Palm, -(TTDS)₃-(gE)₂-Stea, -(TTDS)₂-(gE)₄-Stea, -(TTDS)₄-(gE)₃-Stea, -(TTDS)₃-(gE)₄-Palm, -(TTDS)₄-(gE)₃-Palm, -(TTDS)₃-(gE)₃-Myr and -(TTDS)₃-(gE)₄-Myr, wherein gE represents γ-glutamic acid, Palm represents Palmitoyl, and Stea represents Stearoyl;
S represents serine;
X₂₇ represents an amino acid selected from the group consisting of threonine, glutamine, arginine and lysine;
X₂₈ represents an amino acid selected from the group consisting of tryptophan, phenylalanine, 5-Chlorotryptophan, a,-Methyl-phenylalanine, 4-Fluoro-phenylalanine and 5-Fluorotryptophan;
X₂₉ represents an amino acid selected from the group consisting of serine, D-serine, 2-amino-isobutyric acid, Nε-acetyl-lysine, threonine and valine;
X₃₀ represents an amino acid selected from the group consisting of 2-amino-isobutyric acid, α-methyl-lysine, D-lysine, lysine, homolysine and arginine;
X₃₂ represents an amino acid selected from the group consisting of lysine, alanine, arginine and Nε-acetyl-lysine; and
X₃₃ represents an amino acid selected from lysine, Nε-acetyl-lysine and arginine;
or a salt or solvate thereof.

43. The method of any one of claims 37-42, wherein the relaxin analogue comprises an amino acid sequence selected from the group consisting of: SEQ ID NO: 1 to 97.

44. The method of any one of claims 37-43, wherein Z is selected from the group consisting of: -(TTDS)₂-(gE)₃-Palm, -(TTDS)₃-(gE)₃-Palm, (PEG₂DGA)₃-(gE)₃-Palm, -(PEG₂)₄-(gE)₃-Palm, -(TTDS)₂-(gE)₂-Palm, (TTDS)₂-(gE)₃-Stea, -(TTDS)₃-(gE)₃-Stea, -(PEG₂DGA)₃-(gE)₃-Stea, (PEG₂)₃-(gE)₃-Palm, -(PEG₂)₄-(gE)₃-Stea, (PEG₂)₅-(gE)₃-Palm, -(TTDS)₃-(gE)₄-Stea, (TTDS)₂-(gE)₄-Palm, -(TTDS)₂-(gE)₄-Stea, (TTDS)₄-(gE)₃-Stea, -(TTDS)₃-(gE)₄-Palm, -(TTDS)₄-(gE)₃-Palm, (TTDS)₃-(gE)₃-Myr and -(TTDS)₃-(gE)₄-Myr.

45. The method of any one of claims 37-44, wherein the relaxin analogue comprises an amino acid sequence selected from the group consisting of SEQ ID NO: 3, 6, 7, 9-12, 20-22, 26, 28, 30-34, 45, 47-49, 51, 54-62, 64, 67-69, 71-86, 91, 93 and 96.

46. The method of any one of claims 37-44, wherein the relaxin analogue comprises an amino acid sequence selected from the group consisting of SEQ ID NO: 3, 6, 7, 20, 26, 30-34, 45, 48, 49, 51, 54-61, 67, 71, 73, 75-79, 81, 83-92 and 97.

47. The method of any one of claims 37-44, wherein the relaxin analogue comprises an amino acid sequence selected from the group consisting of SEQ ID NO: 3, SEQ ID NO: 6, SEQ ID NO: 7, AND SEQ ID NO: 20.

48. The method of any one of claims 37-44, wherein the relaxin analogue comprises the amino acid sequence of SEQ ID NO: 3.

49. A pharmaceutical composition comprising separately or together a relaxin analogue, a vasopressin analogue, and one or more pharmaceutically acceptable excipients.

50. The composition of claim 49, wherein the vasopressin analogue is a V1a agonist.

51. The composition of claim 49, wherein the vasopressin analogue is terlipressin.

52. The composition of any one of claims 49-51, wherein the relaxin analogue is an RXFP1 peptidyl agonist.

53. The composition of any one of claims 49-52, wherein the relaxin analogue is a modified relaxin B chain peptide comprising formula (I) (SEQ ID NO: 105):
Nₜₑᵣ-Ac-X₁₀-E-G-R-E-X₁₅-V-R-X₁₈-X₁₉-I-X₂₁-X₂₂-E-G-X₂₅-
S-X₂₇-X₂₈-X₂₉-X₃₀-X₃₁-X₃₂-X₃₃-NH₂-Cₜₑᵣ,
wherein:
Nₜₑᵣ represents the N-terminal end of the peptide;
Cₜₑᵣ represents the C-terminal end of the peptide;
Ac represents acetyl group;
X₁₀ represents an amino acid selected from the group consisting of leucine, 2-amino-isobutyric acid, Nε-acetyl-lysine and α-methyl-leucine;
E represents glutamic acid;
G represents glycine;
R represents arginine;
X₁₅ represents an amino acid selected form the group consisting of lysine, arginine, homolysine, homoarginine, ornithine, glutamine, phenylalanine and leucine;
V represents valine;
X₁₈ represents an amino acid selected from the group consisting of alanine, 2-amino-isobutyric acid, leucine, Nε-acetyl-lysine and glutamine;
X₁₉ represents an amino acid selected from the group consisting of lysine, Nε-acetyl-lysine, citrulline, glutamine, alanine and 2-amino-isobutyric acid;
I represents isoleucine;
X₂₁ represents an amino acid selected from the group consisting of 2-amino-isobutyric acid and alanine;
X₂₂ represents an amino acid selected from the group consisting of 2-amino-isobutyric acid and isoleucine;
X₂₅ represents the following structure: 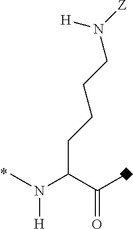
in which:
* represents a covalent bond with the glycine preceding X₂₅ in formula (I);
♦ represents a covalent bond with the serine following X₂₅ in formula (I); and
Z represents a group of formula (II):
-[(PEGₓₓ)_{b}(gE)_{c}C_{d}],
b and c independently represent 1, 2, 3, 4 or 5;
PEGₓₓ independently represents a polyethylene glycol derivative selected from the group consisting of PEG₂, PEG₂DGA, and TTDS;
gE represents γ-glutamic acid; and
C_{d} represents a linear saturated C12-C22 acyl group;
S represents serine;
X₂₇ represents an amino acid selected from the group consisting of threonine, lysine, arginine and glutamine;
X₂₈ represents an amino acid selected from the group consisting of tryptophan, phenylalanine, 5-fluoro-tryptophan, 5-chloro-tryptophan, 5-methoxy-tryptophan, tyrosine, 4-fluoro-phenylalanine, 1-naphtylalanine, 2-naphtylalanine, α-methyl-tryptophan, α-methyl-phenylalanine and 5-hydroxy-tryptophan;
X₂₉ represents an amino acid selected from the group consisting of serine, D-serine,
2-amino-isobutyric acid, threonine, α-methyl-serine, Nε-acetyl-lysine and valine;
X₃₀ represents an amino acid selected from the group consisting of 2-amino-isobutyric acid, α-methyl-lysine, D-lysine, lysine, homolysine, ornithine, arginine and α-methyl-arginine;
X₃₁ represents an amino acid selected from the group consisting of arginine, Nω-methyl-arginine, alanine, Nω,Nω′-dimethyl-arginine and citrulline;
X₃₂ represents an amino acid selected from the group consisting of lysine, alanine, arginine, Nε-acetyl-lysine and Nε,Nε,Nε-tri-methyl-lysine; and
X₃₃ represents an amino acid selected from lysine, Nε-acetyl-lysine, leucine, arginine and alanine;
or a salt or solvate thereof.

54. The composition of claim 53, wherein b represents 2, 3, 4 or 5 and c represents 2, 3 or 4; or a salt or solvate thereof.

55. The composition of claim 53 or 54, wherein C_{d} represents a linear saturated C₁₂-C₂₂ acyl group, for example a linear saturated acyl group selected from the group consisting of C₁₂ (Lau), C₁₄ (Myr), C₁₅ (Penta), C₁₆ (Palm), C₁₇ (Hepta), C₁₈ (Stea), C₂₀ (Eico) and C₂₂ (Doco) acyl group. In one embodiment C_{d} represents a linear saturated acyl group selected from the group consisting of C₁₂ (Lau), C₁₄ (Myr), C₁₅ (Penta), C₁₆ (Palm), C₁₇ (Hepta) or C₁₈ (Stea) acyl group, for example a linear saturated C₁₄, C₁₆ or C₁₈ acyl group, or for example a linear C₁₆ or C₁₈ acyl group; or a salt or solvate thereof.

56. The composition of claim 55, wherein C_{d} represents a linear saturated acyl group selected from the group consisting of: C₁₂ (Lau), C₁₄ (Myr), C₁₅ (Penta), C₁₆ (Palm), C₁₇ (Hepta) and C₁₈ (Stea) acyl group;
or a salt or solvate thereof.

57. The composition of claim 56, wherein C_{d} represents a linear C₁₆ or C₁₈ acyl group;
or a salt or solvate thereof.

58. The composition of any one of claims 53-57, wherein the relaxin analogue comprises formula (Ib):
Nₜₑᵣ-Ac-X₁₀-E-G-R-E-X₁₅-V-R-X₁₈-X₁₉-I-X₂₁-X-₂₂-E-G-X₂₅-
S-X₂₇-X₂₈-X₂₉-X₃₀-R-X₃₂-X₃₃-NH₂-Cₜₑᵣ,
wherein:
Nₜₑᵣ represents the N-terminal end of the peptide;
Cₜₑᵣ represents the C-terminal end of the peptide;
Ac represents acetyl group;
X₁₀ represents an amino acid selected form the group consisting of leucine, Nε-acetyl-lysine and 2-amino-isobutyric acid;
E represents glutamic acid;
G represents glycine;
R represents arginine;
X₁₅ represents an amino acid selected form the group consisting of lysine, arginine, homolysine, glutamine, phenylalanine and leucine;
V represents valine;
X₁₅ represents an amino acid selected from the group consisting of alanine, 2-amino-isobutyric acid and Nε-acetyl-lysine;
X₁₉ represents an amino acid selected from the group consisting of lysine, Nε-acetyl-lysine, glutamine and citrulline; I represents isoleucine;
X₂₁ represents an amino acid selected from the group consisting of 2-amino-isobutyric acid and alanine;
X₂₂ represents an amino acid selected from the group consisting of 2-amino-isobutyric acid and isoleucine;
X₂₅ represents the following structure: 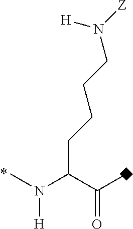
in which:
* represents a covalent bond with the glycine preceding X₂₅ in formula (Ia);
♦ represents a covalent bond with the serine following X₂₅ in formula (Ia); and Z is selected from the group consisting of a -(TTDS)₂-(gE)₃-Palm, -(TTDS)₃-(gE)₃-Palm, -(PEG₂DGA)₃-(gE)₃-Palm, -(PEG₂)₄-(gE)₃-Palm, -(TTDS)₂-(gE)₂-Palm, -(TTDS)₂-(gE)₃-Stea, -(TTDS)₃-(gE)₃-Stea, -(PEG₂DGA)₃-(gE)₃-Stea, -(PEG₂DGA)₃-(gE)₄-Stea, -(PEG₂)₃-(gE)₃-Palm, -(PEG₂)₄-(gE)₃-Stea, -(PEG₂)₅-(gE)₃-Palm, -(PEG₂)₅-(gE)₄-Palm, -(TTDS)₃-(gE)₄-Stea, -(TTDS)₂-(gE)₄-Palm, -(TTDS)₃-(gE)₂-Stea, -(TTDS)₂-(gE)₄-Stea, -(TTDS)₄-(gE)₃-Stea, -(TTDS)₃-(gE)₄-Palm, -(TTDS)₄-(gE)₃-Palm, -(TTDS)₃-(gE)₃-Myr and -(TTDS)₃-(gE)₄-Myr, wherein gE represents γ-glutamic acid, Palm represents Palmitoyl, and Stea represents Stearoyl;
S represents serine;
X₂₇ represents an amino acid selected from the group consisting of threonine, glutamine, arginine and lysine;
X₂₈ represents an amino acid selected from the group consisting of tryptophan, phenylalanine, 5-Chlorotryptophan, α-Methyl-phenylalanine, 4-Fluoro-phenylalanine and 5-Fluorotryptophan;
X₂₉ represents an amino acid selected from the group consisting of serine, D-serine, 2-amino-isobutyric acid, Nε-acetyl-lysine, threonine and valine;
X₃₀ represents an amino acid selected from the group consisting of 2-amino-isobutyric acid, α-methyl-lysine, D-lysine, lysine, homolysine and arginine;
X₃₂ represents an amino acid selected from the group consisting of lysine, alanine, arginine and Nε-acetyl-lysine; and
X₃₃ represents an amino acid selected from lysine, Nε-acetyl-lysine and arginine;
or a salt or solvate thereof.

59. The composition of any one of claims 53-58, wherein the relaxin analogue comprises an amino acid sequence selected from the group consisting of: SEQ ID NO: 1 to 97.

60. The composition of any one of claims 53-59, wherein Z is selected from the group consisting of: -(TTDS)₂-(gE)₃-Palm, -(TTDS)₃-(gE)₃-Palm, (PEG₂DGA)₃-(gE)₃-Palm, -(PEG₂)₄-(gE)₃-Palm, -(TTDS)₂-(gE)₂-Palm, (TTDS)₂-(gE)₃-Stea, -(TTDS)₃-(gE)₃-Stea, -(PEG₂DGA)₃-(gE)₃-Stea, (PEG₂)₃-(gE)₃-Palm, -(PEG₂)₄-(gE)₃-Stea, (PEG₂)₅-(gE)₃-Palm, -(TTDS)₃-(gE)₄-Stea, (TTDS)₂-(gE)₄-Palm, -(TTDS)₂-(gE)₄-Stea, (TTDS)₄-(gE)₃-Stea, -(TTDS)₃-(gE)₄-Palm, -(TTDS)₄-(gE)₃-Palm, (TTDS)₃-(gE)₃-Myr and -(TTDS)₃-(gE)₄-Myr.

61. The composition of any one of claims 53-60, wherein the relaxin analogue comprises an amino aced sequence selected from the group consisting of SEQ ID NO: 3, 6, 7, 9-12, 20-22, 26, 28, 30-34, 45, 47-49, 51, 54-62, 64, 67-69, 71-86, 91, 93 and 96.

62. The composition of any one of claims 53-60, wherein the relaxin analogue comprises an amino acid sequence selected from the group consisting of SEQ ID NO: 3, 6, 7, 20, 26, 30-34, 45, 48, 49, 51, 54-61, 67, 71, 73, 75-79, 81, 83-92 and 97.

63. The composition of any one of claims 53-60, wherein the relaxin analogue comprises an amino acid sequence selected from the group consisting of SEQ ID NO: 3, SEQ ID NO: 6, SEQ ID NO: 7, AND SEQ ID NO: 20.

64. The composition of any one of claims 53-60, wherein the relaxin analogue comprises the amino acid sequence of SEQ ID NO: 3.

65. A kit comprising a relaxin analogue in a pharmaceutically acceptable composition and a vasopressin analogue in a pharmaceutically acceptable composition.

66. The kit of claim 65, wherein the vasopressin analogue is a V1a agonist.

67. The kit of claim 65, wherein the vasopressin analogue is terlipressin.

68. The kit of claim 65, wherein the relaxin analogue is an RXFP1 peptidyl agonist.

69. The kit of any one of claims 65-68, wherein the relaxin analogue is a modified relaxin B chain peptide comprising formula (I) (SEQ ID NO: 105):
Nₜₑᵣ-Ac-X₁₀-E-G-R-E-X₁₅-V-R-X₁₈-X₁₉-I-X₂₁-X₂₂-E-G-X₂₅-
S-X₂₇-X₂₈-X₂₉-X₃₀-X₃₁-X₃₂-X₃₃-NH₂-Cₜₑᵣ,
wherein:
Nₜₑᵣ represents the N-terminal end of the peptide;
Cₜₑᵣ represents the C-terminal end of the peptide;
Ac represents acetyl group;
X₁₀ represents an amino acid selected from the group consisting of leucine, 2-amino-isobutyric acid, Nε-acetyl-lysine and α-methyl-leucine;
E represents glutamic acid;
G represents glycine;
R represents arginine;
X₁₅ represents an amino acid selected form the group consisting of lysine, arginine, homolysine, homoarginine, ornithine, glutamine, phenylalanine and leucine;
V represents valine;
X₁₈ represents an amino acid selected from the group consisting of alanine, 2-amino-isobutyric acid, leucine, Nε-acetyl-lysine and glutamine;
X₁₉ represents an amino acid selected from the group consisting of lysine, Nε-acetyl-lysine, citrulline, glutamine, alanine and 2-amino-isobutyric acid;
I represents isoleucine;
X₂₁ represents an amino acid selected from the group consisting of 2-amino-isobutyric acid and alanine;
X₂₂ represents an amino acid selected from the group consisting of 2-amino-isobutyric acid and isoleucine;
X₂₅ represents the following structure: 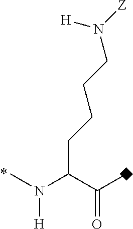
in which:
* represents a covalent bond with the glycine preceding X₂₅ in formula (I);
♦ represents a covalent bond with the serine following X₂₅ in formula (I); and
Z represents a group of formula (II):
-[(PEGₓₓ)_{b}(gE)_{c}C_{d}],
b and c independently represent 1, 2, 3, 4 or 5;
PEGₓₓ independently represents a polyethylene glycol derivative selected from the group consisting of PEG₂, PEG₂DGA, and TTDS;
gE represents γ-glutamic acid; and
C_{d} represents a linear saturated C₁₂-C₂₂ acyl group;
S represents serine;
X₂₇ represents an amino acid selected from the group consisting of threonine, lysine, arginine and glutamine;
X₂₈ represents an amino acid selected from the group consisting of tryptophan, phenylalanine, 5-fluoro-tryptophan, 5-chloro-tryptophan, 5-methoxy-tryptophan, tyrosine, 4-fluoro-phenylalanine, 1-naphtylalanine, 2-naphtylalanine, α-methyl-tryptophan, α-methyl-phenylalanine and 5-hydroxy-tryptophan;
X₂₉ represents an amino acid selected from the group consisting of serine, D-serine,
2-amino-isobutyric acid, threonine, α-methyl-serine, Nε-acetyl-lysine and valine;
X₃₀ represents an amino acid selected from the group consisting of 2-amino-isobutyric acid, α-methyl-lysine, D-lysine, lysine, homolysine, ornithine, arginine and α-methyl-arginine;
X₃₁ represents an amino acid selected from the group consisting of arginine, Nω-methyl-arginine, alanine, Nω,Nω′-dimethyl-arginine and citrulline;
X₃₂ represents an amino acid selected from the group consisting of lysine, alanine, arginine, Nε-acetyl-lysine and Nε,Nε,Nε-tri-methyl-lysine; and
X₃₃ represents an amino acid selected from lysine, Nε-acetyl-lysine, leucine, arginine and alanine;
or a salt or solvate thereof.

70. The kit of claim 69, wherein b represents 2, 3, 4 or 5 and c represents 2, 3 or 4; or a salt or solvate thereof.

71. The kit of claim 69 or 70, wherein C_{d} represents a linear saturated C₁₂-C₂₂ acyl group, for example a linear saturated acyl group selected from the group consisting of C₁₂ (Lau), C₁₄ (Myr), C₁₅ (Penta), C₁₆ (Palm), C₁₇ (Hepta), C₁₈ (Stea), C₂₀ (Eico) and C₂₂ (Doco) acyl group. In one embodiment C_{d} represents a linear saturated acyl group selected from the group consisting of C₁₂ (Lau), C₁₄ (Myr), C₁₅ (Penta), C₁₆ (Palm), C₁₇ (Hepta) or C₁₈ (Stea) acyl group, for example a linear saturated C₁₄, C₁₆ or C₁₈ acyl group, or for example a linear C₁₆ or C₁₈ acyl group; or a salt or solvate thereof.

72. The kit of claim 71, wherein C_{d} represents a linear saturated acyl group selected from the group consisting of: C₁₂ (Lau), C₁₄ (Myr), C₁₅ (Penta), C₁₆ (Palm), C₁₇ (Hepta) and C₁₈ (Stea) acyl group;
or a salt or solvate thereof.

73. The kit of claim 72, wherein C_{d} represents a linear C₁₆ or C₁₈ acyl group;
or a salt or solvate thereof.

74. The kit of any one of claims 69-73, wherein the relaxin analogue comprises formula (Ib):
Nₜₑᵣ-Ac-X₁₀-E-G-R-E-X₁₅-V-R-X₁₈-X₁₉-I-X₂₁-X-₂₂-E-G-X₂₅-
S-X₂₇-X₂₈-X₂₉-X₃₀-R-X₃₂-X₃₃-NH₂-Cₜₑᵣ,
wherein:
Nₜₑᵣ represents the N-terminal end of the peptide;
Cₜₑᵣ represents the C-terminal end of the peptide;
Ac represents acetyl group;
X₁₀ represents an amino acid selected form the group consisting of leucine, Nε-acetyl-lysine and 2-amino-isobutyric acid;
E represents glutamic acid;
G represents glycine;
R represents arginine;
X₁₅ represents an amino acid selected form the group consisting of lysine, arginine, homolysine, glutamine, phenylalanine and leucine;
V represents valine;
X₁₈ represents an amino acid selected from the group consisting of alanine, 2-amino-isobutyric acid and Nε-acetyl-lysine;
X₁₉ represents an amino acid selected from the group consisting of lysine, Nε-acetyl-lysine, glutamine and citrulline; I represents isoleucine;
X₂₁ represents an amino acid selected from the group consisting of 2-amino-isobutyric acid and alanine;
X₂₂ represents an amino acid selected from the group consisting of 2-amino-isobutyric acid and isoleucine;
X₂₅ represents the following structure: 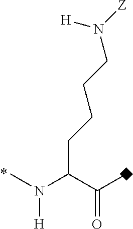
in which:
* represents a covalent bond with the glycine preceding X₂₅ in formula (Ia);
♦ represents a covalent bond with the serine following X₂₅ in formula (Ia); and
Z is selected from the group consisting of a -(TTDS)₂-(gE)₃-Palm, -(TTDS)₃-(gE)₃-Palm, -(PEG₂DGA)₃-(gE)₃-Palm, -(PEG₂)₄-(gE)₃-Palm, (TTDS)₂-(gE)₂-Palm, -(TTDS)₂-(gE)₃-Stea, -(TTDS)₃-(gE)₃-Stea, -(PEG₂DGA)₃-(gE)₃-Stea, -(PEG₂DGA)₃-(gE)₄-Stea, -(PEG₂)₃-(gE)₃-Palm, -(PEG₂)₄-(gE)₃-Stea, -(PEG₂)₅-(gE)₃-Palm, -(PEG₂)₅-(gE)₄-Palm, -(TTDS)₃-(gE)₄-Stea, -(TTDS)₂-(gE)₄-Palm, -(TTDS)₃-(gE)₂-Stea, -(TTDS)₂-(gE)₄-Stea, -(TTDS)₄-(gE)₃-Stea, -(TTDS)₃-(gE)₄-Palm, -(TTDS)₄-(gE)₃-Palm, -(TTDS)₃-(gE)₃-Myr and -(TTDS)₃-(gE)₄-Myr, wherein gE represents γ-glutamic acid, Palm represents Palmitoyl, and Stea represents Stearoyl;
S represents serine;
X₂₇ represents an amino acid selected from the group consisting of threonine, glutamine, arginine and lysine;
X₂₈ represents an amino acid selected from the group consisting of tryptophan, phenylalanine, 5-Chlorotryptophan, α-Methyl-phenylalanine, 4-Fluoro-phenylalanine and 5-Fluorotryptophan;
X₂₉ represents an amino acid selected from the group consisting of serine, D-serine, 2-amino-isobutyric acid, Nε-acetyl-lysine, threonine and valine;
X₃₀ represents an amino acid selected from the group consisting of 2-amino-isobutyric acid, α-methyl-lysine, D-lysine, lysine, homolysine and arginine;
X₃₂ represents an amino acid selected from the group consisting of lysine, alanine, arginine and Nε-acetyl-lysine; and
X₃₃ represents an amino acid selected from lysine, Nε-acetyl-lysine and arginine;
or a salt or solvate thereof.

75. The kit of any one of claims 69-74, wherein the relaxin analogue comprises an amino acid sequence selected from the group consisting of: SEQ ID NO: 1 to 97.

76. The kit of any one of claims 69-75, wherein Z is selected from the group consisting of: -(TTDS)₂-(gE)₃-Palm, -(TTDS)₃-(gE)₃-Palm, (PEG₂DGA)₃-(gE)₃-Palm, -(PEG₂)₄-(gE)₃-Palm, -(TTDS)₂-(gE)₂-Palm, (TTDS)₂-(gE)₃-Stea, -(TTDS)₃-(gE)₃-Stea, -(PEG₂DGA)₃-(gE)₃-Stea, (PEG₂)₃-(gE)₃-Palm, -(PEG₂)₄-(gE)₃-Stea, (PEG₂)₅-(gE)₃-Palm, -(TTDS)₃-(gE)₄-Stea, (TTDS)₂-(gE)₄-Palm, -(TTDS)₂-(gE)₄-Stea, (TTDS)₄-(gE)₃-Stea, -(TTDS)₃-(gE)₄-Palm, -(TTDS)₄-(gE)₃-Palm, (TTDS)₃-(gE)₃-Myr and -(TTDS)₃-(gE)₄-Myr.

77. The kit of any one of claims 69-76, wherein the relaxin analogue comprises an amino aced sequence selected from the group consisting of SEQ ID NO: 3, 6, 7, 9-12, 20-22, 26, 28, 30-34, 45, 47-49, 51, 54-62, 64, 67-69, 71-86, 91, 93 and 96.

78. The kit of any one of claims 69-76, wherein the relaxin analogue comprises an amino acid sequence selected from the group consisting of SEQ ID NO: 3, 6, 7, 20, 26, 30-34, 45, 48, 49, 51, 54-61, 67, 71, 73, 75-79, 81, 83-92 and 97.

79. The kit of any one of claims 69-76, wherein the relaxin analogue comprises an amino acid sequence selected from the group consisting of SEQ ID NO: 3, SEQ ID NO: 6, SEQ ID NO: 7, AND SEQ ID NO: 20.

80. The kit of any one of claims 69-76, wherein the relaxin analogue comprises the amino acid sequence of SEQ ID NO: 3.

81. A method of preventing or treating renal failure in an individual in need thereof, comprising co-administering a dose of a relaxin analogue from about 1.0 mg to about 10.0 mg and an effective amount of a vasopressin analogue to the individual.

82. The method of claim 81, wherein the renal failure is selected from the group consisting of: renal dysfunction induced by liver cirrhosis, renal dysfunction induced by liver transplantation, chronic kidney disease, and acute kidney injury.

83. A method of preventing or treating hepatorenal syndrome in an individual in need thereof, comprising co-administering a dose of a relaxin analogue from about 1.0 mg to about 10.0 mg and an effective amount of a vasopressin analogue to the individual.

84. The method of claim 83, wherein the hepatorenal syndrome is HRS-AKI (hepatorenal syndrome type 1).

85. The method of any one of claims 81-84, wherein the relaxin analogue is an RXFP1 agonist.

86. The method of claim 85, wherein the relaxin analogue is a long-acting peptidyl RXFP1 agonist.

87. The method of any one of claims 81-86, wherein from about 1.0 mg to about 3.0 mg of the relaxin analogue is administered to the individual.

88. The method of any one of claims 81-87, wherein about 1.0 mg of the relaxin analogue is administered to the individual.

89. The method of any one of claims 81-87, wherein about 2.0 mg of the relaxin analogue is administered to the individual.

90. The method of any one of claims 81-86, wherein from about 3.0 mg to about 5.0 mg of the relaxin analogue is administered to the individual.

91. The method of any one of claims 81-86 and 90, wherein about 4.0 mg of the relaxin analogue is administered to the individual.

92. The method of any one of claims 81-86, wherein from about 5.0 mg to about 10.0 mg of the relaxin analogue is administered to the individual.

93. The method of any one of claims 81-86 and 92, wherein about 5.0 mg of the relaxin analogue is administered to the individual.

94. The method of any one of claims 81-86 and 92, wherein about 10.0 mg of the relaxin analogue is administered to the individual.

95. The method of any one of claims 81-94, wherein the relaxin analogue is administered intravenously.

96. The method of claim 95, wherein the relaxin analogue is administered intravenously over from about 1 hour to about 10 hours.

97. The method of claim 95 or 96, wherein the relaxin analogue is administered intravenously over from about 2 hours to about 8 hours.

98. The method of any one of claims 95-97, wherein the relaxin analogue is administered intravenously over from about 3 hours to about 6 hours.

99. The method of any one of claims 95-98, wherein the relaxin analogue is administered intravenously over about 4 hours.

100. The method of any one of claims 81-99, further comprising administering an additional dose of the relaxin analogue to the individual.

101. The method of claim 100, wherein the additional dose of the relaxin analogue is administered between 5 hours and 18 hours after administration of the about 1.0 mg to about 10.0 mg of the relaxin analogue.

102. The method of claim 100 or 101, wherein the additional dose of the relaxin analogue is administered between 8 hours and 15 hours after administration of the dose from about 1.0 mg to about 10.0 mg of the relaxin analogue.

103. The method of any one of claims 100-102, wherein the additional dose of the relaxin analogue is administered between 10 hours and 13 hours after administration of the dose from about 1.0 mg to about 10.0 mg of the relaxin analogue.

104. The method of any one of claims 100-103, wherein the additional dose of the relaxin analogue is administered about 12 hours after administration of the dose from about 1.0 mg to about 10.0 mg of the relaxin analogue.

105. The method of any one of claims 100-104, wherein the additional dose of the relaxin analogue is administered subcutaneously to the individual.

106. The method of any one of claims 100-105, wherein the additional dose of the relaxin analogue comprises from about 1 mg to about 50 mg of the relaxin analogue.

107. The method of any one of claims 100-106, wherein the additional dose of the relaxin analogue comprises from about 5 mg to about 15 mg of the relaxin analogue.

108. The method of any one of claims 100-107, wherein the additional dose of the relaxin analogue comprises from about 8 mg to about 12 mg of the relaxin analogue.

109. The method of any one of claims 100-108, wherein the additional dose of the relaxin analogue comprises about 10 mg of the relaxin analogue.

110. The method of any one of claims 81-109, further comprising administering a yet additional dose of the relaxin analogue to the individual.

111. The method of claim 110, wherein the yet additional dose of the relaxin analogue is administered between 18 hours and 30 hours after administration of the about 1.0 mg to about 10.0 mg of the relaxin analogue.

112. The method of claim 110 or 111, wherein the yet additional dose of the relaxin analogue is administered between 20 hours and 26 hours after administration of the dose from about 1.0 mg to about 10.0 mg of the relaxin analogue.

113. The method of any one of claims 110-112, wherein the yet additional dose of the relaxin analogue is administered about 24 hours after administration of the dose from about 1.0 mg to about 10.0 mg of the relaxin analogue.

114. The method of any one of claims 110-113, wherein the yet additional dose of the relaxin analogue is administered subcutaneously to the individual.

115. The method of any one of claims 110-114, wherein the yet additional dose of the relaxin analogue comprises from about 1 mg to about 50 mg of the relaxin analogue.

116. The method of any one of claims 110-115, wherein the yet additional dose of the relaxin analogue comprises from about 2 mg to about 15 mg of the relaxin analogue.

117. The method of any one of claims 110-116, wherein the yet additional dose of the relaxin analogue comprises from about 3 mg to about 8 mg of the relaxin analogue.

118. The method of any one of claims 110-117, wherein the yet additional dose of the relaxin analogue comprises about 5 mg of the relaxin analogue.

119. The method of any one of claims 110-115, wherein the yet additional dose of the relaxin analogue comprises from about 2 mg to about 5 mg of the relaxin analogue.

120. The method of any one of claims 110-115 and 119, wherein the yet additional dose of the relaxin analogue comprises about 2.5 mg of the relaxin analogue.

121. The method of any one of claims 110-115, wherein the yet additional dose of the relaxin analogue comprises from about 6 mg to about 15 mg of the relaxin analogue.

122. The method of any one of claims 110-115 and 119, wherein the yet additional dose of the relaxin analogue comprises from about 8 mg to about 12 mg of the relaxin analogue.

123. The method of any one of claims 110-115 and 119-122, wherein the yet additional dose of the relaxin analogue comprises about 10 mg of the relaxin analogue.

124. The method of any one of claims 110-123, further comprising administering daily the yet additional dose of the relaxin analogue.

125. The method of any one of claims 110-124, wherein the yet additional dose of the relaxin analogue is administered daily from about 10 days to about 20 days.

126. The method of any one of claims 110-124, wherein the yet additional dose of the relaxin analogue is administered daily from about 12 days to about 16 days.

127. The method of any one of claims 110-124, wherein the yet additional dose of the relaxin analogue is administered daily for about 14 days or more.

128. The method of any one of claims 110-124, wherein the yet additional dose of the relaxin analogue is administered daily from about 3 days to about 15 days.

129. The method of any one of claims 110-124, wherein the yet additional dose of the relaxin analogue is administered daily from about 4 days to about 13 days.

130. The method of any one of claims 110-124, wherein the yet additional dose of the relaxin analogue is administered daily from about 5 days to about 11 days.

131. The method of any one of claims 110-124, wherein the yet additional dose of the relaxin analogue is administered daily from about 6 days to about 9 days.

132. The method of any one of claims 81-131, wherein the relaxin analogue has an EC₅₀ for activation RXFP1 in the in vitro OVCAR5 cAMP assay of less than 15 nM, less than 1 nM, less than 0.5 nM, or less than 0.1 nM.

133. The method of any one of claims 81-132, wherein the vasopressin analogue is a Via receptor agonist.

134. The method of any one of claims 81-132, wherein the vasopressin analogue is terlipressin or a pharmaceutically acceptable salt thereof.

135. The method of claim 134, wherein the terlipressin is administered intravenously at a dosage from about 0.5 to about 10 mg.

136. The method of claim 134 or 135, wherein the terlipressin is administered intravenously at a dosage from about 0.5 to about 2 mg every 4 to 6 hours.

137. The method of any one of claims 134-136, wherein the terlipressin is administered intravenously at a dosage of about 1 mg every 6 hours.

138. The method of claim 134 or 135, wherein the terlipressin is administered intravenously at a dosage from about 2 to about 6 mg every 4 to 6 hours.

139. The method of any one of claims 134-135 and 138, wherein the terlipressin is administered intravenously at a dosage of about 4 mg every 6 hours.

140. The method of claim 134 or 135, wherein the terlipressin is administered intravenously at a dosage from about 6 to about 10 mg every 4 to 6 hours.

141. The method of any one of claims 134-135 and 140, wherein the terlipressin is administered intravenously at a dosage of about 6 mg every 6 hours.

142. The method of any one of claims 134-135 and 140, wherein the terlipressin is administered intravenously at a dosage of about 8 mg every 6 hours.

143. The method of any one of claims 137, 139, 141 or 142, wherein terlipressin is administered intravenously via a bolus injection.

144. The method of claim 143, wherein terlipressin is administered intravenously via a bolus injection over from about 1 minute to about 5 minutes.

145. The method of claim 143 or 144, wherein terlipressin is administered intravenously via a bolus injection over from about 2 minutes to about 3 minutes.

146. The method of any one of claims 143-145, wherein terlipressin is administered intravenously via a bolus injection over about 2 minutes.

147. The method of claim 134, wherein the terlipressin is administered intravenously at a dosage from about 1 mg to about 10 mg.

148. The method of claim 134 or 147, wherein the terlipressin is administered intravenously at a dosage from about 1.5 mg to about 4 mg.

149. The method of claim 134 or 147-148, wherein the terlipressin is administered intravenously at a dosage of about 2 mg.

150. The method of claim 134 or 147, wherein the terlipressin is administered intravenously at a dosage from about 5 mg to about 8 mg.

151. The method of claim 134, 147, or 150, wherein the terlipressin is administered intravenously at a dosage of about 6 mg.

152. The method of claim 149 or 151, wherein the terlipressin is administered intravenously over from about 10 hours to about 30 hours.

153. The method of claim 149 or 150, wherein the terlipressin is administered intravenously over from about 20 hours to about 25 hours.

154. The method of any one of claims 149-153, wherein the terlipressin is administered intravenously over about 24 hours.

155. The method of any one of claims 81-154, further comprising administering midodrine or octreotide to the individual.

156. The method of any one of claims 81-154, further comprising administering albumin to the individual.

157. The method of any one of claims 81-156, wherein the dose of the relaxin analogue and the vasopressin analogue are administered simultaneously.

158. The method of any one of claims 81-156, wherein the dose of the relaxin analogue and the vasopressin analogue are administered in a single composition.

159. The method of any one of claims 81-156, wherein the dose of the relaxin analogue and the vasopressin analogue are administered in separate compositions.

160. The method of any one of claims 81-156, wherein the dose of the vasopressin analogue and the relaxin analogue are administered sequentially.

161. The method of any one of claims 81-160, wherein the combination therapy has a synergistic therapeutic effect.

162. The method of any one of claims 81-161, wherein the combination therapy achieves an improved response rate incidence, wherein responders are defined according to at least International Club of Acites (ICA) criteria.

163. The method of claim 162, wherein responders comprise full or partial responders, as defined according to ICA criteria, and are alive without renal replacement therapy (RRT) for at least 30 days after start of treatment.

164. The method of claim 162, wherein responders comprise full or partial responders, as defined according to ICA criteria, and are alive without renal replacement therapy (RRT) for at least 10 days after start of treatment.

165. The method of claim 163 or 164, wherein full responders are defined as two serum creatinine levels returning to a value within 0.3 mg/dL (26.5 micromolar/L) of a baseline serum creatinine value at least 2 hours apart.

166. The method of claim 163 or 164, wherein partial responders are defined as a regression of at least 1 acute kidney injury (AKI) stage with a reduction of serum creatinine greater than or equal to 0.3 mg/dL above a baseline serum creatinine value.

167. The method of any one of claims 81-166, wherein the combination therapy achieves an improved response rate incidence, wherein response rate incidence is measured according to a return of serum creatinine to a value within 0.3 mg/dL (26.5 micro molar/L) of a baseline value.

168. The method of any one of claims 81-166, wherein the combination therapy achieves an improved response rate incidence, wherein response rate incidence is measured according to a regression of acute kidney injury (AKI) stage with a reduction of serum creatinine greater than or equal to 0.3 mg/dL above a baseline value.

169. The method of any one of claims 81-166, wherein the combination therapy achieves an improved response rate incidence, wherein response rate incidence is measured according to two consecutive serum creatinine values at least 2 hours apart being below 1.5 mg/dL.

170. The method of any one of claims 81-169, wherein the administration of the relaxin analogue mitigates adverse effects associated with vasopressin analogue treatment in the individual.

171. The method of any one of claims 81-169, wherein the administration of the vasopressin analogue reduces risk of hypotension in the individual associated with treatment with the relaxin analogue.

172. The method of any one of claims 81-169, wherein the administration of the vasopressin analogue increases renal pressure in the individual.

173. A method of treating renal failure in an individual in need thereof, comprising: administering a relaxin analogue to the individual, wherein the individual previously has been administered a vasopressin analogue.

174. A method of treating hepatorenal syndrome in an individual with liver cirrhosis comprising a) administering a relaxin analogue to the individual, wherein the individual previously has been administered a vasopressin analogue.

175. The method of claim 173 or claim 174, wherein the individual was previously deemed a non-responder to the vasopressin analogue.

176. The method of claim 173 or claim 174, wherein the administration of the relaxin analogue mitigates adverse effects associated with vasopressin analogue treatment in the individual.

177. A method of preventing or treating renal failure in an individual in need thereof, comprising:
A) intravenously administering to the individual about a 4.0 mg dose of a relaxin analogue;
B) on a same day that step (A) is performed, subcutaneously administering to the individual about a 5.0 mg dose of the relaxin analogue; and
C) on a subsequent day different from the day that steps (A) and (B) are performed, subcutaneously administering, to the individual, about a 10 mg dose of the relaxin analogue.

178. A method of preventing or treating hepatorenal syndrome in an individual in need thereof, comprising:
A) intravenously administering to the individual about a 4.0 mg dose of a relaxin analogue;
B) on a same day that step (A) is performed, subcutaneously administering to the individual about a 5.0 mg dose of the relaxin analogue; and
C) on a subsequent day different from the day that steps (A) and (B) are performed, subcutaneously administering, to the individual, about a 10 mg dose of the relaxin analogue.

179. The method of claim 177 or 178, further comprising repeating step (c) on a daily basis for up to 13 days.

180. The method of any one of claims 177-179, further comprising: on the same day that step (A) and step (b) are performed, intravenously administering a 1 mg bolus of terlipressin every 6 hours.

181. The method of claim 180, further comprising repeating intravenously administering a 1 mg bolus of terlipressin every 6 hours for up to 14 days.
